(19) United States
(12) Patent Application Publication
(10) Pub. No.: US 2024/0400579 A1
(43) Pub. Date: Dec. 5, 2024

(54) SPIROCYCLIC MODULATORS OF CHOLESTEROL BIOSYNTHESIS AND THEIR USE FOR PROMOTING REMYELINATION

(71) Applicants: GENENTECH, INC., SOUTH SAN FRANCISCO, CA (US); CONVELO THERAPEUTICS, INC., CLEVELAND, OH (US)

(72) Inventors: William VERNIER, South San Francisco, CA (US); Matthew VOLGRAF, South San Francisco, CA (US); Marie-Gabrielle BRAUN, South San Francisco, CA (US); Georgette Marie CASTANEDO, South San Francisco, CA (US); Paul Anthony GIBBONS, South San Francisco, CA (US); James Guy BREITENBUCHER, Valley View, OH (US); Drew ADAMS, Valley View, OH (US); Brad LANG, Valley View, OH (US)

(21) Appl. No.: 18/670,520

(22) Filed: May 21, 2024

Related U.S. Application Data
(63) Continuation of application No. PCT/US2022/080358, filed on Nov. 22, 2022.
(60) Provisional application No. 63/282,362, filed on Nov. 23, 2021.

(51) Int. Cl. C07D 495/10; A61K 31/407; A61K 31/4155; A61K 31/438; A61K 31/4439; A61K 31/506

(52) U.S. Cl. CPC C07D 495/10; A61K 31/407; A61K 31/4155; A61K 31/438; A61K 31/4439; A61K 31/506

(57) ABSTRACT

The subject matter described herein is directed to myelin-promoting compounds of Formula I and pharmaceutical salts thereof, methods of preparing the compounds, pharmaceutical compositions comprising the compounds, and methods of administering the compounds for the treatment of myelin-related disorders.

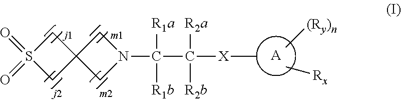

### CROSS REFERENCE TO RELATED APPLICATIONS

This application is a bypass continuation of international application no. PCT/US2022/080358 filed Nov. 22, 2022, which claims the benefit of priority to the U.S. Provisional Patent Application No. 63/282,362, filed on Nov. 23, 2021, the contents of which is incorporated by reference herein in its entirety for all purposes.

### FIELD

The subject matter described herein is directed to myelin-promoting compounds of Formula I, methods of making the compounds, their pharmaceutical compositions, and their use in the treatment of myelin-related disorders.

### BACKGROUND

Myelin-related disorders are disorders that result in abnormalities of the myelin sheath (e.g., dysmyelination, demyelination and hypomyelination) in a subject's neural cells, e.g., CNS neurons including their axons. Loss or degradation of the myelin sheath in such disorders produces a slowing or cessation of nerve cell conduction. The resulting myelin related disorders are characterized by deficits in sensation, motor function, cognition, or other physiological functions. Myelin related disorders include, but are not limited to, multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy and radiation-induced demyelination.

MS is the most common myelin-related disorder affecting several million people globally and is estimated to result in about 18,000 deaths per year. MS is a complex neurological disease characterized by deterioration of central nervous system (CNS) myelin. Myelin, composed in its majority by lipids (70% lipids, 30% protein), protects axons and makes saltatory conduction possible, which speeds axonal electric impulse. Demyelination of axons in chronic MS can result in axon degeneration and neuronal cell death. Additionally, MS destroys oligodendrocytes, the highly specialized CNS cells that generate and maintain myelin. A repair process, called remyelination, takes place in early phases of the disease, but over time, the oligodendrocytes are unable to completely rebuild and restore the myelin sheath. Repeated attacks lead to successively less effective remyelination, until a scar-like plaque is built up around the damaged axons. These scars are the origin of the symptoms.

At present, there is no cure for myelin-related disorders, and no current therapy prevents progression in MS. Accordingly, there is a need for new therapeutic approaches to the treatment of myelin-related disorders, including the promotion of remyelination. The subject matter described herein addresses this unmet need.

### BRIEF SUMMARY

In certain embodiments, the subject matter described herein is directed to a compound of Formula I or a pharmaceutically acceptable salt thereof.

In certain embodiments, the subject matter described herein is directed to a pharmaceutical composition comprising a compound of Formula I or a pharmaceutically acceptable salt thereof.

In certain embodiments, the subject matter described herein is directed to methods of treating a disorder in a subject in need thereof, wherein the disorder is a myelin-related disorder, comprising administering to the subject an effective amount of a compound of Formula I, a pharmaceutically acceptable salt thereof, or a pharmaceutical composition comprising a compound of Formula I, or a pharmaceutically acceptable salt thereof.

In certain embodiments, the subject matter described herein is directed to a compound of Formula I, or a pharmaceutically acceptable salt thereof, or a pharmaceutical composition comprising a compound of Formula I, or a pharmaceutically acceptable salt thereof, for use in treating a myelin-related disorder.

In certain embodiments, the subject matter described herein is directed to methods of promoting myelination in a subject in need thereof, comprising administering to the subject a therapeutically effective amount of a compound of Formula I, or a pharmaceutically acceptable salt thereof, or a pharmaceutical composition comprising a compound of Formula I, or a pharmaceutically acceptable salt thereof.

In certain embodiments, the subject matter described herein is directed to the use of a compound of Formula I, or a pharmaceutically acceptable salt thereof, or a pharmaceutical composition comprising a compound of Formula I, or a pharmaceutically acceptable salt thereof, in the manufacture of a medicament for treating a myelin-related disorder.

In certain embodiments, the subject matter described herein is directed to methods of preparing compounds of Formula I, or a pharmaceutically acceptable salt thereof.

Other embodiments are also described.

### DETAILED DESCRIPTION

Described herein are myelin-promoting compounds of Formula I, methods of making the compounds, their pharmaceutical compositions, and their use in the treatment of myelin-related disorders.

The enhancement and/or inducement of the accumulation of A8,9-unsaturated sterol intermediates of the cholesterol biosynthesis pathway in oligodendrocyte progenitor cells (OPCs) can induce oligodendrocyte generation. Enhancement and/or inducement of the accumulation of A8,9-unsaturated sterol intermediates can be provided by modulating and/or inhibiting enzymes within the cholesterol biosynthesis pathway in OPCs that inhibit A8,9-unsaturated sterol intermediate accumulation and/or for which the A8,9-unsaturated sterol intermediates are substrates as well as directly and/or indirectly administering A8,9-unsaturated sterol intermediates to the OPCs. Enhancement and/or inducement of the accumulation of A8,9-unsaturated sterol intermediates can promote OPC differentiation, survival, proliferation and/or maturation and treat disease and/or disorders in subjects where myelination is beneficial to the subject.

As such, in some embodiments an agent, such as a compound of Formula I, or a pharmaceutically acceptable salt thereof, that can enhance and/or induce accumulation of A8,9-unsaturated sterol intermediates of the cholesterol biosynthesis pathway in the OPCs can be administered to a subject and/or the OPCs at an amount effective to promote and/or induce OPC differentiation, proliferation and/or maturation as well as oligodendrocyte generation. In certain embodiments, the agent, for example a compound of Formula I, or a pharmaceutically acceptable salt thereof, is a compound that inhibits enzyme mediated synthesis of one or more sterol intermediates in the cholesterol biosynthesis pathway of the OPCs and/or promotes accumulation of A8,9-unsaturated sterol intermediates.

In certain embodiments, the compound of Formula I, or a pharmaceutically acceptable salt thereof, can modulate and/or inhibit one or more enzyme-mediated conversion steps of the cholesterol biosynthesis pathway, such as in the pathway from lanosterol to cholesterol, for example, between lanosterol and/or lathosterol; modulating and/or inhibiting one or more of these steps in OPCs may promote and/or induce oligodendrocyte generation. For example, the compound of Formula I can inhibit CYP51, sterol 14-reductase (TM7SF2 and/or LBR), SC4MOL, NSDHL, and/or EBP enzyme mediated synthesis of sterol intermediates in the cholesterol biosynthesis pathway. In certain embodiments, the compound of Formula I can inhibit CYP51, sterol 14-reductase and/or EBP. In certain embodiments, the compound of Formula I can inhibit EBP.

For example, in certain embodiments, the compound of Formula I used in the methods described herein can inhibit enzyme mediated conversion of zymostenol to lathosterol through the inhibition of emopamil binding protein (EBP) isomerase enzyme activity. Alternatively, in certain embodiments, the compound of Formula I used in the methods described herein can inhibit sterol C14 reductase enzyme activity or CYP51 enzyme activity in the cholesterol biosynthesis pathway.

Emopamil Binding Protein (EBP) is an enzyme responsible for one of the final steps in the production of cholesterol. Specifically, EBP converts zymostenol to lathosterol, where other enzymes then modify lathosterol to produce cholesterol. EBP is also referred to as A8-A7-sterol isomerase, 3-beta-hydroxysteroid-Delta(8),Delta(7)-isomerase, CDPX2, CHO2, CPX, or CPXD).

Without being bound by a particular theory, it is believed that compounds of Formula I or a pharmaceutically acceptable salt thereof can inhibit EBP mediated conversion of zymostenol to lathosterol in the cholesterol biosynthesis pathway of OPCs resulting in enhancement and/or inducement of the accumulation of A8,9-unsaturated sterol intermediates. In some embodiments, enhancement and/or inducement of the accumulation of A8,9-unsaturated sterol intermediates can promote OPC differentiation, survival, proliferation and/or maturation and treat disease and/or disorders in subjects where myelination or myelinization is beneficial to the subject. This mechanism of promoting myelination is distinct from the primary action of immunomodulatory agents that are often used to treat myelin-related disorders.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. Unless otherwise defined, all technical and scientific terms used herein have the same meaning as commonly understood by one of ordinary skill in this field. All publications, patent applications, patents, and other references mentioned herein are incorporated by reference in their entirety. In the event that one or more of the incorporated literature, patents, and similar materials differs from or contradicts this application, including but not limited to defined terms, term usage, described techniques, or the like, this application controls.

### I. Definitions

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through or perpendicular across the end of a line in a structure indicates a specified point of attachment of a group. Unless chemically or structurally required, no directionality or stereochemistry is indicated or implied by the order in which a chemical group is written or named.

The prefix “Cᵤ-Cᵥ” indicates that the following group has from u to v carbon atoms. For example, “C₁-C₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±50%. In certain other embodiments, the term “about” includes the indicated amount 20%. In certain other embodiments, the term “about” includes the indicated amount ±10%. In other embodiments, the term “about” includes the indicated amount ±5%. In certain other embodiments, the term “about” includes the indicated amount ±1%. In certain other embodiments, the term “about” includes the indicated amount ±0.5% and in certain other embodiments, 0.1%. Such variations are appropriate to perform the disclosed methods or employ the disclosed compositions. Also, to the term “about x” includes description of “x”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁-C₂₀ alkyl), 1 to 12 carbon atoms (i.e., C₁-C₁₂ alkyl), 1 to 8 carbon atoms (i.e., C₁-C₈ alkyl), 1 to 6 carbon atoms (i.e., C₁-C₆ alkyl), 1 to 4 carbon atoms (i.e., C₁-C₄ alkyl), or 1 to 3 carbon atoms (i.e., C₁-C₃ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl or aralkyl, the last-mentioned group contains the atom by which the moiety is attached to the rest of the molecule.

“Alkenyl” refers to an alkyl group containing at least one carbon-carbon double bond and having from 2 to 20 carbon atoms (i.e., C₂-C₂₀ alkenyl), 2 to 8 carbon atoms (i.e., C₂-C₈ alkenyl), 2 to 6 carbon atoms (i.e., C₂-C₆ alkenyl) or 2 to 4 carbon atoms (i.e., C₂-C₄ alkenyl). Examples of alkenyl groups include, e.g., ethenyl, propenyl, butadienyl (including 1,2-butadienyl and 1,3-butadienyl).

“Alkynyl” refers to an alkyl group containing at least one carbon-carbon triple bond and having from 2 to 20 carbon atoms (i.e., C₂-C₂₀ alkynyl), 2 to 8 carbon atoms (i.e., C₂-C₈ alkynyl), 2 to 6 carbon atoms (i.e., C₂-C₆ alkynyl) or 2 to 4 carbon atoms (i.e., C₂-C₄ alkynyl). The term “alkynyl” also includes those groups having one triple bond and one double bond.

“Alkoxy” refers to the group “alkyl-O—” (e.g., C₁-C₃ alkoxy or C₁-C₆ alkoxy). Examples of alkoxy groups include, e.g., methoxy, ethoxy, n-propoxy, iso-propoxy, n-butoxy, tert-butoxy, sec-butoxy, n-pentoxy, n-hexoxy and 1,2-dimethylbutoxy.

“Alkylthio” refers to the group “alkyl-S—”.

“Acyl” refers to a group —C(O)R^{y}, wherein R^{y} is hydrogen, alkyl, alkenyl, alkynyl, cycloalkyl, heterocyclyl, aryl, heteroalkyl or heteroaryl; each of which may be optionally substituted, as defined herein. Examples of acyl include, e.g., formyl, acetyl, cyclohexylcarbonyl, cyclohexylmethyl-carbonyl and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein, or R^{y} and R^{z} are taken together to form a heterocyclyl; which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl and anthryl. Aryl, however, does not encompass or overlap in any way with heteroaryl defined below. If one or more aryl groups are fused with a heteroaryl, the resulting ring system is heteroaryl regardless of the point of attachment. If one or more aryl groups are fused with a heterocyclyl, the resulting ring system is heterocyclyl regardless of the point of attachment.

“Arylalkyl” or “Aralkyl” refers to the group “aryl-alkyl-”, such as (C₆-C₁₀ aryl)-C₁-C₃ alkyl. A non-limiting example of arylalkyl is benzyl.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl or heteroaryl; each of which may be optionally substituted, as defined herein.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃-C₂₀ cycloalkyl), 3 to 12 ring carbon atoms (i.e., C₃-C₁₂cycloalkyl), 3 to 10 ring carbon atoms (i.e., C₃-C₁₀ cycloalkyl), 3 to 8 ring carbon atoms (i.e., C₃-C₈ cycloalkyl), 3 to 7 ring carbon atoms (i.e., C₃-C₇ cycloalkyl), or 3 to 6 ring carbon atoms (i.e., C₃-C₆ cycloalkyl). Monocyclic groups include, for example, cyclopropyl, cyclobutyl, cyclopentyl, cyclohexyl, cycloheptyl and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl and the like. Further, the term cycloalkyl is intended to encompass any moiety comprising a non-aromatic alkyl ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl. As used herein, “halocycloalkyl,” such as C₃-C₇ halocycloalkyl, refers to a C₃-C₇ cycloalkyl group that is substituted with one or more halogens.

“Cycloalkylalkyl” refers to the group “cycloalkyl-alkyl-”, such as (C₃-C₆ cycloalkyl)-C₁-C₃ alkyl.

“Guanidino” refers to —NR^{y}C(═NR^{z})(NR^{y}R^{z}), wherein each R^{y} and R^{z} are independently hydrogen, alkyl, alkenyl, alkynyl, cycloalkyl, heterocyclyl, aryl, heteroalkyl or heteroaryl; each of which may be optionally substituted, as defined herein.

“Hydrazino” refers to —NHNH₂.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

“Halogen” or “halo” refers to atoms occupying group VIIA of the periodic table, such as fluoro (fluorine), chloro (chlorine), bromo (bromine) or iodo (iodine).

“Haloalkyl” refers to an unbranched or branched alkyl group as defined above, wherein one or more (e.g., 1 to 6, or 1 to 3) hydrogen atoms are replaced by a halogen. For example, halo-C₁-C₃ alkyl refers to an alkyl group of 1 to 3 carbons wherein at least one hydrogen atom is replaced by a halogen. Halo-C₁-C₆ alkyl refers to an alkyl group of 1 to 6 carbons wherein at least one hydrogen atom is replaced by a halogen. Where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like.

“Haloalkoxy” refers to an alkoxy group as defined above, wherein one or more (e.g., 1 to 6, or 1 to 3) hydrogen atoms are replaced by a halogen. For example, halo-C₁-C₃ alkoxy refers to an alkoxy group of 1 to 3 carbons wherein at least one hydrogen atom is replaced by a halogen. Halo-C₁-C₆ alkoxy refers to an alkoxy group of 1 to 6 carbons wherein at least one hydrogen atom is replaced by a halogen Non-limiting examples of haloalkoxy are —OCH₂CF₃, —OCF₂H, and —OCF₃.

“Hydroxyalkyl” refers to an alkyl group as defined above, wherein one or more (e.g., 1 to 6, or 1 to 3) hydrogen atoms are replaced by a hydroxy group (e.g., hydroxy-C₁-C₃-alkyl, hydroxy-C₁-C₆-alkyl). The term “hydroxy-C₁-C₃ alkyl” refers to a one to three carbon alkyl chain where one or more hydrogens on any carbon is replaced by a hydroxy group, in particular, one hydrogen on one carbon of the chain is replaced by a hydroxy group. The term “hydroxy-C₁-C₆ alkyl” refers to a one to six carbon alkyl chain where one or more hydrogens on any carbon is replaced by a hydroxy group, in particular, one hydrogen on one carbon of the chain is replaced by a hydroxy group. Non-limiting examples of hydroxyalkyl include —CH₂OH, —CH₂CH₂OH, and —C(CH₃)₂CH₂OH.

“Heteroalkyl” refers to an alkyl group in which one or more of the carbon atoms (and any associated hydrogen atoms) are each independently replaced with the same or different heteroatomic group, provided the point of attachment to the remainder of the molecule is through a carbon atom. In certain embodiments, the heteroalkyl can have 1 to 3 carbon atoms (e.g., C₁-C₃ heteroalkyl) or 1 to 6 carbon atoms (e.g., C₁-C₆ heteroalkyl), and one or more (e.g., 1, 2, or 3) heteroatoms or heteroatomic groups. The term “heteroalkyl” includes unbranched or branched saturated chain having carbon and heteroatoms. By way of example, 1, 2, or 3 carbon atoms of the alkyl group in the “heteroalkyl” may be independently replaced with the same or different heteroatomic group. Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.) and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein). In certain embodiments, heteroalkyl can have 1 to 20 carbon atoms, 1 to 15 carbon atoms, 1 to 12 carbon atoms, 1 to 10 carbon atoms, 1 to 8 carbon atoms, or 1 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₁₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems (i.e., 9-10 membered heteroaryl), 5-10 membered ring systems (i.e., 5-10 membered heteroaryl), 5-7 membered ring systems (i.e., 5-7 membered heteroaryl), 5-6 membered ring systems (i.e., 5-6 membered heteroaryl), or 4-6 membered ring systems (i.e., 4-6 membered heteroaryl), each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic group, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”, such as (5- to 10-membered monocyclic heteroaryl)-C₁-C₃ alkyl.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro. Any non-aromatic ring containing at least one heteroatom is considered a heterocyclyl, regardless of the attachment (i.e., can be bound through a carbon atom or a heteroatom). Further, the term heterocyclyl is intended to encompass a moiety comprising any non-aromatic ring containing at least one heteroatom, which ring may be fused to an aryl or heteroaryl ring, regardless of the attachment to the remainder of the molecule. The term heterocyclyl is also intended to encompass a moiety comprising a cycloalkyl ring which is fused to a heteroaryl ring, regardless of the attachment to the remainder of the molecule. Additionally, the term heterocyclyl is intended to encompass a moiety comprising a cycloalkyl ring which is fused to a heterocyclyl ring, regardless of the attachment to the remainder of the molecule. As used herein, heterocyclyl has 2 to 20 ring carbon atoms (i.e., C₂-C₂₀ heterocyclyl), 2 to 12 ring carbon atoms (i.e., C₂-C₁₂ heterocyclyl), 2 to 10 ring carbon atoms (i.e., C₂-C₁₀ heterocyclyl), 2 to 8 ring carbon atoms (i.e., C₂-C₈ heterocyclyl), 3 to 12 ring carbon atoms (i.e., C₃-C₁₂ heterocyclyl), 3 to 8 ring carbon atoms (i.e., C₃-C₈ heterocyclyl), or 3 to 6 ring carbon atoms (i.e., C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. When the heterocyclyl ring contains 4- to 6-ring atoms, it is also referred to herein as a 4- to 6-membered heterocyclyl. Also disclosed herein are 5- or 6-membered heterocyclyls, having 5 or 6 ring atoms, respectively, and 5- to 10-membered heterocyclyls, having 5 to 10 ring atoms. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), tetrahydropyranyl, thiomorpholinyl, thiamorpholinyl, 1-oxo-thiomorpholinyl and 1,1-dioxo-thiomorpholinyl. In certain embodiments, the term “heterocyclyl” can include “spiroheterocyclyl” when there are two positions for substitution on the same carbon atom, wherein at least one ring of the spiro system comprises at least one heteroatom. Examples of the spiro-heterocyclyl rings include, e.g., bicyclic and tricyclic ring systems, such as 2-oxa-7-azaspiro[3.5]nonanyl, 2-oxa-6-azaspiro[3.4]octanyl and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl and isoindolinyl, where the heterocyclyl can be bound via either ring of the fused system.

“Heterocyclylalkyl” refers to the group “heterocyclyl-alkyl-.”

“Oxime” refers to the group —CR^{y}(═NOH) wherein R^{y} is hydrogen, alkyl, alkenyl, alkynyl, cycloalkyl, heterocyclyl, aryl, heteroalkyl or heteroaryl; each of which may be optionally substituted, as defined herein.

“Oxo” refers to the group (═O).

“Cyano” refers to the group (—CN).

“N-oxide” refers to the group (—NO).

“Thiol” refers to the group (—SH).

“Sulfonyl” refers to the group —S(O)₂R^{y}, where R^{y} is hydrogen, alkyl, alkenyl, alkynyl, cycloalkyl, heterocyclyl, aryl, heteroalkyl or heteroaryl; each of which may be optionally substituted, as defined herein. A non-limiting example of a sulfonyl group is —SO₂(C₁-C₆ alkyl), which is herein referred to as alkylsulfonyl. Examples of sulfonyl are methylsulfonyl, ethylsulfonyl, phenylsulfonyl and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl or heteroaryl; each of which may be optionally substituted, as defined herein. Examples of sulfinyl are methylsulfinyl, ethylsulfinyl, phenylsulfinyl and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl or heteroaryl; each of which may be optionally substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5, 1 to 4, or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5, 1 to 4, or 1 to 3) hydrogen atom is replaced by a bond to a non-hydrogen atom such as, but not limited to alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanidino, halo, haloalkyl, haloalkoxy, hydroxyalkyl, heteroalkyl, heteroaryl, heteroarylalkyl, heterocyclyl, heterocyclylalkyl, —NHNH₂, ═NNH₂, imino, imido, hydroxy, oxo, oxime, nitro, sulfonyl, sulfinyl, alkylsulfonyl, alkylsulfinyl, thiocyanate, —S(O)OH, —S(O)₂₀H, sulfonamido, thiol, thioxo, N-oxide or —Si(R^{y})₃, wherein each R^{y} is independently hydrogen, alkyl, alkenyl, alkynyl, heteroalkyl, cycloalkyl, aryl, heteroaryl or heterocyclyl.

In certain embodiments, “substituted” includes any of the above alkyl, alkenyl, alkynyl, cycloalkyl, heterocyclyl, aryl or heteroaryl groups in which one or more (e.g., 1 to 5, 1 to 4, or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(═O)R^{h}, —NR^{g}C(═O)NR^{g}R^{h}, —NR^{g}C(═O)OR^{h}, —NR^{g}S(═O)₁₋₂R^{h}, —C(═O)R^{g}, —C(═O)OR^{g}, —OC(═O)OR^{g}, —OC(═O)R^{g}, —C(═O)NR^{g}R^{h}, —OC(═O)NR^{g}R^{h}, —OR^{g}, —SR^{g}, —S(═O)R^{g}, —S(═O)₂R^{g}, —OS(═O)₁₋₂R^{g}, —S(═O)₁₋₂OR^{g}, —NR^{g}S(═O)₁₋₂NR^{g}R^{h}, ═NSO₂R^{g}, ═NOR^{g}, —S(═O)₁₋₂NR^{g}R^{h}, —SF₅, —SCF₃ or —OCF₃. In certain embodiments, “substituted” also means any of the above groups in which one or more (e.g., 1 to 5, 1 to 4, or 1 to 3) hydrogen atoms are replaced with —C(═O)R^{g}, —C(═O)OR^{g}, —C(═O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5, 1 to 4, or 1 to 3) hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} and Rⁱ are taken together with the atoms to which they are attached to form a heterocyclyl ring optionally substituted with oxo, halo or alkyl optionally substituted with oxo, halo, amino, hydroxyl, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to four. In certain embodiments, as used herein, the phrase “one or more” refers to one to three.

Any compound or structure given herein, is intended to represent unlabeled forms as well as isotopically labeled forms (isotopologues) of the compounds. These forms of compounds may also be referred to as and include “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium. Further, in some embodiments, the corresponding deuterated analog is provided.

In many cases, the compounds of this disclosure are capable of forming acid and/or base salts by virtue of the presence of amino and/or carboxyl groups or groups similar thereto.

Provided also are a pharmaceutically acceptable salt, isotopically enriched analog, deuterated analog, isomer (such as a stereoisomer), and mixture of isomers (such as a mixture of stereoisomers), of the compounds described herein.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include, e.g., hydrochloric acid, hydrobromic acid, sulfuric acid, nitric acid, phosphoric acid and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, aluminum, ammonium, calcium and magnesium salts. Salts derived from organic bases include, but are not limited to, salts of primary, secondary and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃) or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

The term “hydrate” refers to the complex formed by the combining of a compound described herein and water.

A “solvate” refers to an association or complex of one or more solvent molecules and a compound of the disclosure. Examples of solvents that form solvates include, but are not limited to, water, isopropanol, ethanol, methanol, dimethylsulfoxide, ethylacetate, acetic acid and ethanolamine. Solvates include hydrates.

Some of the compounds described herein may exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds described herein, or their pharmaceutically acceptable salts, may include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high performance liquid chromatography (HPLC). When the compounds described herein contain olefinic double bonds or other centres of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposeable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease or condition, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, or deuterated analog thereof means an amount sufficient to effect treatment when administered to a subject, to provide a therapeutic benefit such as amelioration of symptoms or slowing of disease progression. The therapeutically effective amount may vary depending on the subject, and disease or condition being treated, the weight and age of the subject, the severity of the disease or condition, and the manner of administering, which can readily be determined by one of ordinary skill in the art. The effective amount of a compound of the disclosure in such a therapeutic method is, for example, from about 0.01 mg/kg/day to about 1000 mg/kg/day, or from about 0.1 mg/kg/day to about 100 mg/kg/day.

The term “excipient” as used herein refers to an inert or inactive substance that may be used in the production of a drug or pharmaceutical composition, such as a tablet containing a compound as described herein (or pharmaceutically acceptable salt) as an active ingredient. Various substances may be embraced by the term excipient, including without limitation any substance used as a diluent, filler or extender, binder, disintegrant, humectant, coating, emulsifier or dispersing agent, compression/encapsulation aid, cream or lotion, lubricant, solution for parenteral administration, material for chewable tablets, sweetener or flavoring, suspending/gelling agent, or wet granulation agent. Binders may include, e.g., carbomers, povidone, xanthan gum, etc.; coatings may include, e.g., cellulose acetate phthalate, ethylcellulose, gellan gum, maltodextrin, enteric coatings, etc.; compression/encapsulation aids include e.g. calcium carbonate, dextrose, fructose dc (dc—“directly compressible”), honey dc, lactose (anhydrate or monohydrate; optionally in combination with aspartame, cellulose, or microcrystalline cellulose), starch de, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g. dextrose, fructose de, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose de, sorbitol, sucrose de, etc.; and wet granulation agents include, e.g., calcium carbonate, maltodextrin, microcrystalline cellulose, etc. In some cases, the term “excipient” ecompassess pharmaceutically acceptable carriers.

Additional definitions may also be provided below as appropriate.

### II. Compounds

In certain embodiments, the subject matter described herein is directed to a compound of Formula I:

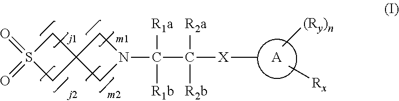

or a pharmaceutically acceptable salt thereof, wherein,
j₁, j₂, m₁, and m₂ are each independently 1 or 2, wherein the sum of j₁ and j₂ and the sum of m₁ and m₂ are each no more than 4, and the total sum of j₁, j₂, m₁, and m₂ is no more than 6;
R₁ₐ, R_{1b}, R₂ₐ, and R_{2b} are each independently selected from the group consisting of hydrogen, halogen, C₁-C₆ alkyl, halo-C₁-C₆ alkyl, halo-C₁-C₆ alkoxy, and C₁-C₆ alkoxy;
X is O or C(R₁₀)(R₂₀);
   R₁₀ and R₂₀ are each independently selected from the group consisting of hydrogen, halogen, C₁-C₆ alkyl, halo-C₁-C₆ alkyl, halo-C₁-C₆ alkoxy, and C₁-C₆ alkoxy;
or,
R₁ₐ and R_{1b}, R₂ₐ and R_{2b}, or R₁₀ and R₂₀, taken together with the atom to which they are attached form a C₃-C₅ cycloalkyl, optionally substituted with halogen, hydroxy, halo-C₁-C₆ alkoxy, C₁-C₆ alkoxy, —SO₂(C₁-C₆ alkyl), —CN, or —NRR′;
Ring A is phenyl, or 5- to 6-membered heteroaryl comprising one, two, or three heteroatoms independently selected from the group consisting of O, N, and S;
each R_{y} is independently halogen, C₁-C₆ alkyl, halo-C₁-C₆ alkyl, hydroxy, C₁-C₆ alkoxy, halo-C₁-C₆ alkoxy, C₃-C₇ cycloalkyl, C₃-C₇halocycloalkyl, —SO₂(C₁-C₆ alkyl), —CN, or —NRR′;
n is 0, 1, 2, 3, or 4;
Rₓ is halogen, hydroxy, C₁-C₁₀ alkyl, halo-C₁-C₆ alkyl, C₁-C₆ alkoxy, halo-C₁-C₆ alkoxy, 5- to 7-membered heterocyclyl, C₃-C₇ cycloalkyl, C₆-C₁₀ aryl, 5- to 6-membered heteroaryl, —SO₂(C₁-C₆ alkyl), —CN, or —NRR′;
   wherein said heterocyclyl, cycloalkyl, aryl, or heteroaryl is optionally substituted with one or more substituents independently selected from the group consisting of halogen, C₁-C₆ alkyl, hydroxy, C₁-C₆ alkoxy, halo-C₁-C₆ alkoxy, halo-C₁-C₆ alkyl, —SO₂(C₁-C₆ alkyl), —CN, and —NR″R′″; and
R, R′, R″, and R′″ are each independently hydrogen, C₁-C₆ alkyl, or halo-C₁-C₆ alkyl.

Useful compounds include all those having variables as described above.

In certain embodiments, compounds include those where one of m₁ and m₂ is 1 and the other is 2 (and the sum is 3). In certain embodiments, compounds include those where m₁ and m₂ are each 1 (and the sum is 2). In certain embodiments, compounds include those where m₁ and m₂ are each 2 (and the sum is 4).

In certain embodiments, compounds include those where one of j₁ and j₂ is 1 and the other is 2 (and the sum is 3). In certain embodiments, compounds include those where j₁ and j₂ are each 1 (and the sum is 2). In certain embodiments, compounds include those where j₁ and j₂ are each 2 (and the sum is 4).

In certain embodiments, the total sum of j₁, j₂, m₁, and m₂ is 5. In certain embodiments, the total sum of j₁, j₂, m₁, and m₂ is 6.

In certain embodiments, compounds include those where j₁, j₂, m₁, and m₂ are as indicated in Table 1A.

TABLE 1A

| Spiro Ring | | | | | | | j₁ + j₂ + |
|---|---|---|---|---|---|---|---|
| Size | j₁ | j₂ | j₁ + j₂ | m₁ | m₂ | m₁ + m₂ | m₁ + m₂ |
| 5,5-fused | 2 | 1 | 3 | 2 | 1 | 3 | 6 |
| 5,5-fused | 1 | 2 | 3 | 2 | 1 | 3 | 6 |
| 4,5-fused | 1 | 1 | 2 | 2 | 1 | 3 | 5 |
| 4,6-fused | 1 | 1 | 2 | 2 | 2 | 4 | 6 |
| 6,4-fused | 2 | 2 | 4 | 1 | 1 | 2 | 6 |

In certain embodiments (Embodiment B), compounds of Formula I are of Formula Ib:

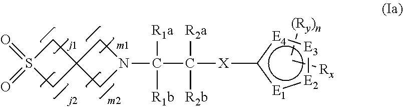

or a pharmaceutically acceptable salt thereof, wherein E₁, E₂, E₃, and E₄ are each independently selected from the group consisting of CH, C—Rₓ, C—R_{y}, N—Rₓ, N—R_{y}, N, NH, O, and S, wherein one, two, or three of E₁, E₂, E₃, and E₄ are N, NH, N—Rₓ, N—R_{y}, O, or S; and j₁, j₂, m₁, m₂, R₁ₐ, R₂ₐ, R_{1b}, R_{2b}, X, R_{y}, Rₓ, and n are as defined for Formula I.

In certain embodiments (Embodiment B), compounds of Formula I are of Formula Ib:

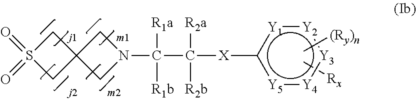

or a pharmaceutically acceptable salt thereof, wherein Y₁, Y₂, Y₃, Y₄, and Y₅ are each independently selected from the group consisting of CH, C—R_{y}, C—Rₓ, N, or S, wherein 1, 2, or 3 of Y₁, Y₂, Y₃, Y₄, and Y₅ are N, or S; and j₁, j₂, m₁, m₂, R₁ₐ, R₂ₐ, R_{1b}, R_{2b}, X, R_{y}, Rₓ, and n are as defined for Formula I.

In certain embodiments of the above Embodiment A, compounds include those of Formula Ia, or a pharmaceutically acceptable salt thereof, where E₁ is CH; E₂ is N; E₃ is N—Rₓ; and E₄ is CH. In certain embodiments, compounds include those of Formula Ia, or a pharmaceutically acceptable salt thereof, where E₁ is CH; E₂ is C—Rₓ; E₃ is N; and E₄ is N—R_{y}.

In certain embodiments of the above Embodiment B, compounds of Formula Ib, or pharmaceutically acceptable salts thereof, include those of Formula Ib, or a pharmaceutically acceptable salt thereof, where Y₁, Y₂, Y₄, and Y₅ are each CH, and Y₃ is C—Rₓ. In certain embodiments, compounds include those of Formula Ib where Y₁, Y₂, and Y₅ are each CH, Y₄ is N, and Y₃ is C—Rₓ. In certain embodiments, compounds include those of Formula Ib where Y₁, Y₂, and Y₅ are each CH, Y₄ is C—R_{y}, and Y₃ is C—Rₓ. In certain embodiments, compounds include those of Formula Ib where Y₁, Y₂, and Y₅ are each CH, Y₄ is C—Rₓ, and Y₃ is N. In certain embodiments, compounds include those of Formula Ib where Y₂ and Y₄ are each N, Y₁ and Y₅ are each CH, and Y₃ is C—Rₓ. In certain embodiments, compounds include those of Formula Ib where Y₅ is N, Y₃ is C—Rₓ, and Y₁, Y₂, and Y₄ are each CH. In certain embodiments, compounds include those of Formula Ib where Y₄ is C—Rₓ, Y₂ is N, and Y₁, Y₃, and Y₅ are each CH. In certain embodiments, compounds include those of Formula Ib where Y₁, Y₂, Y₃, and Y₄, are each CH, and Y₅ is C—Rₓ. In certain embodiments, compounds include those of Formula Ib where Y₁ and Y₅ are CH; Y₄ is C—R_{y}; Y₃ is C—Rₓ; and Y₂ is N. In certain embodiments, compounds include those of Formula Ib where Y₁ is CH; Y₅ is C—R_{y}; Y₄ is N; Y₃ is C—Rₓ; and Y₂ is CH.

In certain embodiments of any of the above, compounds of Formula I, Ia, and Ib, or pharmaceutically acceptable salts thereof, include those where R_{y} is halogen or halo-C₁-C₆ alkyl. In certain embodiments, compounds include those where R_{y} is halogen, C₁-C₃ alkyl, or halo-C₁-C₆ alkyl. In certain embodiments, compounds include those where R_{y} is chloro, trifluoromethyl, fluoro, methyl, or isopropyl. In certain embodiments, compounds include those where R_{y} is selected from the group consisting of chloro, trifluoromethyl, and fluoro. In some embodiments, such compounds are of Formula I, or a pharmaceutically acceptable salt thereof. In other embodiments, such compounds are of Formula Ia, or a pharmaceutically acceptable salt thereof. In still further embodiments, such compounds are of Formula Ib, or a pharmaceutically acceptable salt thereof.

In certain embodiments of any of the above, compounds of Formula I, Ia, and Ib, or a pharmaceutically acceptable salt thereof, include those where Rₓ is halogen or halo-C₁-C₆ alkyl. In certain embodiments, compounds include those where Rₓ is selected from the group consisting of fluoro, trifluoromethyl, difluoromethyl, fluoromethyl, trifluoroethyl, and trifluoromethoxy. In certain embodiments, compounds include those where Rₓ is —CF₃, —CH₂CF₃, —CHF₂, or —CH₂F. In certain embodiments, compounds include those where Rₓ is —CF₃. In some embodiments, such compounds are of Formula I, or a pharmaceutically acceptable salt thereof. In other embodiments, such compounds are of Formula Ia, or a pharmaceutically acceptable salt thereof. In still further embodiments, such compounds are of Formula Ib, or a pharmaceutically acceptable salt thereof.

In certain embodiments of any of the above, compounds of Formula I, Ia, and Ib, or a pharmaceutically acceptable salt thereof, include those where Rₓ is C₃-C₇ cycloalkyl optionally substituted with one or more substituents independently selected from the group consisting of halogen, C₁-C₆ alkyl, hydroxy, C₁-C₆ alkoxy, halo-C₁-C₆ alkoxy, and halo-C₁-C₆ alkyl. In certain embodiments, compounds include those where Rₓ is selected from the group consisting of cyclopropyl, cyclobutyl, cyclopentyl, and cyclohexyl, optionally substituted with one or two substituents, each independently selected from the group consisting of fluoro, methyl, hydroxy, and trifluoromethyl. In certain embodiments, compounds include those where Rₓ is cyclohexyl substituted with one or two substituents, each independently selected from the group consisting of fluoro, methyl hydroxy, and trifluoromethyl. In certain embodiments, compounds include those where Rₓ is

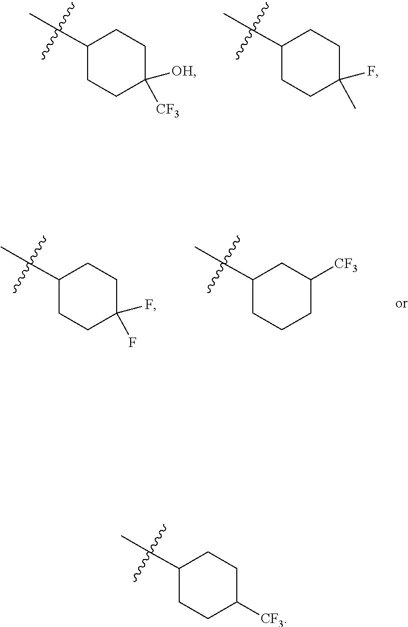

In certain embodiments, compounds include those where Rₓ is unsubstituted cyclohexyl, unsubstituted cyclopropyl, or unsubstituted cyclobutyl. In certain embodiments, compounds include those where Rₓ is cyclopropyl substituted once or twice with methyl. In certain embodiments, compounds include those where Rₓ is

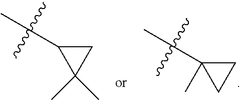

In some embodiments, such compounds are of Formula I, or a pharmaceutically acceptable salt thereof. In other embodiments, such compounds are of Formula Ia, or a pharmaceutically acceptable salt thereof. In still further embodiments, such compounds are of Formula Ib, or a pharmaceutically acceptable salt thereof.

In certain embodiments of any of the above, compounds of Formula I, Ia, and Ib, or a pharmaceutically acceptable salt thereof, include those where Rₓ is a 5- to 7-membered heterocyclyl. In certain embodiments, compounds include those where Rₓ is a 6-membered heterocyclyl. In certain embodiments, compounds include those where Rₓ is tetrahydropyranyl. In certain embodiments, compounds include those where Rₓ is

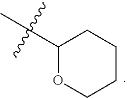 [SMILES: *C1CCCCO1]

In some embodiments, such compounds are of Formula I, or a pharmaceutically acceptable salt thereof. In other embodiments, such compounds are of Formula Ia, or a pharmaceutically acceptable salt thereof. In still further embodiments, such compounds are of Formula Ib, or a pharmaceutically acceptable salt thereof.

In certain embodiments of any of the above, compounds of Formula I, Ia, and Ib, or a pharmaceutically acceptable salt thereof, include those where Rₓ is C₁-C₁₀ alkyl. In certain embodiments, compounds include those where Rₓ is selected from the group consisting of propyl, butyl, pentyl, hexyl, and heptyl. In certain embodiments, compounds include those where Rₓ is selected from the group consisting of n-pentyl, tert-pentyl, neopentyl, isopentyl, and sec-pentyl. In certain embodiments, compounds include those where Rₓ is tert-pentyl. In some embodiments, such compounds are of Formula I, or a pharmaceutically acceptable salt thereof. In other embodiments, such compounds are of Formula Ia, or a pharmaceutically acceptable salt thereof. In still further embodiments, such compounds are of Formula Ib, or a pharmaceutically acceptable salt thereof.

In certain embodiments of any of the above, compounds of Formula I, Ia, and Ib, or a pharmaceutically acceptable salt thereof, include those where Rₓ is pyridinyl optionally substituted with halo-C₁-C₆ alkyl. In certain embodiments, compounds include those where Rₓ is

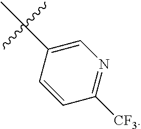 [SMILES: *c1ccc(C(F)(F)F)nc1]

In some embodiments, such compounds are of Formula I, or a pharmaceutically acceptable salt thereof. In other embodiments, such compounds are of Formula Ia, or a pharmaceutically acceptable salt thereof. In still further embodiments, such compounds are of Formula Ib, or a pharmaceutically acceptable salt thereof.

In certain embodiments of any of the above, compounds of Formula I, Ia, and Ib, or a pharmaceutically acceptable salt thereof, include those where X is O.

In certain embodiments of any of the above, compounds of Formula I, Ia, and Ib, or a pharmaceutically acceptable salt thereof, include those where X is C(R₁₀)(R₂₀), and R₁₀ and R₂₀ are each independently hydrogen or fluoro. In certain embodiments, compounds include those where R₁₀ and R₂₀ are each hydrogen.

In certain embodiments of any of the above, compounds of Formula I, Ia, and Ib, or a pharmaceutically acceptable salt thereof, include those where R₂ₐ and R_{2b} are each independently selected from the group consisting of hydrogen, C₁-C₆ alkoxy, halogen, and C₁-C₆ alkyl. In certain embodiments, compounds include those where R₂ₐ and R_{2b} are each independently methoxy, fluoro, hydrogen or methyl. In certain embodiments, compounds include those where R₂ₐ is hydrogen and R_{2b} is methyl. In certain embodiments, compounds include those where R₂ₐ and R_{2b} are each methyl. In certain embodiments, compounds include those where R₂ₐ and R_{2b} are each hydrogen.

In certain embodiments of any of the above, compounds of Formula I, Ia, and Ib, or a pharmaceutically acceptable salt thereof, include those where R₁ₐ and R_{1b} are each independently hydrogen or methyl. In certain embodiments, compounds include those where R₁ₐ and R_{1b} are each hydrogen.

In certain embodiments (Embodiment A1), the compound of Formula I is of Formula Ia, or a pharmaceutically acceptable salt thereof, wherein:
E₁, E₂, E₃, and E₄ are each independently selected from the group consisting of CH, C—Rₓ, C—R_{y}, N—Rₓ, N—R_{y}, N, and NH, wherein one, two, or three of E₁, E₂, E₃, and E₄ are N, NH, N—Rₓ, or N—R_{y};
one of m₁ and m₂ is 1 and the other is 2, or m₁ and m₂ are each 2;
j₁ and j₂ are each 1;
n is 0 or 1;
Rₓ is halo-C₁-C₆ alkyl;
R_{y} is C₁-C₆ alkyl;
X is O or C(R₁₀)(R₂₀), wherein R₁₀ and R₂₀ are each hydrogen; and
R₁ₐ, R_{1b}, R₂ₐ, and R_{2b} are each independently hydrogen or C₁-C₃ alkyl.

In certain embodiments of the above Embodiment A1, compounds of Formula Ia, or pharmaceutically acceptable salts thereof, include those wherein R₁ₐ and R_{1b} are each hydrogen; R₂ₐ is hydrogen and R_{2b} is methyl; and X is CH₂; or R₁ₐ R_{1b}, R₂ₐ, and R_{2b} are each hydrogen; and X is O. In certain embodiments of the above embodiment, compounds include those wherein E₁ is CH; E₂ is N; E₃ is N—Rₓ; and E₄ is CH; and Rₓ is trifluoroethyl. In certain embodiments of the above embodiment, compounds include those wherein E₁ is CH; E₂ is C—Rₓ; E₃ is N; and E₄ is N—R_{y}; Rₓ is trifluoromethyl; and R_{y} is isopropyl.

In certain embodiments (Embodiment B1), the compound of Formula I is of Formula Ib, or a pharmaceutically acceptable salt thereof, wherein:
j₁, j₂, m₁, and m₂ are each independently 1 or 2, wherein the sum of j₁ and j₂ and the sum of m₁ and m₂ are each no more than 4, and the total sum of j₁, j₂, m₁, and m₂ is no more than 6;
four of Y₁, Y₂, Y₃, Y₄, and Y₅ are CH, and one is C—Rₓ;
Rₓ is selected from the group consisting of halo-C₁-C₆ alkyl, halogen, halo-C₁-C₆ alkoxy, C₁-C₁₀ alkyl, pyridinyl, and C₃-C₇ cycloalkyl, wherein said C₃-C₇ cycloalkyl or pyridinyl is optionally substituted one or more times with C₁-C₆ alkyl or halo-C₁-C₆ alkyl;
n is 0;
X is C(R₁₀)(R₂₀) or O, wherein R₁₀ and R₂₀ are each independently hydrogen, methyl, or fluoro;
R₁ₐ and R_{1b} are each independently hydrogen or methyl; and
R₂ₐ and R_{2b} are each independently hydrogen, fluoro, methoxy, or methyl; or, R₂ₐ and R_{2b}, together with the carbon atom to which they are attached, form a C₃-C₅ cycloalkyl.

In certain embodiments of the above Embodiment B1, compounds of Formula (Ib), or pharmaceutically acceptable salts thereof, include those where Y₁, Y₂, Y₄, and Y₅ are each CH; and Y₃ is C—Rₓ. In certain embodiments of the above embodiment, compounds include those where Rₓ is trifluoromethyl, difluoromethyl, pyridinyl, trifluoromethoxy, fluoro, pentyl, cyclopropyl, or cyclobutyl, wherein said cyclopropyl or cyclobutyl is optionally substituted once or twice with C₁-C₃ alkyl; and said pyridinyl is optionally substituted once with halo-C₁-C₆ alkyl. In certain embodiments of the above embodiment, compounds include those where Rₓ is selected from the group consisting of —C(CH₃)₂CH₂CH₃, —CF₃, —CHF₂, —OCF₃, fluoro,

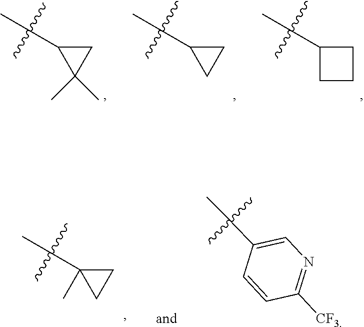

In certain embodiments of the above embodiment, compounds include those where X is O; and (1) R₁ₐ, R_{1b}, R₂ₐ, and R_{2b} are each hydrogen; (2) one of R₁ₐ and R₂ₐ is methyl and the other is hydrogen, and R₂ₐ and R_{2b} are each hydrogen; (3) R₂ₐ and R_{2b} are methyl and R₁ₐ and R_{1b} are hydrogen; or (4) R₁ₐ and R_{1b} are hydrogen and R₂ₐ and R_{2b}, together with the carbon to which they are attached, form a cyclobutyl or cyclopentyl ring. In certain embodiments of the above embodiment, compounds include those where X is C(R₁₀)(R₂₀), wherein: R₁₀ is hydrogen and R₂₀ is fluoro; or, R₁₀ and R₂₀ are each hydrogen; R₁ₐ and R_{1b} are each hydrogen; and R₂ₐ is hydrogen and R_{2b} is fluoro, hydrogen, methyl, or methoxy; or R₂ₐ and R_{2b} are each methyl.

In certain embodiments (Embodiment B2), compounds are of Formula Ib, or pharmaceutically acceptable salts thereof, wherein:
j₁, j₂, m₁, and m₂ are each independently 1 or 2, wherein the sum of j₁ and j₂ and the sum of m₁ and m₂ are each no more than 4, and the total sum of j₁, j₂, m₁, and m₂ is no more than 6;
three of Y₁, Y₂, Y₃, Y₄, and Y₅ are CH, one is C—Rₓ, and the other is C—R_{y};
R_{y} is halogen or halo-C₁-C₆ alkyl;
Rₓ is halogen, halo-C₁-C₆ alkyl, 5- to 7-membered heterocyclyl, or C₃-C₇ cycloalkyl, wherein said heterocyclyl or cycloalkyl is optionally substituted one or more times with halogen, halo-C₁-C₆ alkoxy, hydroxy, or C₁-C₆ alkyl;
n is 1;
X is C(R₁₀)(R₂₀) or O, wherein R₁₀ and R₂₀ are each hydrogen; and
R₁ₐ, R_{1b}, R₂ₐ, and R_{2b} are each independently hydrogen or methyl.

In certain embodiments of the above Embodiment B2, compounds of Formula Ib, or pharmaceutically acceptable salts thereof, include those where Y₁, Y₂, and Y₅ are each CH; Y₄ is C—R_{y}; and Y₃ is C—Rₓ. In certain embodiments of the above embodiment, compounds include those where R_{y} is chloro, fluoro, or trifluoromethyl; and Rₓ is fluoro, chloro, tetrahydropyranyl, or cyclohexyl, wherein said cyclohexyl is substituted once with fluoro or hydroxy and once with trifluoromethyl, methyl, or fluoro. In certain embodiments of the above embodiment, compounds include those where R_{y} is chloro or fluoro; and Rₓ is fluoro, chloro, —CF₃,

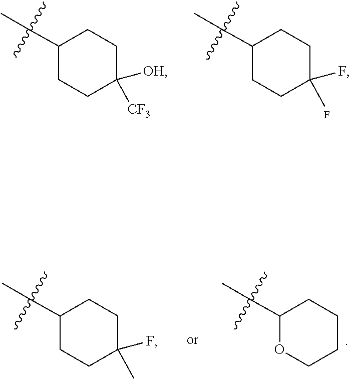

In certain embodiments of the above embodiment, compounds include those where R_{y} is chloro. In certain embodiments of the above embodiment, compounds include those where X is CH₂ or O; R₁ₐ and R_{1b} are each hydrogen; and R₂ₐ and R_{2b} are each independently hydrogen or methyl.

In certain embodiments (Embodiment B3), compounds are of Formula Ib, or pharmaceutically acceptable salts thereof, wherein:
j₁, j₂, m₁, and m₂ are each independently 1 or 2, wherein the sum of j₁ and j₂ and the sum of m₁ and m₂ are each no more than 4, and the total sum of j₁, j₂, m₁, and m₂ is no more than 6;
one or two of Y₁, Y₂, Y₃, Y₄, and Y₅ are N; one of Y₁, Y₂, Y₃, Y₄, and Y₅ is C—Rₓ; and the remainder of Y₁, Y₂, Y₃, Y₄, and Y₅ are each CH or C—R_{y};
n is 0 or 1;
R_{y} is halogen, halo-C₁-C₆ alkyl, or C₁-C₆ alkyl;
Rₓ is halo-C₁-C₆ alkyl, halogen, pyridinyl, or C₃-C₇ cycloalkyl, wherein said pyridinyl or cycloalkyl is optionally substituted 1, 2, or 3 times with halogen or halo-C₁-C₆ alkyl;
X is C(R₁₀)(R₂₀) or O, wherein R₁₀ and R₂₀ are each hydrogen; or R₁₀ is hydrogen and R₂₀ is fluoro;
R₁ₐ and R_{1b} are each hydrogen; and
R₂ₐ and R_{2b} are each independently hydrogen or methyl; or, R₂ₐ and R_{2b}, together with the carbon to which they are attached, come together to form a C₃-C₅ cycloalkyl.

In certain embodiments of the above Embodiment B3, compounds of Formula Ib, or pharmaceutically acceptable salts thereof, include those where Y₁, Y₂, and Y₅ are each CH, Y₄ is N, and Y₃ is C—Rₓ; Y₁, Y₂, and Y₅ are each CH, Y₄ is C—Rₓ, and Y₃ is N; Y₂ and Y₄ are each N, Y₁ and Y₅ are each CH, and Y₃ is C—Rₓ; Y₅ is N, Y₃ is C—Rₓ, and Y₁, Y₂, and Y₄ are each CH; Y₄ is C—Rₓ, Y₂ is N, and Y₁, Y₃, and Y₅ are each CH; Y₅ are CH, Y₄ is C—R_{y}, Y₃ is C—Rₓ, and Y₂ is N; or Y₁ is CH, Y₅ is C—R_{y}, Y₄ is N, Y₃ is C—Rₓ, and Y₂ is CH. In certain embodiments of the above embodiment, compounds include those where Rₓ is trifluoromethyl, fluoro, or cyclohexyl optionally substituted with trifluoromethyl; and R_{y} is chloro, methyl, or trifluoromethyl. In certain embodiments of the above embodiment, compounds include those where Rₓ is —CF₃, fluoro,

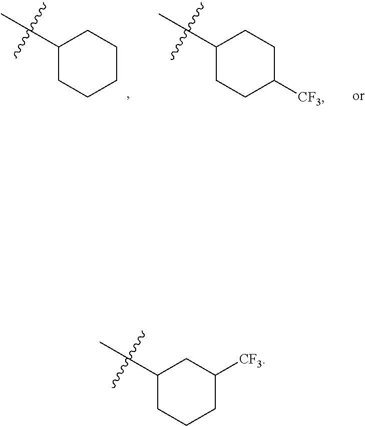 [SMILES: *C1CCCCC1 ; "," ; *C1CCC(C(F)(F)F)CC1 ; "or" ; *C1CCCC(C(F)(F)F)C1]

In certain embodiments (Embodiment B4), compounds are of Formula Ib, or pharmaceutically acceptable salts thereof, wherein:
Y₁, Y₂, Y₄, and Y₅ are each CH, and Y₃ is C—Rₓ;
Rₓ is selected from the group consisting of halo-C₁-C₆ alkyl, halo-C₁-C₆ alkoxy, C₁-C₁₀ alkyl, and C₃-C₇ cycloalkyl, wherein said C₃-C₇ cycloalkyl is optionally substituted one or more times with C₁-C₆ alkyl;
n is 0;
X is C(R₁₀)(R₂₀), wherein R₁₀ and R₂₀ are each independently hydrogen, methyl, or fluoro;
R₁ₐ and R_{1b} are each independently hydrogen or methyl;
R₂ₐ and R_{2b} are each independently hydrogen, fluoro, methoxy, or methyl;
one of m₁ and m₂ is 1 and the other is 2, or m₁ and m₂ are each 2, or m₁ and m₂ are each 1; and,
j₁ and j₂ are each 1, or one of j₁ and j₂ is 1 and the other is 2, or j₁ and j₂ are each 2, provided that the total sum of j₁, j₂, m₁, and m₂ is no more than 6.

In certain embodiments of the above Embodiment B4, compounds of Formula Ib, or pharmaceutically acceptable salts thereof, include those wherein:
Rₓ is selected from the group consisting of trifluoromethyl, trifluoromethoxy, pentyl, cyclopropyl, and cyclobutyl, wherein said cyclopropyl or cyclobutyl is optionally substituted once or twice with methyl;
R₁₀ and R₂₀ are each hydrogen;
R₁ₐ and R_{1b} are each hydrogen;
R₂ₐ and R_{2b} are each independently hydrogen or methyl;
one of m₁ and m₂ is 1 and the other is 2; and,
j₁ and j₂ are each 1,
and pharmaceutically acceptable salts thereof.

In certain embodiments (Embodiment B5), compounds are of Formula Ib, or pharmaceutically acceptable salts thereof, wherein:
Y₁, Y₂, and Y₅ are each CH, Y₄ is C—R_{y}, and Y₃ is C—Rₓ;
R_{y} is halogen;
n is 1;
Rₓ is a 5- to 7-membered heterocyclyl or C₃-C₇ cycloalkyl, optionally substituted one or more times with halogen, halo-C₁-C₆ alkoxy, hydroxy, or C₁-C₆ alkyl;
X is C(R₁₀)(R₂₀), wherein R₁₀ and R₂₀ are each hydrogen;
R₁ₐ and R_{1b} are each hydrogen;
R₂ₐ and R_{2b} are each independently hydrogen or methyl;
one of m₁ and m₂ is 1 and the other is 2; and
j₁ and j₂ are each 1.

In certain embodiments of the above Embodiment B5, compounds of Formula Ib, or pharmaceutically acceptable salts thereof, include those where R_{y} is chloro; and Rₓ is cyclohexyl or a 6-membered heterocyclyl, optionally substituted with one or two substituents, each independently selected from the group consisting of fluoro, trifluoromethyl, methyl, and hydroxy.

In certain embodiments (Embodiment B6), compounds are of Formula Ib, or pharmaceutically acceptable salts thereof, wherein one or two of Y₁, Y₂, Y₃, Y₄, and Y₅ are N; one of Y₁, Y₂, Y₃, Y₄, and Y₅ is C—Rₓ; and the remainder of Y₁, Y₂, Y₃, Y₄, and Y₅ are each CH;
Rₓ is halo-C₁-C₆ alkyl;
n is 0;
X is C(R₁₀)(R₂₀) or O, wherein R₁₀ and R₂₀ are each hydrogen;
R₁ₐ and R_{1b} are each hydrogen;
R₂ₐ and R_{2b} are each independently hydrogen or methyl;
one of m₁ and m₂ is 1 and the other is 2, or m₁ and m₂ are each 2; and
j₁ and j₂ are each 1, or one of j₁ and j₂ is 1 and the other is 2, or j₁ and j₂ are each 2, provided that
the total sum of j₁, j₂, m₁, and m₂ is no more than 6.

In certain embodiments of the above Embodiment B6, compounds of Formula Ib, or pharmaceutically acceptable salts thereof, include those where Rₓ is trifluoromethyl. In certain embodiments of the above embodiment, compounds include those where R₂ₐ is hydrogen and R_{2b} is methyl. In certain embodiments of the above embodiment, compounds include those where R₂ₐ and R_{2b} are each hydrogen. In certain embodiments of the above embodiment, compounds include those where Y₁ is CH, Y₂ is N, Y₃ is C—Rₓ, Y₄ is N or CH, and Y₅ is CH. In certain embodiments of the above embodiment, compounds include those where one of m₁ and m₂ is 1 and the other is 2; and j₁ and j₂ are each 1. In certain embodiments of the above embodiment, compounds include those where m₁ and m₂ are each 2; and j₁ and j₂ are each 1. In certain embodiments of the above embodiment, compounds include those where m₁ and m₂ are each 1; and j₁ and j₂ are each 2.

In certain embodiments (Embodiment B7), compounds are of Formula Ib, or pharmaceutically acceptable salts thereof, wherein:
X is O or C(R₁₀R₂₀), wherein R₁₀ and R₂₀ are each hydrogen;
four of Y₁, Y₂, Y₃, Y₄, and Y₅ are CH, and one is C—Rₓ;
or, three of Y₁, Y₂, Y₃, Y₄, and Y₅ are CH, one is C—Rₓ, and the other is C—R_{y};
Rₓ is selected from the group consisting of halogen, C₁-C₆ alkyl, halo-C₁-C₆ alkoxy, halo-C₁-C₆ alkyl, and C₃-C₇ cycloalkyl;
R_{y} is halogen or halo-C₁-C₆ alkyl;
n is 0 or 1;
R₁ₐ and R_{1b} are each hydrogen;
R₂ₐ and R_{2b} are each independently hydrogen or methyl;
or, R₂ₐ and R_{2b}, together with the carbon atom to which they are attached, form a C₃-C₅ cycloalkyl;
one of m₁ and m₂ is 1 and the other is 2, or m₁ and m₂ are each 1, or m₁ and m₂ are each 2; and
j₁ and j₂ are each 1, or one of j₁ and j₂ is 1 and the other is 2, or j₁ and j₂ are each 2, provided that the total sum of j₁, j₂, m₁, and m₂ is no more than 6.

In certain aspects of the above Embodiment B7, compounds of Formula Ib, or pharmaceutically acceptable salts thereof, include those where Y₁, Y₂, Y₄, and Y₅ are CH, and Y₃ is C—Rₓ. In certain aspects of the above embodiment, compounds include those where Y₁, Y₄, and Y₅ are CH, Y₂ is C—R_{y}, and Y₃ is C—Rₓ. In certain aspects of the above embodiment, compounds include those where Rₓ is trifluoromethyl, trifluoromethoxy, fluoro, or chloro. In certain aspects of the above embodiment, compounds include those where R_{y} is fluoro. In certain aspects of the above embodiment, compounds include those where m₁ and m₂ are each 2 and j₁ and j₂ are each 1. In certain aspects of the above embodiment, compounds include those where m₁ and m₂ are each 1 and j₁ and j₂ are each 2. In certain aspects of the above embodiment, compounds include those where Rₓ is trifluoromethyl. In certain aspects of the above embodiment, compounds include those where X is O. In certain aspects of the above embodiment, compounds include those where X is C(R₁₀R₂₀).

In certain embodiments (Embodiment B8), compounds are of Formula Ib, or pharmaceutically acceptable salts thereof, wherein:
X is O;
four of Y₁, Y₂, Y₃, Y₄, and Y₅ are CH, and one is C—Rₓ;
Rₓ is selected from the group consisting of halogen, C₁-C₆ alkyl, halo-C₁-C₆ alkoxy, halo-C₁-C₆ alkyl, and C₃-C₇ cycloalkyl;
n is 0;
R₁ₐ and R_{1b} are each hydrogen;
R₂ₐ and R_{2b} are each independently hydrogen or methyl;
one of m₁ and m₂ is 1 and the other is 2, or m₁ and m₂ are each 1, or m₁ and m₂ are each 2; and,
j₁ and j₂ are each 1, or one of j₁ and j₂ is 1 and the other is 2, or j₁ and j₂ are each 2, provided that the total sum of j₁, j₂, m₁, and m₂ is no more than 6.

In certain aspects of the above Embodiment B8, compounds of Formula Ib, or pharmaceutically acceptable salts thereof, include those where R₂ₐ and R_{2b} are each methyl; and Rₓ is trifluoromethyl, fluoro, or trifluoromethoxy. In certain aspects of the above embodiment, compounds include those where m₁ and m₂ are each 2; and j₁ and j₂ are each 1. In certain aspects of the above embodiment, compounds include those where Y₁, Y₂, Y₄, and Y₅ are CH, Y₃ is C—Rₓ. In certain aspects of the above embodiment, compounds include those where Rₓ is trifluoromethyl.

In certain embodiments (Embodiment A2), compounds are of Formula Ia, or pharmaceutically acceptable salts thereof, wherein E₁ is CH, E₂ is N, E₃ is N—Rₓ, and E₄ is CH; one of m₁ and m₂ is 2 and the other is 1; j₁ and j₂ are each 1; R₁ₐ and R_{1b} are each hydrogen; R₂ₐ is hydrogen and R_{2b} is methyl; X is C(R₁₀R₂₀) wherein R₁₀ and R₂₀ are each hydrogen; and Rₓ is halo-C₁-C₆ alkyl. In certain embodiments, Rₓ is trifluoroethyl.

In certain embodiments (Embodiment A3), compounds include those of Formula Ia, or a pharmaceutically acceptable salt thereof, wherein E₁ is CH, E₂ is C—R_{y}, E₃ is N, and E₄ is N—Rₓ; one of m₁ and m₂ is 2 and the other is 1, or m₁ and m₂ are each 2; j₁ and j₂ are each 1; R₁ₐ and R_{1b} are each hydrogen;
R₂ₐ is hydrogen and R_{2b} is methyl; X is C(R₁₀R₂₀) wherein R₁₀ and R₂₀ are each hydrogen; Rₓ is C₁-C₆ alkyl and R_{y} is halo-C₁-C₆ alkyl. In certain embodiments, Rₓ is propyl and R_{y} is trifluoromethyl. In certain embodiments, Rₓ is isopropyl.

The subject matter described herein includes the following compounds in Table 1, or pharmaceutically acceptable salts thereof. Individual enantiomers and diastereomers are included in the table below by compound name, and their corresponding structures can be readily determined therefrom. In Table 1, the asterix (*) indicates an isolated isomer or isolated group of isomers, but that the stereochemistry has not been assigned. In some instances, the enantiomers or diastereomers of the present disclosure may be identified by their respective properties, for example, retention times by chiral HPLC, NMR peaks, and/or biological activities (e.g., as described further in the Examples), whereas the absolute stereo configurations of one or more chiral centers has not been assigned.

TABLE 1

| Cmpd | | |
|---|---|---|
| No. | Structure | Chemical name |
| 102 | 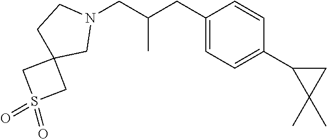 [SMILES: CC(Cc1ccc(C2CC2(C)C)cc1)CN1CCC2(C1)CS(=O)(=O)C2] | 6-3-(4-(2,2- Dimethylcyclopropyl)phenyl)-2- methylpropyl)-2-thia-6- azaspiro[3.4]octane 2,2-dioxide |
| 104 | 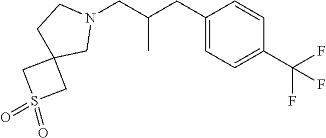 [SMILES: CC(Cc1ccc(C(F)(F)F)cc1)CN1CCC2(C1)CS(=O)(=O)C2] | 6-(2-Methyl-3-(4- (trifluoromethyl)phenyl)propyl)- 2-thia-6-azaspiro[3.4]octane 2,2- dioxide |
| 105 | 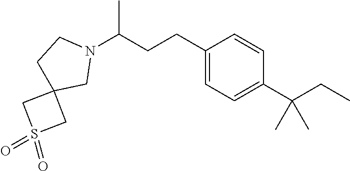 [SMILES: CCC(C)(C)c1ccc(CCC(C)N2CCC3(C2)CS(=O)(=O)C3)cc1] | 6-(4-(4-(tert- Pentyl)phenyl)butan-2-yl)-2- thia-6-azaspiro[3.4]octane 2,2- dioxide |
| 106 | 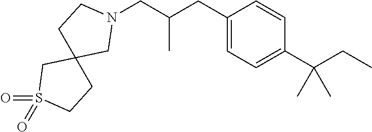 [SMILES: CCC(C)(C)c1ccc(CC(C)CN2CCC3(CCS(=O)(=O)C3)C2)cc1] | 7-2-Methyl-3-(4-(tert- pentyl)phenyl)propyl)-2-thia-7- azaspiro[4.4]nonane 2,2-dioxide |
| 107 | 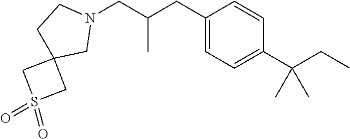 [SMILES: CCC(C)(C)c1ccc(CC(C)CN2CCC3(C2)CS(=O)(=O)C3)cc1] | 6-(2-Methyl-3-(4-(tert- pentyl)phenyl)propyl)-2-thia-6- azaspiro[3.4]octane 2,2-dioxide |
| 108 | 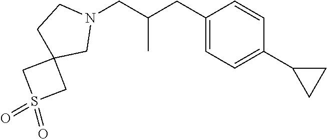 [SMILES: CC(Cc1ccc(C2CC2)cc1)CN1CCC2(C1)CS(=O)(=O)C2] | 6-(3-(4-Cyclopropylphenyl)-2- methylpropyl)-2-thia-6- azaspiro[3.4]octane 2,2-dioxide |
| 109A | 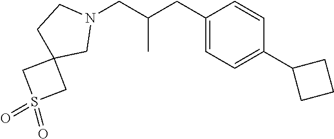 [SMILES: CC(Cc1ccc(C2CCC2)cc1)CN1CCC2(C1)CS(=O)(=O)C2] | 6-(3-(4-cyclobutylphenyl)-2- methylpropyl)-2-thia-6- azaspiro[3.4]octane 2,2-dioxide |
| 109B* 109C* | 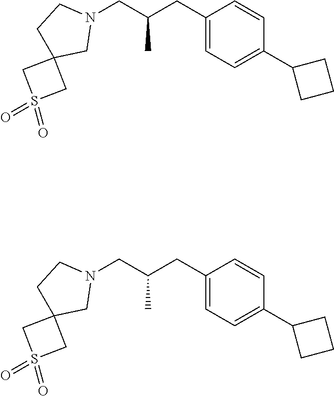 [SMILES: C[C@H](Cc1ccc(C2CCC2)cc1)CN1CCC2(C1)CS(=O)(=O)C2 ; C[C@@H](Cc1ccc(C2CCC2)cc1)CN1CCC2(C1)CS(=O)(=O)C2] | (R)-6-(3-(4-cyclobutylphenyl)-2- methylpropyl)-2-thia-6- azaspiro[3.4]octane 2,2-dioxide (S)-6-(3-(4-cyclobutylphenyl)-2- methylpropyl)-2-thia-6- azaspiro[3.4]octane 2,2-dioxide |
| 110 | 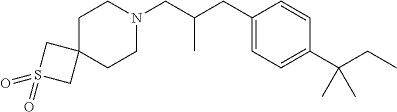 [SMILES: CCC(C)(C)c1ccc(CC(C)CN2CCC3(CC2)CS(=O)(=O)C3)cc1] | 7-(2-Methyl-3-(4-(tert- pentyl)phenyl)propyl)-2-thia-7- azaspiro[3.5]nonane 2,2-dioxide |
| 111A | 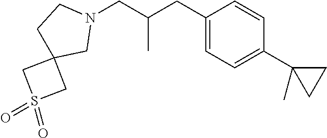 [SMILES: CC(Cc1ccc(C2(C)CC2)cc1)CN1CCC2(C1)CS(=O)(=O)C2] | 6-(2-Methyl-3-(4-(1- methylcyclopropyl)phenyl)propyl)- 2-thia-6-azaspiro[3.4]octane 2,2-dioxide |
| 111B* 111C* | 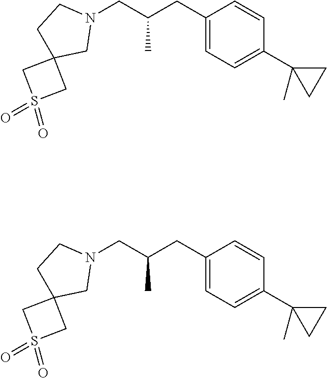 [SMILES: C[C@@H](Cc1ccc(C2(C)CC2)cc1)CN1CCC2(C1)CS(=O)(=O)C2 ; C[C@H](Cc1ccc(C2(C)CC2)cc1)CN1CCC2(C1)CS(=O)(=O)C2] | (R)-6-(2-Methyl-3-(4-(1- methylcyclopropyl)phenyl)propyl)- 2-thia-6-azaspiro[3.4]octane 2,2-dioxide (S)-6-(2-methyl-3-(4-(1- methylcyclopropyl)phenyl)propyl)- 2-thia-6-azaspiro[3.4]octane 2,2-dioxide |
| 112 | 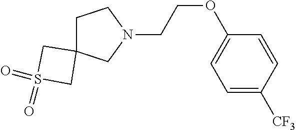 [SMILES: O=S1(=O)CC2(CCN(CCOc3ccc(C(F)(F)F)cc3)C2)C1] | 6-(2-(4- (Trifluoromethyl)phenoxy)ethyl)- 2-thia-6-azaspiro[3.4]octane- 2,2-dioxide |
| 113 | 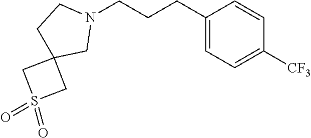 [SMILES: O=S1(=O)CC2(CCN(CCCc3ccc(C(F)(F)F)cc3)C2)C1] | 6-(3-(4- (Trifluoromethyl)phenyl)propyl)- 2-thia-6-azaspiro[3.4]octane- 2,2-dioxide |
| 114A* 114B* | 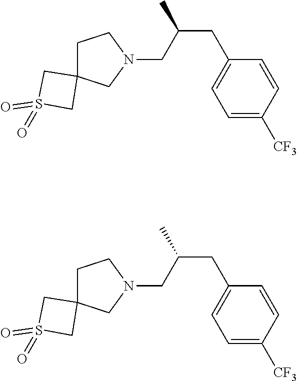 [SMILES: C[C@@H](Cc1ccc(C(F)(F)F)cc1)CN1CCC2(C1)CS(=O)(=O)C2 ; C[C@H](Cc1ccc(C(F)(F)F)cc1)CN1CCC2(C1)CS(=O)(=O)C2] | (S)-6-(2-Methyl-3-(4- (trifluoromethyl)phenyl)propyl)- 2-thia-6-azaspiro[3.4]octane 2,2- dioxide (R)-6-(2-Methyl-3-(4- (trifluoromethyl)phenyl)propyl)- 2-thia-6-azaspiro[3.4]octane 2,2- dioxide |
| 115A* 115B* | 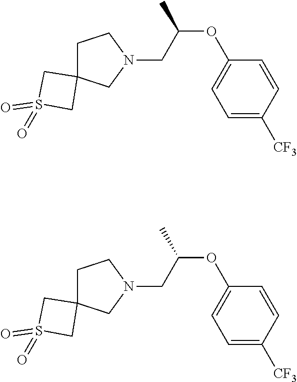 [SMILES: C[C@H](CN1CCC2(C1)CS(=O)(=O)C2)Oc1ccc(C(F)(F)F)cc1 ; C[C@@H](CN1CCC2(C1)CS(=O)(=O)C2)Oc1ccc(C(F)(F)F)cc1] | (R)-6-(2-(4- (Trifluoromethyl)phenoxy)propyl)- 2-thia-6-azaspiro[3.4]octane 2,2-dioxide (S)-6-(2-(4- (Trifluoromethyl)phenoxy)propyl)- 2-thia-6-azaspiro[3.4]octane 2,2-dioxide |
| 116A* 116B* | 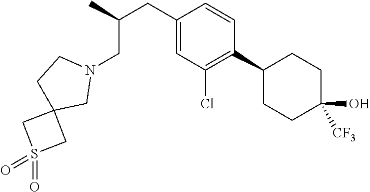 [SMILES: C[C@@H](Cc1ccc([C@H]2CC[C@](O)(C(F)(F)F)CC2)c(Cl)c1)CN1CCC2(C1)CS(=O)(=O)C2] | 6-((S)-3-(3-Chloro-4-((1s,4R)-4- hydroxy-4- (trifluoromethyl)cyclohexyl)phenyl)- 2-methylpropyl)-2-thia-6- azaspiro[3.4]octane 2,2-dioxide 6-((R)-3-(3-chloro-4-((1s,4S)-4- hydroxy-4- (trifluoromethyl)cyclohexyl)phenyl)- 2-methylpropyl)-2-thia-6- azaspiro[3.4]octane 2,2-dioxide 6-((S)-3-(3-Chloro-4-((1s,4S)-4- hydroxy-4- |
| | | (trifluoromethyl)cyclohexyl)phenyl)- |
| | | 2-methylpropyl)-2-thia-6- |
| | | azaspiro[3.4]octane 2,2-dioxide |
| | | 6-((R)-3-(3-chloro-4-((1r,4R)-4- |
| | | hydroxy-4- |
| | | (trifluoromethyl)cyclohexyl)phenyl)- |
| | | 2-methylpropyl)-2-thia-6- |
| | | azaspiro[3.4]octane 2,2-dioxide |
| | 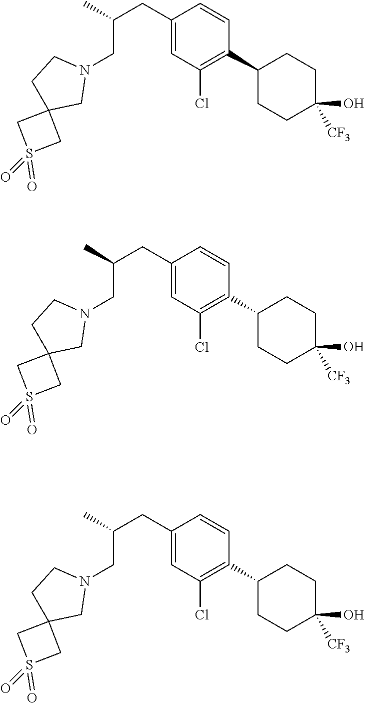 [SMILES: C[C@H](Cc1ccc([C@H]2CC[C@](O)(C(F)(F)F)CC2)c(Cl)c1)CN1CCC2(C1)CS(=O)(=O)C2 ; C[C@@H](Cc1ccc([C@H]2CC[C@@](O)(C(F)(F)F)CC2)c(Cl)c1)CN1CCC2(C1)CS(=O)(=O)C2 ; C[C@H](Cc1ccc([C@H]2CC[C@@](O)(C(F)(F)F)CC2)c(Cl)c1)CN1CCC2(C1)CS(=O)(=O)C2] | |
| 117A* 117B* | 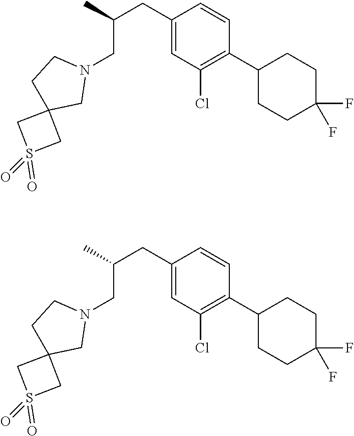 [SMILES: C[C@@H](Cc1ccc(C2CCC(F)(F)CC2)c(Cl)c1)CN1CCC2(C1)CS(=O)(=O)C2 ; C[C@H](Cc1ccc(C2CCC(F)(F)CC2)c(Cl)c1)CN1CCC2(C1)CS(=O)(=O)C2] | (S)-6-(3-(3-Chloro-4-(4,4- difluorocyclohexyl)phenyl)-2- methylpropyl)-2-thia-6- azaspiro[3.4]octane 2,2-dioxide (R)-6-(3-(3-Chloro-4-(4,4- difluorocyclohexyl)phenyl)-2- methylpropyl)-2-thia-6- azaspiro[3.4]octane 2,2-dioxide |
| 118A* 118B* | 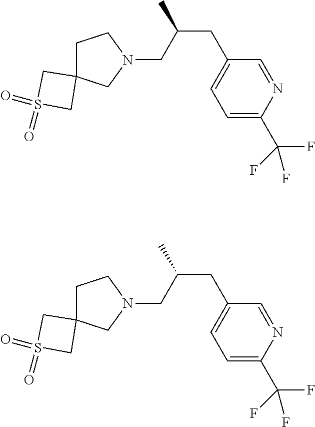 [SMILES: C[C@@H](Cc1ccc(C(F)(F)F)nc1)CN1CCC2(C1)CS(=O)(=O)C2 ; C[C@H](Cc1ccc(C(F)(F)F)nc1)CN1CCC2(C1)CS(=O)(=O)C2] | (S)-6-(2-Methyl-3-(6- (trifluoromethyl)pyridin-3- yl)propyl)-2-thia-6- azaspiro[3.4]octane 2,2-dioxide (R)-6-(2-Methyl-3-(6- (trifluoromethyl)pyridin-3- yl)propyl)-2-thia-6- azaspiro[3.4]octane 2,2-dioxide |
| 119A* 119B* | 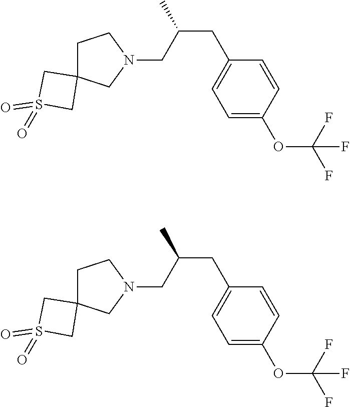 [SMILES: C[C@H](Cc1ccc(OC(F)(F)F)cc1)CN1CCC2(C1)CS(=O)(=O)C2 ; C[C@@H](Cc1ccc(OC(F)(F)F)cc1)CN1CCC2(C1)CS(=O)(=O)C2] | (S)-6-(2-Methyl-3-(4- (trifluoromethoxy)phenyl)propyl)- 2-thia-6-azaspiro[3.4]octane 2,2-dioxide (R)-6-(2-Methyl-3-(4- (trifluoromethoxy)phenyl)propyl)- 2-thia-6-azaspiro[3.4]octane 2,2-dioxide |
| 120 | 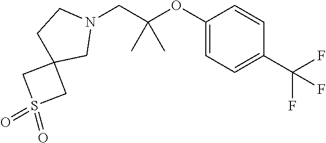 [SMILES: CC(C)(CN1CCC2(C1)CS(=O)(=O)C2)Oc1ccc(C(F)(F)F)cc1] | 6-(2-Methyl-2-(4- (trifluoromethyl)phenoxy)propyl)- 2-thia-6-azaspiro[3.4]octane 2,2-dioxide |
| 121A* 121B* | 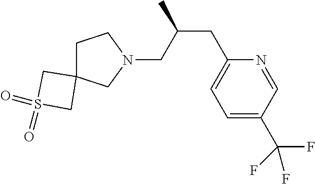 [SMILES: C[C@@H](Cc1ccc(C(F)(F)F)cn1)CN1CCC2(C1)CS(=O)(=O)C2] | (S)-6-(2-Methyl-3-(5- (trifluoromethyl)pyridin-2- yl)propyl)-2-thia-6- azaspiro[3.4]octane 2,2-dioxide (R)-6-(2-Methyl-3-(5- (trifluoromethyl)pyridin-2- yl)propyl)-2-thia-6- azaspiro[3.4]octane 2,2-dioxide |
| | 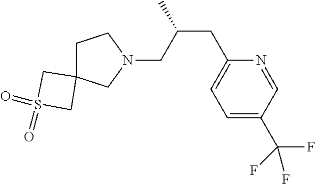 [SMILES: C[C@H](Cc1ccc(C(F)(F)F)cn1)CN1CCC2(C1)CS(=O)(=O)C2] | |
| 122A* 122B* | 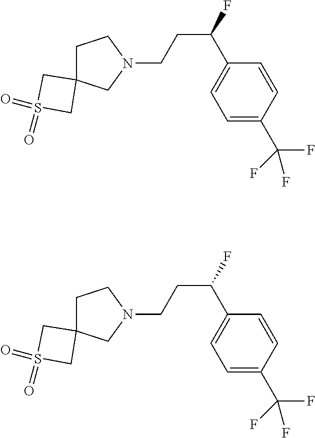 [SMILES: O=S1(=O)CC2(CCN(CC[C@@H](F)c3ccc(C(F)(F)F)cc3)C2)C1 ; O=S1(=O)CC2(CCN(CC[C@H](F)c3ccc(C(F)(F)F)cc3)C2)C1] | (R)-6-(3-Fluoro-3-(4- (trifluoromethyl)phenyl)propyl)- 2-thia-6-azaspiro[3.4]octane 2,2- dioxide (S)-6-(3-Fluoro-3-(4- (trifluoromethyl)phenyl)propyl)- 2-thia-6-azaspiro[3.4]octane 2,2- dioxide |
| 123A* 123B* | 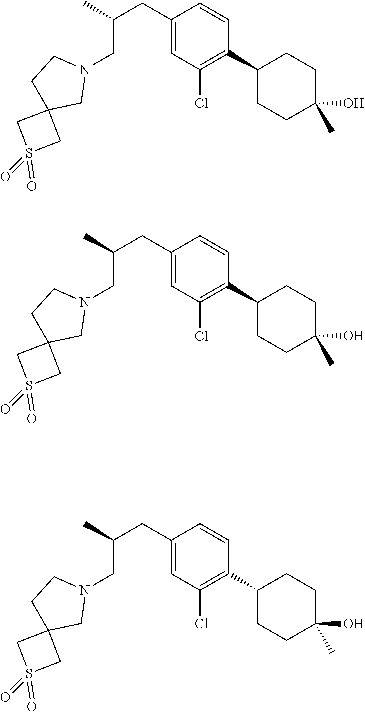 [SMILES: C[C@H](Cc1ccc([C@H]2CC[C@](C)(O)CC2)c(Cl)c1)CN1CCC2(C1)CS(=O)(=O)C2 ; C[C@@H](Cc1ccc([C@H]2CC[C@](C)(O)CC2)c(Cl)c1)CN1CCC2(C1)CS(=O)(=O)C2 ; C[C@@H](Cc1ccc([C@H]2CC[C@](C)(O)CC2)c(Cl)c1)CN1CCC2(C1)CS(=O)(=O)C2] | 6-((R)-3-(3-Chloro-4-((1r,4R)-4- hydroxy-4- methylcyclohexyl)phenyl)-2- methylpropyl)-2-thia-6- azaspiro[3.4]octane 2,2-dioxide 6-((S)-3-(3-chloro-4-((1r,4S)-4- hydroxy-4- methylcyclohexyl)phenyl)-2- methylpropyl)-2-thia-6- azaspiro[3.4]octane 2,2-dioxide 6-((S)-3-(3-Chloro-4-((1r,4S)-4- hydroxy-4- methylcyclohexyl)phenyl)-2- methylpropyl)-2-thia-6- azaspiro[3.4]octane 2,2-dioxide |
| | 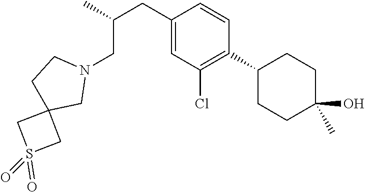 [SMILES: C[C@H](Cc1ccc([C@H]2CC[C@](C)(O)CC2)c(Cl)c1)CN1CCC2(C1)CS(=O)(=O)C2] | 6-((R)-3-(3-chloro-4-((1r,4R)-4- hydroxy-4- methylcyclohexyl)phenyl)-2- methylpropyl)-2-thia-6- azaspiro[3.4]octane 2,2-dioxide |
| 124A* 124B* | 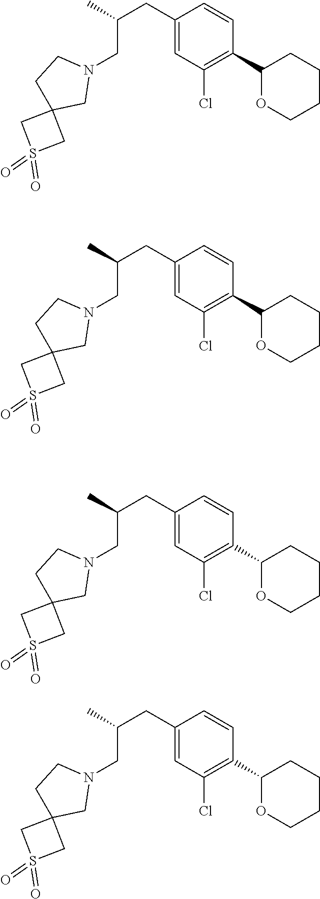 | 6-((R)-3-(3-Chloro-4-((R)- tetrahydro-2H-pyran-2- yl)phenyl)-2-methylpropyl)-2- thia-6-azaspiro[3.4]octane 2,2- dioxide 6-((S)-3-(3-chloro-4-((R)- tetrahydro-2H-pyran-2- yl)phenyl)-2-methylpropyl)-2- thia-6-azaspiro[3.4]octane 2,2- dioxide 6-((R)-3-(3-Chloro-4-((S)- tetrahydro-2H-pyran-2- yl)phenyl)-2-methylpropyl)-2- thia-6-azaspiro[3.4]octane 2,2- dioxide 6-((S)-3-(3-chloro-4-((S)- tetrahydro-2H-pyran-2- yl)phenyl)-2-methylpropyl)-2- thia-6-azaspiro[3.4]octane 2,2- dioxide |
| 125 | 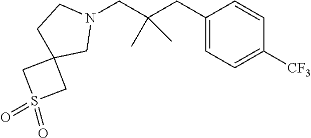 [SMILES: CC(C)(Cc1ccc(C(F)(F)F)cc1)CN1CCC2(C1)CS(=O)(=O)C2] | 6-(2,2-Dimethyl-3-(4- (trifluoromethyl)phenyl)propyl)- 2-thia-6-azaspiro[3.4]octane-2,2- dioxide |
| 126A* 126B* | 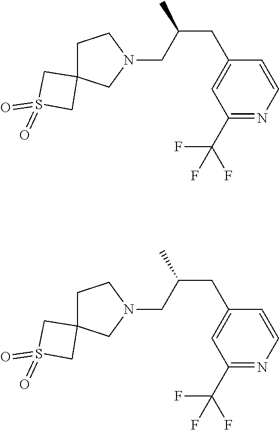 [SMILES: C[C@@H](Cc1ccnc(C(F)(F)F)c1)CN1CCC2(C1)CS(=O)(=O)C2 ; C[C@H](Cc1ccnc(C(F)(F)F)c1)CN1CCC2(C1)CS(=O)(=O)C2] | (S)-6-(2-Methyl-3-(2- (trifluoromethyl)pyridin-4- yl)propyl)-2-thia-6- azaspiro[3.4]octane 2,2-dioxide (R)-6-(2-Methyl-3-(2- (trifluoromethyl)pyridin-4- yl)propyl)-2-thia-6- azaspiro[3.4]octane 2,2-dioxide |
| 127 | 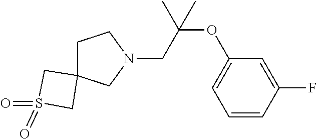 [SMILES: CC(C)(CN1CCC2(C1)CS(=O)(=O)C2)Oc1cccc(F)c1] | 6-(2-(3-Fluorophenoxy)-2- methylpropyl)-2-thia-6- azaspiro[3.4]octane-2,2-dioxide |
| 128A* 128B* | 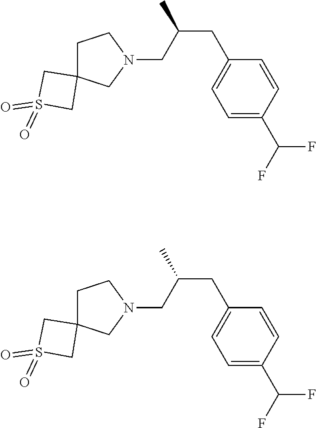 [SMILES: C[C@@H](Cc1ccc(C(F)F)cc1)CN1CCC2(C1)CS(=O)(=O)C2 ; C[C@H](Cc1ccc(C(F)F)cc1)CN1CCC2(C1)CS(=O)(=O)C2] | (S)-6-(3-(4- (Difluoromethyl)phenyl)-2- methylpropyl)-2-thia-6- azaspiro[3.4]octane 2,2-dioxide (R)-6-(3-(4- (Difluoromethyl)phenyl)-2- methylpropyl)-2-thia-6- azaspiro[3.4]octane 2,2-dioxide |
| 129 | 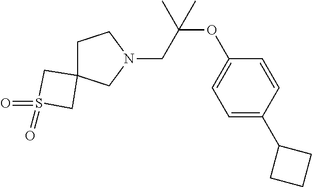 [SMILES: CC(C)(CN1CCC2(C1)CS(=O)(=O)C2)Oc1ccc(C2CCC2)cc1] | 6-(2-(4-Cyclobutylphenoxy)-2- methylpropyl)-2-thia-6- azaspiro[3.4]octane-2,2-dioxide |
| 130 | 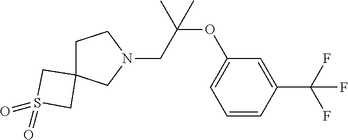 [SMILES: CC(C)(CN1CCC2(C1)CS(=O)(=O)C2)Oc1cccc(C(F)(F)F)c1] | 6-(2-Methyl-2-(3- (trifluoromethyl)phenoxy)propyl)- 2-thia-6-azaspiro[3.4]octane- 2,2-dioxide |
| 131 | 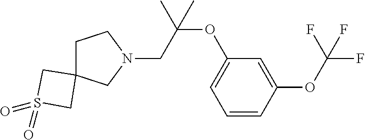 [SMILES: CC(C)(CN1CCC2(C1)CS(=O)(=O)C2)Oc1cccc(OC(F)(F)F)c1] | 6-(2-Methyl-2-(3- (trifluoromethoxy)phenoxy)propyl)- 2-thia-6-azaspiro[3.4]octane 2,2-dioxide |
| 132 | 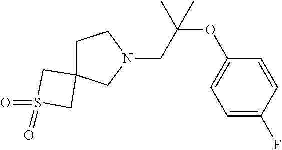 [SMILES: CC(C)(CN1CCC2(C1)CS(=O)(=O)C2)Oc1ccc(F)cc1] | 6-(2-(4-Fluorophenoxy)-2- methylpropyl)-2-thia-6- azaspiro[3.4]octane 2,2-dioxide |
| 133 | 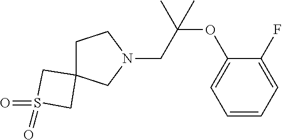 [SMILES: CC(C)(CN1CCC2(C1)CS(=O)(=O)C2)Oc1ccccc1F] | 6-(2-(2-Fluorophenoxy)-2- methylpropyl)-2-thia-6- azaspiro[3.4]octane 2,2-dioxide |
| 135 | 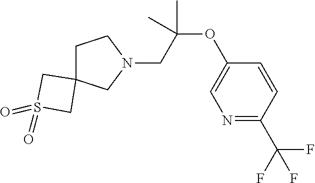 [SMILES: CC(C)(CN1CCC2(C1)CS(=O)(=O)C2)Oc1ccc(C(F)(F)F)nc1] | 6-(2-Methyl-2-((6- (trifluoromethyl)pyridin-3- yl)oxy)propyl)-2-thia-6- azaspiro[3.4]octane-2,2-dioxide |
| 136 | 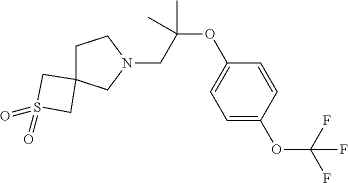 [SMILES: CC(C)(CN1CCC2(C1)CS(=O)(=O)C2)Oc1ccc(OC(F)(F)F)cc1] | 6-(2-Methyl-2-(4- (trifluoromethoxy)phenoxy)propyl)- 2-thia-6-azaspiro[3.4]octane 2,2-dioxide |
| 138 | 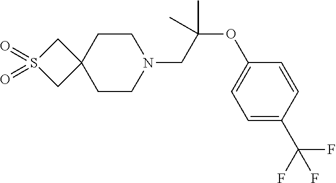 [SMILES: CC(C)(CN1CCC2(CC1)CS(=O)(=O)C2)Oc1ccc(C(F)(F)F)cc1] | 7-(2-Methyl-2-(4- (trifluoromethyl)phenoxy)propyl)- 2-thia-7-azaspiro[3.5]nonane 2,2-dioxide |
| 139 | 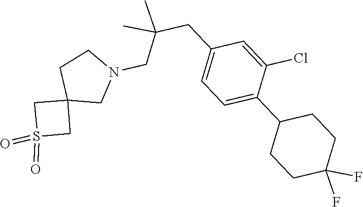 [SMILES: CC(C)(Cc1ccc(C2CCC(F)(F)CC2)c(Cl)c1)CN1CCC2(C1)CS(=O)(=O)C2] | 6-(3-(3-Chloro-4-(4,4- difluorocyclohexyl)phenyl)-2,2- dimethylpropyl)-2-thia-6- azaspiro[3.4]octane 2,2-dioxide |
| 140 | 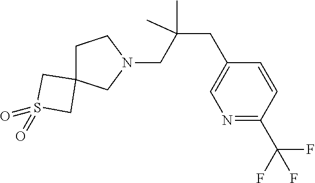 [SMILES: CC(C)(Cc1ccc(C(F)(F)F)nc1)CN1CCC2(C1)CS(=O)(=O)C2] | 6-(2,2-Dimethyl-3-(6- (trifluoromethyl)pyridin-3- yl)propyl)-2-thia-6- azaspiro[3.4]octane-2,2-dioxide |
| 141A* 141B* | 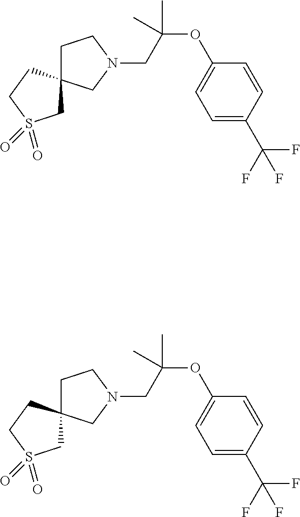 [SMILES: CC(C)(CN1CC[C@@]2(CCS(=O)(=O)C2)C1)Oc1ccc(C(F)(F)F)cc1 ; CC(C)(CN1CC[C@]2(CCS(=O)(=O)C2)C1)Oc1ccc(C(F)(F)F)cc1] | (R)-7-(2-Methyl-2-(4- (trifluoromethyl)phenoxy)propyl)- 2-thia-7-azaspiro[4.4]nonane 2,2-dioxide (S)-7-(2-Methyl-2-(4- (trifluoromethyl)phenoxy)propyl)- 2-thia-7-azaspiro[4.4]nonane 2,2-dioxide |
| 142 | 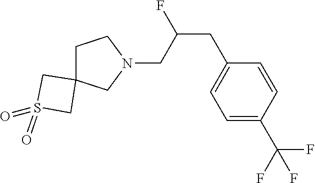 [SMILES: O=S1(=O)CC2(CCN(CC(F)Cc3ccc(C(F)(F)F)cc3)C2)C1] | 6-(2-Fluoro-3-(4- (trifluoromethyl)phenyl)propyl)- 2-thia-6-azaspiro[3.4]octane 2,2- dioxide |
| 143 | 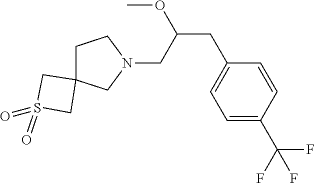 [SMILES: COC(Cc1ccc(C(F)(F)F)cc1)CN1CCC2(C1)CS(=O)(=O)C2] | 6-(2-Methoxy-3-(4- (trifluoromethyl)phenyl)propyl)- 2-thia-6-azaspiro[3.4]octane 2,2- dioxide |
| 144 | 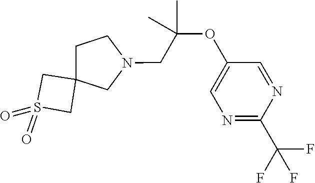 [SMILES: CC(C)(CN1CCC2(C1)CS(=O)(=O)C2)Oc1cnc(C(F)(F)F)nc1] | 6-(2-Methyl-2-((2- (trifluoromethyl)pyrimidin-5- yl)oxy)propyl)-2-thia-6- azaspiro[3.4]octane-2,2-dioxide |
| 145A* 145B* | 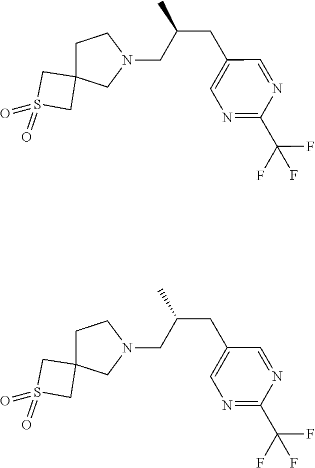 [SMILES: C[C@@H](Cc1cnc(C(F)(F)F)nc1)CN1CCC2(C1)CS(=O)(=O)C2 ; C[C@H](Cc1cnc(C(F)(F)F)nc1)CN1CCC2(C1)CS(=O)(=O)C2] | (S)-6-(2-Methyl-3-(2- (trifluoromethyl)pyrimidin-5- yl)propyl)-2-thia-6- azaspiro[3.4]octane 2,2-dioxide (R)-6-(2-Methyl-3-(2- (trifluoromethyl)pyrimidin-5- yl)propyl)-2-thia-6- azaspiro[3.4]octane 2,2-dioxide |
| 148A* 148B* | 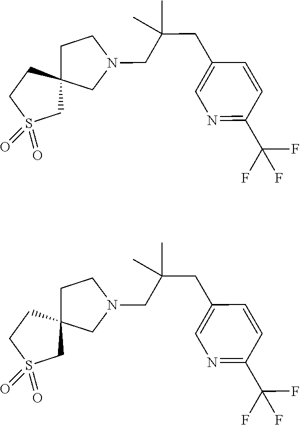 [SMILES: CC(C)(Cc1ccc(C(F)(F)F)nc1)CN1CC[C@]2(CCS(=O)(=O)C2)C1 ; CC(C)(Cc1ccc(C(F)(F)F)nc1)CN1CC[C@@]2(CCS(=O)(=O)C2)C1] | (S)-7-(2,2-Dimethyl-3-(6- (trifluoromethyl)pyridin-3- yl)propyl)-2-thia-7- azaspiro[4.4]nonane 2,2-dioxide (R)-7-(2,2-Dimethyl-3-(6- (trifluoromethyl)pyridin-3- yl)propyl)-2-thia-7- azaspiro[4.4]nonane 2,2-dioxide |
| 149 | 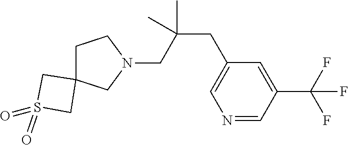 [SMILES: CC(C)(Cc1cncc(C(F)(F)F)c1)CN1CCC2(C1)CS(=O)(=O)C2] | 6-(2,2-Dimethyl-3-(5- (trifluoromethyl)pyridin-3- yl)propyl)-2-thia-6- azaspiro[3.4]octane-2,2-dioxide |
| 150 | 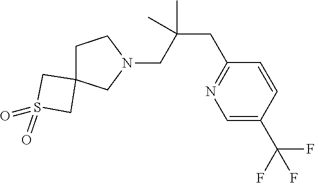 [SMILES: CC(C)(Cc1ccc(C(F)(F)F)cn1)CN1CCC2(C1)CS(=O)(=O)C2] | 6-(2,2-Dimethyl-3-(5- (trifluoromethyl)pyridin-2- yl)propyl)-2-thia-6- azaspiro[3.4]octane-2,2-dioxide |
| 152 | 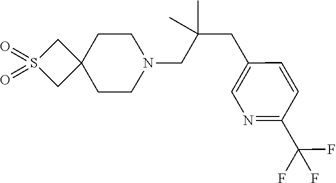 [SMILES: CC(C)(Cc1ccc(C(F)(F)F)nc1)CN1CCC2(CC1)CS(=O)(=O)C2] | 7-(2,2-Dimethyl-3-(6- (trifluoromethyl)pyridin-3- yl)propyl)-2-thia-7- azaspiro[3.5]nonane-2,2-dioxide |
| 153 | 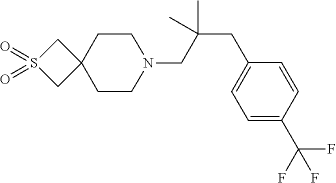 [SMILES: CC(C)(Cc1ccc(C(F)(F)F)cc1)CN1CCC2(CC1)CS(=O)(=O)C2] | 7-(2,2-Dimethyl-3-(4- (trifluoromethyl)phenyl)propyl)- 2-thia-7-azaspiro[3.5]nonane- 2,2-dioxide |
| 154A* 154B* | 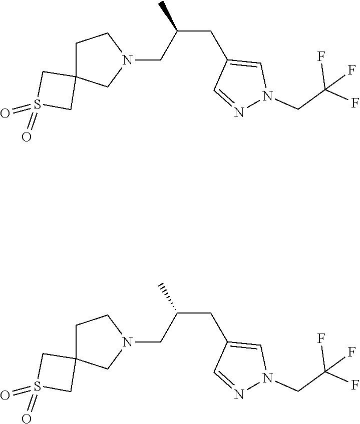 [SMILES: C[C@@H](Cc1cnn(CC(F)(F)F)c1)CN1CCC2(C1)CS(=O)(=O)C2 ; C[C@H](Cc1cnn(CC(F)(F)F)c1)CN1CCC2(C1)CS(=O)(=O)C2] | (S)-6-(2-Methyl-3-(1-(2,2,2- trifluoroethyl)-1H-pyrazol-4- yl)propyl)-2-thia-6- azaspiro[3.4]octane 2,2-dioxide (R)-6-(2-Methyl-3-(1-(2,2,2- trifluoroethyl)-1H-pyrazol-4- yl)propyl)-2-thia-6- azaspiro[3.4]octane 2,2-dioxide |
| 155 | 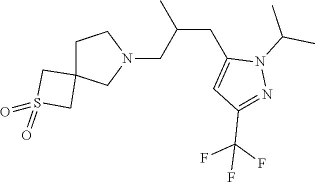 [SMILES: CC(Cc1cc(C(F)(F)F)nn1C(C)C)CN1CCC2(C1)CS(=O)(=O)C2] | (S)-6-(3-(1-Isopropyl-3- (trifluoromethyl)-1H-pyrazol-5- yl)-2-methylpropyl)-2-thia-6- azaspiro[3.4]octane 2,2-dioxide (R)-6-(3-(1-isopropyl-3- (trifluoromethyl)-1H-pyrazol-5- yl)-2-methylpropyl)-2-thia-6- azaspiro[3.4]octane 2,2-dioxide |
| 156 | 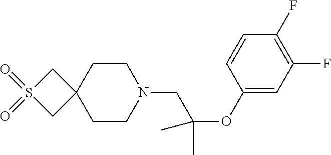 [SMILES: CC(C)(CN1CCC2(CC1)CS(=O)(=O)C2)Oc1ccc(F)c(F)c1] | 7-(2-(3,4-Difluorophenoxy)-2- methylpropyl)-2-thia-7- azaspiro[3.5]nonane 2,2-dioxide |
| 157 | 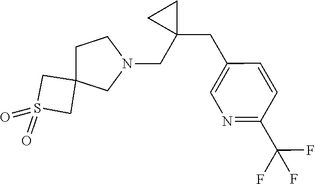 [SMILES: O=S1(=O)CC2(CCN(CC3(Cc4ccc(C(F)(F)F)nc4)CC3)C2)C1] | 6-((1-((6- (Trifluoromethyl)pyridin-3- yl)methyl)cyclopropyl)methyl)- 2-thia-6-azaspiro[3.4]octane 2,2- dioxide |
| 158 | 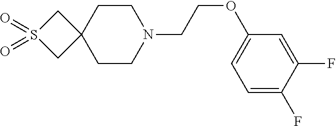 [SMILES: O=S1(=O)CC2(CCN(CCOc3ccc(F)c(F)c3)CC2)C1] | 7-(2-(3,4- Difluorophenoxy)ethyl)-2-thia- 7-azaspiro[3.5]nonane 2,2- dioxide |
| 201 | 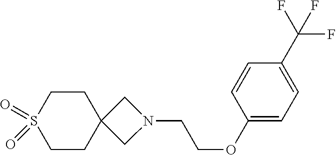 [SMILES: O=S1(=O)CCC2(CC1)CN(CCOc1ccc(C(F)(F)F)cc1)C2] | 2-(2-(4- (Trifluoromethyl)phenoxy)ethyl)- 7-thia-2-azaspiro[3.5]nonane 7,7-dioxide |
| 202 | 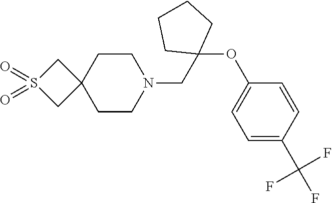 [SMILES: O=S1(=O)CC2(CCN(CC3(Oc4ccc(C(F)(F)F)cc4)CCCC3)CC2)C1] | 7-((1-(4- (Trifluoromethyl)phenoxy) cyclopentyl)methyl)-2-thia-7- azaspiro[3.5]nonane 2,2-dioxide |
| 203* | 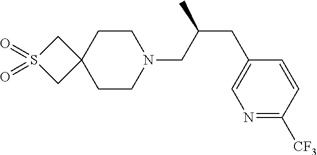 [SMILES: C[C@@H](Cc1ccc(C(F)(F)F)nc1)CN1CCC2(CC1)CS(=O)(=O)C2] | (S)-7-(2-Methyl-3-(6- (trifluoromethyl)pyridin-3- yl)propyl)-2-thia-7- azaspiro[3.5]nonane 2,2-dioxide |
| 204 | 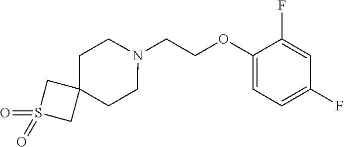 [SMILES: O=S1(=O)CC2(CCN(CCOc3ccc(F)cc3F)CC2)C1] | 7-(2-(2,4- Difluorophenoxy)ethyl)-2-thia- 7-azaspiro[3.5]nonane 2,2- dioxide |
| 205 | 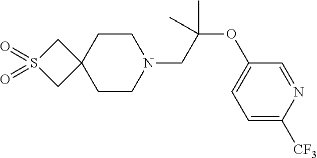 [SMILES: CC(C)(CN1CCC2(CC1)CS(=O)(=O)C2)Oc1ccc(C(F)(F)F)nc1] | 7-(2-Methyl-2-((6- (trifluoromethyl)pyridin-3- yl)oxy)propyl)-2-thia-7- azaspiro[3.5]nonane 2,2-dioxide |
| 206 | 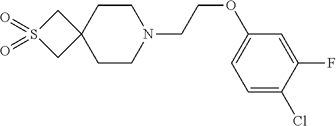 [SMILES: O=S1(=O)CC2(CCN(CCOc3ccc(Cl)c(F)c3)CC2)C1] | 7-(2-(4-Chloro-3- fluorophenoxy)ethyl)-2-thia-7- azaspiro[3.5]nonane 2,2-dioxide |
| 207A* 207B* | 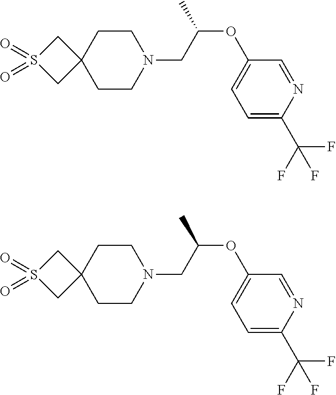 [SMILES: C[C@@H](CN1CCC2(CC1)CS(=O)(=O)C2)Oc1ccc(C(F)(F)F)nc1 ; C[C@H](CN1CCC2(CC1)CS(=O)(=O)C2)Oc1ccc(C(F)(F)F)nc1] | (S)-7-(2-((6- (Trifluoromethyl)pyridin-3- yl)oxy)propyl)-2-thia-7- azaspiro[3.5]nonane 2,2-dioxide (R)-7-(2-((6- (Trifluoromethyl)pyridin-3- yl)oxy)propyl)-2-thia-7- azaspiro[3.5]nonane 2,2-dioxide |
| 208 | 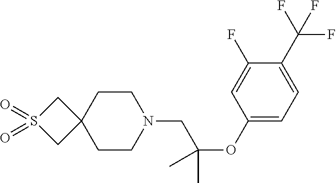 [SMILES: CC(C)(CN1CCC2(CC1)CS(=O)(=O)C2)Oc1ccc(C(F)(F)F)c(F)c1] | 7-(2-(3-Fluoro-4- (trifluoromethyl)phenoxy)-2- methylpropyl)-2-thia-7- azaspiro[3.5]nonane 2,2-dioxide |
| 209 | 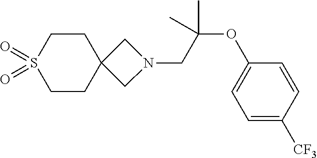 [SMILES: CC(C)(CN1CC2(CCS(=O)(=O)CC2)C1)Oc1ccc(C(F)(F)F)cc1] | 2-(2-Methyl-2-(4- (trifluoromethyl)phenoxy)propyl)- 7-thia-2-azaspiro[3.5]nonane 7,7-dioxide |
| 210 | 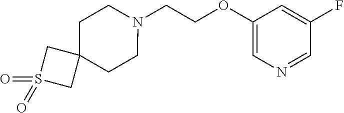 [SMILES: O=S1(=O)CC2(CCN(CCOc3cncc(F)c3)CC2)C1] | 7-(2-((5-Fluoropyridin-3- yl)oxy)ethyl)-2-thia-7- azaspiro[3.5]nonane 2,2-dioxide |
| 212A* 212B* | 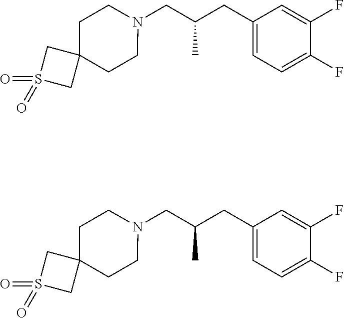 [SMILES: C[C@@H](Cc1ccc(F)c(F)c1)CN1CCC2(CC1)CS(=O)(=O)C2 ; C[C@H](Cc1ccc(F)c(F)c1)CN1CCC2(CC1)CS(=O)(=O)C2] | (S)-7-(3-(3,4-Difluorophenyl)-2- methylpropyl)-2-thia-7- azaspiro[3.5]nonane 2,2-dioxide (R)-7-(3-(3,4-difluorophenyl)-2- methylpropyl)-2-thia-7- azaspiro[3.5]nonane 2,2-dioxide |
| 213 | 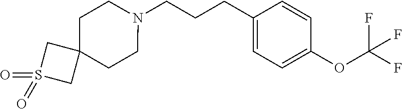 [SMILES: O=S1(=O)CC2(CCN(CCCc3ccc(OC(F)(F)F)cc3)CC2)C1] | 7-(3-(4- (Trifluoromethoxy)phenyl)propyl)- 2-thia-7-azaspiro[3.5]nonane 2,2-dioxide |
| 214 | 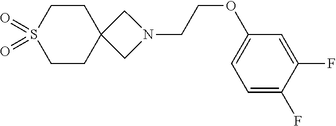 [SMILES: O=S1(=O)CCC2(CC1)CN(CCOc1ccc(F)c(F)c1)C2] | 2-(2-(3,4- Difluorophenoxy)ethyl)-7-thia- 2-azaspiro[3.5]nonane 7,7- dioxide |
| 215A* 215B* | 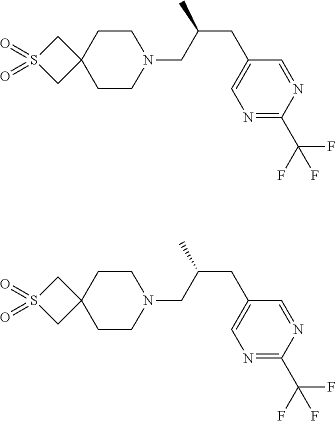 [SMILES: C[C@@H](Cc1cnc(C(F)(F)F)nc1)CN1CCC2(CC1)CS(=O)(=O)C2 ; C[C@H](Cc1cnc(C(F)(F)F)nc1)CN1CCC2(CC1)CS(=O)(=O)C2] | (S)-7-(2-Methyl-3-(2- (trifluoromethyl)pyrimidin-5- yl)propyl)-2-thia-7- azaspiro[3.5]nonane 2,2-dioxide (R)-7-(2-methyl-3-(2- (trifluoromethyl)pyrimidin-5- yl)propyl)-2-thia-7- azaspiro[3.5]nonane 2,2-dioxide |
| 216A* 216B* | 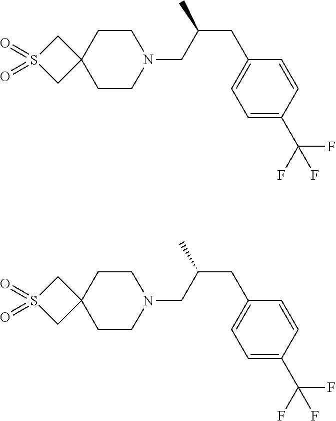 [SMILES: C[C@@H](Cc1ccc(C(F)(F)F)cc1)CN1CCC2(CC1)CS(=O)(=O)C2 ; C[C@H](Cc1ccc(C(F)(F)F)cc1)CN1CCC2(CC1)CS(=O)(=O)C2] | (S)-7-(2-Methyl-3-(4- (trifluoromethyl)phenyl)propyl)- 2-thia-7-azaspiro[3.5]nonane 2,2-dioxide (R)-7-(2-Methyl-3-(4- (trifluoromethyl)phenyl)propyl)- 2-thia-7-azaspiro[3.5]nonane 2,2-dioxide |
| 217* | 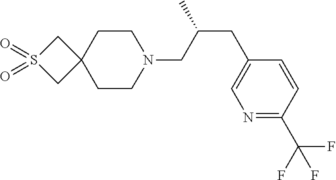 [SMILES: C[C@H](Cc1ccc(C(F)(F)F)nc1)CN1CCC2(CC1)CS(=O)(=O)C2] | (R)-7-(2-Methyl-3-(6- (trifluoromethyl)pyridin-3- yl)propyl)-2-thia-7- azaspiro[3.5]nonane 2,2-dioxide |
| 218 | 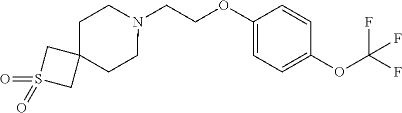 [SMILES: O=S1(=O)CC2(CCN(CCOc3ccc(OC(F)(F)F)cc3)CC2)C1] | 7-(2-(4- (Trifluoromethoxy)phenoxy)ethyl)- 2-thia-7-azaspiro[3.5]nonane 2,2-dioxide |
| 219 | 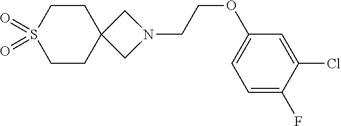 [SMILES: O=S1(=O)CCC2(CC1)CN(CCOc1ccc(F)c(Cl)c1)C2] | 2-(2-(3-Chloro-4- fluorophenoxy)ethyl)-7-thia-2- azaspiro[3.5]nonane 7,7-dioxide |
| 220 | 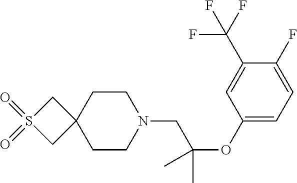 [SMILES: CC(C)(CN1CCC2(CC1)CS(=O)(=O)C2)Oc1ccc(F)c(C(F)(F)F)c1] | 7-(2-(4-Fluoro-3- (trifluoromethyl)phenoxy)-2- methylpropyl)-2-thia-7- azaspiro[3.5]nonane 2,2-dioxide |
| 221A* 221B* | 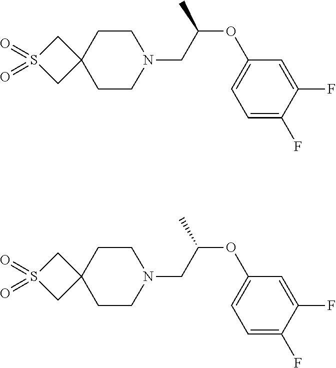 [SMILES: C[C@H](CN1CCC2(CC1)CS(=O)(=O)C2)Oc1ccc(F)c(F)c1 ; C[C@@H](CN1CCC2(CC1)CS(=O)(=O)C2)Oc1ccc(F)c(F)c1] | (S)-7-(2-(3,4- Difluorophenoxy)propyl)-2-thia- 7-azaspiro[3.5]nonane 2,2- dioxide (R)-7-(2-(3,4- difluorophenoxy)propyl)-2-thia- 7-azaspiro[3.5]nonane 2,2- dioxide |
| 222 | 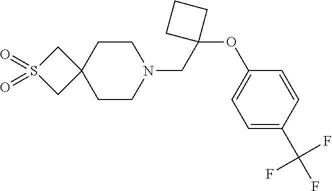 [SMILES: O=S1(=O)CC2(CCN(CC3(Oc4ccc(C(F)(F)F)cc4)CCC3)CC2)C1] | 7-((1-(4- (Trifluoromethyl)phenoxy) cyclobutyl)methyl)-2-thia-7- azaspiro[3.5]nonane 2,2-dioxide |
| 223 | 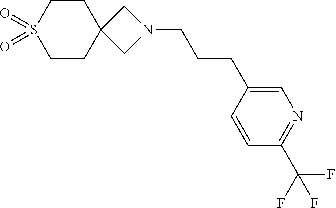 [SMILES: O=S1(=O)CCC2(CC1)CN(CCCc1ccc(C(F)(F)F)nc1)C2] | 2-(3-(6- (Trifluoromethyl)pyridin-3- yl)propyl)-7-thia-2- azaspiro[3.5]nonane 7,7-dioxide |
| 225 | 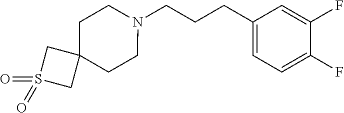 [SMILES: O=S1(=O)CC2(CCN(CCCc3ccc(F)c(F)c3)CC2)C1] | 7-(3-(3,4- Difluorophenyl)propyl)-2-thia-7- azaspiro[3.5]nonane 2,2-dioxide |
| 226 | 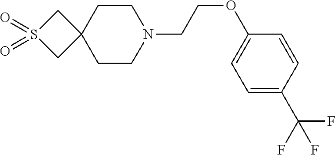 [SMILES: O=S1(=O)CC2(CCN(CCOc3ccc(C(F)(F)F)cc3)CC2)C1] | 7-(2-(4- (Trifluoromethyl)phenoxy)ethyl)- 2-thia-7-azaspiro[3.5]nonane 2,2-dioxide |
| 227A* 227B* | 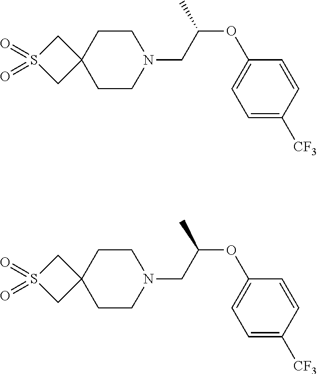 [SMILES: C[C@@H](CN1CCC2(CC1)CS(=O)(=O)C2)Oc1ccc(C(F)(F)F)cc1 ; C[C@H](CN1CCC2(CC1)CS(=O)(=O)C2)Oc1ccc(C(F)(F)F)cc1] | (S)-7-(2-(4- (Trifluoromethyl)phenoxy)propyl)- 2-thia-7-azaspiro[3.5]nonane 2,2-dioxide (R)-7-(2-(4- (trifluoromethyl)phenoxy)propyl)- 2-thia-7-azaspiro[3.5]nonane 2,2-dioxide |
| 228 | 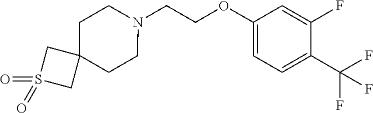 [SMILES: O=S1(=O)CC2(CCN(CCOc3ccc(C(F)(F)F)c(F)c3)CC2)C1] | 7-(2-(3-Fluoro-4- (trifluoromethyl)phenoxy)ethyl)- 2-thia-7-azaspiro[3.5]nonane 2,2-dioxide |
| 229 | 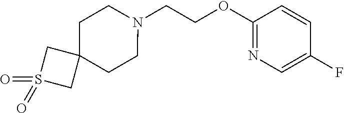 [SMILES: O=S1(=O)CC2(CCN(CCOc3ccc(F)cn3)CC2)C1] | 7-(2-((5-Fluoropyridin-2- yl)oxy)ethyl)-2-thia-7- azaspiro[3.5]nonane 2,2-dioxide |
| 230 | 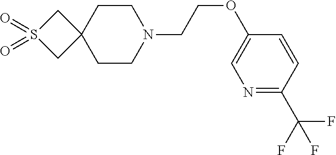 [SMILES: O=S1(=O)CC2(CCN(CCOc3ccc(C(F)(F)F)nc3)CC2)C1] | 7-(2-((6- (Trifluoromethyl)pyridin-3- yl)oxy)ethyl)-2-thia-7- azaspiro[3.5]nonane 2,2-dioxide |
| 231 | 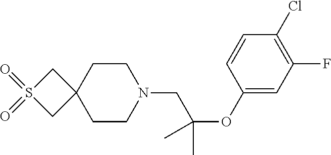 [SMILES: CC(C)(CN1CCC2(CC1)CS(=O)(=O)C2)Oc1ccc(Cl)c(F)c1] | 7-(2-(4-Chloro-3- fluorophenoxy)-2- methylpropyl)-2-thia-7- azaspiro[3.5]nonane 2,2-dioxide |
| 232A* 232B* | 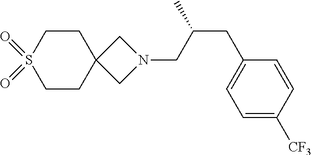 [SMILES: C[C@H](Cc1ccc(C(F)(F)F)cc1)CN1CC2(CCS(=O)(=O)CC2)C1] | (R)-2-(2-methyl-3-(4- (trifluoromethyl)phenyl)propyl)- 7-thia-2-azaspiro[3.5]nonane 7,7-dioxide |
| | 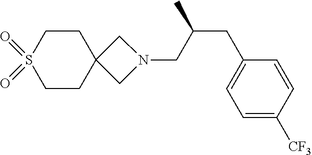 [SMILES: C[C@@H](Cc1ccc(C(F)(F)F)cc1)CN1CC2(CCS(=O)(=O)CC2)C1] | (S)-2-(2-methyl-3-(4- (trifluoromethyl)phenyl)propyl)- 7-thia-2-azaspiro[3.5]nonane 7,7-dioxide |
| 233 | 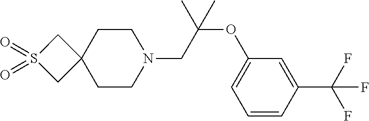 [SMILES: CC(C)(CN1CCC2(CC1)CS(=O)(=O)C2)Oc1cccc(C(F)(F)F)c1] | 7-(2-Methyl-2-(3- (trifluoromethyl)phenoxy)propyl)- 2-thia-7-azaspiro[3.5]nonane 2,2-dioxide |
| 234A* 234B* | 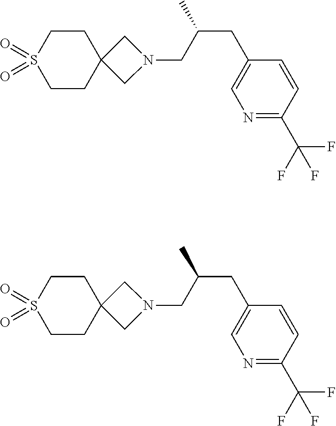 [SMILES: C[C@H](Cc1ccc(C(F)(F)F)nc1)CN1CC2(CCS(=O)(=O)CC2)C1 ; C[C@@H](Cc1ccc(C(F)(F)F)nc1)CN1CC2(CCS(=O)(=O)CC2)C1] | (R)-2-(2-methyl-3-(6- (trifluoromethyl)pyridin-3- yl)propyl)-7-thia-2- azaspiro[3.5]nonane 7,7-dioxide (S)-2-(2-methyl-3-(6- (trifluoromethyl)pyridin-3- yl)propyl)-7-thia-2- azaspiro[3.5]nonane 7,7-dioxide |
| 235A* 235B* | 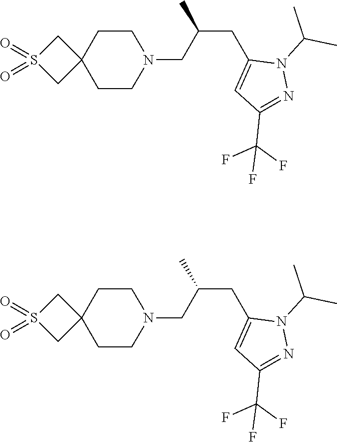 [SMILES: CC(C)n1nc(C(F)(F)F)cc1C[C@H](C)CN1CCC2(CC1)CS(=O)(=O)C2 ; CC(C)n1nc(C(F)(F)F)cc1C[C@@H](C)CN1CCC2(CC1)CS(=O)(=O)C2] | (R)-6-(3-(1-Isopropyl-3- (trifluoromethyl)-1H-pyrazol-5- yl)-2-methylpropyl)-2-thia-6- azaspiro[3.4]octane 2,2-dioxide (S)-6-(3-(1-isopropyl-3- (trifluoromethyl)-1H-pyrazol-5- yl)-2-methylpropyl)-2-thia-6- azaspiro[3.4]octane 2,2-dioxide |
| 236 | 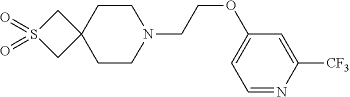 [SMILES: O=S1(=O)CC2(CCN(CCOc3ccnc(C(F)(F)F)c3)CC2)C1] | 7-(2-((2- (Trifluoromethyl)pyridin-4- yl)oxy)ethyl)-2-thia-7- azaspiro[3.5]nonane 2,2-dioxide |
| 237A* 237B* | 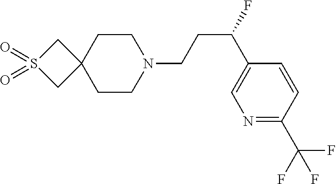 [SMILES: O=S1(=O)CC2(CCN(CC[C@H](F)c3ccc(C(F)(F)F)nc3)CC2)C1] | (S)-7-(3-Fluoro-3-(6- (trifluoromethyl)pyridin-3- yl)propyl)-2-thia-7- azaspiro[3.5]nonane 2,2-dioxide |
| | 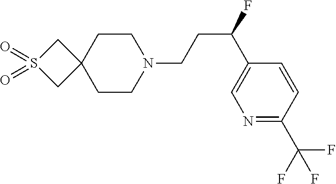 [SMILES: O=S1(=O)CC2(CCN(CC[C@@H](F)c3ccc(C(F)(F)F)nc3)CC2)C1] | (R)-7-(3-fluoro-3-(6- (trifluoromethyl)pyridin-3- yl)propyl)-2-thia-7- azaspiro[3.5]nonane 2,2-dioxide |
| 238 | 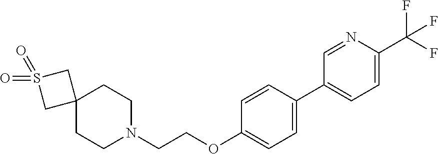 [SMILES: O=S1(=O)CC2(CCN(CCOc3ccc(-c4ccc(C(F)(F)F)nc4)cc3)CC2)C1] | 7-(2-(4-(6- (Trifluoromethyl)pyridin-3- yl)phenoxy)ethyl)-2-thia-7- azaspiro[3.5]nonane 2,2-dioxide |
| 239 | 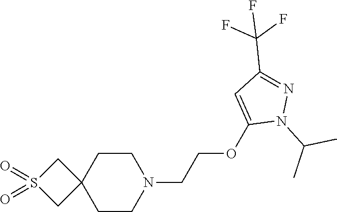 [SMILES: CC(C)n1nc(C(F)(F)F)cc1OCCN1CCC2(CC1)CS(=O)(=O)C2] | 7-[2-[4-[6-(Trifluoromethyl)-3- pyridyl]phenoxy]ethyl]-26-thia- 7-azaspiro[3.5]nonane 2,2- dioxide |
| 240 | 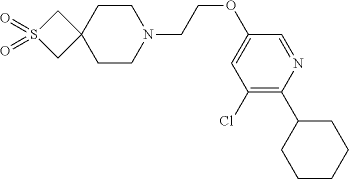 [SMILES: O=S1(=O)CC2(CCN(CCOc3cnc(C4CCCCC4)c(Cl)c3)CC2)C1] | 7-[2-[(5-Chloro-6-cyclohexyl-3- pyridyl)oxy]ethyl]-26-thia-7- azaspiro[3.5]nonane 2,2-dioxide |
| 241 | 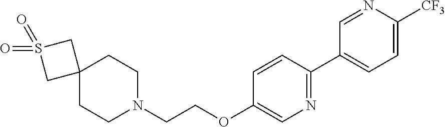 [SMILES: O=S1(=O)CC2(CCN(CCOc3ccc(-c4ccc(C(F)(F)F)nc4)nc3)CC2)C1] | 7-[2-[[6-[6-(Trifluoromethyl)-3- pyridyl]-3-pyridyl]oxy]ethyl]- 26-thia-7-azaspiro[3.5]nonane 2,2-dioxide |
| 242 | 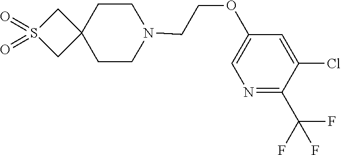 [SMILES: O=S1(=O)CC2(CCN(CCOc3cnc(C(F)(F)F)c(Cl)c3)CC2)C1] | 7-[2-[[5-Chloro-6- (trifluoromethyl)-3- pyridyl]oxy]ethyl]-26-thia-7- azaspiro[3.5]nonane 2,2-dioxide |
| 243 | 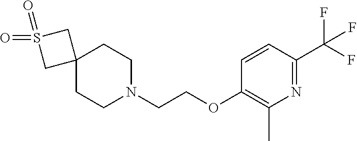 [SMILES: Cc1nc(C(F)(F)F)ccc1OCCN1CCC2(CC1)CS(=O)(=O)C2] | 7-[2-[2-Methyl-6- (trifluoromethyl)-3- pyridyl]oxylethyl]-26-thia-7- azaspiro[3.5]nonane 2,2-dioxide |
| 244 | 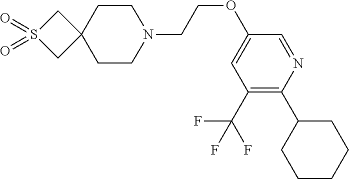 [SMILES: O=S1(=O)CC2(CCN(CCOc3cnc(C4CCCCC4)c(C(F)(F)F)c3)CC2)C1] | 7-(2-((6-Cyclohexyl-5- (trifluoromethyl)pyridin-3- yl)oxy)ethyl)-2-thia-7- azaspiro[3.5]nonane 2,2-dioxide |
| 245A* 245B* | 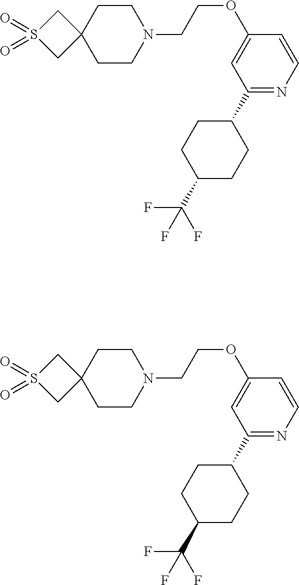 [SMILES: O=S1(=O)CC2(CCN(CCOc3ccnc([C@H]4CC[C@@H](C(F)(F)F)CC4)c3)CC2)C1 ; O=S1(=O)CC2(CCN(CCOc3ccnc([C@H]4CC[C@H](C(F)(F)F)CC4)c3)CC2)C1] | 7-(2-((2-((cis)-4- (Trifluoromethyl)cyclohexyl) pyridin-4-yl)oxy)ethyl)-2-thia-7- azaspiro[3.5]nonane 2,2-dioxide 7-(2-((2-((trans)-4- (trifluoromethyl)cyclohexyl) pyridin-4-yl)oxy)ethyl)-2-thia-7- azaspiro[3.5]nonane 2,2-dioxide |
| 246 | 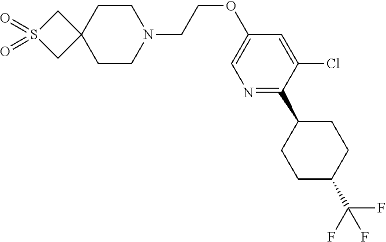 [SMILES: O=S1(=O)CC2(CCN(CCOc3cnc([C@H]4CC[C@H](C(F)(F)F)CC4)c(Cl)c3)CC2)C1] | 7-(2-((5-Chloro-6-((trans)-4- (trifluoromethyl)cyclohexyl) pyridin-3-yl)oxy)ethyl)-2-thia-7- azaspiro[3.5]nonane 2,2-dioxide |
| 247A* 247B* | 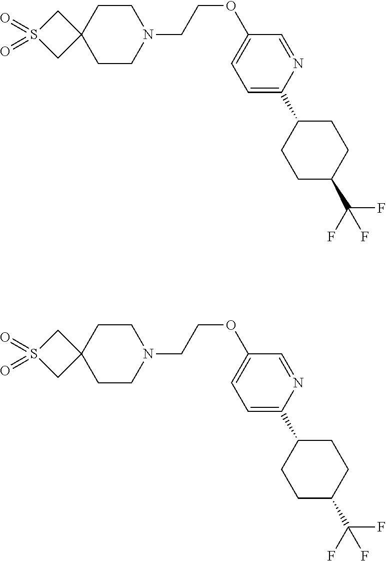 [SMILES: O=S1(=O)CC2(CCN(CCOc3ccc([C@H]4CC[C@H](C(F)(F)F)CC4)nc3)CC2)C1 ; O=S1(=O)CC2(CCN(CCOc3ccc([C@H]4CC[C@@H](C(F)(F)F)CC4)nc3)CC2)C1] | 7-(2-((6-((trans)-4- (Trifluoromethyl)cyclohexyl) pyridin-3-yl)oxy)ethyl)-2-thia-7- azaspiro[3.5]nonane 2,2-dioxide 7-(2-((6-((cis)-4- (trifluoromethyl)cyclohexyl) pyridin-3-yl)oxy)ethyl)-2-thia-7- azaspiro[3.5]nonane 2,2-dioxide |
| 248A | 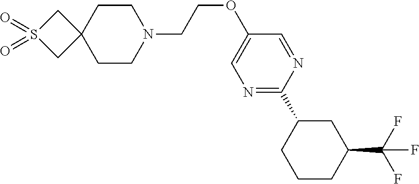 [SMILES: O=S1(=O)CC2(CCN(CCOc3cnc([C@H]4CCC[C@H](C(F)(F)F)C4)nc3)CC2)C1] | 7-(2-((2-((trans)-3- (Trifluoromethyl)cyclohexyl) pyrimidin-5-yl)oxy)ethyl)-2-thia-7- azaspiro[3.5]nonane 2,2-dioxide |
| 248B | 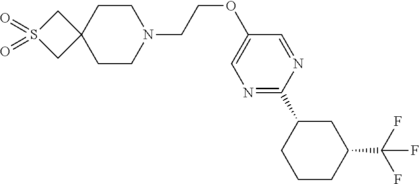 [SMILES: O=S1(=O)CC2(CCN(CCOc3cnc([C@H]4CCC[C@@H](C(F)(F)F)C4)nc3)CC2)C1] | 7-(2-((2-((cis)-3- (trifluoromethyl)cyclohexyl) pyrimidin-5-yl)oxy)ethyl)-2-thia- 7-azaspiro[3.5]nonane 2,2-dioxide |
| 249 | 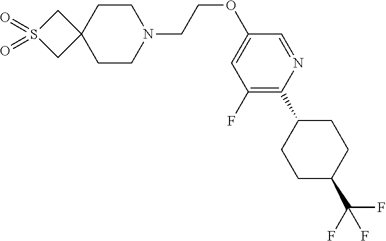 [SMILES: O=S1(=O)CC2(CCN(CCOc3cnc([C@H]4CC[C@H](C(F)(F)F)CC4)c(F)c3)CC2)C1] | 7-(2-((5-Fluoro-6-((trans)-4- (trifluoromethyl)cyclohexyl) pyridin-3-yl)oxy)ethyl)-2-thia-7- azaspiro[3.5]nonane 2,2-dioxide |

### III. Pharmaceutical Compositions and Modes of Administration

Compounds provided herein are usually administered in the form of pharmaceutical compositions. Thus, provided herein are also pharmaceutical compositions that comprise one or more of the compounds described herein or a pharmaceutically acceptable salt, a stereoisomer, or a mixture of stereoisomers thereof and one or more pharmaceutically acceptable excipients. Suitable pharmaceutically acceptable excipients may include, for example, inert solid diluents and fillers, liquid diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or tablet, such as enteric coated tablets. In making the pharmaceutical compositions that include at least one compound described herein or a pharmaceutically acceptable salt, a stereoisomer, or a mixture of stereoisomers thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propythydroxy-benzoates; sweetening agents; and flavoring agents.

The compositions that include at least one compound described herein or a pharmaceutically acceptable salt, a stereoisomer, or a mixture of stereoisomers thereof can be formulated so as to provide quick, sustained, or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, a stereoisomer, or a mixture of stereoisomers thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject. A dose may be administered once a day (QID), twice per day (BID), or more frequently, depending on the pharmacokinetic and pharmacodynamic properties, including absorption, distribution, metabolism, and excretion of the particular compound. In addition, toxicity factors may influence the dosage and administration regimen. When administered orally, the pill, capsule, or tablet may be ingested daily or less frequently for a specified period of time. The regimen may be repeated for a number of cycles of therapy.

### IV. Methods of Treatment

Described herein are methods for promoting myelination of central nervous system neurons in a subject suffering from a myelin-related disorder, the method comprising administering to the subject a therapeutically effective amount of a compound of Formula I, or a pharmaceutical composition comprising the same. In certain embodiments, the subject matter disclosed herein is directed to a compound of Formula I or a pharmaceutically acceptable salt thereof for promoting myelination of central nervous system neurons in a subject suffering from a myelin-related disorder. In another embodiment, the subject matter described herein is directed to the use of a compound of Formula I or a pharmaceutically acceptable salt thereof for the manufacture of a medicament for promoting myelination of central nervous system neurons in a subject suffering from a myelin-related disorder.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, inhibits enzyme mediated synthesis of one or more sterol intermediates in the cholesterol biosynthesis pathway.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, promotes accumulation of A8,9-unsaturated sterol intermediates in the cholesterol biosynthesis pathway.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, inhibits one or more of CYP51, sterol-14-reductase, or EBP enzyme mediated synthesis of sterol intermediates in the cholesterol biosynthesis pathway.

In certain embodiments, in the methods for promoting myelination of central nervous system neurons in a subject suffering from a myelin-related disorder, the compound of Formula I, or a pharmaceutically acceptable salt thereof, or a pharmaceutical composition comprising the same, induces, promotes, and/or modulates oligodendrocyte precursor cell (OPC) differentiation, proliferation and/or maturation. In certain embodiments, the induction of OPC differentiation is characterized by an increase in myelin basic protein (MBP) expression.

In certain embodiments, the subject matter described herein is directed to a method of treating a disorder in a subject in need thereof, the method comprising administering to the subject in need thereof a therapeutically effective amount of a compound of Formula I or a pharmaceutically acceptable salt thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof.

In certain embodiments, the subject matter disclosed herein is directed to a compound of Formula I or a pharmaceutically acceptable salt thereof, for use in treating a disorder in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof.

In certain embodiments, the subject matter disclosed herein is directed to the use of a compound of Formula I, or a pharmaceutically acceptable salt thereof, in the manufacture of a medicament for treating a disorder in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof.

In certain embodiments, the subject matter disclosed herein is directed to a method of promoting myelination in a subject in need thereof, comprising administering to the subject a therapeutically effective amount of a compound of Formula I, or a pharmaceutically acceptable salt thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof.

In certain embodiments, the subject matter disclosed herein is directed to a compound of Formula I, or a pharmaceutically acceptable salt thereof, or a pharmaceutical composition comprising the compound, for use in promoting myelination in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof.

In certain embodiments, the subject matter disclosed herein is directed to use of a compound of Formula I, or a pharmaceutically acceptable salt thereof, or a pharmaceutical composition comprising the compound, in the manufacture of a medicament for promoting myelination in a subject in need thereof. In certain embodiments, the subject has a myelin-related disorder. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof.

In certain embodiments, the subject matter disclosed herein is directed to a method of inducing endogenous oligodendrocyte precursor cell (OPC) differentiation in a subject in need thereof, the method comprising administering to the subject a therapeutically effective amount of a compound of Formula I, or a pharmaceutically acceptable salt thereof, or a pharmaceutical composition comprising the same. In certain embodiments, the subject is suffering from a myelin-related disorder. In certain embodiments, the myelin-related disorder is multiple sclerosis.

Such myelin-related disorders include, but are not limited to, multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, metachromatic leukodystrophy, trigeminal neuralgia, acute disseminated encephalitis, Guillian-Barre syndrome, Charcot-Marie-Tooth disease, Bell's palsy and radiation-induced demyelination.

The compound of Formula I or a pharmaceutically acceptable salt thereof can be administered alone or in combination with another agent to a subject suffering from a myelin-related disorder to promote myelination of neurons (e.g., neuronal axons). A myelin-related disorder can include any disease, condition (e.g., those occurring from traumatic spinal cord injury and cerebral infarction), or disorder resulting in abnormalities of the myelin sheath. Abnormalities can be caused by loss of myelin referred to as demyelination, dysfunctional myelin referred to as dysmyelination or failure to form enough myelin referred to as hypomyelination. A myelin related disorder as used herein can arise from a genetic disorder or from a variety of neurotoxic insults. In some embodiments, the compound of Formula I is a compound of Formula Ia, or a pharmaceutically acceptable salt thereof. In other embodiments, the compound of Formula I is a compound of Formula Ib, or a pharmaceutically acceptable salt thereof.

“Demyelination” as used herein, refers to the act of demyelinating, or the damage or loss of part or all of the myelin sheath insulating the nerves, and is the hallmark of myelin-related disorders. In certain embodiments, demyelination refers to the damage or loss of part or all of the myelin sheath insulating a subset of nerves in an individual, such as, for example, one or more nerves localized in a particular area of the body (e.g., neurons in the brain or spinal cord, or both brain and spinal cord; or the optic nerve).

Myelination of neurons requires oligodendrocytes. The term “myelination”, as used herein, refers to the generation of the nerve's myelin sheath by replacing myelin producing cells or restoring their function. The neurons that undergo remyelination may be in the brain, spinal cord, or both the brain and spinal cord.

“Promoting Myelination” as used herein refers to increasing the rate of myelin production rather than a mere net increase in the amount of myelin as compared to a baseline level of myelin production rate in a subject. An increase in the rate of myelin production can be determined using imaging techniques or functional measurements. In some embodiments, myelination is promoted by increasing the differentiation of OPCs, increasing the accumulation of 8,9-unsaturated sterol intermediates in the biosynthetic pathway, increasing the formation of OPCs, or any combinations thereof. Such activities may be evaluated, for example, using one or more in vitro assays, such as those described herein or known to one of skill in the art.

A “baseline level of myelin production rate” as used herein, refers to the rate of myelin production in subject being treated before the onset of treatment.

### V. Methods of Preparing Compounds of Formula I and Pharmaceutically Acceptable Salts Thereof

Compounds can be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g., Volume 3; Liebigs Annalen der Chemie, (9):1910-16, (1985); Helvetica Chimica Acta, 41:1052-60, (1958); Arzneimittel-Forschung, 40(12):1328-31, (1990), each of which are expressly incorporated by reference. Starting materials are generally available from commercial sources such as Aldrich Chemicals (Milwaukee, WI) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database).

Synthetic chemistry transformations and protecting group methodologies (protection and deprotection) useful in synthesizing compounds and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof.

Compounds may be prepared singly or as compound libraries comprising at least 2, for example 5 to 1,000 compounds, or 10 to 100 compounds. Libraries of compounds of Formula I, or pharmaceutically acceptable salts thereof, may be prepared by a combinatorial ‘split and mix’ approach or by multiple parallel syntheses using either solution phase or solid phase chemistry, by procedures known to those skilled in the art. Thus, according to a further aspect, there is provided a compound library comprising at least 2 compounds, or pharmaceutically acceptable salts thereof.

### EXAMPLES

The Examples provide exemplary methods for preparing compounds. Those skilled in the art will appreciate that other synthetic routes may be used to synthesize the compounds. Although specific starting materials and reagents are depicted and discussed in the Schemes, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art. The asterix (*) indicates an isolated isomer or isolated group of isomers, but that the stereochemistry has not been assigned.

### Example 102: 6-((2S)-3-(4-(2,2-Dimethylcyclopropyl)phenyl)-2-methylpropyl)-2-thia-6-azaspiro[3.4]octane 2,2-dioxide and 6-((2R)-3-(4-(2,2-dimethylcyclopropyl)phenyl)-2-methylpropyl)-2-thia-6-azaspiro[3.4]octane 2,2-dioxide

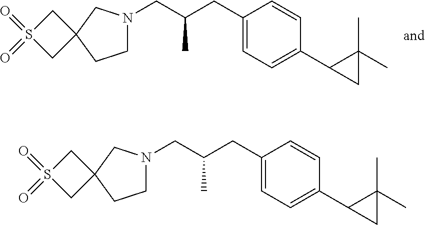 [SMILES: C[C@H](Cc1ccc(C2CC2(C)C)cc1)CN1CCC2(C1)CS(=O)(=O)C2 ; C[C@@H](Cc1ccc(C2CC2(C)C)cc1)CN1CCC2(C1)CS(=O)(=O)C2]

### Step 1: Synthesis of 1-iodo-4-(2-methylprop-1-enyl)benzene

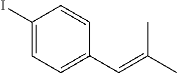

To a mixture of isopropyl(triphenyl)phosphonium iodide (11.2 g, 25.9 mmol) in THF (50 mL) was added tBuOK (2.90 g, 25.86 mmol) at 0° C. The mixture was stirred at 0° C. for 30 min, and then 4-iodobenzaldehyde (5 g, 21.55 mmol) was added at 0° C., the mixture was stirred at 25° C. for 16 h. The mixture was filtered and concentrated under vacuum. The residue was purified by silica gel chromatography (petroleum ether/ethyl acetate=100/1-80/1). 1-iodo-4-(2-methylprop-1-enyl)benzene (4.5 g, 17.4 mmol, 80.9% yield) was obtained. ¹H NMR (400 MHz, CDCl₃): δ ppm; 7.65-7.61 (m, 2H), 6.97 (d, J=8.3 Hz, 2H), 6.18 (s, 1H), 1.90 (d, J=1.2 Hz, 3H), 1.84 (d, J=1.0 Hz, 3H).

### Step 2: Synthesis of 1-(2,2-dimethylcyclopropyl)-4-iodo-benzene

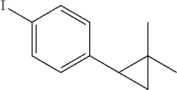

To a mixture of ZnEt₂ (1 M solution in hexanes, 4.74 mL) in DCM (10 mL) was added diidomethane (1.27 g, 4.74 mmol, 382.53 μL) at 0° C. under N₂. The mixture was stirred at 0° C. for 30 min, then 1-iodo-4-(2-methylprop-1-enyl)benzene (500 mg, 1.94 mmol) was added and the mixture was stirred at 0° C. for 1 h. The residue was poured into water (20 mL). The mixture was filtered. The aqueous phase was extracted with ethyl acetate (50 mL×3). The combined organic phase was washed with brine (50 mL), dried with anhydrous Na₂SO₄, filtered and concentrated under vacuum. The residue was purified by prep-HPLC (column: Phenomenex luna C18 150×40 mm, 15 m; mobile phase: [water (0.1% TFA)-ACN] 75%-100%, 10 min) to provide the title compound (130 mg, 477 μmol, 24.6% yield). ¹H NMR (400 MHz, CDCl₃): δ ppm 7.61-7.55 (m, 2H), 6.91 (d, J=8.2 Hz, 2H), 1.81 (dd, J=6.2, 8.1 Hz, 1H), 1.22 (s, 3H), 0.83-0.74 (m, 5H).

### Step 3: Synthesis of (2R)-3-(4-(2,2-dimethylcyclopropyl)phenyl)-2-methylpropanal and (2S)-3-(4-(2,2-dimethylcyclopropyl)phenyl)-2-methylpropanal

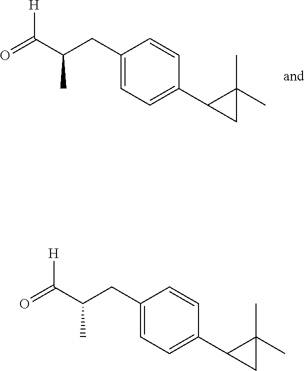 [SMILES: C[C@@H](C=O)Cc1ccc(C2CC2(C)C)cc1 ; C[C@H](C=O)Cc1ccc(C2CC2(C)C)cc1]

To a mixture of 1-(2,2-dimethylcyclopropyl)-4-iodo-benzene (160 mg, 588 μmol), triethylamine (89.2 mg, 881.9 μmol, 123 μL) and 2-methylprop-2-en-1-ol (50.9 mg, 706 μmol, 60 μL) in CH₃CN (2 mL) was added Pd(OAc)₂ (13.20 mg, 58.80 μmol) under N₂. The mixture was stirred at 80° C. for 16 h under N₂. The mixture was concentrated in reduced pressure. The residue was poured into water (10 mL). The aqueous phase was extracted with ethyl acetate (10 mL×3). The combined organic phase was washed with brine (8 mL), dried with anhydrous Na₂SO₄, filtered and concentrated under vacuum. The residue was purified by prep-HPLC (Waters Xbridge C18; 150×50 mm, 10 m; [water (10 mM NH₄HCO₃)-ACN]; B %: 52%-82%, 10 min) to provide the title compound (53 mg, 245 μmol, 41.6% yield). ¹H NMR (400 MHz, CDCl₃): δ ppm 9.72 (d, J=1.6 Hz, 1H), 7.12-7.04 (m, 4H), 3.05 (dd, J=5.6, 13.4 Hz, 1H), 2.71-2.55 (m, 2H), 1.87-1.81 (m, 1H), 1.22 (s, 3H), 1.08 (d, J=6.8 Hz, 3H), 0.79 (s, 3H), 0.78-0.74 (m, 2H).

### Step 4: Synthesis of the Title Compounds

A mixture of 2-thia-7-azaspiro[3.4]octane 2,2-dioxide (40 mg, 202 μmol, HCl) and triethylamine (20.5 mg, 202 μmol, 28.2 L) in DCM (2 mL) was stirred at 25° C. for 30 min. 3-[4-(2,2-dimethylcyclopropyl)phenyl]-2-methyl-propanal (52.52 mg, 242.8 mol), HOAc (24.30 mg, 404.7 μmol, 23.15 μL) was added. The mixture was stirred at 25° C. for 1 h, then NaBH(OAc)₃ (85.77 mg, 404.69 μmol) was added and the mixture was stirred at 25° C. for 1 h. The mixture was concentrated under reduced pressure. The residue was purified by HPLC (Phenomenex luna C18, 150×25 mm, 10 m; [water (0.1% TFA)-ACN]; 27%-57%, 10 min) to provide a racemic mixture of the title compound (44.5 mg, 93.5 μmol, 46% yield).

Compound 102 (mixture): ¹H NMR (400 MHz, CD₃OD): δ ppm 7.43 (t, J=7.7 Hz, 1H), 7.27 (d, J=7.9 Hz, 1H), 7.18 (dd, J=1.6, 10.4 Hz, 1H), 4.21 (br s, 1H), 4.01 (br d, J=8.8 Hz, 1H), 3.86-3.66 (m, 3H), 3.42 (br d, J=6.8 Hz, 2H), 3.06 (s, 3H), 2.65-2.50 (m, 1H), 2.47-2.30 (m, 4H), 2.29-2.02 (m, 6H), 1.97-1.59 (m, 5H). LCMS (ESI) [M+H]⁺=362.2.

### Example 104: (S)-6-(2-Methyl-3-(4-(trifluoromethyl)phenyl)propyl)-2-thia-6-azaspiro[3.4]octane 2,2-dioxide and (R)-6-(2-methyl-3-(4-(trifluoromethyl)phenyl)propyl)-2-thia-6-azaspiro[3.4]octane 2,2-dioxide

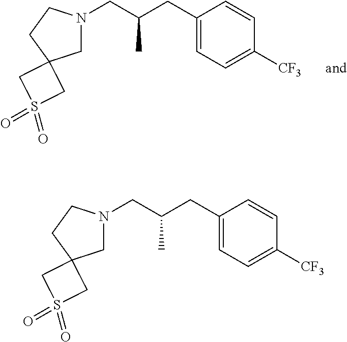 [SMILES: C[C@H](Cc1ccc(C(F)(F)F)cc1)CN1CCC2(C1)CS(=O)(=O)C2 ; C[C@@H](Cc1ccc(C(F)(F)F)cc1)CN1CCC2(C1)CS(=O)(=O)C2]

### Step 1: Synthesis of (R)-2-methyl-3-(4-(trifluoromethyl)phenyl)propanal and (S)-2-methyl-3-(4-(trifluoromethyl)phenyl)propanal

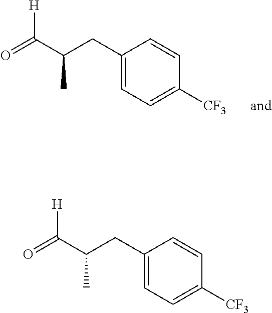

To a mixture of 1-iodo-4-(trifluoromethyl)benzene (1 g, 3.68 mmol, 541 μL), 2-methylprop-2-en-1-ol (331 mg, 5 mmol, 389 μL) and Pd(OAc)₂ (25 mg, 110 μmol) in ACN (10 mL) was added triethylamine (465 mg, 5 mmol, 640 μL). The mixture was purged with N₂ for 10 seconds and stirred at 80° C. for 16 h. The reaction was concentrated under reduced pressure. The residue was dissolved with petroleum ether (20 mL) and washed with water (20 mL). The organic layer was concentrated under reduced pressure. The residue was purified by silica column chromatography (petroleum ether/ethyl acetate=10:1) to provide the title compound (170 mg, 786 μmol, 21% yield). ¹H NMR (400 MHz, CDCl₃): δ ppm 9.73 (d, J=1.2 Hz, 1H), 7.56 (d, J=8.1 Hz, 2H), 7.30 (d, J=7.9 Hz, 2H), 3.22-3.11 (m, 1H), 2.76-2.62 (m, 2H), 1.12 (d, J=7.0 Hz, 3H).

### Step 2: Synthesis of the Title Compounds

To a mixture of 2-thia-7-azaspiro[3.4]octane-2,2-dioxide hydrochloride (20 mg, 101 μmol) in DCM (1 mL) was added triethylamine (9 mg, 91 μmol, 13 L) and the mixture was stirred at 25° C. for 15 min. Then (+/−) 2-methyl-3-[4-(trifluoromethyl)phenyl]propanal (25 mg, 114 μmol) was added to the mixture and the mixture was stirred at 25° C. for 1 h. NaBH(OAc)₃ (43 mg, 202 μmol) was added to the mixture at 0° C. and the mixture was stirred at 25° C. for 16 h. The reaction mixture was concentrated under reduced pressure. The residue was purified by HPLC (Nano-micro Kromasil C18, 80×25 mm, 3 m; 25-45% acetonitrile in a 0.1% trifluoracetic acid solution in water, 7 min gradient) to provide the title compound as a racemic mixture (17 mg, 34% yield).

Compound 104 (mixture): ¹H NMR (400 MHz, CD₃OD): δ ppm 7.63 (d, J=8.19 Hz, 2H), 7.44 (d, J=8.19 Hz, 2H), 4.36-4.17 (m, 4H), 4.10-3.40 (m, 2H), 3.29-3.11 (m, 4H), 2.88 (dd, J=13.39, 5.69 Hz, 1H), 2.66-2.38 (m, 3H), 2.36-2.20 (m, 1H), 1.02 (d, J=6.60 Hz, 3H). LCMS (ESI) [M+H]⁺=362.1

### Example 105: (S)-6-(4-(4-(tert-Pentyl)phenyl)butan-2-yl)-2-thia-6-azaspiro[3.4]octane 2,2-dioxide and (R)-6-(4-(4-(tert-pentyl)phenyl)butan-2-yl)-2-thia-6-azaspiro[3.4]octane 2,2-dioxide

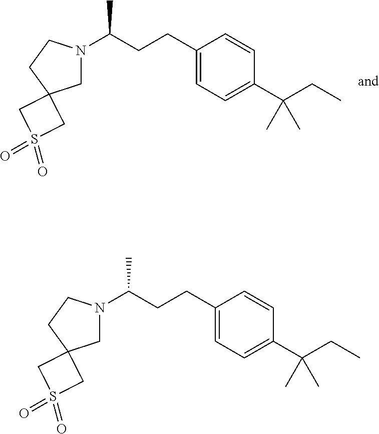 [SMILES: CCC(C)(C)c1ccc(CC[C@H](C)N2CCC3(C2)CS(=O)(=O)C3)cc1 ; CCC(C)(C)c1ccc(CC[C@@H](C)N2CCC3(C2)CS(=O)(=O)C3)cc1]

### Step 1: Synthesis of 4-[4-(1,1-dimethylpropyl)phenyl]butan-2-one

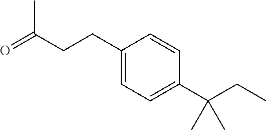 [SMILES: CCC(C)(C)c1ccc(CCC(C)=O)cc1]

To a stirred solution of 1-(1,1-dimethylpropyl)-4-iodo-benzene (1 g, 3.65 mmol) in ACN (5 mL) was added but-3-en-2-ol (329 mg, 4.56 mmol, 395 L), Pd(OAc)₂ (25 mg, 109 umol, 0.03 eq) and triethylamine (461 mg, 4.56 mmol, 635 L), the mixture was purged with N₂ for 1 minute, and the mixture was stirred at 80° C. for 16 h under N₂. The reaction mixture was poured into water (10 mL). The aqueous phase was extracted with ethyl acetate (10 mL×2). The combined organic phase was dried with anhydrous Na₂SO₄, filtered and concentrated under vacuum. The residue was purified by silica flash column chromatography (10-12% ethyl acetate in petroleum ether) to provide the title compound (300 mg, 1.37 mmol, 38% yield). ¹H-NMR (400 MHz, CDCl₃): δ ppm 7.24 (d, J=8.4 Hz, m, 2H), 7.11 (d, J=8.4 Hz, 2H), 2.94-2.83 (m, 2H), 2.80-2.72 (m, 2H), 2.15 (bs, 3H), 1.68-1.60 (m, 2H), 1.28-1.26 (bs, 6H), 0.71-0.65 (m, 3H).

### Step 2: Synthesis of the Title Compounds

To a stirred mixture of 2-thia-7-azaspiro[3.4]octane 2,2-dioxide hydrochloride (20 mg, 101 μmol) in DCM (1 mL) was added triethylamine (9 mg, 91 μmol, 12 μL) and the resulting mixture was stirred at 25° C. for 15 min. 4-[4-(1,1-dimethylpropyl)phenyl]butan-2-one (24 mg, 111 μmol) was added, and the mixture was stirred at 2 h for 25° C. NaBH(OAc)₃ (43 mg, 202 μmol) was added, and the mixture was stirred at 25° C. for 16 h. The reaction mixture was concentrated under vacuum. The crude residue was purified by HPLC (Nano-micro Kromasil C18 80×25 mm, 3 m column; 30-48% acetonitrile in a 0.1% trifluoroacetic acid solution in water, 7 min gradient) to provide the title compound as a racemic mixture (28 mg, 57% yield).

Compound 105 (mixture): ¹H NMR (400 MHz, CD₃OD): δ ppm 7.29 (d, J=8.4 Hz, 2H), 7.18 (d, J=8.4 Hz, 2H), 4.45-4.06 (m, 5H), 3.95-3.83 (m, 1H), 3.73-3.63 (m, 1H), 3.59-3.49 (m, 1H), 3.39-3.35 (m, 1H), 2.80 (ddd, J=14.13, 9.44, 5.07 Hz, 1H), 2.67-2.57 (m, 1H), 2.54-2.36 (m, 2H), 2.21-2.09 (m, 1H), 1.92-1.80 (m, 1H), 1.65 (q, J=7.42 Hz, 2H), 1.44 (d, J=6.50 Hz, 3H), 1.32-1.15 (m, 6H), 0.65 (t, J=7.44 Hz, 3H). LCMS (ESI) [M+H]⁺=364.2.

### Example 106: (R)-7-((S)-2-Methyl-3-(4-(tert-pentyl)phenyl)propyl)-2-thia-7-azaspiro[4.4]nonane 2,2-dioxide, (S)-7-((R)-2-methyl-3-(4-(tert-pentyl)phenyl)propyl)-2-thia-7-azaspiro[4.4]nonane 2,2-dioxide, (S)-7-((S)-2-methyl-3-(4-(tert-pentyl)phenyl)propyl)-2-thia-7-azaspiro[4.4]nonane 2,2-dioxide, and (R)-7-((R)-2-methyl-3-(4-(tert-pentyl)phenyl)propyl)-2-thia-7-azaspiro[4.4]nonane 2,2-dioxide

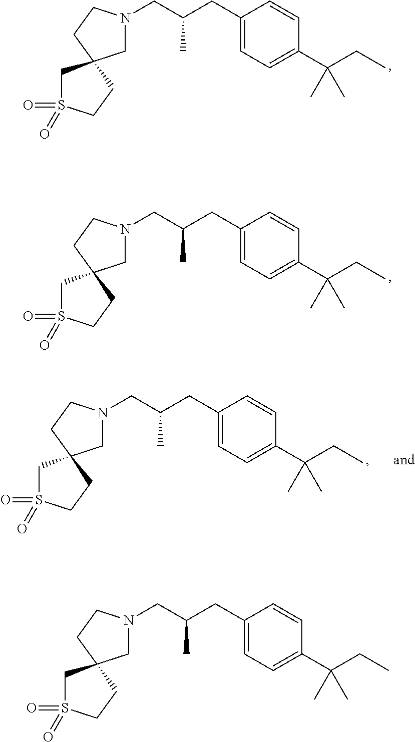 [SMILES: CCC(C)(C)c1ccc(C[C@H](C)CN2CC[C@@]3(CCS(=O)(=O)C3)C2)cc1 ; CCC(C)(C)c1ccc(C[C@@H](C)CN2CC[C@]3(CCS(=O)(=O)C3)C2)cc1 ; CCC(C)(C)c1ccc(C[C@H](C)CN2CC[C@]3(CCS(=O)(=O)C3)C2)cc1 ; CCC(C)(C)c1ccc(C[C@@H](C)CN2CC[C@@]3(CCS(=O)(=O)C3)C2)cc1]

The title compounds were synthesized using the synthetic route of example 103, utilizing (+/−) 2-thia-7-azaspiro[4.4]nonane 2,2-dioxide hydrochloride to provide a mixture of diastereomers.

Compound 106 (mixture): ¹H NMR (400 MHz, CD₃OD) δ ppm 7.30 (d, J=8.31 Hz, 2H), 7.15 (d, J=8.19 Hz, 2H), 3.99-3.70 (m, 2H), 3.45-3.32 (m, 1H), 3.30-3.09 (m, 7H), 2.79-2.68 (m, 1H), 2.49 (dd, J=13.69, 8.31 Hz, 1H), 2.44-2.08 (m, 5H), 1.65 (q, J=7.38 Hz, 2H), 1.27 (s, 6H), 1.03 (dd, J=6.66, 1.53 Hz, 3H), 0.66 (t, J=7.46 Hz, 3H). LCMS (ESI) [M+H]⁺=378.2.

### Example 107: (R)-6-(2-Methyl-3-(4-(tert-pentyl)phenyl)propyl)-2-thia-6-azaspiro[3.4]octane 2,2-dioxide and (S)-6-(2-methyl-3-(4-(tert-pentyl)phenyl)propyl)-2-thia-6-azaspiro[3.4]octane 2,2-dioxide

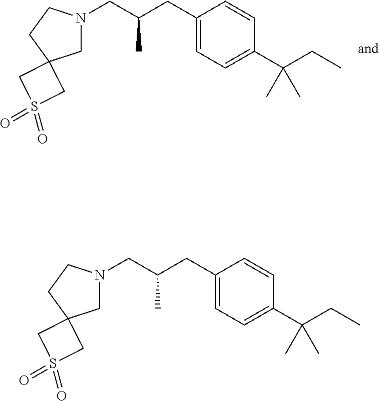 [SMILES: CCC(C)(C)c1ccc(C[C@@H](C)CN2CCC3(C2)CS(=O)(=O)C3)cc1 ; CCC(C)(C)c1ccc(C[C@H](C)CN2CCC3(C2)CS(=O)(=O)C3)cc1]

The title compounds were synthesized using the synthetic route of Example 103, utilizing 2-thia-7-azaspiro[3.4]octane 2,2-dioxide hydrochloride to provide a racemic mixture.

Compound 107 (mixture): ¹H NMR (400 MHz, CD₃OD): δ ppm 7.30 (d, J=8.25 Hz, 2H), 7.15 (d, J=8.25 Hz, 2H), 4.40-4.14 (m, 5H), 4.07-3.94 (m, 1H), 3.84-3.73 (m, 1H), 3.62-3.42 (m, 1H), 3.22-3.15 (m, 2H), 2.73 (dd, J=13.63, 6.13 Hz, 1H), 2.49 (br dd, J=13.51, 8.38 Hz, 3H), 2.27-2.16 (m, 1H) 1.66 (q, J=7.38 Hz, 2H), 1.32-1.24 (m, 6H), 1.03 (d, J=6.63 Hz, 3H), 0.66 (t, J=7.44 Hz, 3H). LCMS (ESI) [M+H]⁺=364.2.

### Example 108: (R)-6-(3-(4-Cyclopropylphenyl)-2-methylpropyl)-2-thia-6-azaspiro[3.4]octane 2,2-dioxide and (S)-6-(3-(4-cyclopropylphenyl)-2-methylpropyl)-2-thia-6-azaspiro[3.4]octane 2,2-dioxide

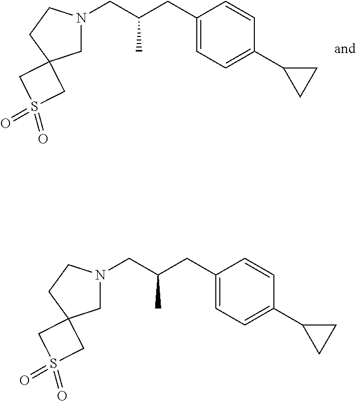 [SMILES: C[C@@H](Cc1ccc(C2CC2)cc1)CN1CCC2(C1)CS(=O)(=O)C2 ; C[C@H](Cc1ccc(C2CC2)cc1)CN1CCC2(C1)CS(=O)(=O)C2]

### Step 1: Synthesis of 1-cyclopropyl-4-iodo-benzene

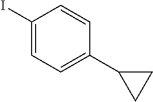

To a solution of 4-cyclopropylaniline (0.5 g, 3.75 mmol) in H₂O (5 mL) was added H₂SO₄ (5.06 g, 51.6 mmol, 2.75 mL) slowly at 0° C. Then NaNO₂ (259.01 mg, 3.75 mmol) which was dissolved with H₂O (2 mL) was added slowly at 0° C. The resulting mixture was added into a solution of potassium iodide (1.25 g, 7.51 mmol) in H₂O (2 mL) and the mixture was stirred at 60° C. for 2 h. The mixture was diluted with water (10 mL) and the resulting mixture was extracted with DCM (10 mL×3). The combined organic phase was dried over anhydrous Na₂SO₄, filtered and concentrated at reduced pressure. The residue was purified by prep-TLC (petroleum ether:ethyl acetate=20:1) to the title compound (650 mg, 2.66 mmol, 71% yield). ¹H-NMR (400 MHz, CDCl₃): δ ppm 7.59-7.53 (m, 2H), 6.87-6.79 (m, 2H), 1.89-1.80 (m, 1H), 1.00-0.94 (m, 2H), 0.69-0.64 (m, 2H).

### Step 2: Synthesis of (R)-3-(4-cyclopropylphenyl)-2-methylpropanal and (S)-3-(4-cyclopropylphenyl)-2-methylpropanal

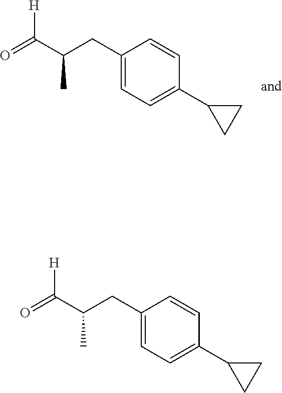

To a mixture of 1-cyclopropyl-4-iodo-benzene (0.4 g, 1.64 mmol) and (+/−) 2-methylprop-2-en-1-ol (177.26 mg, 2.46 mmol, 208.05 μL) in acetonitrile (2 mL) was added triethylamine (249 mg, 2.46 mmol, 342 μL) and Pd(OAc)₂ (11.04 mg, 49.17 μmol) under N₂. The mixture was stirred at 80° C. for 16 h. The mixture was diluted with EtOAc (30 mL) and the resulting mixture was washed with brine (10 mL×3), dried over anhydrous Na₂SO₄, filtered and concentrated at reduced pressure. The residue was purified by HPLC (column: Waters Xbridge 150×25 mm, 5 m; mobile phase: [water (10 mM NH₄HCO₃)-ACN]; 38%-68%, 10 min). The fraction was extracted with EtOAc (30 mL×3). The combined organic phase was washed with brine (30 mL×3), dried over anhydrous Na₂SO₄, filtered and concentrated at reduced pressure to provide the title compound (150 mg, 797 μmol, 48.62% yield). ¹H NMR (400 MHz, CDCl₃): δ ppm 9.72 (d, J=1.6 Hz, 1H), 7.09-7.04 (m, 2H), 7.03-6.98 (m, 2H), 3.04 (dd, J=5.8, 13.4 Hz, 1H), 2.70-2.53 (m, 2H), 1.87 (tt, J=5.1, 8.4 Hz, 1H), 1.09 (d, J=6.9 Hz, 3H), 0.99-0.89 (m, 2H), 0.72-0.64 (m, 2H).

### Step 3: Synthesis of the Title Compounds

To a mixture of 2-thia-7-azaspiro[3.4]octane 2,2-dioxide hydrochloride (30 mg, 152 μmol) in DCM (3 mL) was added triethylamine (23.03 mg, 227.6 mol, 31.68 μL). The mixture was stirred at 30° C. for 30 min, then (+/−) 3-(4-cyclopropylphenyl)-2-methyl-propanal (34.28 mg, 182.1 μmol) and NaBH(OAc)₃ (96.49 mg, 455.3 μmol) was added and the mixture was stirred at 30° C. for 1 h. The mixture was concentrated at reduced pressure. The residue was purified by HPLC (Phenomenex luna C18; 150×40 mm, 15 m; [water (0.1% TFA)-ACN] 20%-50%, 10 min) to give the title compound as a racemic mixture (28.3 mg, 41.67% yield).

Compound 108 (mixture): ¹H NMR (400 MHz, CD₃OD): δ ppm 7.08 (d, J=8.4, 2H), 7.02 (d, J=8.4, 2H), 4.38-4.17 (m, 4H), 4.10-3.69 (m, 2H), 3.55-3.33 (m, 2H), 3.16 (d, J=7.2 Hz, 2H), 2.69 (dd, J=6.3, 13.5 Hz, 1H), 2.48 (br dd, J=8.2, 13.6 Hz, 3H), 2.19 (qd, J=6.9, 13.9 Hz, 1H), 1.92-1.83 (m, 1H), 1.02 (d, J=6.6 Hz, 3H), 0.97-0.91 (m, 2H), 0.66-0.61 (m, 2H). LCMS (ESI) [M+H]⁺=333.9.

### Examples 109A, 109B*, and 109C*: (R)-6-(3-(4-cyclobutylphenyl)-2-methylpropyl)-2-thia-6-azaspiro[3.4]octane 2,2-dioxide and (S)-6-(3-(4-cyclobutylphenyl)-2-methylpropyl)-2-thia-6-azaspiro[3.4]octane 2,2-dioxide

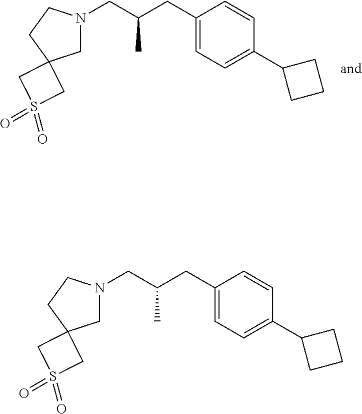 [SMILES: C[C@H](Cc1ccc(C2CCC2)cc1)CN1CCC2(C1)CS(=O)(=O)C2 ; C[C@@H](Cc1ccc(C2CCC2)cc1)CN1CCC2(C1)CS(=O)(=O)C2]

### Step 1. Synthesis of (R)-3-(4-cyclobutylphenyl)-2-methylpropanal and (S)-3-(4-cyclobutylphenyl)-2-methylpropanal

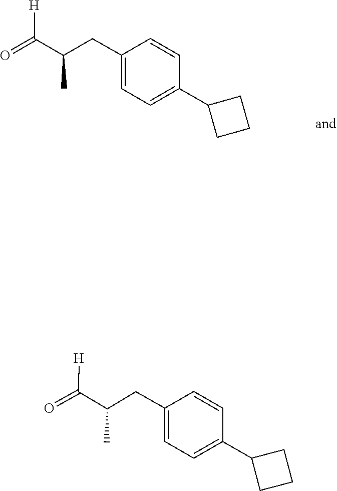

To a mixture of 1-cyclobutyl-4-iodo-benzene (200 mg, 775 μmol), triethylamine (98 mg, 969 mol, 135 μL) and 2-methylprop-2-en-1-ol (67 mg, 930 μmol, 79 μL) in acetonitrile (2 mL) was added Pd(OAc)₂ (17.40 mg, 77.49 μmol) under N₂. The mixture was stirred at 80° C. for 16 h under N₂. The mixture was filtered and concentrated under vacuum. The residue was purified by HPLC (Waters Xbridge C18, 150×50 mm, 10 m; [water (10 mM NH₄HCO₃)-ACN]; 48%-78%, 10 min gradient) to provide the title compound (65.0 mg, 41.5% yield).

### Step 2: Synthesis of the Title Compounds

A mixture of 2-thia-7-azaspiro[3.4]octane-2,2-dioxide hydrochloride (40 mg, 202 μmol) and triethylamine (20.48 mg, 202.3 μmol, 28.16 μL) in DCM (2 mL) was stirred at 25° C. for 30 min. 3-(4-cyclobutylphenyl)-2-methyl-propanal (49.12 mg, 242.8 mol), HOAc (12.15 mg, 202.3 μmol, 11.57 μL) was added. The mixture was stirred at 25° C. for 30 min, then NaBH(OAc)₃ (85.77 mg, 404.7 μmol) was added and stirred for 1 hours. The mixture was filtered and concentrated under vacuum. The residue was purified by HPLC (Phenomenex luna C18, 150×25 mm, 10 m; [water (0.1% TFA)-ACN]; 24%-54%, 10 min) to provide the title compound as a racemic mixture (59 mg, 63% yield).

Compound 109A (mixture): ¹H NMR (400 MHz, CD₃OD) δ ppm 7.18 (d, J=8.1 Hz, 2H), 7.14 (d, J=8.1 Hz, 2H), 4.38-4.29 (m, 1H), 4.22 (br s, 1H), 4.01 (br s, 1H), 3.81 (br s, 1H), 3.53 (quin, J=8.6 Hz, 1H), 3.45-3.32 (m, 1H), 3.30-3.12 (m, 3H), 2.71 (dd, J=6.2, 13.6 Hz, 1H), 2.50 (br dd, J=8.1, 13.5 Hz, 3H), 2.38-2.28 (m, 2H), 2.26-2.16 (m, 1H), 2.16-1.97 (m, 3H), 1.90-1.81 (m, 1H), 1.03 (d, J=6.6 Hz, 3H). LCMS (ESI) [M+H]⁺=348.1.

The racemic mixture from example 109A, was separated by chiral SFC (Daicel Chiralcel OJ-H (250 mm×30 mm, 5 m) 0.1% NH₃H₂O-EtOH, 40%) affording the first eluting peak, Compound 109B, as a pure single undefined/unassigned enantiomer of the above titled compound, and affording the second eluting peak, Compound 109C, as a pure single undefined/unassigned enantiomer of the above titled compound.

Compound 109B*: ¹H NMR (400 MHz, CD₃OD) δ ppm 7.18 (d, J=8.1 Hz, 2H), 7.14 (d, J=8.1 Hz, 2H), 4.38-4.29 (m, 1H), 4.22 (br s, 1H), 4.01 (br s, 1H), 3.81 (br s, 1H), 3.53 (quin, J=8.6 Hz, 1H), 3.45-3.32 (m, 1H), 3.30-3.12 (m, 3H), 2.71 (dd, J=6.2, 13.6 Hz, 1H), 2.50 (br dd, J=8.1, 13.5 Hz, 3H), 2.38-2.28 (m, 2H), 2.26-2.16 (m, 1H), 2.16-1.97 (m, 3H), 1.90-1.81 (m, 1H), 1.03 (d, J=6.6 Hz, 3H). LCMS (ESI) [M+H]⁺=348.1.

Compound 109C*: ¹H NMR (400 MHz, CD₃OD) δ ppm 7.18 (d, J=8.1 Hz, 2H), 7.14 (d, J=8.1 Hz, 2H), 4.38-4.29 (m, 1H), 4.22 (br s, 1H), 4.01 (br s, 1H), 3.81 (br s, 1H), 3.53 (quin, J=8.6 Hz, 1H), 3.45-3.32 (m, 1H), 3.30-3.12 (m, 3H), 2.71 (dd, J=6.2, 13.6 Hz, 1H), 2.50 (br dd, J=8.1, 13.5 Hz, 3H), 2.38-2.28 (m, 2H), 2.26-2.16 (m, 1H), 2.16-1.97 (m, 3H), 1.90-1.81 (m, 1H), 1.03 (d, J=6.6 Hz, 3H). LCMS (ESI) [M+H]⁺=348.1.

### Example 110: (R)-7-(2-Methyl-3-(4-(tert-pentyl)phenyl)propyl)-2-thia-7-azaspiro[3.5]nonane 2,2-dioxide and (S)-7-(2-methyl-3-(4-(tert-pentyl)phenyl)propyl)-2-thia-7-azaspiro[3.5]nonane 2,2-dioxide

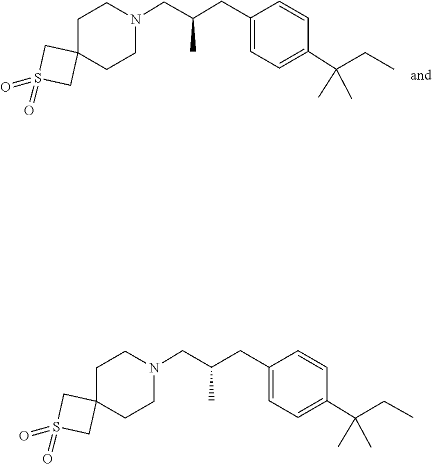 [SMILES: CCC(C)(C)c1ccc(C[C@@H](C)CN2CCC3(CC2)CS(=O)(=O)C3)cc1 ; CCC(C)(C)c1ccc(C[C@H](C)CN2CCC3(CC2)CS(=O)(=O)C3)cc1]

The title compounds were synthesized using the synthetic route of Example 103, utilizing 2 thia-7-azaspiro[3.5]nonane-2,2-dioxide hydrochloride to provide a racemic mixture.

Compound 110 (mixture): ¹H NMR (400 MHz, CD₃OD): δ ppm 7.30 (d, J=8.31 Hz, 2H), 7.15 (d, J=8.31 Hz, 2H), 4.04 (bs, 4H), 3.66-3.51 (m, 2H), 3.17-2.99 (m, 3H), 2.96-2.84 (m, 1H), 2.75-2.65 (m, 1H), 2.57-2.47 (m, 1H), 2.35-2.21 (m, 3H), 2.16-2.04 (m, 2H), 1.69-1.62 (m, 3H), 1.27 (s, 6H), 1.03 (d, J=6.60 Hz, 3H), 0.66 (t, J=7.46 Hz, 3H). LCMS (ESI) [M+H]⁺=378.2.

### Examples 111A, 111B*, and 111C*: (R)-6-(2-Methyl-3-(4-(1-methylcyclopropyl)phenyl)propyl)-2-thia-6-azaspiro[3.4]octane 2,2-dioxide and (S)-6-(2-methyl-3-(4-(1-methylcyclopropyl)phenyl)propyl)-2-thia-6-azaspiro[3.4]octane 2,2-dioxide

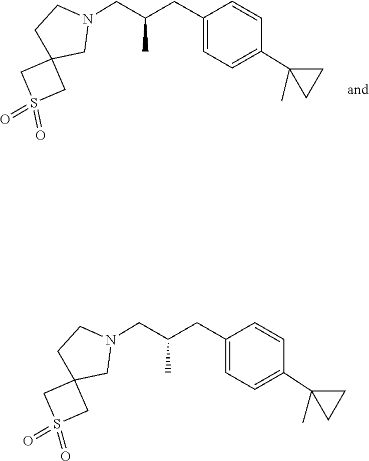 [SMILES: C[C@H](Cc1ccc(C2(C)CC2)cc1)CN1CCC2(C1)CS(=O)(=O)C2 ; C[C@@H](Cc1ccc(C2(C)CC2)cc1)CN1CCC2(C1)CS(=O)(=O)C2]

### Step 1: Synthesis of 1-iodo-4-(1-methylcyclopropyl)benzene

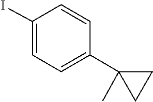

To a mixture of ZnEt₂ (3.04 g, 24.6 mmol) in DCM (20 mL) was added CH₂I₂ (8.78 g, 32.8 mmol, 2.64 mL) at 0° C. under N₂. The mixture was stirred at 0° C. for 30 min, then 1-iodo-4-isopropenyl-benzene (2.0 g, 8.2 mmol) was added at 0° C. and stirred at 0° C. for 1 h. The residue was poured into water (50 mL). The mixture was filtered. The aqueous phase was extracted with ethyl acetate (50 mL×3). The combined organic phase was washed with brine (50 mL), dried with anhydrous Na₂SO₄, filtered and concentrated in vacuum. The crude product was purified by reversed-phase HPLC (0.1% TFA condition) to provide the title compound (1.1 g, 4.3 mmol, 52% yield). ¹H NMR (400 MHz, CDCl₃): δ ppm 7.59 (br d, J=7.8 Hz, 2H), 7.00 (br d, J=7.7 Hz, 2H), 1.38 (s, 3H), 0.83 (br s, 2H), 0.74 (br s, 2H).

### Step 2: Synthesis of (R)-2-methyl-3-(4-(1-methylcyclopropyl)phenyl)propanal and ( )-2-methyl-3-(4-(1-methylcyclopropyl)phenyl)propanal

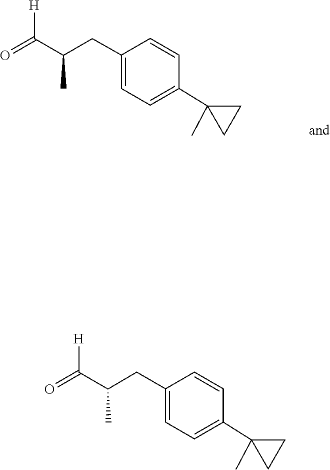

To a mixture of 1-iodo-4-(1-methylcyclopropyl)benzene (200 mg, 775 μmol) and 2-methylprop-2-en-1-ol (69.84 mg, 968.6 μmol, 81.98 μL), triethylamine (135 μL, 969 μmol) in acetonitrile (1 mL) was added Pd(OAc)₂ (17.40 mg, 77.49 μmol) under N₂. The mixture was stirred at 80° C. for 16 h under N₂. The mixture was poured into water (5 mL). The aqueous phase was extracted with ethyl acetate (8 mL×3). The combined organic phase was washed with brine (8 mL), dried with anhydrous Na₂SO₄, filtered and concentrated under vacuum. The residue was purified by HPLC (Waters Xbridge C18 150×50 mm, 10 m; [water (10 mM NH₄HCO₃)-ACN]; 45%-75%, 10 min gradient) to provide the title compound (80 mg, 51% yield).

### Step 3: Synthesis of the Title Compounds

A mixture of 2-thia-7-azaspiro[3.4]octane-2,2-dioxide hydrochloride (30.0 mg, 152 μmol) and TEA (15.36 mg, 151.8 μmol, 21.12 μL) in DCM (2 mL) was stirred at 25° C. for 30 min, then 2-methyl-3-[4-(1-methylcyclopropyl)phenyl]propanal (30.70 mg, 151.8 μmol), HOAc (18.23 mg, 303.5 μmol, 17.36 μL) was added and stirred at 25° C. for 1 h. NaBH(OAc)₃ (64.33 mg, 303.5 μmol) was added. The mixture was stirred at 25° C. for 1 h. The mixture was filtered and concentrated under reduced pressure. The residue was purified by HPLC (Phenomenex luna C18 150×40 mm, 15 m; [water (0.1% TFA)-ACN]; 22%-52%, 10 min gradient) to provide the title compound as a racemic mixture (8.4 mg, 69% yield).

Compound 111A (mixture): ¹H NMR (400 MHz, CD₃OD) δ ppm 7.21 (d, J=8.0 Hz, 2H), 7.12 (d, J=8.0 Hz, 2H), 4.40-4.14 (m, 4H), 3.98 (br s, 1H), 3.79 (br s, 1H), 3.52 (br s, 1H), 3.16 (d, J=7.2 Hz, 2H), 2.70 (dd, J=6.4, 13.6 Hz, 1H), 2.49 (br dd, J=8.1, 13.6 Hz, 3H), 2.27-2.13 (m, 1H), 1.38 (s, 3H), 1.02 (d, J=6.6 Hz, 3H), 0.85-0.79 (m, 2H), 0.74-0.70 (m, 2H). LCMS (ESI) [M+H]⁺=348.1.

The racemic mixture from example 111A, was separated by chiral SFC (Daicel Chiralcel OJ-H (250 mm×30 mm, 5 m) 0.1% NH₃H₂O-EtOH, 40%) affording the first eluting peak, Compound 111B, as a pure single undefined/unassigned enantiomer of the above titled compound, and affording the second eluting peak, Compound 111C, as a pure single undefined/unassigned enantiomer of the above titled compound

Compound 111B*: ¹H NMR (400 MHz, CD₃OD) δ ppm 7.21 (d, J=8.0 Hz, 2H), 7.12 (d, J=8.0 Hz, 2H), 4.40-4.14 (m, 4H), 3.98 (br s, 1H), 3.79 (br s, 1H), 3.52 (br s, 1H), 3.16 (d, J=7.2 Hz, 2H), 2.70 (dd, J=6.4, 13.6 Hz, 1H), 2.49 (br dd, J=8.1, 13.6 Hz, 3H), 2.27-2.13 (m, 1H), 1.38 (s, 3H), 1.02 (d, J=6.6 Hz, 3H), 0.85-0.79 (m, 2H), 0.74-0.70 (m, 2H). Spec. M+H: 348.1.

Compound 111C*: ¹H NMR (400 MHz, CD₃OD) δ ppm 7.21 (d, J=8.0 Hz, 2H), 7.12 (d, J=8.0 Hz, 2H), 4.40-4.14 (m, 4H), 3.98 (br s, 1H), 3.79 (br s, 1H), 3.52 (br s, 1H), 3.16 (d, J=7.2 Hz, 2H), 2.70 (dd, J=6.4, 13.6 Hz, 1H), 2.49 (br dd, J=8.1, 13.6 Hz, 3H), 2.27-2.13 (m, 1H), 1.38 (s, 3H), 1.02 (d, J=6.6 Hz, 3H), 0.85-0.79 (m, 2H), 0.74-0.70 (m, 2H). LCMS (ESI) [M+H]⁺=348.1.

### Example 112: 6-(2-(4-(Trifluoromethyl)phenoxy)ethyl)-2-thia-6-azaspiro[3.4]octane-2,2-dioxide

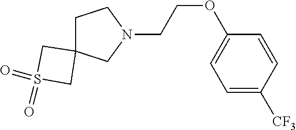 [SMILES: O=S1(=O)CC2(CCN(CCOc3ccc(C(F)(F)F)cc3)C2)C1]

### Step 1: Synthesis of 1-(2-bromoethoxy)-4-(trifluoromethyl)benzene

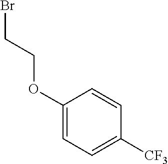 [SMILES: FC(F)(F)c1ccc(OCCBr)cc1]

To a solution of 4-(trifluoromethyl)phenol (1.0 g, 6.17 mmol) in N,N-dimethylformamide (10 mL) was added 1,2-dibromoethane (4.8 g, 25.6 mmol), K₂CO₃ (2.2 g, 15.9 mmol) and KI (100 mg, 0.60 mmol) at 20-30° C. Then the mixture was heated to 80° C. and stirred for 15 h. The reaction was quenched with water (50 mL), and extracted with ethyl acetate (40 mL×2). The combined organic phase was washed with brine (25 mL×3), dried over anhydrous Na₂SO₄, filtered and concentrated under reduced pressure to give the crude residue, which was purified by silica column chromatography (100% petroleum ether) to provide the title compound (690 mg, 2.56 mmol, 42% yield). ¹H NMR (400 MHz, CDCl₃): δ ppm 7.57 (d, J=8.8 Hz, 2H), 6.99 (d, J=8.8 Hz, 2H), 4.37-4.32 (m, 2H), 3.69-3.63 (m, 2H).

### Step 2: Synthesis of the Title Compound

1-(2-Bromoethoxy)-4-(trifluoromethyl)benzene (48 mg, 0.18 mmol) and 2-thia-6-azaspiro[3.4]octane 2,2-dioxide hydrochloride (30 mg, 0.15 mmol) were dissolved in acetonitrile (1.5 mL), and N,N-diisopropylethylamine (0.12 mL, 0.73 mmol) was added at 20˜ 30° C. The reaction mixture was stirred at 80° C. for 5 h. This reaction was quenched with water (10 mL), and extracted with ethyl acetate (30 mL×2). The combined organic phase was washed with brine (25 mL×3), dried over anhydrous Na₂SO₄, filtered and concentrated under reduced pressure to give the crude product, which was purified by reverse phase chromatography and lyophilized to provide the title compound (25.4 mg, 0.072 mmol, 40% yield).

Compound 112: ¹H NMR (400 MHz, CDCl₃): δ ppm 7.56 (d, J=8.4 Hz, 2H), 6.96 (d, J=8.4 Hz, 2H), 4.21 (t, J=5.6 Hz, 2H), 4.08 (s, 4H), 3.00-2.92 (m, 4H), 2.84 (t, J=7.6 Hz, 2H), 2.19 (t, J=7.6 Hz, 2H). LCMS (ESI) [M+H]⁺=350.0.

### Example 113: 6-(3-(4-(Trifluoromethyl)phenyl)propyl)-2-thia-6-azaspiro[3.4]octane-2,2-dioxide

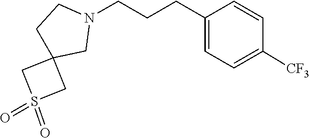 [SMILES: O=S1(=O)CC2(CCN(CCCc3ccc(C(F)(F)F)cc3)C2)C1]

### Step 1: Synthesis of (E)-methyl 3-(4-(trifluoromethyl)phenyl)acrylate

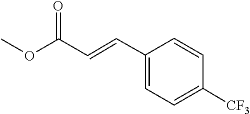 [SMILES: COC(=O)/C=C/c1ccc(C(F)(F)F)cc1]

To a solution of methyl diethylphosphonoacetate (2.1 mL, 11.5 mmol) in tetrahydrofuran (20 mL) was added sodium hydride (0.46 g, 11.49 mmol, 60% in mineral oil) in portions at 0° C. under nitrogen and stirred for 0.5 hour. 4-(Trifluoromethyl)benzaldehyde (1.0 g, 5.74 mmol) was added at 0° C. Then the reaction mixture was stirred at 20° C. for 1 h. The reaction was quenched by saturated ammonium chloride solution (30 mL), extracted with ethyl acetate (50 mL×3). The combined organic phase was washed with brine (50 mL×3), dried over anhydrous Na₂SO₄, filtered and concentrated under reduced pressure give the crude residue, which was purified by silica column chromatography (0-10% ethyl acetate in petroleum ether) to provide the title compound (760 mg, 3.30 mmol, 57.5% yield). LCMS (ESI) [M+H]⁺=231.0.

### Step 2: Synthesis of (E)-3-(4-(trifluoromethyl)phenyl)prop-2-en-1-ol

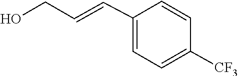 [SMILES: OC/C=C/c1ccc(C(F)(F)F)cc1]

To a solution of methyl (E)-3-[4-(trifluoromethyl)phenyl]prop-2-enoate (0.76 g, 3.3 mmol) in tetrahydrofuran (15 mL) was added LiBH₄ (180 mg, 8.25 mmol) at 0° C. The reaction was then warmed to 20° C. and stirred for 16 h under nitrogen. The reaction was quenched by saturated sodium bicarbonate (10 mL). The mixture was extracted with dichloromethane (50 ml×3). The combined organic layers were washed with brine (25 ml×3), dried over anhydrous Na₂SO₄, filtered and concentrated under vacuum to provide the crude title compound (650 mg, 3.22 mmol, 97% yield).

### Step 3: Synthesis of (E)-3-(4-(trifluoromethyl)phenyl)allyl methanesulfonate

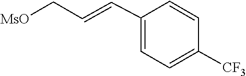 [SMILES: CS(=O)(=O)OC/C=C/c1ccc(C(F)(F)F)cc1]

(E)-3-[4-(Trifluoromethyl)phenyl]prop-2-en-1-ol (60.0 mg, 0.30 mmol) and N,N-diisopropylethylamine (0.15 mL, 0.89 mmol) were dissolved in dichloromethane (3 mL). Methanesulfonyl chloride (0.03 mL, 0.33 mmol) was added at 0° C. The mixture was stirred at 20° C. for 16 h. The reaction was quenched with water (10 mL) and extracted with dichloromethane (10 mL×3). The combined organic layer was dried over anhydrous sodium sulfate, filtered and concentrated to provide the title compound (70 mg, 0.25 mmol, 84.2% yield), which was used directly for the next step without further purification.

### Step 4: Synthesis of (E)-6-(3-(4-(trifluoromethyl)phenyl)allyl)-2-thia-6-azaspiro[3.4]octane-2,2-dioxide

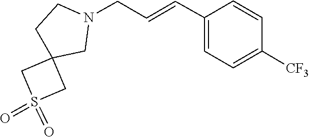 [SMILES: O=S1(=O)CC2(CCN(C/C=C/c3ccc(C(F)(F)F)cc3)C2)C1]

N,N-Diisopropylethylamine (0.11 mL, 0.64 mmol) and (E)-3-(4-(trifluoromethyl)phenyl) allyl methanesulfonate (60.0 mg, 0.21 mmol) were dissolved in acetonitrile (5 mL), and 2-thia-6-azaspiro[3.4]octane-2,2-dioxide hydrochloride (50.0 mg, 0.25 mmol) was added at 25° C. The reaction mixture was stirred at 80° C. for 16 h. After concentration under vacuum, the residue was purified by silica column chromatography (0-50% ethyl acetate in petroleum ether) to provide the title compound (40 mg, 0.12 mmol, 54% yield). LCMS (ESI) [M+H]⁺=346.1.

### Step 5: Synthesis of the Title Compound

To a solution of (E)-6-(3-(4-(trifluoromethyl)phenyl)allyl)-2-thia-6-azaspiro[3.4]octane 2,2-dioxide (30.0 mg, 0.09 mmol) in ethanol (10 mL) was added platinum(IV)oxide/carbon (2 mg, 0.01 mmol). The mixture was stirred at 25° C. under H₂ (15 psi) for 2 h. The mixture was filtered and concentrated to give the crude residue, which was purified by reverse phase chromatography and lyophilized to provide the title compound as the formic acid salt (4.4 mg, 0.012 mmol, 14.3% yield).

Compound 113: ¹H NMR (400 MHz, CD₃OD): δ ppm 8.47 (s, 1H), 7.58 (d, J=8.0 Hz, 2H), 7.41 (d, J=7.6 Hz, 2H), 4.19-4.11 (m, 4H), 3.09 (s, 2H), 2.93 (t, J=7.2 Hz, 2H), 2.79-2.71 (m, 4H), 2.27 (t, J=7.2 Hz, 2H), 1.96-1.88 (m, 2H). LCMS (ESI) [M+H]⁺=347.1.

### Examples 114A* and 114B*: (S)-6-(2-Methyl-3-(4-(trifluoromethyl)phenyl)propyl)-2-thia-6-azaspiro[3.4]octane 2,2-dioxide and (R)-6-(2-Methyl-3-(4-(trifluoromethyl)phenyl)propyl)-2-thia-6-azaspiro[3.4]octane 2,2-dioxide

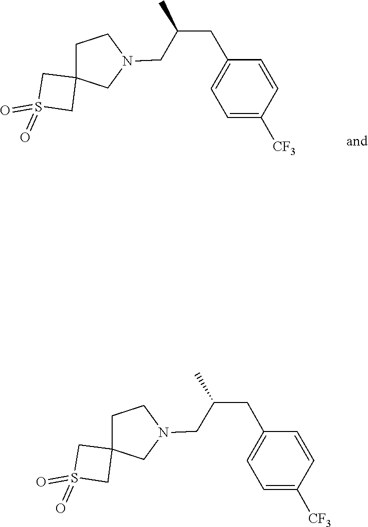 [SMILES: C[C@@H](Cc1ccc(C(F)(F)F)cc1)CN1CCC2(C1)CS(=O)(=O)C2 ; C[C@H](Cc1ccc(C(F)(F)F)cc1)CN1CCC2(C1)CS(=O)(=O)C2]

The title compounds were synthesized using the synthetic route of Example 104, utilizing 2-thia-6-azaspiro[3.4]octane-2,2-dioxide hydrochloride. The racemic mixture was purified by SFC (Daicel Chiralcel OJ-H (250 mm×30 mm, 5 m) 0.1% NH₃H₂O; EtOH; 40%) affording the first eluting peak, Compound 114A, as a pure single undefined/unassigned enantiomer (48 mg, 28.2% yield), and affording the second eluting peak. Compound 114B, as a pure single undefined/unassigned enantiomer (30.4 mg, 17.9% yield)

Compound 114A*: ¹H NMR (400 MHz, CD₃OD): δ ppm 7.56 (d, J=8.0 Hz, 2H), 7.37 (d, J=8.0 Hz, 2H), 4.08 (s, 4H), 2.92-2.85 (m, 1H), 2.80 (s, 2H), 2.72-2.62 (m, 2H), 2.51-2.29 (m, 3H), 2.16 (t, J=7.2 Hz, 2H), 1.97-1.87 (m, 1H), 0.88 (d, J=6.8 Hz, 3H). LCMS (ESI) [M+H]⁺=362.2.

Compound 114B*: ¹H NMR (400 MHz, CD₃OD): δ ppm 7.56 (d, J=8.0 Hz, 2H), 7.36 (d, J=8.0 Hz, 2H), 4.08 (s, 4H), 2.92-2.85 (m, 1H), 2.80 (s, 2H), 2.72-2.62 (m, 2H), 2.49-2.31 (m, 3H), 2.16 (t, J=7.2 Hz, 2H), 1.97-1.87 (m, 1H), 0.87 (d, J=6.4 Hz, 3H). LCMS (ESI) [M+H]⁺=362.2.

### Examples 115A* and 115B*: (R)-6-(2-(4-(Trifluoromethyl)phenoxy)propyl)-2-thia-6-azaspiro[3.4]octane 2,2-dioxide and (S)-6-(2-(4-(Trifluoromethyl)phenoxy)propyl)-2-thia-6-azaspiro[3.4]octane 2,2-dioxide

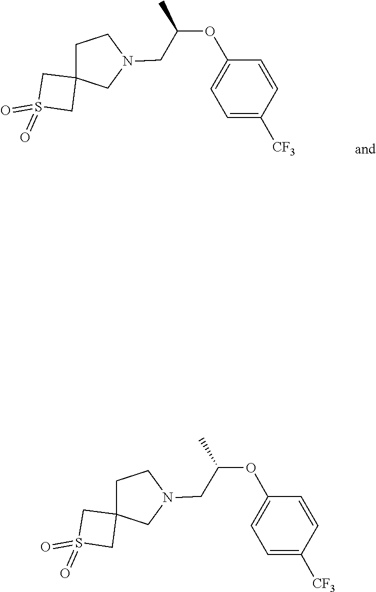 [SMILES: C[C@H](CN1CCC2(C1)CS(=O)(=O)C2)Oc1ccc(C(F)(F)F)cc1 ; C[C@@H](CN1CCC2(C1)CS(=O)(=O)C2)Oc1ccc(C(F)(F)F)cc1]

### Step 1: Synthesis of methyl (R)-2-(4-(trifluoromethyl)phenoxy)propanoate and methyl (S)-2-(4-(trifluoromethyl)phenoxy)propanoate

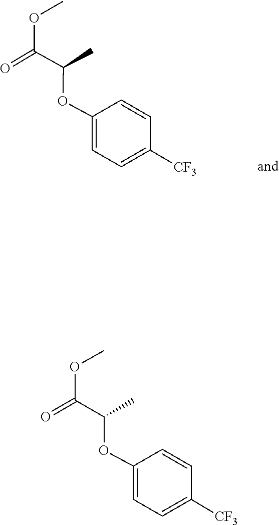 [SMILES: COC(=O)[C@@H](C)Oc1ccc(C(F)(F)F)cc1 ; COC(=O)[C@H](C)Oc1ccc(C(F)(F)F)cc1]

To a solution of 4-(trifluoromethyl)phenol (500 mg, 3.08 mmol) in N,N-dimethylformamide (5 mL) was added methyl 2-bromopropionate (620 mg, 3.71 mmol) and K₂CO₃ (1.3 g, 9.41 mmol) at 25° C. Then the reaction mixture was heated to 80° C. and stirred for 15 hours. The reaction mixture was diluted with water (80 mL), and extracted with ethyl acetate (40 mL×2). The combined organic phase was washed with brine, dried over Na₂SO₄, filtered and concentrated under reduced pressure to give the crude product, which was purified by silica column chromatography (0-10% ethyl acetate in petroleum ether) to provide the title compound (620 mg, 81% yield). ¹H NMR (400 MHz, CDCl₃): δ ppm 7.55 (d, J=8.8 Hz, 2H), 6.94 (d, J=8.8 Hz, 2H), 4.82 (q, J=6.4 Hz, 1H), 3.78 (s, 3H), 1.66 (d, J=6.4 Hz, 3H).

### Step 2: Synthesis of (R)-2-(4-(trifluoromethyl)phenoxy)propanal and (S)-2-(4-(trifluoromethyl)phenoxy)propanal

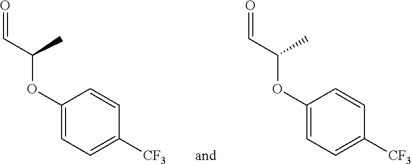

(+/−) Methyl 2-[4-(trifluoromethyl)phenoxy]propanoate (200 mg, 0.81 mmol) was dissolved in dichloromethane (4 mL) under nitrogen atmosphere and stirred at −78° C. Diisobutylaluminum hydride (1.0 mL, 1 mmol, 1 M in toluene) was added dropwise via syringe. The resulting mixture was stirred at −78° C. for 1 hour. The reaction was quenched by saturated NH₄C₁ aqueous solution (5 mL) and diluted with water (10 mL). The mixture was extracted with ethyl acetate (30 mL×2). The combined organic phase was washed with brine, dried over anhydrous Na₂SO₄, filtered and concentrated under reduced pressure to provide the title compound (160 mg, 91% yield) as a mixture of enantiomers, which was used directly for next step without further purification. ¹H NMR (400 MHz, CDCl₃): δ ppm 9.71 (d, J=1.6 Hz, 1H), 7.57 (d, J=8.8 Hz, 2H), 6.96 (d, J=8.4 Hz, 2H), 4.74-4.68 (m, 1H), 1.53 (d, J=7.2 Hz, 3H).

### Step 3: Synthesis of the Title Compounds

To a suspension of 2-thia-6-azaspiro[3.4]octane-2,2-dioxide hydrochloride (65 mg, 0.33 mmol) in anhydrous dichloromethane (3 mL) was added triethylamine (0.08 mL, 0.57 mmol) and stirred at 25° C. for 30 minutes. A solution of (+/−) 2-[4-(trifluoromethyl)phenoxy]propanal (110 mg, 0.50 mmol) in dichloromethane (3 mL) and acetic acid (0.08 mL, 1.4 mmol) were then added. The reaction mixture was stirred for 30 minutes, then NaBH(OAc)₃ (160 mg, 0.75 mmol) was added in portions. The reaction mixture was stirred for another 1 h at 30° C. The reaction was quenched by saturated NaHCO₃ solution (15 mL), and extracted with dichloromethane (30 mL×2). The combined organic phase was washed with brine, dried over anhydrous Na₂SO₄, filtered and concentrated under reduced pressure. The resulting residue was purified by reverse phase chromatography (acetonitrile 55-85%/(0.05% NH₃H₂O+10 mM NH₄HCO₃) in water) to provide the title compound (67 mg, 52.4% yield) as a mixture of enantiomers. The racemic mixture was separated by chiral SFC (Daicel Chiralcel OJ-H (250 mm×30 mm, 5 m); Supercritical CO₂; EtOH+NH₃H₂O=85/15; 60 mL/min) to afford the first eluting peak, Compound 115A (16.8 mg, 23.7% yield), of a single enantiomer of unassigned stereochemistry, and to afford the second eluting peak Compound 115B (19.7 mg, 27.3% yield), as a pure single undefined/unassigned enantiomer.

Compound 115A*: ¹H NMR (400 MHz, CD₃OD): δ ppm 7.57 (d, J=8.8 Hz, 2H), 7.08 (d, J=8.8 Hz, 2H), 4.74-4.66 (m, 1H), 4.08-4.03 (m, 4H), 2.97-2.89 (m, 2H), 2.86-2.71 (m, 4H), 2.15 (d, J=7.2 Hz, 2H), 1.31 (d, J=6.4 Hz, 3H). LCMS (ESI) [M+H]⁺=364.0.

Compound 115B*: ¹H NMR (400 MHz, CD₃OD): δ ppm 7.57 (d, J=8.8 Hz, 2H), 7.08 (d, J=8.8 Hz, 2H), 4.76-4.63 (m, 1H), 4.10-4.06 (m, 4H), 2.97-2.89 (m, 2H), 2.86-2.71 (m, 4H), 2.15 (d, J=7.2 Hz, 2H), 1.31 (d, J=6.0 Hz, 3H). LCMS (ESI) [M+H]⁺=364.0.

### Examples 116A* and 116B*: 6-((S)-3-(3-Chloro-4-((1s,4R)-4-hydroxy-4-(trifluoromethyl)cyclohexyl)phenyl)-2-methylpropyl)-2-thia-6-azaspiro[3.4]octane 2,2-dioxide, 6-((R)-3-(3-chloro-4-((1s,4S)-4-hydroxy-4-(trifluoromethyl)cyclohexyl)phenyl)-2-methylpropyl)-2-thia-6-azaspiro[3.4]octane 2,2-dioxide, 6-((S)-3-(3-Chloro-4-((1s,4S)-4-hydroxy-4-(trifluoromethyl)cyclohexyl)phenyl)-2-methylpropyl)-2-thia-6-azaspiro[3.4]octane 2,2-dioxide, and 6-((R)-3-(3-chloro-4-((1r,4R)-4-hydroxy-4-(trifluoromethyl)cyclohexyl)phenyl)-2-methylpropyl)-2-thia-6-azaspiro[3.4]octane 2,2-dioxide

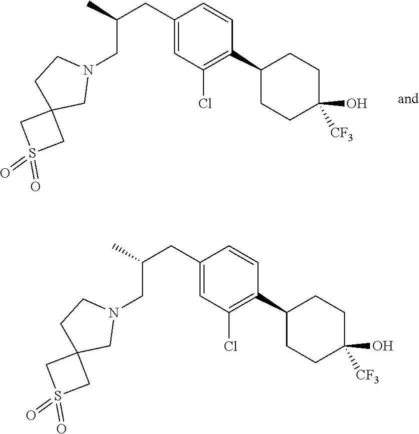 [SMILES: C[C@@H](Cc1ccc([C@H]2CC[C@](O)(C(F)(F)F)CC2)c(Cl)c1)CN1CCC2(C1)CS(=O)(=O)C2 ; C[C@H](Cc1ccc([C@H]2CC[C@](O)(C(F)(F)F)CC2)c(Cl)c1)CN1CCC2(C1)CS(=O)(=O)C2]

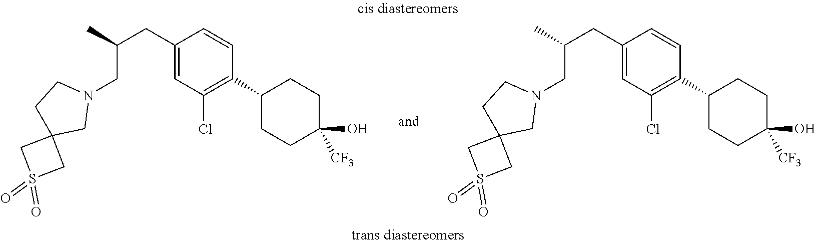 [SMILES: C[C@@H](Cc1ccc([C@H]2CC[C@@](O)(C(F)(F)F)CC2)c(Cl)c1)CN1CCC2(C1)CS(=O)(=O)C2 ; C[C@H](Cc1ccc([C@H]2CC[C@@](O)(C(F)(F)F)CC2)c(Cl)c1)CN1CCC2(C1)CS(=O)(=O)C2]

### Step 1: Synthesis of (E)-ethyl 3-(4-bromo-3-chlorophenyl)-2-methylacrylate

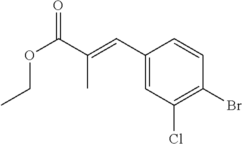 [SMILES: CCOC(=O)/C(C)=C/c1ccc(Br)c(Cl)c1]

To a solution of triethyl 2-phosphonopropionate (21.71 g, 91.13 mmol) in tetrahydrofuran (200 mL) was added sodium hydride (3.65 g, 91.13 mmol, 60% in mineral oil) in portions at 0° C. under nitrogen and stirred for 0.5 hour. 4-Bromo-3-chlorobenzaldehyde (10.0 g, 45.57 mmol) was added at 0° C. and then the reaction mixture was stirred at 30° C. for 2 h. The reaction mixture was quenched with a saturated solution of NH₄C₁ (100 mL), and extracted with ethyl acetate (150 mL×3). The combined organic phase was washed with brine (50 mL×3), dried over anhydrous Na₂SO₄, filtered and concentrated under reduced pressure. The residue was purified by silica column chromatography (0-5% ethyl acetate in petroleum ether) to provide the title compound (13 g, 93% yield). LCMS (ESI) [M+H]⁺=303.0.

### Step 2: Synthesis of (E)-ethyl 3-(3-chloro-4-(1,4-dioxaspiro[4.5]dec-7-en-8-yl)phenyl)-2-methylacrylate

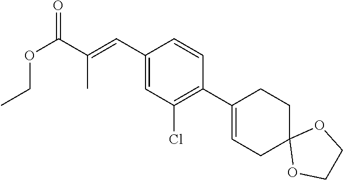 [SMILES: CCOC(=O)/C(C)=C/c1ccc(C2=CCC3(CC2)OCCO3)c(Cl)c1]

The solution of K₂C₀₃ (6.83 g, 49.41 mmol), 1,1′-bis(diphenylphosphino)ferrocene palladium dichloride (1.21 g, 1.65 mmol), ethyl (E)-3-(4-bromo-3-chloro-phenyl)-2-methyl-prop-2-enoate (5.0 g, 16.47 mmol), 1,4-dioxa-spiro[4,5]dec-7-en-8-boronic acid pinacol ester (4.38 g, 16.47 mmol) in water (10 mL) and 1,4-dioxane (60 mL) was stirred at 90° C. under nitrogen atmosphere for 16 h. The solvent was removed under vacuum. The residue was purified by silica column chromatography (0-5% ethyl acetate in petroleum ether) to provide the title compound (5.0 g, 83.7% yield). LCMS (ESI) [M+H]⁺=363.1.

### Step 3: Synthesis of ethyl (R)-3-(3-chloro-4-(1,4-dioxaspiro[4.5]decan-8-yl)phenyl)-2-methylpropanoate and ethyl (S)-3-(3-chloro-4-(1,4-dioxaspiro[4.5]decan-8-yl)phenyl)-2-methylpropanoate

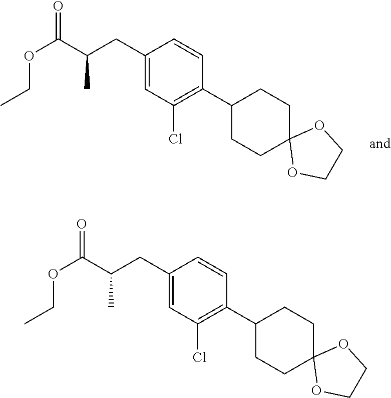 [SMILES: CCOC(=O)[C@H](C)Cc1ccc(C2CCC3(CC2)OCCO3)c(Cl)c1 ; CCOC(=O)[C@@H](C)Cc1ccc(C2CCC3(CC2)OCCO3)c(Cl)c1]

To a solution of ethyl (E)-3-[3-chloro-4-(1,4-dioxaspiro[4.5]dec-7-en-8-yl)phenyl]-2-methyl-prop-2-enoate (500 mg, 1.38 mmol) in ethyl acetate (10 mL) was added platinum(iv)-oxide (31 mg, 0.14 mmol). The mixture was stirred at 25° C. for 2 h under H₂ (15 psi). The reaction mixture was filtered and the filtrate was concentrated to provide the title compound (500 mg, 98.9% yield). LCMS (ESI) [M+H]⁺=367.1.

### Step 4: Synthesis of (R)-3-(3-chloro-4-(1,4-dioxaspiro[4.5]decan-8-yl)phenyl)-2-methylpropan-1-ol and (S)-3-(3-chloro-4-(1,4-dioxaspiro[4.5]decan-8-yl)phenyl)-2-methylpropan-1-ol

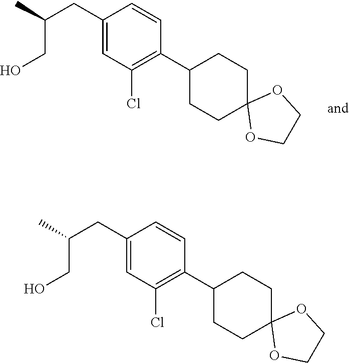 [SMILES: C[C@H](CO)Cc1ccc(C2CCC3(CC2)OCCO3)c(Cl)c1 ; C[C@@H](CO)Cc1ccc(C2CCC3(CC2)OCCO3)c(Cl)c1]

To a solution of ethyl 3-[3-chloro-4-(1,4-dioxaspiro[4.5]decan-8-yl)phenyl]-2-methyl-propanoate (0.5 g, 1.36 mmol) in tetrahydrofuran (15 mL) was added lithium borohydride (0.09 g, 4.09 mmol) at 0° C. The reaction was stirred at 20° C. for 16 h under nitrogen. The reaction mixture was quenched by water (25 mL) and extracted with ethyl acetate (50 mL×3). The combined organics were washed with brine, dried over anhydrous Na₂SO₄, filtered and concentrated to provide the title compound (400 mg, 90.4% yield). LCMS (ESI) [M+H]⁺=325.1.

### Step 5: Synthesis of (R)-3-(3-chloro-4-(1,4-dioxaspiro[4.5]decan-8-yl)phenyl)-2-methylpropanal and (S)-3-(3-chloro-4-(1,4-dioxaspiro[4.5]decan-8-yl)phenyl)-2-methylpropanal

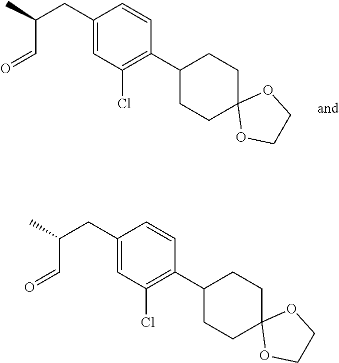 [SMILES: C[C@H](C=O)Cc1ccc(C2CCC3(CC2)OCCO3)c(Cl)c1 ; C[C@@H](C=O)Cc1ccc(C2CCC3(CC2)OCCO3)c(Cl)c1]

To a cold (−75° C.) solution of dimethyl sulfoxide (0.48 g, 6.16 mmol) in dichloromethane (20 mL) was added oxalyl chloride (0.63 g, 4.93 mmol) in dichloromethane (5 mL) dropwise over 10 minutes at −75° C. and continued to stir 30 minutes at −75° C. Then 3-[3-chloro-4-(1,4-dioxaspiro[4.5]decan-8-yl)phenyl]-2-methyl-propan-1-ol (400.0 mg, 1.23 mmol) in dichloromethane (5 mL) was added dropwise over 10 minutes and the mixture was stirred at −75° C. for another 1 h. Triethylamine (0.85 mL, 6.16 mmol) in dichloromethane (5 mL) was added over 15 minutes while maintaining the temperature below −65° C. and stirred at that temperature for 30 minutes, then the reaction mixture was allowed to warm to 0° C. After another 1 h, the reaction was quenched by saturated NH₄C₁ solution (20 mL) and extracted with dichloromethane (50 mL×3). The combined organic phases were washed with saturated sodium bicarbonate (10 mL×3) and brine (10 mL×3), then dried over anhydrous sodium sulfate, filtered and concentrated under reduced pressure to provide the title compound (300 mg, 75.5% yield).

### Step 6: Synthesis of (S)-6-(3-(3-chloro-4-(1,4-dioxaspiro[4.5]decan-8-yl)phenyl)-2-methylpropyl)-2-thia-6-azaspiro[3.4]octane 2,2-dioxide and (R)-6-(3-(3-chloro-4-(1,4-dioxaspiro[4.5]decan-8-yl)phenyl)-2-methylpropyl)-2-thia-6-azaspiro[3.4]octane 2,2-dioxide

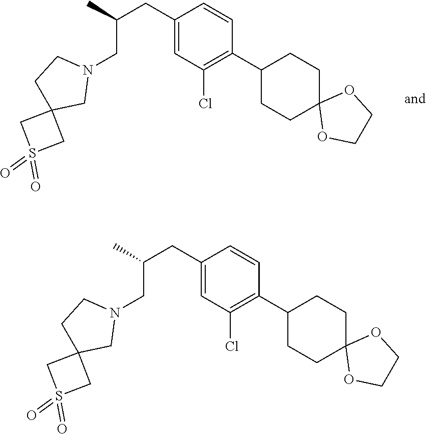 [SMILES: C[C@@H](Cc1ccc(C2CCC3(CC2)OCCO3)c(Cl)c1)CN1CCC2(C1)CS(=O)(=O)C2 ; C[C@H](Cc1ccc(C2CCC3(CC2)OCCO3)c(Cl)c1)CN1CCC2(C1)CS(=O)(=O)C2]

A solution of 2-thia-6-azaspiro[3.4]octane 2,2-dioxide hydrochloride (300.0 mg, 1.52 mmol) and triethylamine (0.21 mL, 1.52 mmol) in dichloromethane (24 mL) was stirred at 25° C. for 30 minutes. Then 3-[3-chloro-4-(1,4-dioxaspiro[4.5]decan-8-yl)phenyl]-2-methyl-propanal (520.0 mg, 1.61 mmol) and acetic acid (0.17 mL, 3.04 mmol) was added to adjust pH=6. The mixture was stirred at 25° C. for another 1 h. Then NaBH(OAc)₃ (643 mg, 3.04 mmol) was added and stirred for 4 h. The reaction mixture was diluted with ethyl acetate (100 mL), and washed with water (30 mL×2), saturated sodium bicarbonate (30 mL×2), dried with anhydrous sodium sulfate, filtered and concentrated. The residue was purified by silica column chromatography (0-50% ethyl acetate in petroleum ether) to provide the title compound (320 mg, 45.1% yield). LCMS (ESI) [M+H]⁺=468.2.

### Step 7: Synthesis of (S)-4-(2-chloro-4-(3-(2,2-dioxido-2-thia-6-azaspiro[3.4]octan-6-yl)-2-methylpropyl)phenyl)cyclohexan-1-one and (R)-4-(2-chloro-4-(3-(2,2-dioxido-2-thia-6-azaspiro[3.4]octan-6-yl)-2-methylpropyl)phenyl)cyclohexan-1-one

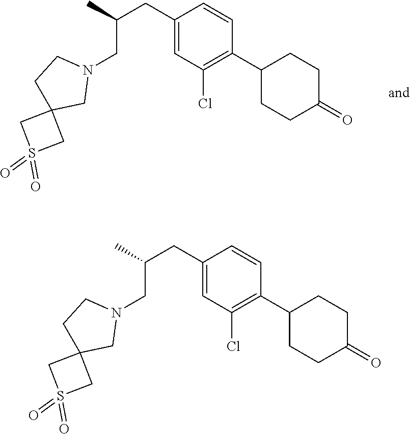 [SMILES: C[C@@H](Cc1ccc(C2CCC(=O)CC2)c(Cl)c1)CN1CCC2(C1)CS(=O)(=O)C2 ; C[C@H](Cc1ccc(C2CCC(=O)CC2)c(Cl)c1)CN1CCC2(C1)CS(=O)(=O)C2]

To a solution of 6-(3-(3-chloro-4-(1,4-dioxaspiro[4.5]decan-8-yl)phenyl)-2-methylpropyl)-2-thia-6-azaspiro[3.4]octane 2,2-dioxide (320.0 mg, 0.68 mmol) in water (8 mL) was added HCl (4 mL, 48 mmol, 12 N) and stirred at 25° C. for 5 h. Saturated sodium bicarbonate was added to adjust pH=8 and extracted with dichloromethane (50 mL×3). The combined organic phase was dried over anhydrous Na₂SO₄, filtered and concentrated under reduced pressure. The residue was purified by silica column chromatography (0-50% ethyl acetate in petroleum ether) to provide the title compound (240 mg, 82.8% yield). LCMS (ESI) [M+H]⁺=424.2.

### Step 8: Synthesis of the Title Compounds

To a solution of 4-(2-chloro-4-(3-(2,2-dioxido-2-thia-6-azaspiro[3.4]octan-6-yl)-2-methylpropyl)phenyl)cyclohexanone (150.0 mg, 0.35 mmol) in tetrahydrofuran (5 mL) was added (trifluoromethyl)trimethylsilane (251 mg, 1.77 mmol). The mixture was stirred at 0° C. for 5 minutes, tetrabutylammoniumfluoride (0.04 mL, 0.04 mmol, 1M in tetrahydrofuran) was then added and the mixture was stirred at 25° C. for 1 hour under nitrogen. The reaction mixture was diluted with dichloromethane (50 mL), and washed with saturated ammonium chloride solution (20 mL×3). The dichloromethane phase was dried over anhydrous Na₂SO₄, filtered and concentrated under vacuum. The residue was purified by silica column chromatography (0-50% ethyl acetate in petroleum ether) to provide the title compound as a mixture of diastereomers (100 mg, 53.8% yield). The cis/trans diastereomers of the mixture were then separated by SFC (Daicel Chiralpak AD (250 mm×30 mm, 10 μm); CO₂, 0.1% NH 3H₂O in EtOH 65/35; 70 m/min). Compound 116A, the first eluting peak, was a racemic mixture of one of the cyclohexane diastereomers of undefined/unassigned relative stereochemistry (37.5 mg, 35.6% yield). Compound 116B, the second eluting peak, was a racemic mixture of one of the cyclohexane diastereomers of undefined/unassigned relative stereochemistry (41.5 mg, 40.7% yield).

Compound 116A* (mixture of diastereomers): ¹H NMR (400 MHz, CDCl₃): δ ppm 7.16-7.14 (m, 2H), 7.01-6.99 (m, 1H), 4.08-4.01 (m, 4H), 3.05-3.12 (m, 1H), 2.82-2.76 (m, 2H), 2.73-2.63 (m, 3H), 2.39-2.26 (m, 5H), 2.14 (t, J=7.2 Hz, 2H), 2.09 (s, 1H), 1.91-1.86 (m, 3H), 1.76-1.74 (m, 4H), 0.86 (d, J=6.4 Hz, 3H). LCMS (ESI) [M+H]⁺=494.1.

Compound 116B* (mixture of diastereomers): ¹H NMR (400 MHz, CDCl₃): δ ppm 7.16-7.14 (m, 2H), 7.01-6.99 (m, 1H), 4.08-4.01 (m, 4H), 3.05-3.12 (m, 1H), 2.82-2.63 (m, 5H), 2.38-2.26 (m, 5H), 2.14 (t, J=7.2 Hz, 2H), 2.08 (s, 1H), 1.91-1.86 (m, 3H), 1.76-1.74 (m, 4H), 0.86 (d, J=6.8 Hz, 3H). LCMS (ESI) [M+H]⁺=494.1.

### Examples 117A* and 117B*: (S)-6-(3-(3-Chloro-4-(4,4-difluorocyclohexyl)phenyl)-2-methylpropyl)-2-thia-6-azaspiro[3.4]octane 2,2-dioxide and (R)-6-(3-(3-Chloro-4-(4,4-difluorocyclohexyl)phenyl)-2-methylpropyl)-2-thia-6-azaspiro[3.4]octane 2,2-dioxide

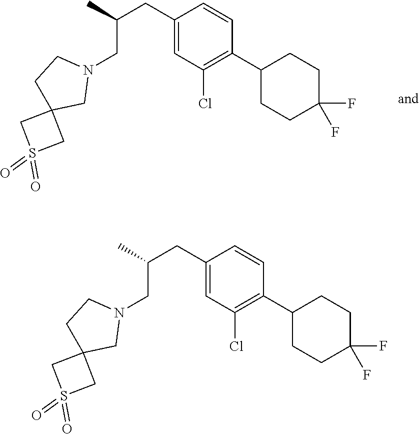 [SMILES: C[C@@H](Cc1ccc(C2CCC(F)(F)CC2)c(Cl)c1)CN1CCC2(C1)CS(=O)(=O)C2 ; C[C@H](Cc1ccc(C2CCC(F)(F)CC2)c(Cl)c1)CN1CCC2(C1)CS(=O)(=O)C2]

To a stirred solution of 4-(2-chloro-4-(3-(2,2-dioxido-2-thia-6-azaspiro[3.4]octan-6-yl)-2-methylpropyl)phenyl)cyclohexanone (from example 38; 80.0 mg, 0.19 mmol) in dichloromethane (5 mL) at 0° C. was added diethylaminosulfur trifluoride (0.25 mL, 1.89 mmol). The reaction mixture was stirred at 20° C. for 1 h. The reaction was quenched by saturated sodium bicarbonate solution (15 mL) and extracted with ethyl acetate (25 ml×3). The combined organics were washed with water and brine, dried over anhydrous Na₂SO₄, filtered and concentrated under vacuum. The residue was purified by silica column chromatography (0-50% ethyl acetate in petroleum ether) to provide a racemic mixture of the title compound (70 mg, 74% yield). LCMS (ESI) [M+H]⁺=446.1. The mixture was separated by chiral SFC (Daicel Chiralpak AD (250 mm×50 mm, 10 m); CO₂, 0.1% NH₃H₂O in EtOH, 75/25; 60 mL/min). Compound 117A, the first eluting peak, was obtained as a single enantiomer of undefined/unassigned absolute stereochemistry (17.7 mg, 23.5% yield). Compound 117B, the second eluting peak, was obtained as a single enantiomer of undefined/unassigned absolute stereochemistry (17.6 mg, 24.6% yield).

Compound 117A*: ¹H NMR (400 MHz, CDCl₃): δ ppm 7.17-7.15 (m, 2H), 7.02-7.00 (m, 1H), 4.08-4.05 (m, 4H), 3.11-3.04 (m, 1H), 2.85-2.60 (m, 5H), 2.39-2.23 (m, 5H), 2.14 (t, J=7.2 Hz, 2H), 1.96-1.83 (m, 5H), 1.79-1.69 (m, 2H), 0.86 (d, J=6.4 Hz, 3H). LCMS (ESI) [M+H]⁺=446.1.

Compound 117B*: ¹H NMR (400 MHz, CDCl₃): δ ppm 7.17-7.15 (m, 2H), 7.02-7.00 (m, 1H), 4.08-4.05 (m, 4H), 3.11-3.04 (m, 1H), 2.82-2.61 (m, 5H), 2.39-2.23 (m, 5H), 2.14 (t, J=7.2 Hz, 2H), 1.96-1.83 (m, 5H), 1.79-1.69 (m, 2H), 0.86 (d, J=6.4 Hz, 3H). LCMS (ESI) [M+H]⁺=446.1.

### Examples 118A* and 118B*: (S)-6-(2-Methyl-3-(6-(trifluoromethyl)pyridin-3-yl)propyl)-2-thia-6-azaspiro[3.4]octane 2,2-dioxide and (R)-6-(2-Methyl-3-(6-(trifluoromethyl)pyridin-3-yl)propyl)-2-thia-6-azaspiro[3.4]octane 2,2-dioxide

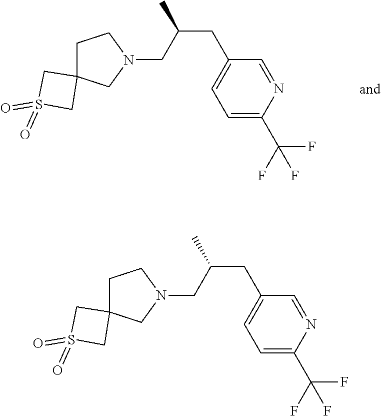 [SMILES: C[C@@H](Cc1ccc(C(F)(F)F)nc1)CN1CCC2(C1)CS(=O)(=O)C2 ; C[C@H](Cc1ccc(C(F)(F)F)nc1)CN1CCC2(C1)CS(=O)(=O)C2]

The title compounds were synthesized using the synthetic procedure described for Example 113 utilizing methyl-2-(diethylphosphono)proprionate and 2-trifluoromethyl-5-pyridinealdehyde in step 1. The racemic mixture of the above titled compound was separated by SFC (Daicel Chiralpak AD (250 mm×30 mm, 10 μm); CO₂, 0.1% DEA in EtOH, 5-40%; 25 m/min). Compound 118A, the first eluting peak, was obtained as a single enantiomer of undefined/unassigned absolute stereochemistry (32 mg, 52% yield). Compound 118B, the second eluting peak, was obtained as a single enantiomer of undefined/unassigned absolute stereochemistry (24.8 mg, 37.8% yield).

Compound 118A*: ¹H NMR (400 MHz, CD₃OD): δ ppm 8.55 (d, J=1.6 Hz, 1H), 7.89 (dd, J=8.0, 1.2 Hz, 1H), 7.75 (d, J=8.0 Hz, 1H), 4.07 (d, J=4.4 Hz, 4H), 3.06-2.88 (m, 2H), 2.80 (s, 2H), 2.73-2.63 (m, 2H), 2.61-2.55 (m, 1H), 2.44-2.32 (m, 2H), 2.17-2.15 (m, 2H), 2.05-1.96 (m, 1H), 0.89 (d, J=6.8 Hz, 3H). LCMS (ESI) [M+H]⁺=363.1.

Compound 118B*: ¹H NMR (400 MHz, CD₃OD): δ ppm 8.55 (d, J=1.6 Hz, 1H), 7.89 (dd, J=8.0, 1.2 Hz, 1H), 7.75 (d, J=8.0 Hz, 1H), 4.07 (d, J=4.4 Hz, 4H), 3.06-2.88 (m, 2H), 2.80 (s, 2H), 2.73-2.63 (m, 2H), 2.61-2.55 (m, 1H), 2.44-2.32 (m, 2H), 2.17-2.15 (m, 2H), 2.05-1.96 (m, 1H), 0.89 (d, J=6.8 Hz, 3H). LCMS (ESI) [M+H]⁺=363.1.

### Examples 119A* and 119B*: (S)-6-(2-Methyl-3-(4-(trifluoromethoxy)phenyl)propyl)-2-thia-6-azaspiro[3.4]octane 2,2-dioxide and (R)-6-(2-Methyl-3-(4-(trifluoromethoxy)phenyl)propyl)-2-thia-6-azaspiro[3.4]octane 2,2-dioxide

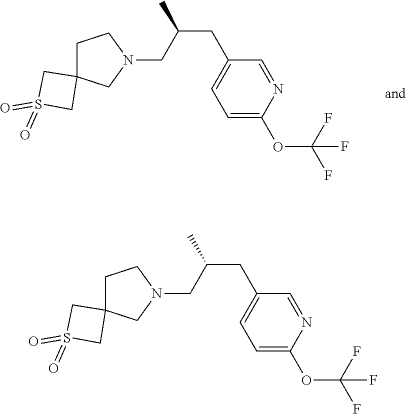 [SMILES: C[C@@H](Cc1ccc(OC(F)(F)F)nc1)CN1CCC2(C1)CS(=O)(=O)C2 ; C[C@H](Cc1ccc(OC(F)(F)F)nc1)CN1CCC2(C1)CS(=O)(=O)C2]

The title compounds were synthesized using the synthetic procedure described for Example 113, utilizing methyl-2-(diethylphosphono)proprionate and 4-trifluoromethoxy benzaldehyde in step 1. The racemic mixture of the above titled compound was separated by SFC (Daicel Chiralpak OJ (250 mm×30 mm, 10 μm); CO₂, 0.1% DEA in EtOH, 5-40%; 28 m/min). Compound 119A, the first eluting peak, was obtained as a single enantiomer of undefined/unassigned absolute stereochemistry (33.7 mg, 60% yield). Compound 119B, the second eluting peak, was obtained as a single enantiomer of undefined/unassigned absolute stereochemistry (12 mg, 21% yield).

Compound 119A*: ¹H NMR (400 MHz, CD₃OD): δ ppm 7.26 (d, J=8.0 Hz, 2H), 7.17 (d, J=8.0 Hz, 2H), 4.08 (s, 4H), 2.87-2.81 (m, 1H), 2.80 (s, 2H), 2.73-2.63 (m, 2H), 2.44-2.36 (m, 2H), 2.35-2.29 (m, 1H), 2.16 (t, J=7.2 Hz, 2H), 2.00-1.86 (m, 1H), 0.87 (d, J=6.4 Hz, 3H). LCMS (ESI) [M+H]⁺=378.1.

Compound 119B*: 1H NMR (400 MHz, CD₃OD): δ ppm 7.25 (d, J=8.0 Hz, 2H), 7.16 (d, J=8.0 Hz, 2H), 4.08 (s, 4H), 2.86-2.81 (m, 1H), 2.80 (s, 2H), 2.71-2.63 (m, 2H), 2.43-2.36 (m, 2H), 2.35-2.29 (m, 1H), 2.16 (t, J=7.2 Hz, 2H), 2.00-1.86 (m, 1H), 0.87 (d, J=6.8 Hz, 3H). LCMS (ESI) [M+H]⁺=378.1.

### Example 120: 6-(2-Methyl-2-(4-(trifluoromethyl)phenoxy)propyl)-2-thia-6-azaspiro[3.4]octane 2,2-dioxide

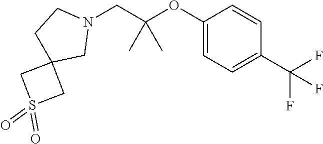 [SMILES: CC(C)(CN1CCC2(C1)CS(=O)(=O)C2)Oc1ccc(C(F)(F)F)cc1]

### Step 1: Synthesis of methyl 2-methyl-2-[4-(trifluoromethyl)phenoxy]propanoate

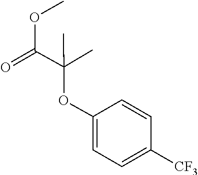 [SMILES: COC(=O)C(C)(C)Oc1ccc(C(F)(F)F)cc1]

To a solution of 4-(trifluoromethyl)phenol (1.2 g, 7.4 mmol) in N,N-dimethylformamide (20 mL) was added K₂CO₃ (3 g, 22.21 mmol) and methyl 2-bromo-2-methylpropanoate (1.1 g, 5.91 mmol). The mixture was stirred at 80° C. for 15 h. The reaction was quenched by saturated NH₄C₁ solution (40 mL) and exacted with ethyl acetate (40 mL×3). The combined organics were washed with brine (30 mL×3), dried over anhydrous Na₂SO₄, filtered and concentrated under vacuum. The residue was purified by silica chromatography (0-30% ethyl acetate in petroleum ether) to provide the title compound (1.5 g, 77% yield). ¹H NMR (400 MHz, CDCl₃): δ ppm 7.51 (d, J=8.4 Hz, 2H), 6.87 (d, J=8.4 Hz, 2H), 3.77 (s, 3H), 1.65 (s, 6H).

### Step 2: Synthesis of 2-methyl-2-[4-(trifluoromethyl)phenoxy]propanal

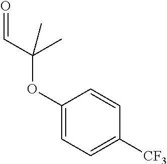 [SMILES: CC(C)(C=O)Oc1ccc(C(F)(F)F)cc1]

To a solution of methyl 2-methyl-2-[4-(trifluoromethyl)phenoxy]propanoate (200 mg, 0.76 mmol) in dichloromethane (2 mL) at −78° C. under nitrogen, diisobutylaluminum hydride (1 mL, 1 mmol, 1 N in toluene) was added and then stirred at −78° C. for 2 h. The reaction was quenched by saturated NH₄C₁ solution (10 mL) and extracted with ethyl acetate (10 mL×3). The combined organics were washed with brine (10 mL×3), dried over anhydrous Na₂SO₄, filtered and removed under vacuum to provide the title compound (160 mg, 90.3% yield). ¹H NMR (400 MHz, CDCl₃): δ ppm 9.82 (s, 1H), 7.54-7.48 (m, 2H), 6.91-6.87 (m, 2H), 1.49 (s, 6H).

### Step 3: Synthesis of the Title Compound

To a solution of 2-methyl-2-[4-(trifluoromethyl)phenoxy]propanal (160 mg, 0.688 mmol) in methanol (4 mL) was added 2-thia-6-azaspirol[3.4]octane 2,2-dioxide hydrochloride (40 mg, 0.2 mmol) and NaBH₃CN (64 mg, 1.0 mmol). The mixture was stirred at 80° C. for 4 h. The reaction mixture was purified by reverse phase chromatography (acetonitrile 1-28%/0.2% formic acid in water) to provide the title compound (20 mg, 30% yield).

Compound 120: ¹H NMR (400 MHz, CDCl₃): δ ppm 7.53 (d, J=8.4 Hz, 2H), 7.05 (d, J=8.4 Hz, 2H), 4.09 (s, 4H), 3.13-2.92 (m, 4H), 2.80 (brs, 2H), 2.20-2.18 (m, 2H), 1.36 (s, 6H). LCMS (ESI) [M+H]⁺=378.0.

### Examples 121A* and 121B*: (S)-6-(2-Methyl-3-(5-(trifluoromethyl)pyridin-2-yl)propyl)-2-thia-6-azaspiro[3.4]octane 2,2-dioxide and (R)-6-(2-Methyl-3-(5-(trifluoromethyl)pyridin-2-yl)propyl)-2-thia-6-azaspiro[3.4]octane 2,2-dioxide

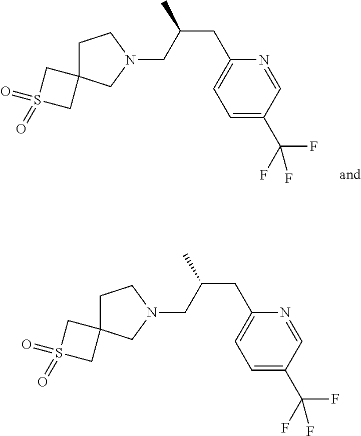 [SMILES: C[C@@H](Cc1ccc(C(F)(F)F)cn1)CN1CCC2(C1)CS(=O)(=O)C2 ; C[C@H](Cc1ccc(C(F)(F)F)cn1)CN1CCC2(C1)CS(=O)(=O)C2]

The title compounds were synthesized using the synthetic procedure described for Example 113, utilizing methyl-2-(diethylphosphono)proprionate and 5-(trifluoromethyl)-2-pyridinecarboxaldehyde in step 1. The racemic mixture of the above titled compound was separated by SFC (Daicel Chiralpak OD (250 mm×30 mm, 10 μm); CO₂, 0.1% NH₄OH in EtOH, 45%; 60 mL/min). Compound 121A, the first eluting peak, was obtained as a single enantiomer of undefined/unassigned absolute stereochemistry (5.1 mg, 10% yield). Compound 121B, the second eluting peak, was obtained as a single enantiomer of undefined/unassigned absolute stereochemistry (5.8 mg, 11% yield).

Compound 121A*: ¹H NMR (400 MHz, CD₃OD): δ ppm 8.77 (s, 1H), 8.03 (dd, J=8.0, 2.0 Hz, 1H), 7.50 (d, J=8.0 Hz, 1H), 4.05 (s, 4H), 3.30-3.29 (m, 1H), 3.07-3.03 (m, 1H), 2.83-2.77 (m, 2H), 2.70-2.61 (m, 3H), 2.44-2.38 (m, 2H), 2.25-2.17 (m, 1H), 2.13 (t, J=6.8 Hz, 2H), 0.89 (d, J=6.8 Hz, 3H). LCMS (ESI) [M+H]⁺=363.1.

Compound 121B*: ¹H NMR (400 MHz, CD₃OD): δ ppm 8.77 (s, 1H), 8.03 (dd, J=8.0, 2.0 Hz, 1H), 7.50 (d, J=8.0 Hz, 1H), 4.05 (s, 4H), 3.30-3.29 (m, 1H), 3.07-3.03 (m, 1H), 2.83-2.77 (m, 2H), 2.70-2.61 (m, 3H), 2.44-2.38 (m, 2H), 2.25-2.17 (m, 1H), 2.13 (t, J=6.8 Hz, 2H), 0.89 (d, J=6.8 Hz, 3H). LCMS (ESI) [M+H]⁺=363.1.

### Examples 122A* and 122B*: (R)-6-(3-Fluoro-3-(4-(trifluoromethyl)phenyl)propyl)-2-thia-6-azaspiro[3.4]octane 2,2-dioxide and (S)-6-(3-Fluoro-3-(4-(trifluoromethyl)phenyl)propyl)-2-thia-6-azaspiro[3.4]octane 2,2-dioxide

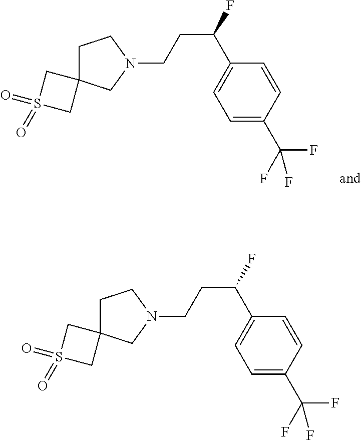 [SMILES: O=S1(=O)CC2(CCN(CC[C@@H](F)c3ccc(C(F)(F)F)cc3)C2)C1 ; O=S1(=O)CC2(CCN(CC[C@H](F)c3ccc(C(F)(F)F)cc3)C2)C1]

### Step 1: Synthesis of (S)-1-(4-(trifluoromethyl)phenyl)but-3-en-1-ol and (R)-1-(4-(trifluoromethyl)phenyl)but-3-en-1-ol

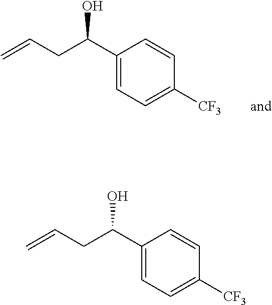

To a stirred solution of 4-(trifluoromethyl)benzaldehyde (2.00 g, 11.5 mmol) in tetrahydrofuran (20 mL) at 0° C. was added 1M solution of allylmagnesium bromide (15 mL, 15 mmol, 1 M in hexane) dropwise. Then the reaction mixture was stirred at 0° C. for 2 h. The reaction mixture was quenched by saturated NH₄Cl solution (40 mL), and extracted with ethyl acetate (50 mL×2). The combined organic phase was washed with brine, dried over anhydrous Na₂SO₄, filtered and concentrated under reduced pressure to give the crude, which was purified by silica chromatography (0-70% ethyl acetate in petroleum ether) to provide the title compound (2.24 g, 10.4 mmol, 90.2% yield) as a mixture of enantiomers. ¹H NMR (400 MHz, DMSO-d₆): δ ppm 7.67 (d, J=8.4 Hz, 2H), 7.54 (d, J=8.4 Hz, 2H), 5.79-5.73 (m, 1H), 5.01-4.93 (m, 2H), 4.71-4.68 (m, 1H), 2.43-2.38 (m, 2H).

### Step 2: Synthesis of (S)-1-(1-fluorobut-3-en-1-yl)-4-(trifluoromethyl)benzene and (R)-1-(1-fluorobut-3-en-1-yl)-4-(trifluoromethyl)benzene

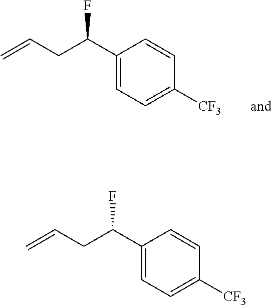

To a stirred solution of 1-[4-(trifluoromethyl)phenyl]but-3-en-1-ol (2.0 g, 9.25 mmol) in dichloromethane (10 mL) at −78° C. was added diethylaminosulfurtrifluoride (4.0 mL, 27.7 mmol) and the mixture was stirred at 20° C. for 3 h. The reaction was quenched by saturated NaHCO₃ solution (15 mL) and extracted with ethyl acetate (30 mL×2). The combined organic phase was washed with brine (30 mL), dried over Na₂SO₄, filtered and concentrated under vacuum. The residue was purified by silica column chromatography (0-30% ethyl acetate in petroleum ether) to provide the title compound (1.86 g, 8.53 mmol, 92.2% yield) as a mixture of enantiomers. ¹H NMR (400 MHz, DMSO-d₆): δ ppm 7.76 (d, J=8.4 Hz, 2H), 7.60 (d, J=8.4 Hz, 2H), 5.81-5.69 (m, 2H), 5.15-5.08 (m, 2H), 2.72-2.63 (m, 2H).

### Step 3: Synthesis of (S)-3-fluoro-3-(4-(trifluoromethyl)phenyl)propanal and (R)-3-fluoro-3-(4-(trifluoromethyl)phenyl)propanal

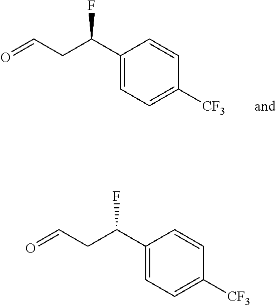 [SMILES: O=CC[C@@H](F)c1ccc(C(F)(F)F)cc1 ; O=CC[C@H](F)c1ccc(C(F)(F)F)cc1]

To a mixture of 1-(1-fluorobut-3-enyl)-4-(trifluoromethyl)benzene (600.0 mg, 2.75 mmol) in dichloromethane (20 mL) at −78° C. was bubbled a stream of ozone (15 psi) for 30 minutes. The mixture was quenched with dimethyl sulfide (854.4 mg, 13.75 mmol) and stirred at 25° C. for 60 minutes. The reaction mixture was washed with water (20 mL×3). The organic layers were dried over anhydrous Na₂SO₄, filtered and concentrated under vacuum to give the title compound (400 mg, 66.1% yield) as a mixture of enantiomers. ¹H NMR (400 MHz, CDCl₃): δ ppm 9.86 (s, 1H), 7.70 (d, J=8.0 Hz, 2H), 7.50 (d, J=8.0 Hz, 2H), 6.18-6.02 (m, 1H), 3.25-3.19 (m, 1H), 2.99-2.91 (m, 1H).

### Step 4: Synthesis of the Title Compounds

To a solution of 3-fluoro-3-[4-(trifluoromethyl)phenyl]propanal (217 mg, 0.99 mmol) in methanol (2 mL) was added 2-thia-6-azaspiro[3.4]octane-2,2-dioxide hydrochloride (65 mg, 0.33 mmol) and NaBH₃CN (103 mg, 1.64 mmol). The mixture was stirred at 70° C. for 3 hours. TLC indicated the reaction was completed. The reaction mixture was purified by reverse phase chromatography (C18; acetonitrile 30-60%/0.1% NH₄OH in water) to provide the title compound (40 mg, 33% yield) as a mixture of enantiomers. The racemic mixture (40 mg, 0.109 mmol) was purified by chiral SFC (Daicel Chiralcel OJ-H (150 mm×4.6 mm, 5 m) CO₂, ethanol, 40%). Example 122A, the first eluting peak, was obtained as a single enantiomer of unassigned stereochemistry (19.6 mg, 49% yield). Example 122B, the second eluting peak, was afforded as a single enantiomer of unassigned stereochemistry (16.6 mg, 42% yield).

Compound 122A*: ¹H NMR (400 MHz, CD₃OD): δ ppm 7.70 (d, J=8.0 Hz, 2H), 7.56 (d, J=8.0 Hz, 2H), 5.74-5.59 (m, 1H), 4.09 (s, 4H), 2.85 (s, 2H), 2.74-2.65 (m, 4H), 2.21-2.04 (m, 4H). LCMS (ESI) [M+H]⁺=366.0.

Compound 122B*: ¹H NMR (400 MHz, CD₃OD): δ ppm 7.70 (d, J=8.4 Hz, 2H), 7.56 (d, J=8.4 Hz, 2H), 5.74-5.59 (m, 1H), 4.09 (s, 4H), 2.85 (s, 2H), 2.73-2.61 (m, 4H), 2.21-2.11 (m, 4H). LCMS (ESI) [M+H]⁺=366.0.

### Examples 123A* and 123B*: 6-((R)-3-(3-Chloro-4-((1r,4R)-4-hydroxy-4-methylcyclohexyl)phenyl)-2-methylpropyl)-2-thia-6-azaspiro[3.4]octane 2,2-dioxide, 6-((S)-3-(3-chloro-4-((1r,4S)-4-hydroxy-4-methylcyclohexyl)phenyl)-2-methylpropyl)-2-thia-6-azaspiro[3.4]octane 2,2-dioxide, 6-((S)-3-(3-Chloro-4-((1r,4S)-4-hydroxy-4-methylcyclohexyl)phenyl)-2-methylpropyl)-2-thia-6-azaspiro[3.4]octane 2,2-dioxide, and 6-((R)-3-(3-chloro-4-((1r,4R)-4-hydroxy-4-methylcyclohexyl)phenyl)-2-methylpropyl)-2-thia-6-azaspiro[3.4]octane 2,2-dioxide

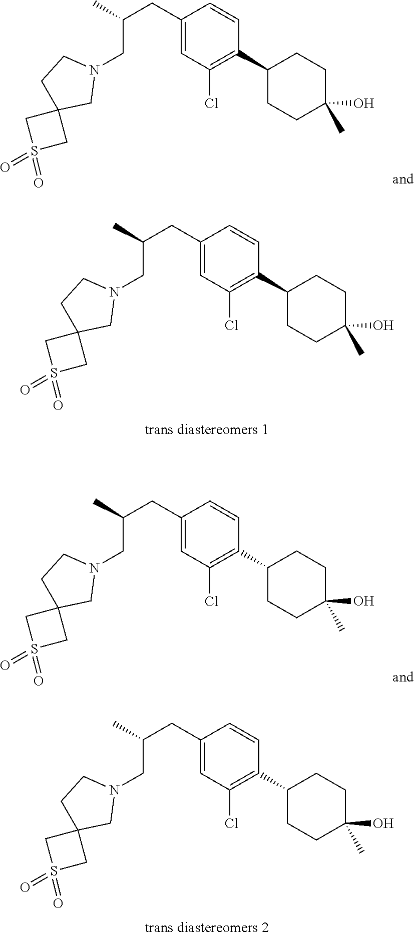 [SMILES: C[C@H](Cc1ccc([C@H]2CC[C@](C)(O)CC2)c(Cl)c1)CN1CCC2(C1)CS(=O)(=O)C2 ; C[C@@H](Cc1ccc([C@H]2CC[C@](C)(O)CC2)c(Cl)c1)CN1CCC2(C1)CS(=O)(=O)C2 ; C[C@@H](Cc1ccc([C@H]2CC[C@](C)(O)CC2)c(Cl)c1)CN1CCC2(C1)CS(=O)(=O)C2 ; C[C@H](Cc1ccc([C@H]2CC[C@](C)(O)CC2)c(Cl)c1)CN1CCC2(C1)CS(=O)(=O)C2]

Methylmagnesiumbromide (0.39 mL, 1.18 mmol, 3 M in toluene) was added to a solution of 4-(2-chloro-4-(3-(2,2-dioxido-2-thia-6-azaspiro[3.4]octan-6-yl)-2-methylpropyl)phenyl) cyclohexanone (from example 38; 100 mg, 0.24 mmol) in tetrahydrofuran (5 mL) at 0° C. The mixture was stirred at 0° C. for 3 h. The reaction was quenched by saturated ammonium chloride solution (5 mL) and extracted with dichloromethane (25 mL×3). The combined dichloromethane fractions were dried over anhydrous Na₂SO₄, filtered and concentrated under vacuum. The residue was purified by reverse phase chromatography (C18; water (0.05% NH₃H₂O+10 mM NH₄HCO₃)-ACN, 65%-95%) to provide Compound 123A as the first eluting peak, which is a mixture of diastereomers of undefined/unassigned relative stereochemistry (8.6 mg, 8.2% yield), and to provide Compound 123B as the second eluting peak as a mixture of diastereomers of undefined/unassigned relative stereochemistry (10.5 mg, 10% yield).

Compound 123A*: ¹H NMR (400 MHz, CDCl₃): δ ppm 7.17-7.14 (m, 2H), 6.99 (dd, J=8.0, 2.0 Hz, 1H), 4.08-4.04 (m, 4H), 3.02-2.95 (m, 1H), 2.82-2.61 (m, 5H), 2.39-2.28 (m, 3H), 2.14 (t, J=7.2 Hz, 2H), 1.86-1.83 (m, 5H), 1.70-1.64 (m, 2H), 1.54-1.50 (m, 2H), 1.40 (s, 1H), 1.35 (s, 3H), 0.86 (d, J=6.4 Hz, 3H). LCMS (ESI) [M+H]⁺=440.1.

Compound 123B*: ¹H NMR (400 MHz, CDCl₃): δ ppm 7.23 (d, J=8.0 Hz, 1H), 7.13 (d, J=1.6 Hz, 1H), 7.00 (dd, J=8.0, 1.6 Hz, 1H), 4.08-4.01 (m, 4H), 2.95-2.89 (m, 1H), 2.82-2.60 (m, 5H), 2.39-2.25 (m, 3H), 2.13 (t, J=7.2 Hz, 2H), 1.86-1.60 (m, 7H), 1.54-1.50 (m, 2H), 1.30 (s, 3H), 1.17 (s, 1H), 0.86 (d, J=6.4 Hz, 3H). LCMS (ESI) [M+H]⁺=440.1.

### Examples 124A* and 124B*: 6-((R)-3-(3-Chloro-4-((R)-tetrahydro-2H-pyran-2-yl)phenyl)-2-methylpropyl)-2-thia-6-azaspiro[3.4]octane 2,2-dioxide, 6-((S)-3-(3-chloro-4-((R)-tetrahydro-2H-pyran-2-yl)phenyl)-2-methylpropyl)-2-thia-6-azaspiro[3.4]octane 2,2-dioxide, 6-((R)-3-(3-Chloro-4-((S)-tetrahydro-2H-pyran-2-yl)phenyl)-2-methylpropyl)-2-thia-6-azaspiro[3.4]octane 2,2-dioxide, and 6-((S)-3-(3-chloro-4-((S)-tetrahydro-2H-pyran-2-yl)phenyl)-2-methylpropyl)-2-thia-6-azaspiro[3.4]octane 2,2-dioxide

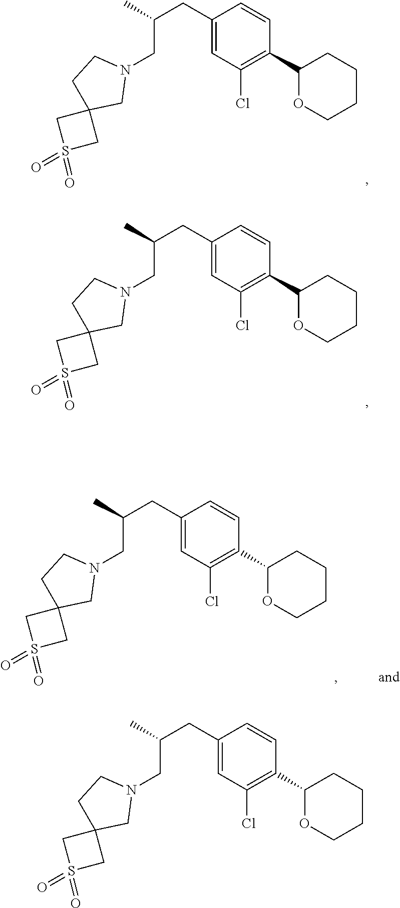

The title compounds were synthesized using the synthetic procedure described for Example 116, utilizing 3,4-dibydro-6-(4,4,5,5-tetramethyli-1,3,2-dioxaborolan-2-yl)-2-pyran in step 2 and omitting steps 7 and 8. The diasteromeric mixture was purified by reverse phase chromatography (C18; water (0.05% NH₃H₂O+10 mM NH₄HCO₃)-ACN, 30%) to provide Compound 124A* as the first eluting peak, obtained as a mixture of diastereomers of undefined/unassigned relative stereochemistry (9.8 mg, 23% yield), and to provide Compound 124B* as the second eluting peak. The title compounds were obtained as a mixture of diastereomers of undefined/unassigned relative stereochemistry (13.7 mg, 31.6% yield).

Compound 124A*: ¹H NMR (400 MHz, CD₃OD): δ ppm 7.51-7.49 (m, 1H), 7.24-7.12 (m, 2H), 4.71-4.67 (m, 1H), 4.12 (s, 4H), 3.65-3.55 (m, 1H), 3.22 (s, 1H), 2.89-2.82 (m, 2H), 2.77-2.69 (m, 2H), 2.23-2.19 (m, 2H), 2.05-1.94 (m, 2H), 1.94-1.84 (m, 4H), 1.74-1.59 (m, 3H), 1.48-1.34 (m, 2H), 1.31-1.25 (m, 3H). LCMS (ESI) [M+H]⁺=410.1.

Compound 124B*: ¹H NMR (400 MHz, CD₃OD): δ ppm 7.50 (d, J=8.0 Hz, 1H), 7.25-7.22 (m, 2H), 4.71-4.67 (m, 1H), 4.12 (s, 4H), 3.65-3.55 (m, 1H), 3.21 (s, 2H), 2.86 (s, 2H), 2.74 (t, J=7.2 Hz, 2H), 2.21 (t, J=7.2 Hz, 2H), 1.95-1.91 (m, 3H), 1.82-1.61 (m, 3H), 1.54-1.35 (m, 2H), 1.31-1.25 (m, 3H). LCMS (ESI) [M+H]⁺=410.1.

### Example 125: 6-(2,2-Dimethyl-3-(4-(trifluoromethyl)phenyl)propyl)-2-thia-6-azaspiro[3.4]octane-2,2-dioxide

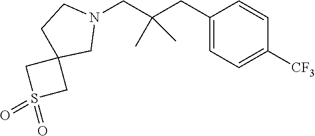 [SMILES: CC(C)(Cc1ccc(C(F)(F)F)cc1)CN1CCC2(C1)CS(=O)(=O)C2]

### Step 1: Synthesis of 2,2-dimethyl-3-(4-(trifluoromethyl)phenyl)propanal

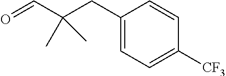 [SMILES: CC(C)(C=O)Cc1ccc(C(F)(F)F)cc1]

To a mixture of tribromoindium (297 mg, 0.84 mmol) and 4-(trifluoromethyl)benzyl bromide (2.0 g, 8.37 mmol) dissolved in dichloromethane (20 mL), 2-methyl-1-(trimethylsiloxy)-1-propene (3.62 g, 25.1 mmol) was added and stirred at 20° C. for 16 h. The reaction was quenched by saturated sodium bicarbonate (50 mL) and extracted with dichloromethane (50 mL×3). The combined organic phase was washed with brine, dried over anhydrous Na₂SO₄, filtered and concentrated under vacuum. The residue was purified by silica column chromatography (0-10% ethyl acetate in petroleum ether) to provide the title compound (500 mg, 26% yield). LCMS (ESI) [M+H]⁺=231.0.

### Step 2: Synthesis of the Title Compound

2-Thia-6-azaspiro[3.4]octane 2,2-dioxide hydrochloride (30 mg, 0.15 mmol) and triethylamine (15 mg, 0.15 mmol) were added to dichloromethane (4 mL) and stirred at 25° C. for 30 minutes. 2,2-Dimethyl-3-[4-(trifluoromethyl)phenyl]propanal (35 mg, 0.15 mmol) and acetic acid (18 mg, 0.3 mmol) were added to above mixture and stirred at 25° C. for another 1 h. Then NaBH(OAc)₃ (64 mg, 0.3 mmol) was added and the reaction mixture was stirred for 1 hour. The reaction was quenched by saturated sodium bicarbonate (10 mL) and extracted with dichloromethane (15 mL×3). The combined organic phase was dried over Na₂SO₄, filtered and concentrated under reduced pressure to give the crude, which was purified by reverse phase chromatography (water (0.05% NH₃H₂O+10 mM NH₄HCO₃)-ACN, 70%-100%) to provide the title compound (9.5 mg, 16.5% yield).

Compound 125: ¹H NMR (400 MHz, CD₃OD): δ ppm 7.55 (d, J=8.0 Hz, 2H), 7.35 (d, J=8.0 Hz, 2H), 4.09 (s, 4H), 2.96 (s, 2H), 2.85 (t, J=7.2 Hz, 2H), 2.67 (s, 2H), 2.39 (s, 2H), 2.15 (t, J=7.2 Hz, 2H), 0.88 (s, 6H). LCMS (ESI) [M+H]⁺=375.1.

### Examples 126A* and 126B*: (S)-6-(2-Methyl-3-(2-(trifluoromethyl)pyridin-4-yl)propyl)-2-thia-6-azaspiro[3.4]octane 2,2-dioxide and (R)-6-(2-Methyl-3-(2-(trifluoromethyl)pyridin-4-yl)propyl)-2-thia-6-azaspiro[3.4]octane 2,2-dioxide

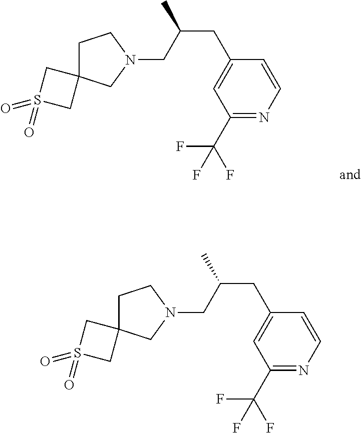 [SMILES: C[C@@H](Cc1ccnc(C(F)(F)F)c1)CN1CCC2(C1)CS(=O)(=O)C2 ; C[C@H](Cc1ccnc(C(F)(F)F)c1)CN1CCC2(C1)CS(=O)(=O)C2]

### Step 1: Synthesis of methyl (E)-2-methyl-3-[2-(trifluoromethyl)-4-pyridyl]propenoate

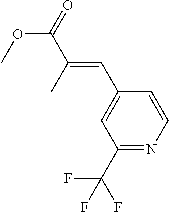 [SMILES: COC(=O)/C(C)=C/c1ccnc(C(F)(F)F)c1]

To a solution of 4-iodo-2-(trifluoromethyl)pyridine (1.1 g, 4 mmol) in anhydrous DMF (11 mL) was added methyl methacrylate (1.21 g, 12 mmol), NaHCO₃ (813 mg, 9.7 mmol) and Pd(Cy*Phine)₂C₁₂ (103 mg, 0.08 mmol). The reaction mixture was stirred at 80° C. for 16 h. The reaction was quenched by saturated NH₄C₁ (20 mL) and extracted with ethyl acetate (50 mL×2). The combined organic layer was washed with brine (30 mL×3), dried over anhydrous sodium sulfate, filtered and concentrated under vacuum. The residue was purified by silica column chromatography (0-10% ethyl acetate in petroleum ether) to provide the title compound (210 mg, 21% yield).

### Step 2: Synthesis of methyl (R)-2-methyl-3-(2-(trifluoromethyl)pyridin-4-yl)propanoate and methyl (S)-2-methyl-3-(2-(trifluoromethyl)pyridin-4-yl)propanoate

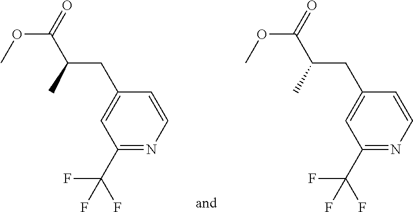 [SMILES: COC(=O)[C@H](C)Cc1ccnc(C(F)(F)F)c1 ; COC(=O)[C@@H](C)Cc1ccnc(C(F)(F)F)c1]

To a solution of methyl (E)-2-methyl-3-[2-(trifluoromethyl)-4-pyridyl]prop-2-enoate (210 mg, 0.86 mmol) in ethyl acetate (7 mL) was added PtO₂/C (20 mg, 0.09 mmol). The suspension was degassed under vacuum and purged with H₂ (15 psi) three times. The mixture was stirred under H₂ (15 psi) at 25° C. for 1 h. The reaction mixture was filtered and the filtrate was concentrated under vacuum. The residue was further purified by silica column chromatography (0-20% ethyl acetate in petroleum ether) to provide the title compound (180 mg, 85% yield) as a mixture of enantiomers. LCMS (EIS): [M+H]⁺=248.1.

### Step 3: Synthesis of (S)-2-methyl-3-(2-(trifluoromethyl)pyridin-4-yl)propanal and (R)-2-methyl-3-(2-(trifluoromethyl)pyridin-4-yl)propanal

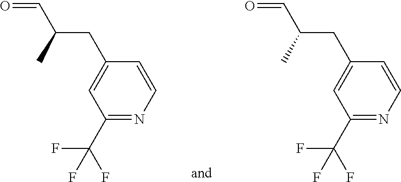

To a solution of methyl 2-methyl-3-[2-(trifluoromethyl)-4-pyridyl]propanoate (180 mg, 0.728 mmol) in dry dichloromethane (4 mL) was added diisobutylaluminum hydride (1 mL, 1 mmol, 1 M in DCM) dropwise at −78° C. and stirred at that temperature for 1 h. The reaction mixture was quenched by saturated ammonium chloride solution (3 mL) and extracted with ethyl acetate (25 mL×2). The combined organic phase was dried over anhydrous Na₂SO₄, filtered and concentrated under vacuum. The residue was purified by silica column chromatography (0-20% ethyl acetate in petroleum ether) to provide the title compound (110 mg, 70% yield) as a mixture of enantiomers.

### Step 4: Synthesis of the Title Compounds

The mixture of 2-thia-6-azaspiro[3.4]octane 2,2-dioxide hydrochloride (80 mg, 0.4 mmol) and Triethylamine (82 mg, 0.81 mmol) in dichloromethane (10 mL) was stirred at 25° C. for 30 minutes, then 2-methyl-3-[2-(trifluoromethyl)-4-pyridyl]propanal (88 mg, 0.4 mmol) and acetic acid (24 mg, 0.4 mmol) were added. The mixture was stirred at 25° C. for 1 h, NaBH(OAc)₃ (172 mg, 0.81 mmol) was added and stirred at 25° C. for another 1 h. The reaction was quenched by saturated solution of NaHCO₃ and extracted with ethyl acetate (50 mL×2). The combined organic phase was washed with brine, dried over anhydrous sodium sulfate, filtered and concentrated under vacuum. The residue was purified by reverse phase chromatography (water (0.05% NH₃H₂O)-ACN, 40%˜70%) to provide the title compound (70 mg, 48% yield) as a mixture of enantiomers. LCMS (ESI) [M+H]⁺=363.2. The racemic mixture (70 mg, 0.19 mmol) was purified by chiral SFC (Daicel Chiralpak AD-H (250 mm×30 mm, 5 m); CO₂, 0.1% NH₃H₂O in EtOH; 15%, 60 mL/min) affording Compound 126A as the first eluting peak (16.2 mg, 23% yield) as a pure enantiomer of undetermined absolute stereochemistry, and affording Compound 126B as the second eluting peak (16.5 mg, 24% yield) as a pure enantiomer of undetermined absolute stereochemistry

Compound 126A*: ¹H NMR (400 MHz, CDCl₃): δ ppm 8.62 (d, J=4.8 Hz, 1H), 7.49 (s, 1H), 7.28 (d, J=4.8 Hz, 1H), 4.11-3.99 (m, 4H), 2.95-2.62 (m, 5H), 2.51-2.47 (m, 1H), 2.39-2.28 (m, 2H), 2.16-2.08 (m, 2H), 1.99-1.92 (m, 1H), 0.88 (d, J=6.4 Hz, 3H). LCMS (ESI) [M+H]⁺=363.2.

Compound 126B*: ¹H NMR (400 MHz, CDCl₃): δ ppm 8.62 (d, J=4.8 Hz, 1H), 7.49 (s, 1H), 7.29 (d, J=4.8 Hz, 1H), 4.10-4.00 (m, 4H), 2.93-2.59 (m, 5H), 2.51-2.47 (m, 1H), 2.37-2.26 (m, 2H), 2.18-2.07 (m, 2H), 1.98-1.91 (m, 1H), 0.88 (d, J=6.4 Hz, 3H). LCMS (ESI) [M+H]⁺=363.2.

### Example 127: 6-(2-(3-Fluorophenoxy)-2-methylpropyl)-2-thia-6-azaspiro[3.4]octane-2,2-dioxide

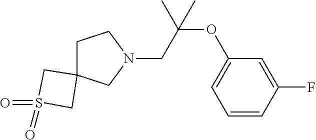 [SMILES: CC(C)(CN1CCC2(C1)CS(=O)(=O)C2)Oc1cccc(F)c1]

The title compound was synthesized using the synthetic procedure described for Example 120, utilizing 3-fluorophenol in step 1 (28.9 mg, 44% yield).

Compound 127: ¹H NMR (400 MHz, CD₃OD): δ ppm 8.33 (brs, 1H), 7.35-7.21 (m, 1H), 6.89-6.73 (m, 3H), 4.13 (s, 4H), 3.16 (s, 2H), 3.01 (t, J=7.2 Hz, 2H), 2.88 (s, 2H), 2.21 (t, J=7.2 Hz, 2H), 1.33 (s, 6H). LCMS (ESI) [M+H]⁺=328.1.

### Examples 128A* and 128B*: (S)-6-(3-(4-(Difluoromethyl)phenyl)-2-methylpropyl)-2-thia-6-azaspiro[3.4]octane 2,2-dioxide and (R)-6-(3-(4-(Difluoromethyl)phenyl)-2-methylpropyl)-2-thia-6-azaspiro[3.4]octane 2,2-dioxide

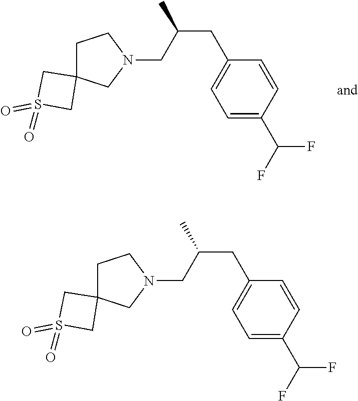 [SMILES: C[C@@H](Cc1ccc(C(F)F)cc1)CN1CCC2(C1)CS(=O)(=O)C2 ; C[C@H](Cc1ccc(C(F)F)cc1)CN1CCC2(C1)CS(=O)(=O)C2]

The title compounds were synthesized using the synthetic procedure described for Example 103, utilizing 4-difluoromethyl-iodobenzene and 2-thia-6-azaspiro[3.4]octane-2,2-dioxide hydrochloride. The crude diastereomeric mixture of the title compound was separated by chiral SFC (OJ-3, 150×4.6 mm, 3 μm; CO₂-EtOH, 5-40%). Compound 128A was afforded as the first eluting peak (10.5 mg, 26% yield), as a single enantiomer of undefined/unassigned absolute stereochemistry, and Compound 128B was obtained as the second eluting peak (9.9 mg, 25% yield), as a single enantiomer of undefined/unassigned absolute stereochemistry.

Compound 128A*: ¹H NMR (400 MHz, CD₃OD): δ ppm 7.44 (d, J=8.0 Hz, 2H), 7.29 (d, J=8.0 Hz, 2H), 6.90-6.54 (m, 1H), 4.09 (d, J=1.6 Hz, 4H), 2.89-2.82 (m, 3H), 2.77-2.67 (m, 2H), 2.47-2.32 (m, 3H), 2.17 (t, J=7.2 Hz, 2H), 2.02-1.92 (m, 1H), 0.88 (d, J=6.4 Hz, 3H). LCMS (ESI) [M+H]⁺=343.9.

Compound 128B*: ¹H NMR (400 MHz, CD₃OD): δ ppm 7.44 (d, J=8.0 Hz, 2H), 7.29 (d, J=8.0 Hz, 2H), 6.89-6.54 (m, 1H), 4.09 (d, J=1.6 Hz, 4H), 2.88-2.81 (m, 3H), 2.76-2.66 (m, 2H), 2.46-2.32 (m, 3H), 2.17 (t, J=7.2 Hz, 2H), 2.02-1.93 (m, 1H), 0.88 (d, J=6.4 Hz, 3H). LCMS (ESI) [M+H]⁺=343.9.

### Example 129: 6-(2-(4-Cyclobutylphenoxy)-2-methylpropyl)-2-thia-6-azaspiro[3.4]octane-2,2-dioxide

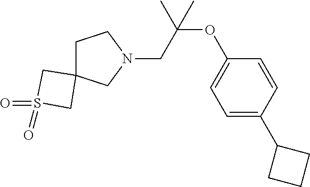 [SMILES: CC(C)(CN1CCC2(C1)CS(=O)(=O)C2)Oc1ccc(C2CCC2)cc1]

### Step 1: Synthesis of 4-cyclobutylphenol

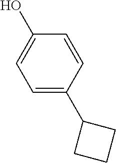 [SMILES: Oc1ccc(C2CCC2)cc1]

To a mixture of 4-bromophenol (1.0 g, 5.78 mmol) and bromocyclobutane (780 mg, 5.78 mmol) in N,N-dimethylacetamide (10 mL) was added 4,4′-di-tert-butyl-2,2′-dipyridyl (155 mg, 0.58 mmol), 4-ethylpyridine (309 mg, 2.89 mmol), manganese (635 mg, 11.56 mmol), KI (959 mg, 5.78 mmol) and NiCl₂ dimethoxyethane adduct (127 mg, 0.58 mmol) in glove box under argon. The mixture was stirred at 80° C. for 16 h. The mixture was diluted with ethyl acetate (200 mL) and washed with brine (30 mL×3), dried over anhydrous Na₂SO₄, filtered and concentrated under reduced pressure. The residue was purified by silica column chromatography (0-25% ethyl acetate in petroleum ether) to provide the title compound (500 mg, 58.4% yield).

### Step 2: Synthesis of methyl 2-(4-cyclobutylphenoxy)-2-methylpropanoate

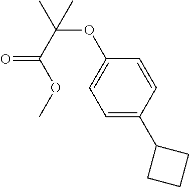 [SMILES: COC(=O)C(C)(C)Oc1ccc(C2CCC2)cc1]

A suspension of 4-cyclobutylphenol (400 mg, 2.7 mmol), methyl 2-bromo-2-methylpropanoate (733 mg, 4.05 mmol), and K₂CO₃ (1119 mg, 8.10 mmol) in N,N-dimethylformamide (8 mL) was stirred at 80° C. for 16 h. Ethyl acetate (500 mL) was added and the resulting mixture was washed with brine (100 mL×3), dried over anhydrous Na₂SO₄, filtered and concentrated in vacuo. The residue was purified by silica column chromatography (0-5% ethyl acetate in petroleum ether) to provide the title compound (180 mg, 26.9% yield). LCMS (ESI) [M+H]⁺=249.1.

### Step 3: Synthesis of 2-(4-cyclobutylphenoxy)-2-methylpropanal

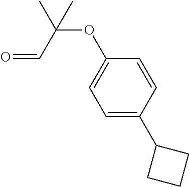 [SMILES: CC(C)(C=O)Oc1ccc(C2CCC2)cc1]

To a solution of methyl 2-(4-cyclobutylphenoxy)-2-methyl-propanoate (100 mg, 0.4 mmol) in dichloromethane (4 mL) was added diisobutylaluminum hydride (0.6 mL, 0.6 mmol, 1 M in DCM) at −78° C. The reaction was stirred at −78° C. for 1 h. This reaction was quenched by potassium tartrate aqueous solution (5 mL). The resulting solution was extracted with ethyl acetate (50 mL×2) and the combined organic phase was washed with brine, dried over anhydrous Na₂SO₄, filtered and concentrated in vacuo to provide the title compound (100 mg, 90% yield).

### Step 4: Synthesis of the Title Compound

A solution of 2-thia-6-azaspiro[3.4]octane 2,2-dioxide hydrochloride (30 mg, 0.15 mmol) and triethylamine (0.02 mL, 0.15 mmol) in anhydrous dichloromethane (4 mL) was stirred at 25° C. for 30 minutes. Then 2-(4-cyclobutylphenoxy)-2-methyl-propanal (40 mg, 0.18 mmol) and acetic acid (0.02 mL, 0.3 mmol) were added and stirred at 25° C. for 1 h. NaBH(OAc)₃ (64 mg, 0.3 mmol) was added and stirred at 25° C. for 1 h. The reaction mixture was diluted with ethyl acetate (60 mL) and the resulting mixture was washed with water (30 mL×2). The organic layer was washed with saturated sodium bicarbonate (30 mL), dried over anhydrous Na₂SO₄, filtered and concentrated in vacuo. The crude residue was purified by silica column chromatography (0-50% ethyl acetate in petroleum ether) to provide the pure title compound (32.3 mg, 57.4% yield).

Compound 129: ¹H NMR (400 MHz, CDCl₃): δ ppm 7.11 (d, J=8.4 Hz, 2H), 6.89 (d, J=8.4 Hz, 2H), 4.10-4.03 (m, 4H), 3.55-3.46 (m, 1H), 3.02 (s, 2H), 2.90 (t, J=7.2 Hz, 2H), 2.71 (s, 2H), 2.36-2.29 (m, 2H), 2.17-2.07 (m, 4H), 2.03-1.96 (m, 1H), 1.88-1.81 (m, 1H), 1.26 (s, 6H). LCMS (ESI) [M+H]⁺=364.3.

### Example 130: 6-(2-Methyl-2-(3-(trifluoromethyl)phenoxy)propyl)-2-thia-6-azaspiro[3.4]octane-2,2-dioxide

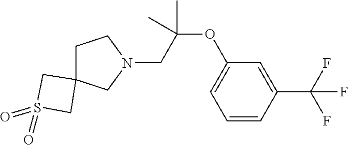 [SMILES: CC(C)(CN1CCC2(C1)CS(=O)(=O)C2)Oc1cccc(C(F)(F)F)c1]

The title compound was synthesized using the synthetic procedure described for Example 120, utilizing 3-trifluoromethylphenol in step 1 (22.5 mg, 38.9% yield).

Compound 130: ¹H NMR (400 MHz, CD₃OD): δ ppm 7.50-7.46 (m, 1H), 7.39-7.37 (m, 1H), 7.29-7.27 (m, 2H), 4.13-4.10 (m, 4H), 3.17 (s, 2H), 3.03 (t, J=7.2 Hz, 2H), 2.92 (s, 2H), 2.22 (t, J=7.2 Hz, 2H), 1.34 (s, 6H). LCMS (ESI) [M+H]⁺=378.1.

### Example 131: 6-(2-Methyl-2-(3-(trifluoromethoxy)phenoxy)propyl)-2-thia-6-azaspiro[3.4]octane 2,2-dioxide

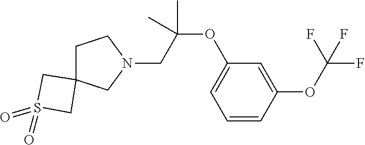 [SMILES: CC(C)(CN1CCC2(C1)CS(=O)(=O)C2)Oc1cccc(OC(F)(F)F)c1]

The title compound was synthesized using the synthetic procedure described for example 120, utilizing 3-trifluoromethoxyphenol in step 1 (12.5 mg, 21% yield).

Compound 131: ¹H NMR (400 MHz, CDCl₃): δ ppm 7.30-7.29 (m, 1H), 7.01-6.90 (m, 2H), 6.85 (s, 1H), 4.07 (s, 4H), 3.02-2.91 (m, 4H), 2.74 (s, 2H), 2.16-2.11 (m, 2H), 1.31 (s, 6H). LCMS (ESI) [M+H]⁺=394.0.

### Example 132: 6-(2-(4-Fluorophenoxy)-2-methylpropyl)-2-thia-6-azaspiro[3.4]octane 2,2-dioxide

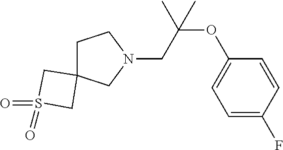 [SMILES: CC(C)(CN1CCC2(C1)CS(=O)(=O)C2)Oc1ccc(F)cc1]

The title compound was synthesized using the synthetic procedure described for example 120, utilizing 4-fluorophenol in step 1 (11 mg, 16% yield).

Compound 132: ¹H NMR (400 MHz, CD₃OD): δ ppm 7.20-7.00 (m, 4H), 4.09 (s, 4H), 3.04 (s, 2H), 2.90 (t, J=7.2 Hz, 2H), 2.77 (s, 2H), 2.16 (t, J=7.2 Hz, 2H), 1.28 (s, 6H). LCMS (ESI) [M+H]⁺=328.1.

### Example 133: 6-(2-(2-Fluorophenoxy)-2-methylpropyl)-2-thia-6-azaspiro[3.4]octane 2,2-dioxide

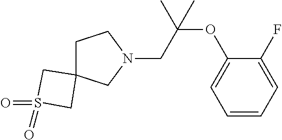 [SMILES: CC(C)(CN1CCC2(C1)CS(=O)(=O)C2)Oc1ccccc1F]

The title compound was synthesized using the synthetic procedure described for example 120, utilizing 2-fluorophenol in step 1 (15 mg, 23% yield).

Compound 133: ¹H NMR (400 MHz, CD₃OD): δ ppm 7.15-7.06 (m, 4H), 4.09 (s, 4H), 3.04 (s, 2H), 2.90 (t, J=7.2 Hz, 2H), 2.77 (s, 2H), 2.16 (t, J=7.2 Hz, 2H), 1.28 (s, 6H). LCMS (ESI) [M+H]⁺=328.1.

### Example 135: 6-(2-Methyl-2-((6-(trifluoromethyl)pyridin-3-yl)oxy)propyl)-2-thia-6-azaspiro[3.4]octane-2,2-dioxide

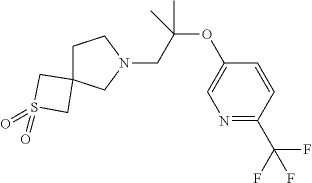 [SMILES: CC(C)(CN1CCC2(C1)CS(=O)(=O)C2)Oc1ccc(C(F)(F)F)nc1]

The title compound was synthesized using the synthetic procedure described for example 120, utilizing 2-trifluoromethyl-5-hydroxypyridine in step 1 (30.2 mg, 51.5% yield).

Compound 135: ¹H NMR (400 MHz, CDCl₃): δ ppm 8.39 (s, 1H), 7.61 (d, J=8.4 Hz, 1H), 7.41 (dd, J=8.4, 2.4 Hz, 1H), 4.10-4.02 (m, 4H), 3.02 (s, 2H), 2.90 (t, J=7.2 Hz, 2H), 2.79 (s, 2H), 2.15 (t, J=7.2 Hz, 2H), 1.59 (s, 2H), 1.37 (s, 6H). LCMS (ESI) [M+H]⁺=379.1.

### Example 136: 6-(2-Methyl-2-(4-(trifluoromethoxy)phenoxy)propyl)-2-thia-6-azaspiro[3.4]octane 2,2-dioxide

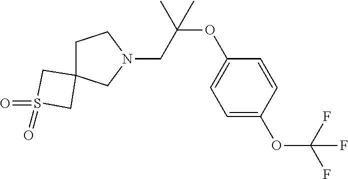 [SMILES: CC(C)(CN1CCC2(C1)CS(=O)(=O)C2)Oc1ccc(OC(F)(F)F)cc1]

The title compound was synthesized using the synthetic procedure described for example 120, utilizing 4-trifluoromethoxyphenol in step 1 (26.3 mg, 30.4% yield).

Compound 136: ¹H NMR (400 MHz, CDCl₃): δ ppm 7.12 (d, J=8.4 Hz, 2H), 6.98-6.94 (m, 2H), 4.07 (s, 4H), 3.02 (s, 2H), 2.90 (t, J=7.2 Hz, 2H), 2.72 (s, 2H), 2.14 (t, J=7.2 Hz, 2H), 1.28 (s, 6H). LCMS (ESI) [M+H]⁺=394.2.

### Example 138: 7-(2-Methyl-2-(4-(trifluoromethyl)phenoxy)propyl)-2-thia-7-azaspiro[3.5]nonane 2,2-dioxide

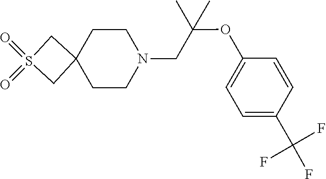 [SMILES: CC(C)(CN1CCC2(CC1)CS(=O)(=O)C2)Oc1ccc(C(F)(F)F)cc1]

The title compound was synthesized using the synthetic procedure described for example 120, utilizing 4-trifluoromethylphenol in step 1, and 2-thia-7-azaspiro[3.5]nonane-2,2-dioxide hydrochloride in step 3 (21.4 mg, 30.5% yield).

Compound 138: ¹H NMR (400 MHz, CDCl₃): δ ppm 7.52 (d, J=8.4 Hz, 2H), 7.05 (d, J=8.4 Hz, 2H), 3.86 (s, 4H), 2.60 (brs, 4H), 2.53 (s, 2H), 1.92 (t, J=5.2 Hz, 4H), 1.32 (s, 6H). LCMS (ESI) [M+H]⁺=392.1.

### Example 139: 6-(3-(3-Chloro-4-(4,4-difluorocyclohexyl)phenyl)-2,2-dimethylpropyl)-2-thia-6-azaspiro[3.4]octane 2,2-dioxide

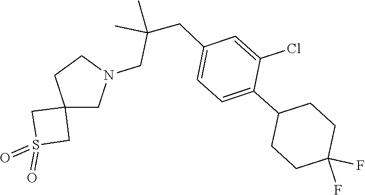 [SMILES: CC(C)(Cc1ccc(C2CCC(F)(F)CC2)c(Cl)c1)CN1CCC2(C1)CS(=O)(=O)C2]

### Step 1: Synthesis of 4-(bromomethyl)-2-chloro-1-(4,4-difluorocyclohexyl)benzene

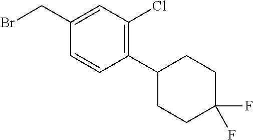 [SMILES: FC1(F)CCC(c2ccc(CBr)cc2Cl)CC1]

To the mixture of 3-chloro-4-(4,4-difluorocyclohexyl)phenyl]methanol (1000 mg, 3.84 mmol) in dichloromethane (10 mL) was added phosphorous tribromide (0.15 mL, 1.53 mmol) at 0° C. and the reaction mixture was stirred at 25° C. for 2 h. The reaction was quenched with NaHCO₃ solution (50 mL) and extracted with dichloromethane (60 mL×3). The combined organic layers were washed with brine (50 mL×3), dried over anhydrous Na₂SO₄, filtered and concentrated under reduced pressure. The residue was purified by silica flash chromatography (0-10% ethyl acetate in petroleum ether) to provide the title compound (600 mg, 48.3% yield).

### Step 2: Synthesis of methyl 3-(3-chloro-4-(4,4-difluorocyclohexyl)phenyl)-2,2-dimethylpropanoate

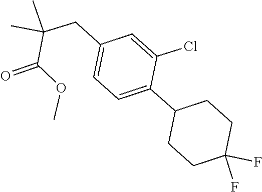 [SMILES: COC(=O)C(C)(C)Cc1ccc(C2CCC(F)(F)CC2)c(Cl)c1]

To a solution of methyl isobutyrate (26 mL, 3.29 mmol) in tetrahydrofuran (8 mL) was added lithium diisopropylamide (1.55 mL, 3.09 mmol) at −78° C. under N₂ at −75° C. and stirred at that temperature for 1 h. Then 4-(bromomethyl)-2-chloro-1-(4,4-difluorocyclohexyl) benzene (500 mg, 1.55 mmol) in tetrahydrofuran (2 mL) was added slowly at −75° C. and then stirred at −75° C. for 1 h. The reaction mixture was quenched with saturated NH₄Cl (20 mL) solution and extracted with ethyl acetate (50 mL×3). The combined organics were washed with brine (20 mL×2), dried over anhydrous sodium sulfate, filtered and concentrated. The residue was purified by silica flash chromatography (0-5% ethyl acetate in petroleum ether) to provide the title compound (250 mg, 46.9% yield).

### Step 3: Synthesis of 3-(3-chloro-4-(4,4-difluorocyclohexyl)phenyl)-2,2-dimethylpropanal

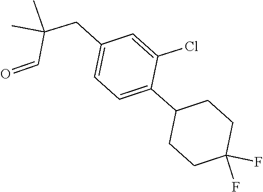 [SMILES: CC(C)(C=O)Cc1ccc(C2CCC(F)(F)CC2)c(Cl)c1]

To a solution of methyl 3-[3-chloro-4-(4,4-difluorocyclohexyl)phenyl]-2,2-dimethyl-propanoate (240 mg, 0.70 mmol) in dichloromethane (7 mL) was added diisobutylalumanylium hydride (1.04 mL, 1.04 mmol, 1M in toluene) at −75° C. and stirred at −75° C. for 2 hours. The reaction mixture was quenched by potassium sodium tartrate solution (10 mL)/water (10 mL) and extracted with ethyl acetate (50 mL×3). The combined organic layers were washed with brine (30 mL×3), dried over anhydrous Na₂SO₄, filtered and concentrated. The residue was purified by silica flash chromatography (0-5% ethyl acetate in petroleum ether) to provide the title compound (35 mg, 16% yield).

### Step 4: Synthesis of the Title Compound

The solution of 2-thia-6-azaspiro[3.4]octane 2,2-dioxide hydrochloride (30 mg, 0.15 mmol) and triethylamine (15 mg, 0.15 mmol) in dichloromethane (3 mL) was stirred at 25° C. for 30 minutes. Then 3-[3-chloro-4-(4,4-difluorocyclohexyl)phenyl]-2,2-dimethyl-propanal (35 mg, 0.12 mmol) and acetic acid (18 mg, 0.30 mmol) was added. The mixture was stirred at 25° C. for 1 hour. Then NaBH(OAc)₃ (64 mg, 0.30 mmol) was added and stirred at 25° C. for another 1 hour. The reaction mixture was quenched with saturated sodium bicarbonate (10 mL) and extracted with dichloromethane (15 mL×3). The combined organic layers were dried over sodium sulfate, filtered and concentrated. The residue was purified by silica flash chromatography (0-30% ethyl acetate in petroleum ether) to provide the title compound (21.3 mg, 29.3% yield). LCMS (ESI) [M+H]⁺=460.2.

Compound 139: ¹H NMR (400 MHz, CD₃OD): δ ppm 7.24-7.19 (m, 2H), 7.07 (d, J=7.6 Hz, 1H), 4.09 (s, 4H), 3.16-3.10 (m, 2H), 2.93 (s, 2H), 2.84 (t, J=7.2 Hz, 2H), 2.54 (s, 2H), 2.35 (s, 2H), 2.17-2.13 (m, 4H), 2.06-1.87 (m, 4H), 1.79-1.73 (m, 2H), 0.86 (s, 6H). LCMS (ESI) [M+H]⁺=460.2.

### Example 140: 6-(2,2-Dimethyl-3-(6-(trifluoromethyl)pyridin-3-yl)propyl)-2-thia-6-azaspiro[3.4]octane-2,2-dioxide

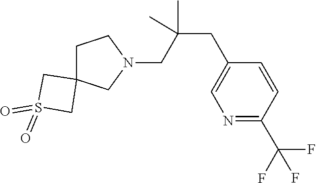 [SMILES: CC(C)(Cc1ccc(C(F)(F)F)nc1)CN1CCC2(C1)CS(=O)(=O)C2]

### Step 1: Synthesis of methyl 2,2-dimethyl-3-(6-(trifluoromethyl)pyridin-3-yl)propanoate

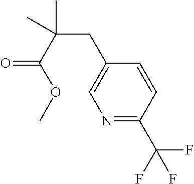 [SMILES: COC(=O)C(C)(C)Cc1ccc(C(F)(F)F)nc1]

To a solution of methyl isobutyrate (271 mg, 2.66 mmol) in tetrahydrofuran (5 mL) was added lithium diisopropylamide (1.25 mL, 2.5 mmol, 2M) at −78° C. and stirred at −78° C. for 1 h. Then 5-(bromomethyl)-2-(trifluoromethyl)pyridine (300 mg, 1.25 mmol) was added slowly at −78° C. and stirred for 2 h. The reaction was quenched by NH₄Cl aq. (15 mL) and extracted with ethyl acetate (25 mL×3). The combined organics were washed with brine (25 mL), dried over sodium sulfate, filtered and concentrated. The residue was purified by silica column chromatography (0-20% ethyl acetate in petroleum ether) to provide the title compound (300 mg, 81.2% yield). LCMS (ESI) [M+H]⁺=262.2.

### Step 2: Synthesis of 2,2-dimethyl-3-(6-(trifluoromethyl)pyridin-3-yl)propanal

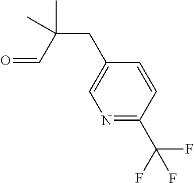 [SMILES: CC(C)(C=O)Cc1ccc(C(F)(F)F)nc1]

To a solution of methyl 2,2-dimethyl-3-[6-(trifluoromethyl)-3-pyridyl]propanoate (300 mg, 1.15 mmol) in dichloromethane (10 mL) was added diisobutylaluminum hydride (1.72 mL, 1.72 mmol, 1M in DCM) at −78° C. and stirred at that temperature for 2 h. The reaction was quenched by potassium sodium tartrate aqueous solution (5 mL). The resulting solution was extracted with ethyl acetate (25 mL×3). The combined organic phase was washed with water and brine, dried over anhydrous Na₂SO₄, filtered and concentrated in vacuo. The residue was purified by silica column chromatography (0-20% ethyl acetate in petroleum ether) to provide the title compound (80 mg, 30.1% yield). LCMS (ESI) [M+H]⁺=232.1.

### Step 3: Synthesis of the Title Compound

A solution of 2-thia-6-azaspiro[3.4]octane-2,2-dioxide hydrochloride (30 mg, 0.15 mmol) and triethylamine (0.02 mL, 0.15 mmol) in anhydrous dichloromethane (6 mL) was stirred at 25° C. for 30 minutes. Then 2,2-dimethyl-3-[6-(trifluoromethyl)-3-pyridyl]propanal (35 mg, 0.15 mmol) and acetic acid (0.02 mL, 0.30 mmol) were added and stirred at 25° C. for 1 h. Then NaBH(OAc)₃ (64 mg, 0.30 mmol) was added and stirred at 25° C. for another 1 h. The reaction mixture was diluted with ethyl acetate (50 mL), and washed with water. The organic layer was washed with saturated sodium bicarbonate and concentrated under reduced pressure. The residue was purified by silica column chromatography (0-50% ethyl acetate in petroleum ether) to provide the title compound (30 mg, 49.9% yield).

Compound 140: ¹H NMR (400 MHz, CDCl₃): δ ppm 8.51 (s, 1H), 7.64-7.60 (m, 2H), 4.10-4.03 (m, 4H), 2.97 (s, 2H), 2.86 (t, J=7.2 Hz, 2H), 2.64 (s, 2H), 2.38 (s, 2H), 2.15 (t, J=7.2 Hz, 2H), 0.87 (s, 6H). LCMS (ESI) [M+H]⁺=377.2.

### Examples 141A* and 141B*: (R)-7-(2-Methyl-2-(4-(trifluoromethyl)phenoxy)propyl)-2-thia-7-azaspiro[4.4]nonane 2,2-dioxide and (S)-7-(2-Methyl-2-(4-(trifluoromethyl)phenoxy)propyl)-2-thia-7-azaspiro[4.4]nonane 2,2-dioxide

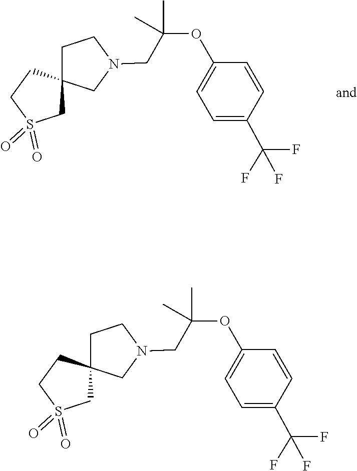 [SMILES: CC(C)(CN1CC[C@@]2(CCS(=O)(=O)C2)C1)Oc1ccc(C(F)(F)F)cc1 ; CC(C)(CN1CC[C@]2(CCS(=O)(=O)C2)C1)Oc1ccc(C(F)(F)F)cc1]

The title compounds were synthesized using the synthetic procedure described for Example 120, utilizing 4-trifluoromethylphenol in step 1, and 2-thia-7-azaspiro[4.4] nonane-2,2-dioxide hydrochloride in step 3. The title compounds were isolated as a racemic mixture (35 mg) and the enantiomers were separated by chiral SFC (Daicel Chiralpak AD-H (250 mm×30 mm, 5 m)); CO₂, 0.1% NH₃H₂O/EtOH=25:75; 60 mL/min) affording Compound 141A as the first eluting peak (8.8 mg, 22.9% yield) as a single enantiomer of undefined/unassigned absolute stereochemistry, and providing Compound 141B as the second eluting peak (8.8 mg, 22.9% yield) as a single enantiomer of undefined/unassigned absolute stereochemistry.

Compound 141A*: ¹H NMR (400 MHz, CDCl₃): δ ppm 7.54 (d, J=6.8 Hz, 2H), 7.06 (d, J=6.8 Hz, 2H), 3.25-3.18 (m, 2H), 3.17-2.96 (m, 4H), 2.85-2.65 (m, 4H), 2.27-2.24 (m, 2H), 2.09-1.78 (m, 2H), 1.35 (s, 6H). LCMS (ESI) [M+H]⁺=392.1.

Compound 141B*: ¹H NMR (400 MHz, CDCl₃): δ 7.54 (d, J=6.8 Hz, 2H), 7.06 (d, J=6.8 Hz, 2H), 3.25-3.18 (m, 2H), 3.17-2.96 (m, 4H), 2.85-2.65 (m, 4H), 2.27-2.24 (m, 2H), 2.09-1.78 (m, 2H), 1.35 (s, 6H). LCMS (ESI) [M+H]⁺=392.1.

### Example 142: (S)-6-(2-Fluoro-3-(4-(trifluoromethyl)phenyl)propyl)-2-thia-6-azaspiro[3.4]octane 2,2-dioxide and (R)-6-(2-fluoro-3-(4-(trifluoromethyl)phenyl)propyl)-2-thia-6-azaspiro[3.4]octane 2,2-dioxide

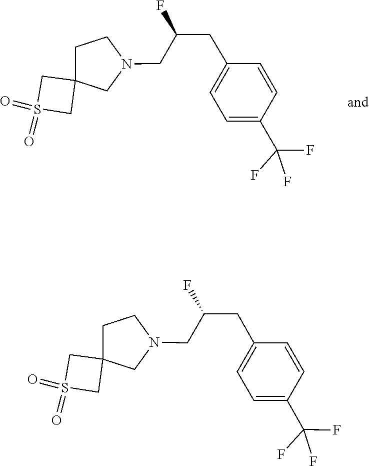 [SMILES: O=S1(=O)CC2(CCN(C[C@@H](F)Cc3ccc(C(F)(F)F)cc3)C2)C1 ; O=S1(=O)CC2(CCN(C[C@H](F)Cc3ccc(C(F)(F)F)cc3)C2)C1]

### Step 1: Synthesis of 2-(4-(trifluoromethyl)benzyl)oxirane

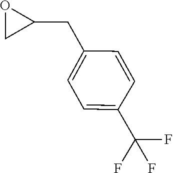 [SMILES: FC(F)(F)c1ccc(CC2CO2)cc1]

To a solution of 1-allyl-4-(trifluoromethyl)benzene (200 mg, 1.07 mmol) in dichloromethane (10 mL) was added 3-chloroperoxybenzoicacid (436 mg, 2.15 mmol, 85% purity) and stirred at 25° C. for 16 h. The reaction was quenched by saturated aqueous NaHCO₃ solution (15 mL) and the organic layer was separated. The aqueous layer was further extracted with dichloromethane, and the combined organic phase was dried over anhydrous MgSO₄, filtered and concentrated under reduced pressure. The resulting residue was purified by silica column chromatography (0-5% ethyl acetate in petroleum ether) to provide the title compound (180 mg, 82.9% yield). ¹H NMR (400 MHz, CDCl₃): δ ppm 7.59 (d, J=8.0 Hz, 2H), 7.39 (d, J=8.0 Hz, 2H), 3.20-3.16 (m, 1H), 3.00-2.88 (m, 2H), 2.83 (t, J=4.4 Hz, 1H), 2.55 (dd, J=4.8, 2.8 Hz, 1H).

### Step 2: Synthesis of (R)-6-(2-hydroxy-3-(4-(trifluoromethyl)phenyl)propyl)-2-thia-6-azaspiro[3.4]octane 2,2-dioxide and (S)-6-(2-hydroxy-3-(4-(trifluoromethyl)phenyl)propyl)-2-thia-6-azaspiro[3.4]octane 2,2-dioxide

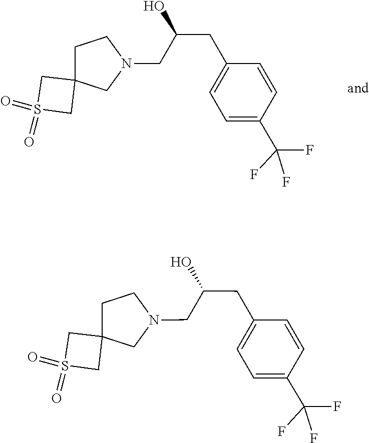 [SMILES: O=S1(=O)CC2(CCN(C[C@@H](O)Cc3ccc(C(F)(F)F)cc3)C2)C1 ; O=S1(=O)CC2(CCN(C[C@H](O)Cc3ccc(C(F)(F)F)cc3)C2)C1]

To a solution of 2-[[4-(trifluoromethyl)phenyl]methyl]oxirane (150 mg, 0.74 mmol) in ethanol (5 mL) was added triethylamine (0.31 mL, 2.23 mmol) and 2-thia-6-azaspiro[3.4]octane 2,2-dioxide hydrochloride (166 mg, 0.84 mmol), and stirred at 80° C. for 16 h. After concentrating under reduced pressure, the residue was purified by silica column chromatography (0-65% ethyl acetate in petroleum ether) to provide the title compound (130 mg, 46.8% yield). LCMS (ESI) [M+H]⁺=364.1.

### Step 3: Synthesis of the Title Compounds

To a solution of 6-(2-hydroxy-3-(4-(trifluoromethyl)phenyl)propyl)-2-thia-6-azaspiro[3.4]octane-2,2-dioxide (50 mg, 0.14 mmol) in tetrahydrofuran (2 mL) was added perfluorobutanesulfonyl fluoride (83 mg, 0.28 mmol) and 2-tert-butyl-1,1,3,3-tetramethylguanidine (71 mg, 0.41 mmol) and stirred at room temperature for 16 h. The reaction was concentrated under reduced pressure and purified by silica column chromatography (0-70% ethyl acetate in petroleum ether) to provide the title compound (11.5 mg, 22.6% yield) as a racemic mixture.

Compound 142 (mixture): ¹H NMR (400 MHz, CDCl₃): δ 7.58 (d, J=8.0 Hz, 2H), 7.35 (d, J=8.0 Hz, 2H), 4.89-4.74 (m, 1H), 4.07 (s, 4H), 3.05 (d, J=5.2 Hz, 1H), 3.00 (t, J=6.4 Hz, 1H), 2.95-2.88 (m, 2H), 2.81-2.69 (m, 4H), 2.17 (t, J=7.2 Hz, 2H). LCMS (ESI) [M+H]⁺=366.2.

### Example 143: (S)-6-(2-Methoxy-3-(4-(trifluoromethyl)phenyl)propyl)-2-thia-6-azaspiro[3.4]octane 2,2-dioxide and (R)-6-(2-methoxy-3-(4-(trifluoromethyl)phenyl)propyl)-2-thia-6-azaspiro[3.4]octane 2,2-dioxide

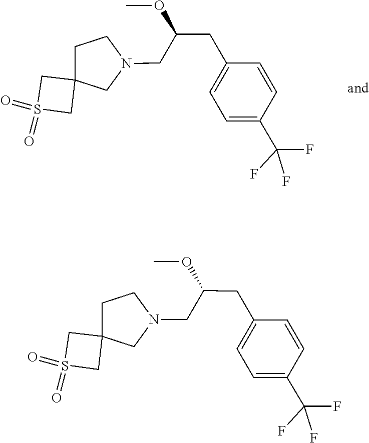 [SMILES: CO[C@@H](Cc1ccc(C(F)(F)F)cc1)CN1CCC2(C1)CS(=O)(=O)C2 ; CO[C@H](Cc1ccc(C(F)(F)F)cc1)CN1CCC2(C1)CS(=O)(=O)C2]

To a solution of 6-(2-hydroxy-3-(4-(trifluoromethyl)phenyl)propyl)-2-thia-6-azaspiro[3.4]octane 2,2-dioxide (from example 142, 80 mg, 0.22 mmol) in dichloromethane (4 mL) was added diethylaminosulfur trifluoride (0.15 mL, 1.1 mmol) at −78° C. and stirred for 1 h at the same temperature. Methanol (5 mL) was added and stirred at 25° C. for another 1 h. The reaction mixture was diluted with saturated sodium bicarbonate (10 mL) and extracted with dichloromethane (25 mL×3). The combined organic layer was dried over anhydrous Na₂SO₄, filtered and concentrated in vacuo. The residue was purified by silica column chromatography (0-100% ethyl acetate in petroleum ether) to provide the title compound (19.8 mg, 22.9% yield) as a racemic mixture of enantiomers.

Compound 143 (mixture): ¹H NMR (400 MHz, CDCl₃): δ 7.55 (d, J=8.0 Hz, 2H), 7.32 (d, J=8.0 Hz, 2H), 4.06 (s, 4H), 3.54-3.47 (m, 1H), 3.36 (s, 3H), 2.94-2.87 (m, 3H), 2.84-2.80 (m, 1H), 2.73 (t, J=7.2 Hz, 2H), 2.53 (d, J=5.6 Hz, 2H), 2.14 (t, J=7.2 Hz, 2H). LCMS (ESI) [M+H]⁺=378.2.

### Example 144: 6-(2-Methyl-2-((2-(trifluoromethyl)pyrimidin-5-yl)oxy)propyl)-2-thia-6-azaspiro[3.4]octane-2,2-dioxide

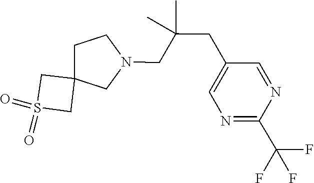 [SMILES: CC(C)(Cc1cnc(C(F)(F)F)nc1)CN1CCC2(C1)CS(=O)(=O)C2]

The title compound was synthesized using the synthetic procedure described for example 120, utilizing 2-trifluoromethyl-5-hydroxypyrimidine in step 1 (27 mg, 22.5% yield).

Compound 144: ¹H NMR (400 MHz, CDCl₃): δ ppm 8.56 (s, 2H), 4.10-4.03 (m, 4H), 3.02 (s, 2H), 2.90 (t, J=7.2 Hz, 2H), 2.82 (s, 2H), 2.16 (t, J=7.2 Hz, 2H), 1.42 (s, 6H). LCMS (ESI) [M+H]⁺=380.1.

### Examples 145A* and 145B*: (S)-6-(2-Methyl-3-(2-(trifluoromethyl)pyrimidin-5-yl)propyl)-2-thia-6-azaspiro[3.4]octane 2,2-dioxide and (R)-6-(2-Methyl-3-(2-(trifluoromethyl)pyrimidin-5-yl)propyl)-2-thia-6-azaspiro[3.4]octane 2,2-dioxide

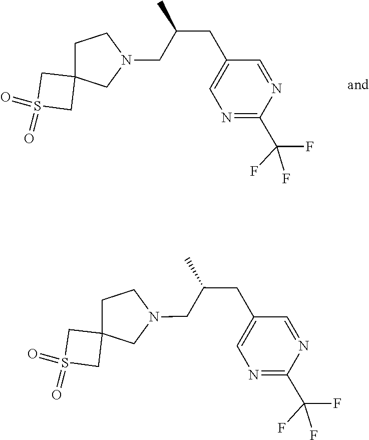 [SMILES: C[C@@H](Cc1cnc(C(F)(F)F)nc1)CN1CCC2(C1)CS(=O)(=O)C2 ; C[C@H](Cc1cnc(C(F)(F)F)nc1)CN1CCC2(C1)CS(=O)(=O)C2]

The title compounds were prepared using the synthetic procedure described for Example 126, utilizing 2-trifluoromethy-4-iodopyrimidine in step 1. The crude racemic mixture was separated by chiral SFC (Chiral Pak AD-3, 150 mm×4.6 mm, 3 μm, CO₂-0.1% NH₃H₂O in EtOH, 5-40%). Compound 145A* was obtained as the first eluting peak (4 mg, 12% yield), as a single enantiomer of undefined/unassigned absolute stereochemistry. Compound 145B* was obtained as the second eluting peak (7 mg, 22% yield), as a single enantiomer of undefined/unassigned absolute stereochemistry.

Compound 145A*: ¹H NMR (400 MHz, CD₃OD): δ ppm 8.84 (s, 2H), 4.09 (s, 4H), 2.94-2.87 (m, 2H), 2.78-2.68 (m, 2H), 2.66-2.61 (m, 1H), 2.58-2.40 (m, 2H), 2.17-2.07 (m, 2H), 1.37-1.31 (m, 2H), 0.94 (d, J=6.4 Hz, 3H). LCMS (ESI) [M+H]⁺=364.1.

Compound 145B*: ¹H NMR (400 MHz, CD₃OD): δ ppm 8.84 (s, 2H), 4.05 (s, 4H), 2.93-2.89 (m, 1H), 2.78 (s, 2H), 2.66-2.61 (m, 3H), 2.41 (d, J=8.8 Hz, 1H), 2.35 (d, J=6.0 Hz, 1H), 2.17-2.11 (m, 3H), 0.92 (d, J=6.8 Hz, 3H). LCMS (ESI) [M+H]⁺=364.1.

### Examples 148A* and 148B*: (S)-7-(2,2-Dimethyl-3-(6-(trifluoromethyl)pyridin-3-yl)propyl)-2-thia-7-azaspiro[4.4]nonane 2,2-dioxide and (R)-7-(2,2-Dimethyl-3-(6-(trifluoromethyl)pyridin-3-yl)propyl)-2-thia-7-azaspiro[4.4]nonane 2,2-dioxide

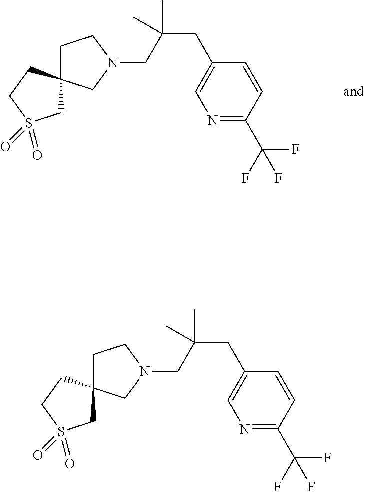 [SMILES: CC(C)(Cc1ccc(C(F)(F)F)nc1)CN1CC[C@]2(CCS(=O)(=O)C2)C1 ; CC(C)(Cc1ccc(C(F)(F)F)nc1)CN1CC[C@@]2(CCS(=O)(=O)C2)C1]

The title compounds were synthesized using the synthetic procedure described for Example 140, utilizing 2-thia-7-azaspiro[4.4]nonane-2,2-dioxide in step 3. The crude racemic mixture was separated by chiral SFC (Daicel Chiralpak IG-H (250 mm×30 mm, 5 m); CO₂-0.1% NH₃H₂O in EtOH, 55%; 80 mL/min) to provide Compound 148A* as the first eluting peak (63 mg, 40% yield), as a single enantiomer of undefined/unassigned absolute stereochemistry, and Compound 148B* as the second eluting peak (64 mg, 40% yield), as a single enantiomer of undefined/unassigned absolute stereochemistry.

Compound 148A*: ¹H NMR (400 MHz, CDCl₃): δ ppm 8.53 (s, 1H), 7.63-7.61 (m, 2H), 3.31-3.07 (m, 4H), 2.92-2.89 (m, 2H), 2.78-2.72 (m, 1H), 2.66-2.57 (m, 3H), 2.33 (s, 2H), 2.27-2.18 (m, 2H), 2.03-2.01 (m, 1H), 1.92-1.87 (m, 1H), 0.88 (s, 6H). LCMS (ESI) [M+H]⁺=391.0.

Compound 148B*: ¹H NMR (400 MHz, CDCl₃): δ ppm 8.53 (s, 1H), 7.63-7.61 (m, 2H), 3.31-3.07 (m, 4H), 2.92-2.89 (m, 2H), 2.78-2.72 (m, 1H), 2.66-2.57 (m, 3H), 2.33 (s, 2H), 2.27-2.18 (m, 2H), 2.03-2.01 (m, 1H), 1.92-1.87 (m, 1H), 0.88 (s, 6H). LCMS (ESI) [M+H]⁺=391.0.

### Example 149: 6-(2,2-Dimethyl-3-(5-(trifluoromethyl)pyridin-3-yl)propyl)-2-thia-6-azaspiro[3.4]octane-2,2-dioxide

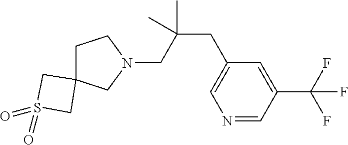 [SMILES: CC(C)(Cc1cncc(C(F)(F)F)c1)CN1CCC2(C1)CS(=O)(=O)C2]

### Step 1: Synthesis of [5-(trifluoromethyl)-3-pyridyl]methanol

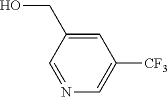 [SMILES: OCc1cncc(C(F)(F)F)c1]

5-(Trifluoromethyl)nicotinic acid (2200 mg, 11.51 mmol) was dissolved in borane-THF (58 mL, 58 mmol, 1M in THF) and the mixture was stirred at 25° C. for 16 h. The reaction was quenched by methanol (60 mL). The reaction mixture was heated at 70° C. for 4 h. The resulting mixture was concentrated under reduced pressure to afford the crude title compound (1900 mg, 10.7 mmol, 93.2% yield). LCMS (ESI) [M+H]⁺=178.1. ¹H NMR (400 MHz, CDCl₃) δ 8.82 (s, 1H), 8.79 (s, 1H), 8.00 (s, 1H), 4.85 (s, 2H).

### Step 2: Synthesis of 3-(bromomethyl)-5-(trifluoromethyl)pyridine

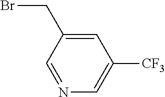 [SMILES: FC(F)(F)c1cncc(CBr)c1]

To a solution of [5-(trifluoromethyl)-3-pyridyl]methanol (900 mg, 5.08 mmol) in dichloromethane (18 mL) was added phosphorous tribromide (0.97 mL, 10.16 mmol) at 0° C. The reaction mixture was stirred at 25° C. for 2 h. The reaction was quenched with NaHCO₃ solution (50 mL). The mixture was extracted with dichloromethane (60 mL×3). The combined organic layer were washed with water (50 mL×3), dried over anhydrous Na₂SO₄, filtered and concentrated under reduced pressure. The crude residue was purified by silica column chromatography (0-30% ethyl acetate in petroleum ether) to provide the title compound (500 mg, 2.08 mmol, 41% yield). LCMS (ESI) [M+H]⁺=240.1.

### Step 3: Synthesis of methyl 2,2-dimethyl-3-[5-(trifluoromethyl)-3-pyridyl]propanoate

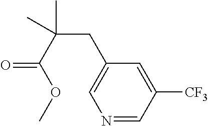 [SMILES: COC(=O)C(C)(C)Cc1cncc(C(F)(F)F)c1]

To a cooled (−75° C.) solution of methyl isobutyrate (26.04 mL, 3.33 mmol) in tetrahydrofuran (15 mL) was added lithiumdiisopropylamide (2 mL, 4.0 mmol, 2 M in THF) under nitrogen and the reaction mixture was stirred at −75° C. for 5 h. Then 3-(bromomethyl)-5-(trifluoromethyl)pyridine (500 mg, 2.08 mmol) was added slowly at −75° C. and stirred at that temperature for 2 h. The reaction was quenched by saturated NH₄Cl solution (10 mL) and extracted with ethyl acetate (20 mL×3). The combined organic layers were washed with water (10 mL) and brine (10 mL*2), dried over anhydrous sodium sulfate, filtered and concentrated under reduced pressure. The residue was purified by silica column chromatography [0-20% ethyl acetate in petroleum ether] to provide the title compound (400 mg, 1.53 mmol, 74%). LCMS (ESI) [M+H]⁺=262.1.

### Step 4: Synthesis of 2,2-dimethyl-3-[5-(trifluoromethyl)-3-pyridyl]propan-1-ol

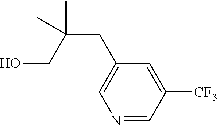 [SMILES: CC(C)(CO)Cc1cncc(C(F)(F)F)c1]

To a solution of methyl 2,2-dimethyl-3-[5-(trifluoromethyl)-3-pyridyl]propanoate (120 mg, 0.46 mmol) in dichloromethane (5 mL) was added diisobutylalumanylium hydride (0.69 mL, 0.69 mmol, 1M in toluene) at −75° C. and the mixture was stirred at −75° C. for 2 h. The reaction was quenched by potassium sodium tartrate solution (10 mL) and extracted with ethyl acetate (20 mL×3). The combined organic layers dried over anhydrous Na₂SO₄, filtered and concentrated under reduced pressure. The residue was purified by silica column chromatography (0-10% ethyl acetate in petroleum ether) to provide the title compound (38 mg, 0.163 mmol, 35.5% yield). LCMS (ESI) [M+H]⁺=234.1.

### Step 5: Synthesis of [2,2-dimethyl-3-[5-(trifluoromethyl)-3-pyridyl]propyl] methanesulfonate

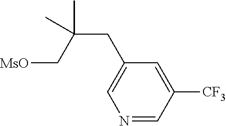 [SMILES: CC(C)(COS(C)(=O)=O)Cc1cncc(C(F)(F)F)c1]

To a solution of 2,2-dimethyl-3-[5-(trifluoromethyl)-3-pyridyl]propan-1-ol (38 mg, 0.16 mmol) and N,N-diisopropylethylamine (0.06 mL, 0.36 mmol) in dichloromethane (4 mL) was added methanesulfonyl chloride (0.02 mL, 0.21 mmol) at 0° C. and stirred at 25° C. for 1 h. The mixture was diluted in water (10 mL) and extracted with dichloromethane (10 mL×3). The combined organic layers were dried over anhydrous Na₂SO₄, filtered and concentrated under reduced pressure to provide the title compound (50 mg, 0.161 mmol, 98.6% yield). LCMS (ESI) [M+H]⁺=312.1.

### Step 6: Synthesis of the Title Compound

To a solution of 2-thia-6-azaspiro[3.4]octane 2,2-dioxide (39 mg, 0.24 mmol) and K₂CO₃ (110 mg, 0.80 mmol) in N,N-dimethylacetamide (5 mL) was added [2,2-dimethyl-3-[5-(trifluoromethyl)-3-pyridyl]propyl] methanesulfonate (50 mg, 0.161 mmol) and KI (53 mg, 0.32 mmol). The mixture was stirred in microwave at 140° C. for 1 h. The reaction mixture was quenched with water (10 mL) and extracted with ethyl acetate (10 mL×3). The combined organic layers were washed with brine, dried over anhydrous sodium sulfate, filtered and concentrated under reduced pressure. The residue was purified by silica column chromatography [0%˜10% methanol in dichloromethane] to provide the title compound (12.4 mg, 0.032 mmol, 19.9% yield).

Compound 149: ¹H NMR (400 MHz, CD₃OD): δ ppm 8.73 (d, J=1.2 Hz, 1H), 8.64 (d, J=1.6 Hz, 1H), 7.96 (s, 1H), 4.10 (s, 4H), 2.96 (s, 2H), 2.85 (t, J=7.2 Hz, 2H), 2.75 (s, 2H), 2.39 (s, 2H), 2.16 (t, J=7.2 Hz, 2H), 0.89 (s, 6H). LCMS (ESI) [M+H]⁺=377.2.

### Example 150: 6-(2,2-Dimethyl-3-(5-(trifluoromethyl)pyridin-2-yl)propyl)-2-thia-6-azaspiro[3.4]octane-2,2-dioxide

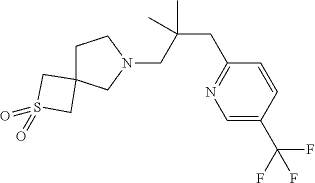 [SMILES: CC(C)(Cc1ccc(C(F)(F)F)cn1)CN1CCC2(C1)CS(=O)(=O)C2]

The title compound was synthesized using the synthetic procedure described for example 149, utilizing 2-hydroxymethyl-5-trifluoromethylpyridine in step 1, and 2-thia-6-azaspiro[3.4]octane-2,2-dioxide in step 5 (61.3 mg, 36.1% yield).

Compound 150: ¹H NMR (400 MHz, CDCl₃) δ ppm 8.81 (s, 1H), 7.82 (d, J=8.4 Hz, 1H), 7.24 (d, J=8.4 Hz, 1H), 4.06 (s, 4H), 2.98 (s, 2H), 2.88 (t, J=7.2 Hz, 2H), 2.82 (s, 2H), 2.44 (s, 2H), 2.13 (t, J=7.2 Hz, 2H), 0.90 (s, 6H). LCMS (ESI) [M+H]⁺=377.1.

### Example 152: 7-(2,2-Dimethyl-3-(6-(trifluoromethyl)pyridin-3-yl)propyl)-2-thia-7-azaspiro[3.5]nonane-2,2-dioxide

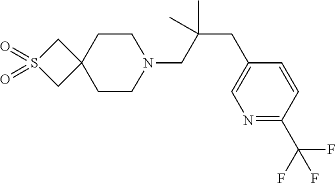 [SMILES: CC(C)(Cc1ccc(C(F)(F)F)nc1)CN1CCC2(CC1)CS(=O)(=O)C2]

The title compound was synthesized using the synthetic procedure described for example 147, utilizing 2-thia-7-azaspiro[3.5]nonane in step 2 (30.6 mg, 31% yield).

Compound 152: ¹H NMR (400 MHz, CDCl₃): δ ppm 8.50 (s, 1H), 7.61 (s, 2H), 3.86 (s, 4H), 2.61 (s, 2H), 2.51 (brs, 4H), 2.18 (s, 2H), 1.95-1.90 (m, 4H), 1.56 (s, 4H), 0.83 (s, 6H). LCMS (ESI) [M+H]⁺=391.2.

### Example 153: 7-(2,2-Dimethyl-3-(4-(trifluoromethyl)phenyl)propyl)-2-thia-7-azaspiro[3.5]nonane-2,2-dioxide

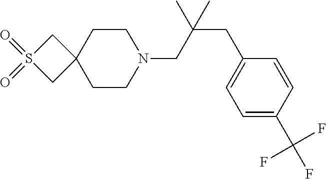 [SMILES: CC(C)(Cc1ccc(C(F)(F)F)cc1)CN1CCC2(CC1)CS(=O)(=O)C2]

The title compound was synthesized using a synthetic procedure similar to described for example 147, utilizing 2-thia-7-azaspiro[3.5]nonane in step 2, and 2,2-dimethyl-3-[4-(trifluoromethyl)phenyl]propanoate in step 1 (30.6 mg, 31% yield).

Compound 153: ¹H NMR (400 MHz, CD₃OD): δ 7.55 (d, J=8.0 Hz, 2H), 7.33 (d, J=8.0 Hz, 2H), 3.88 (s, 4H), 2.64 (s, 2H), 2.55-2.45 (m, 4H), 2.19 (s, 2H), 1.89 (t, J=5.2 Hz, 4H), 0.84 (s, 6H). LCMS (ESI) [M+H]⁺=390.1.

### Examples 154A* and 154B*: (S)-6-(2-Methyl-3-(1-(2,2,2-trifluoroethyl)-1H-pyrazol-4-yl)propyl)-2-thia-6-azaspiro[3.4]octane 2,2-dioxide and (R)-6-(2-Methyl-3-(1-(2,2,2-trifluoroethyl)-1H-pyrazol-4-yl)propyl)-2-thia-6-azaspiro[3.4]octane 2,2-dioxide

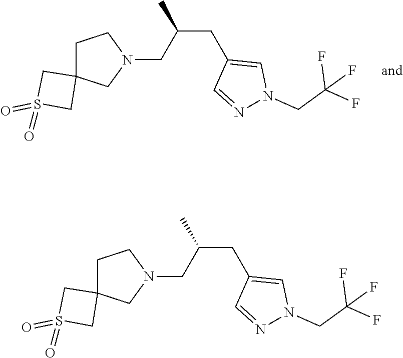 [SMILES: C[C@@H](Cc1cnn(CC(F)(F)F)c1)CN1CCC2(C1)CS(=O)(=O)C2 ; C[C@H](Cc1cnn(CC(F)(F)F)c1)CN1CCC2(C1)CS(=O)(=O)C2]

### Step 1: Synthesis of 1-(2,2,2-trifluoroethyl)-1H-pyrazole-4-carbaldehyde

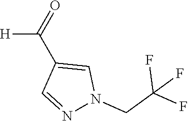 [SMILES: O=Cc1cnn(CC(F)(F)F)c1]

To a solution of 1H-pyrazole-4-carbaldehyde (2000 mg, 20.81 mmol) in N,N-dimethylformamide (20 mL) was added Cs₂CO₃ (2.03 g, 62.44 mmol) and 2,2,2-trifluoroethyltrifluoromethanesulfonate (7.25 g, 31.22 mmol) and stirred at 40° C. for 2 h. The reaction was quenched by water (40 mL) and extracted with ethyl acetate (60 mL×3). The combined organic layers were washed with brine (50 mL×3), dried over anhydrous sodium sulfate, filtered and concentrated under reduced pressure. The residue was purified by silica column chromatography (0-75% ethyl acetate in petroleum ether) to provide the title compound (3.4 g, 91.7% yield). ¹H NMR (400 MHz, CDCl₃) δ 9.93 (s, 1H), 8.07 (d, J=6.4 Hz, 2H), 4.81-4.66 (m, 2H).

### Step 2: Synthesis of (E)-ethyl 2-methyl-3-(1-(2,2,2-trifluoroethyl)-1H-pyrazol-4-yl)acrylate

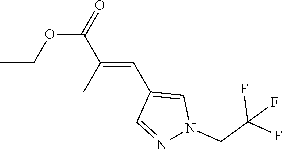 [SMILES: CCOC(=O)/C(C)=C/c1cnn(CC(F)(F)F)c1]

To an ice cooled solution of triethyl 2-phosphonopropionate (5002 mg, 21 mmol) in tetrahydrofuran (60 mL) was added NaH (916 mg, 22.91 mmol, 60% in mineral oil) portions and stirred for 30 minutes. Then 1-(2,2,2-trifluoroethyl)pyrazole-4-carbaldehyde (3.4 g, 19.09 mmol) was added and stirred at 25° C. for 2 h. The reaction was quenched by water (25 mL) and extracted with ethyl acetate (50 mL×3). The combined organic layers were washed with brine (25 mL×3), dried over anhydrous sodium sulfate, filtered and concentrated under reduced pressure. The residue was purified by silica column chromatography (0-50% ethyl acetate in petroleum ether) to provide the title compound (3940 mg, 78.7% yield). LCMS (ESI) [M+H]⁺=263.2.

### Step 3: Synthesis of ethyl (S)-2-methyl-3-(1-(2,2,2-trifluoroethyl)-1H-pyrazol-4-yl)propanoate and ethyl (R)-2-methyl-3-(1-(2,2,2-trifluoroethyl)-1H-pyrazol-4-yl)propanoate

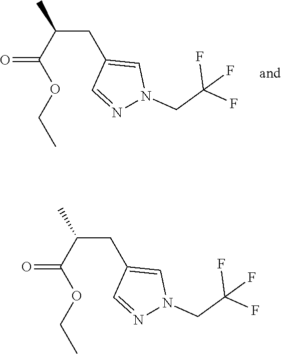 [SMILES: CCOC(=O)[C@@H](C)Cc1cnn(CC(F)(F)F)c1 ; CCOC(=O)[C@H](C)Cc1cnn(CC(F)(F)F)c1]

To a solution of ethyl (E)-2-methyl-3-[1-(2,2,2-trifluoroethyl)pyrazol-4-yl]prop-2-enoate (1000 mg, 3.81 mmol) in ethanol (20 mL) was added 10% palladium on carbon (490 mg, 0.46 mmol) and stirred under H₂ (15 psi) at 25° C. for 2 h. The reaction mixture was filtered and the organic layer was concentrated to provide the title compound (1000 mg, 99.2% yield). LCMS (ESI) [M+H]⁺=265.1.

### Step 4: Synthesis of (R)-2-methyl-3-(1-(2,2,2-trifluoroethyl)-1H-pyrazol-4-yl)propan-1-ol and (S)-2-methyl-3-(1-(2,2,2-trifluoroethyl)-1H-pyrazol-4-yl)propan-1-ol

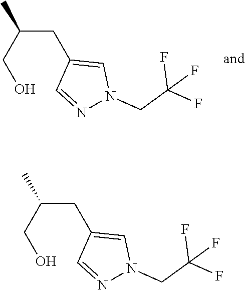

To a stirred suspension of lithiumaluminumhydride (430 mg, 11.35 mmol) in tetrahydrofuran (15 mL) was added a solution of ethyl 2-methyl-3-[1-(2,2,2-trifluoroethyl)pyrazol-4-yl]propanoate (1000 mg, 3.78 mmol) in tetrahydrofuran (8 mL) at 0° C. Then the mixture was stirred at 20° C. for 1 h. The mixture was cooled to 0° C., and quenched by water (1 mL), 15% NaOH aqueous solution (1 mL) and water (3 mL) and then filtered. The organic layer was dried over anhydrous sodium sulfate, filtered and concentrated to provide the title compound (520 mg, 61.8% yield). LCMS (ESI) [M+H]⁺=223.1

### Step 5: Synthesis of (S)-2-methyl-3-(1-(2,2,2-trifluoroethyl)-1H-pyrazol-4-yl)propyl methanesulfonate and (R)-2-methyl-3-(1-(2,2,2-trifluoroethyl)-1H-pyrazol-4-yl)propyl methanesulfonate

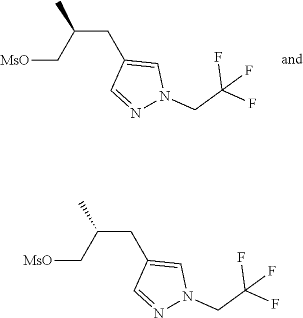 [SMILES: C[C@H](COS(C)(=O)=O)Cc1cnn(CC(F)(F)F)c1 ; C[C@@H](COS(C)(=O)=O)Cc1cnn(CC(F)(F)F)c1]

To an ice cooled solution of 2-methyl-3-[1-(2,2,2-trifluoroethyl)pyrazol-4-yl]propan-1-ol (520 mg, 2.36 mmol) and N,N-diisopropylethylamine (0.96 mL, 5.2 mmol) in dichloromethane (15 mL) was added methanesulfonyl chloride (297 mg, 2.6 mmol) and stirred at 20° C. for 4 h. The reaction mixture was quenched by water (20 mL), extracted with dichloromethane (50 mL×3). The combined organic layers were washed with saturated sodium bicarbonate (20 mL×3) and brine (20 mL), dried over anhydrous sodium sulfate, filtered and concentrated to provide the title compound (700 mg, 98.7% yield), and used directly for next step.

### Step 6: Synthesis of the Title Compounds

To a solution of 2-methyl-3-[1-(2,2,2-trifluoroethyl)pyrazol-4-yl]propylmethanesulfonate (200 mg, 0.68 mmol), 2-thia-6-azaspiro[3.4]octane 2,2-dioxide (116 mg, 0.72 mmol) and N,N-diisopropylethylamine (128 mg, 1 mmol) in acetonitrile (8 mL) was added K₂CO₃ (220 mg, 1.32 mmol) and then stirred at 100° C. for 1 h under microwave. The reaction mixture was diluted with water (10 mL) and extracted with ethyl acetate (15 mL×3). The combined organic layers were washed with brine (10 mL×3), dried over anhydrous sodium sulfate, filtered and concentrated under reduced pressure. The residue was purified by silica column chromatography (0-75% ethyl acetate in petroleum ether) to afford the title compound (150 mg, 61% yield). LCMS (ESI) [M+H]⁺=366.1. The mixture of enantiomers (120 mg, 0.33 mmol) was separated by chiral SFC (SFC-17; Daicel Chiralcel OJ-H (250 mm*30 mm, 5 μm)); 0.1% NH₃H₂O/EtOH=10:90; 60 mL/min). Compound 154A* was obtained as the first peak on SFC (41.8 mg, 33% yield), as a single enantiomer of undefined/unassigned absolute stereochemistry. Compound 154B* was obtained as the second peak on SFC (21.7 mg, 17% yield), as a single enantiomer of undefined/unassigned absolute stereochemistry.

Compound 154A*: ¹H NMR (400 MHz, CD₃OD) δ 7.55 (s, 1H), 7.42 (s, 1H), 4.87-4.83 (m, 2H), 4.12 (s, 4H), 2.91 (brs, 2H), 2.78 (brs, 2H), 2.62 (dd, J=14.2, 5.2 Hz, 1H), 2.52-2.42 (m, 1H), 2.40-2.30 (m, 2H), 2.21 (t, J=7.2 Hz, 2H), 1.88-1.87 (m, 1H), 0.91 (d, J=6.8 Hz, 3H). LCMS (ESI) [M+H]⁺=366.2.

Compound 154B*: ¹H NMR (400 MHz, CD₃OD) δ 7.55 (s, 1H), 7.42 (s, 1H), 4.87-4.83 (m, 2H), 4.12 (s, 4H), 2.91 (brs, 2H), 2.78 (brs, 2H), 2.62 (dd, J=14.2, 5.2 Hz, 1H), 2.52-2.42 (m, 1H), 2.40-2.30 (m, 2H), 2.21 (t, J=7.2 Hz, 2H), 1.88-1.87 (m, 1H), 0.91 (d, J=6.8 Hz, 3H). LCMS (ESI) [M+H]⁺=366.2.

### Example 155: (S)-6-(3-(1-Isopropyl-3-(trifluoromethyl)-1H-pyrazol-5-yl)-2-methylpropyl)-2-thia-6-azaspiro[3.4]octane 2,2-dioxide and (R)-6-(3-(1-isopropyl-3-(trifluoromethyl)-1H-pyrazol-5-yl)-2-methylpropyl)-2-thia-6-azaspiro[3.4]octane 2,2-dioxide

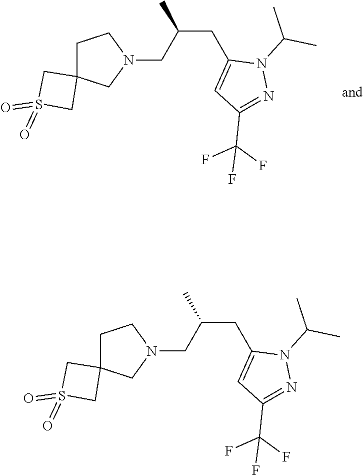 [SMILES: CC(C)n1nc(C(F)(F)F)cc1C[C@H](C)CN1CCC2(C1)CS(=O)(=O)C2 ; CC(C)n1nc(C(F)(F)F)cc1C[C@@H](C)CN1CCC2(C1)CS(=O)(=O)C2]

### Step 1: Synthesis of 1-isopropyl-3-(trifluoromethyl)-1H-pyrazole

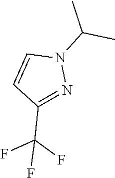

To a stirred suspension of 3-(trifluoromethyl)pyrazole (4 g, 29.39 mmol) in acetonitrile (50 mL) was added 2-iodopropane (15 g, 88.18 mmol) and Cs₂CO₃ (48 g, 146.97 mmol) at 25° C. for 16 h. The reaction mixture was filtered and the organic layer was diluted with water (50 mL). Then the mixture was extracted with MTBE (50 mL×3). The combined organic layer were dried over anhydrous sodium sulfate, filtered and concentrated under reduced pressure to provide the title compound (4.2 g, 80% yield). ¹H NMR (400 MHz, CDCl₃) δ 7.46 (d, J=1.6 Hz, 1H), 6.50 (d, J=2.0 Hz, 1H), 4.61-4.51 (m, 1H), 1.53 (d, J=6.8 Hz, 6H).

### Step 2: Synthesis of 1-isopropyl-3-(trifluoromethyl)-1H-pyrazole-5-carbaldehyde

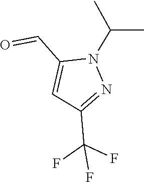

To a stirred solution of 1-isopropyl-3-(trifluoromethyl)pyrazole (1 g, 5.61 mmol) in tetrahydrofuran (20 mL) was added n-butyllithium (3 mL, 7.5 mmol, 2.5 M in hexane) dropwise at −78° C. The reaction mixture was stirred at the same temperature for 1 h. Then N,N-dimethylformamide (1.5 mL, 19.47 mmol) was added dropwise and the reaction was stirred at −78° C. for another 1 h. The reaction mixture was quenched by saturated NH₄C₁ solution (20 mL) and extracted with MTBE (50 mL×3). The combined organic layers were washed with brine (20 mL×2), dried over anhydrous sodium sulfate, filtered and concentrated under reduced pressure to provide the title compound (1.5 g). ¹H NMR (400 MHz, CDCl₃) δ 9.87 (s, 1H), 7.13 (s, 1H), 5.49-5.39 (m, 1H), 1.53 (d, J=6.8 Hz, 6H).

### Step 3: Synthesis of (E)-ethyl 3-(1-isopropyl-3-(trifluoromethyl)-1H-pyrazol-5-yl)-2-methylacrylate

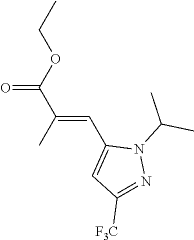 [SMILES: CCOC(=O)/C(C)=C/c1cc(C(F)(F)F)nn1C(C)C]

To an ice cooled solution of triethyl 2-phosphonopropionate (1907 mg, 8.01 mmol) in tetrahydrofuran (10 mL) was added NaH (349 mg, 8.73 mmol, 60% in mineral oil) slowly, the reaction mixture was stirred for 30 min. 2-Isopropyl-5-(trifluoromethyl)pyrazole-3-carbaldehyde (1.5 g, 7.28 mmol) was then added and stirred at 25° C. for another 1 h. The reaction mixture was quenched by NHCl₄ (30 mL) and extracted with ethyl acetate (50 mL×3). The combined organic layers were washed with brine (20 mL×2), dried over anhydrous sodium sulfate, filtered and concentrated under reduced pressure. The residue was purified by silica column chromatography (0-3% ethyl acetate in petroleum ether) to provide the title compound (900 mg, 43% yield), LCMS (ESI) [M+H]⁺=291.1

### Step 4: Ethyl (S)-3-(1-isopropyl-3-(trifluoromethyl)-1H-pyrazol-5-yl)-2-methylpropanoate and ethyl (R)-3-(1-isopropyl-3-(trifluoromethyl)-1H-pyrazol-5-yl)-2-methylpropanoate

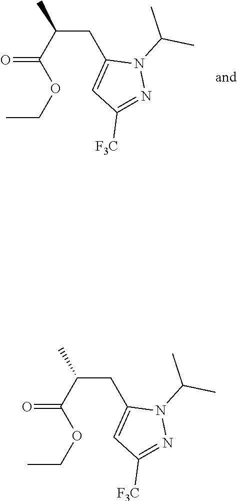 [SMILES: CCOC(=O)[C@@H](C)Cc1cc(C(F)(F)F)nn1C(C)C ; CCOC(=O)[C@H](C)Cc1cc(C(F)(F)F)nn1C(C)C]

To a solution of ethyl (E)-3-[2-isopropyl-5-(trifluoromethyl)pyrazol-3-yl]-2-methyl-prop-2-enoate (900 mg, 3.1 mmol) in methanol (5 mL) was added 10% palladium on carbon (330 mg, 0.31 mmol) and stirred under H₂ (15 psi) at 25° C. for 2 h. The reaction mixture was filtered and the filtrate was concentrated under reduced pressure to afford the title compound (520 mg, 57% yield), LCMS (ESI): [M+H]⁺=293.1.

### Step 5: Synthesis of (S)-3-(1-isopropyl-3-(trifluoromethyl)-1H-pyrazol-5-yl)-2-methylpropan-1-ol and (R)-3-(1-isopropyl-3-(trifluoromethyl)-1H-pyrazol-5-yl)-2-methylpropan-1-ol

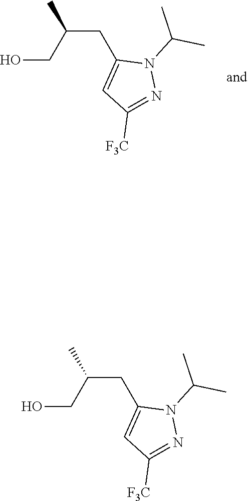 [SMILES: CC(C)n1nc(C(F)(F)F)cc1C[C@H](C)CO ; CC(C)n1nc(C(F)(F)F)cc1C[C@@H](C)CO]

To a stirred solution of ethyl 3-[2-isopropyl-5-(trifluoromethyl)pyrazol-3-yl]-2-methyl-propanoate (720 mg, 2.46 mmol) in tetrahydrofuran (5 mL) at 0° C. was added lithiumaluminumhydride (280 mg, 7.39 mmol). The reaction mixture was stirred at 25° C. for 1 h. The mixture was cooled to 0° C., then water (0.3 mL), 15% NaOH solution (1.2 mL) and water (1.2 mL) was added slowly to quench the reaction. The suspension mixture was filtered and the filtrate was concentrated. The residue was purified by silica column chromatography (0-50% ethyl acetate in petroleum ether) to afford the title compound (500 mg, 81% yield), LCMS (ESI): [M+H]⁺=251.1.

### Step 6: Synthesis of (R)-3-(1-isopropyl-3-(trifluoromethyl)-1H-pyrazol-5-yl)-2-methylpropyl methanesulfonate and (S)-3-(1-isopropyl-3-(trifluoromethyl)-1H-pyrazol-5-yl)-2-methylpropyl methanesulfonate

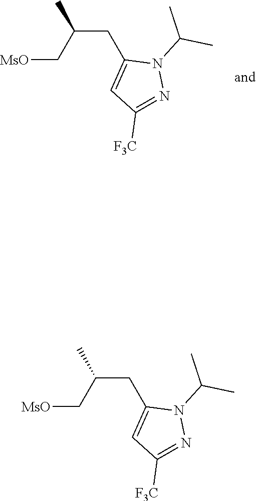 [SMILES: CC(C)n1nc(C(F)(F)F)cc1C[C@H](C)COS(C)(=O)=O ; CC(C)n1nc(C(F)(F)F)cc1C[C@@H](C)COS(C)(=O)=O]

To an ice cooled solution of 3-[2-isopropyl-5-(trifluoromethyl)pyrazol-3-yl]-2-methyl-propan-1-ol (500 mg, 2 mmol) and triethylamine (607 mg, 5.99 mmol) in dichloromethane (5 mL) was added methanesulfonyl chloride (252 mg, 2.2 mmol). The reaction mixture was stirred at 0° C. for 0.5 h. The reaction was quenched with water (5 mL) and extracted with dichloromethane (15 mL×3). The combined organic layers were dried over anhydrous sodium sulfate, filtered and concentrated under vacuum to afford the title compound (650 mg, 99% yield). LCMS (ESI) [M+H]⁺=329.1.

### Step 7: Synthesis of the Title Compounds

To a mixture of [3-[2-isopropyl-5-(trifluoromethyl)pyrazol-3-yl]-2-methyl-propyl] methanesulfonate (150 mg, 0.46 mmol) in acetonitrile (3 mL) was added 2-thia-6-azaspiro[3.4]octane 2,2-dioxide (89 mg, 0.55 mmol), N,N-diisopropylethylamine (89 mg, 0.69 mmol) and potassium iodide (152 mg, 0.91 mmol). The mixture was stirred at 80° C. for 16 h. The resulting solution was concentrated under vacuum. The residue was purified by reverse phase chromatography (water (0.05% NH₃H₂O+10 mM NH₄HCO₃)—ACN, 50%˜80%) to afford the title compound (81.8 mg, 45% yield) as a mixture of enantiomers. LCMS (ESI) [M+H]⁺=394.2.

Compound 155 (mixture): ¹H NMR (400 MHz, CDCl₃) δ 6.23 (s, 1H), 4.50-4.45 (m, 1H), 4.09-4.02 (m, 4H), 2.86-2.78 (m, 3H), 2.74-2.62 (m, 2H), 2.46-2.31 (m, 3H), 2.16 (t, J=6.8 Hz, 2H), 1.95-1.85 (m, 1H), 1.49 (dd, J=6.8, 2.4 Hz, 6H), 0.95 (d, J=6.4 Hz, 3H). LCMS (ESI) [M+H]⁺=394.2.

### Example 156: 7-(2-(3,4-Difluorophenoxy)-2-methylpropyl)-2-thia-7-azaspiro[3.5]nonane 2,2-dioxide

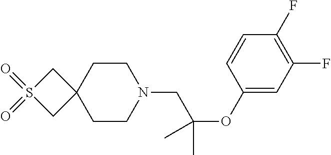 [SMILES: CC(C)(CN1CCC2(CC1)CS(=O)(=O)C2)Oc1ccc(F)c(F)c1]

### Step 1: Synthesis of (3S)-1-[1-[5-(trifluoromethyl)-2-pyridyl]-4-piperidyl]piperidin-3-ol

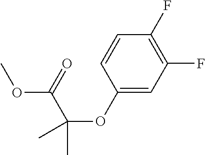 [SMILES: COC(=O)C(C)(C)Oc1ccc(F)c(F)c1]

To a solution of 3,4-difluorophenol (2 g, 15.3 mmol) in N,N-dimethylformamide (15 mL) was added K₂CO₃ (5 g, 34.97 mmol) and methyl 2-bromo-2-methylpropanoate (3 g, 17 mmol). The mixture was stirred at 80° C. for 15 h. Ethyl acetate (90 mL) was added and the resulting mixture was washed with brine (30 mL×3), dried over anhydrous Na₂SO₄, filtered and concentrated. The residue was purified by silica column chromatography (eluting with 0-10% ethyl acetate in petroleum ether) to afford the title compound (2 g, 56.5% yield). LCMS (ESI) [M+H]⁺=231.0

### Step 2: Synthesis of 2-(3,4-difluorophenoxy)-2-methyl-propanoic acid

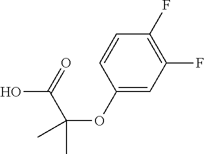

To a solution of methyl 2-(3,4-difluorophenoxy)-2-methyl-propanoate (500 mg, 2.17 mmol) in tetrahydrofuran (10 mL) was added a solution of lithium hydroxy hydrate (456 mg, 10.86 mmol) in water (6 mL). The mixture was stirred at 20° C. for 2 h. The reaction mixture was adjusted to pH 2 with HCl (1M), then diluted with water (10 ml) and extracted with ethyl acetate (30 mL×3). The combined organic layers were dried over anhydrous sodium sulfate, filtered and concentrated to afford the title compound (340 mg, 72.4% yield). ¹H NMR (400 MHz, CD₃OD) δ 7.23-7.08 (m, 1H), 6.90-6.80 (m, 1H), 6.73-6.67 (m, 1H), 1.55 (s, 6H)

### Step 3: Synthesis of 2-(3,4-difluorophenoxy)-1-(2,2-dioxido-2-thia-7-azaspiro[3.5]nonan-7-yl)-2-methylpropan-1-one

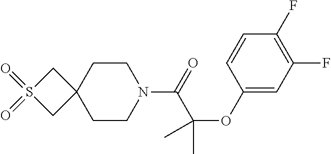 [SMILES: CC(C)(Oc1ccc(F)c(F)c1)C(=O)N1CCC2(CC1)CS(=O)(=O)C2]

To a mixture of 2-(3,4-difluorophenoxy)-2-methyl-propanoic acid (50 mg, 0.23 mmol), N,N-diisopropylethylamine (0.1 mL, 0.58 mmol) and 2-thia-7-azaspiro[3.5]nonane 2,2-dioxide hydrochloride (25 mg, 0.12 mmol) in dichloromethane (2 mL) was added HATU (132 mg, 0.35 mmol). The resulting mixture was stirred at 20° C. for 2 h. The reaction mixture was concentrated and the residue was purified by silica column chromatography (0-60% ethyl acetate in petroleum ether) to the title compound (80 mg, 92.6% yield). LCMS (ESI) [M+H]⁺=374.1.

### Step 4: Synthesis of the Title Compound

To a cold (0° C.) solution of 2-(3,4-difluorophenoxy)-1-(2,2-dioxido-2-thia-7-azaspiro[3.5]nonan-7-yl)-2-methylpropan-1-one (55 mg, 0.15 mmol) in tetrahydrofuran (3 mL) was added borane-THF (7 mL, 7 mmol, 1M). After being stirred for 10 min, the mixture was heated to 70° C. and stirred for 4 h. The reaction mixture was diluted with brine (10 mL) and extracted with ethyl acetate (40 mL×2). The combined organic layers were dried over anhydrous sodium sulfate, filtered and concentrated under reduced pressure. The crude product was purified by reverse phase chromatography (water (0.2% FA)—ACN 60%˜80%) to afford the title compound (33.1 mg, 57.9% yield). LCMS (ESI) [M+H]⁺=360.1.

Compound 156: ¹H NMR (400 MHz, CD₃OD) δ 7.20-7.16 (m, 1H), 6.99-6.96 (m, 1H), 6.84-6.82 (m, 1H), 3.95 (s, 4H), 2.93-2.89 (m, 6H), 2.03 (t, J=5.2 Hz, 4H), 1.31 (s, 6H). LCMS (ESI) [M+H]⁺=360.1.

### Example 157: 6-((1-((6-(Trifluoromethyl)pyridin-3-yl)methyl)cyclopropyl)methyl)-2-thia-6-azaspiro[3.4]octane 2,2-dioxide

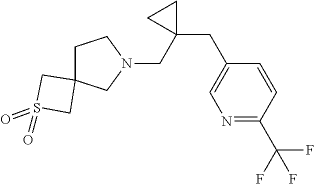 [SMILES: O=S1(=O)CC2(CCN(CC3(Cc4ccc(C(F)(F)F)nc4)CC3)C2)C1]

### Step 1: Synthesis of tert-butyl 1-((6-(trifluoromethyl)pyridin-3-yl)methyl)cyclopropanecarboxylate

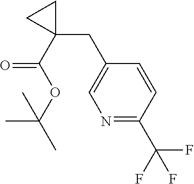 [SMILES: CC(C)(C)OC(=O)C1(Cc2ccc(C(F)(F)F)nc2)CC1]

To a cooled (−75° C.) solution of tert-butyl cyclopropanecarboxylate (533 mg, 3.75 mmol) in tetrahydrofuran (12 mL) was added lithiumdiisopropylamide (2.5 mL, 5 mmol, 2M in THF) dropwise under N₂ and stirred at −75° C. for 6 h. Then 5-(bromomethyl)-2-(trifluoromethyl) pyridine (600 mg, 2.5 mmol) was added slowly at −75° C. under N₂ and stirred at that temperature for 4 h then stirred at 20° C. for 10 h. The reaction mixture was quenched with saturated NH₄Cl solution (20 mL) and extracted with ethyl acetate (20 mL×3). The combined organic layers were washed with brine (20 mL×2), dried over anhydrous sodium sulfate, filtered and concentrated. The residue was purified by silica column chromatography (0-30% ethyl acetate in petroleum ether) to afford the title compound (0.26 g, 34.5% yield). LCMS (ESI) [M+H]⁺=302.2.

### Step 2: Synthesis of 1-((6-(trifluoromethyl)pyridin-3-yl)methyl)cyclopropanecarbaldehyde

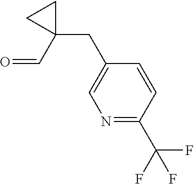 [SMILES: O=CC1(Cc2ccc(C(F)(F)F)nc2)CC1]

To a cooled (−75° C.) solution of tert-butyl 1-[[6-(trifluoromethyl)-3-pyridyl]methyl]cyclopropane carboxylate (260 mg, 0.86 mmol) in dichloromethane (10 mL) was added dropwise diisobutylalumanylium hydride (1.2 mL, 1.2 mmol, 1M in toluene) and the mixture was stirred at −75° C. for 2 h. The reaction mixture was quenched by saturated NH₄C₁ solution (20 mL) and extracted with dichloromethane (20 mL×3). The combined organic layers were dried over sodium sulfate, filtered and concentrated under reduced pressure. The resulting residue was purified by silica column chromatography (0-30% ethyl acetate in petroleum ether) to afford the title compound (120 mg, 52.8% yield). LCMS (ESI) [M+H]⁺=230.1.

### Step 3: Synthesis of the Title Compound

A mixture of 2-thia-6-azaspiro[3.4]octane 2,2-dioxide hydrochloride (40 mg, 0.2 mmol) and triethylamine (20 mg, 0.2 mmol) in dichloromethane (2 mL) was stirred at 25° C. for 30 minutes, and then 1-[[6-(trifluoromethyl)-3-pyridyl]methyl]cyclopropanecarbaldehyde (69 mg, 0.3 mmol) and acetic acid (12 mg, 0.2 mmol) were added. The reaction mixture was stirred at 25° C. for 1 h. NaBH₃CN (128 mg, 0.61 mmol) was added and the reaction mixture was stirred at 25° C. for 8 h. The reaction was quenched by water (30 mL) and extracted with ethyl acetate (30 mL×3). The combined organic layers were washed with brine (30 mL×3), dried over anhydrous Na₂SO₄, filtered and concentrated. The residue was purified by reverse phase chromatography (water (0.225% FA)-ACN) to provide the title compound (44.5 mg, 57.6% yield).

Compound 157: ¹H NMR (400 MHz, CD₃OD) δ 8.61 (s, 1H), 7.95 (d, J=8.0 Hz, 1H), 7.75 (d, J=8.0 Hz, 1H), 4.12 (s, 4H), 2.77 (d, J=8.0 Hz, 4H), 2.65 (t, J=7.2 Hz, 2H), 2.22-2.13 (m, 4H), 0.63 (t, J=5.2 Hz, 2H), 0.41 (t, J=5.2 Hz, 2H). LCMS (ESI) [M+H]⁺=375.2.

### Example 158: 7-(2-(3,4-Difluorophenoxy)ethyl)-2-thia-7-azaspiro[3.5]nonane 2,2-dioxide

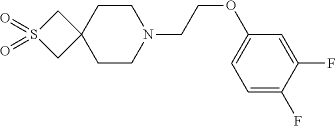 [SMILES: O=S1(=O)CC2(CCN(CCOc3ccc(F)c(F)c3)CC2)C1]

### Step 1: Synthesis of 4-(2-Bromoethoxy)-1,2-difluoro-benzene

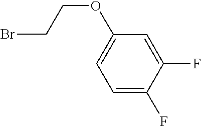 [SMILES: Fc1ccc(OCCBr)cc1F]

To a solution of 3,4-difluorophenol (1.6 g, 12.34 mmol) in acetone (20 mL) was added 1,2-dibromoethane (7 g, 37.01 mmol), K₂CO₃ (4.4 g, 31.84 mmol) and KI (200 mg, 1.2 mmol) at 20° C. The reaction mixture was heated to 60° C. and stirred for 15 h. The reaction mixture was diluted with water (50 mL) and extracted with ethyl acetate (40 mL×2). The combined organic layers were dried over anhydrous sodium sulfate, filtered and concentrated under reduced pressure. The resulting residue was purified by silica column chromatography (100% petroleum ether) to provide the title compound (1.3 g, 44.5% yield). ¹H NMR (400 MHz, CD₃OD) δ 7.22-7.18 (m, 1H), 6.93-6.90 (m, 1H), 6.76-6.74 (m, 1H), 4.30 (t, J=5.6 Hz, 2H), 3.72 (t, J=6.0 Hz, 2H).

### Step 2: Synthesis of the Title Compound

To a solution of 4-(2-bromoethoxy)-1,2-difluoro-benzene (89 mg, 0.38 mmol) and 2-thia-7-azaspiro[3.5]nonane 2,2-dioxide hydrochloride (40 mg, 0.19 mmol) in acetonitrile (4 mL), was added N,N-diisopropylethylamine (0.16 mL, 0.97 mmol) and potassium iodide (3 mg, 0.02 mmol) at 20° C. and stirred at 20° C. for 5 hours. The reaction mixture was diluted with water (30 mL), and extracted with ethyl acetate (30 mL×2). The combined organic layers were dried over anhydrous sodium sulfate, filtered and concentrated in vacuo. The resulting residue was purified by reverse phase chromatography (water (0.2% FA)-acetonitrile, 50%˜80%) to provide the title compound (58 mg, 91.7% yield). LCMS (ESI) [M+H]⁺=332.1.

Compound 158: ¹H NMR (400 MHz, CD₃OD) δ 7.22-7.13 (m, 1H), 6.95-6.92 (m, 1H), 6.77-6.75 (m, 1H), 4.20 (t, J=5.2 Hz, 2H), 3.97 (s, 4H), 3.12 (t, J=5.2 Hz, 2H), 2.91 (brs, 3H), 2.05 (t, J=5.6 Hz, 4H). LCMS (ESI) [M+H]⁺=332.1.

### Example 201: 2-(2-(4-(Trifluoromethyl)phenoxy)ethyl)-7-thia-2-azaspiro[3.5]nonane 7,7-dioxide

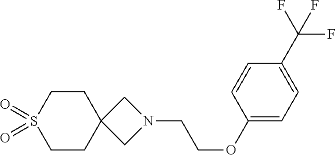 [SMILES: O=S1(=O)CCC2(CC1)CN(CCOc1ccc(C(F)(F)F)cc1)C2]

### Step 1: 1-(2-Bromoethoxy)-4-(trifluoromethyl)benzene

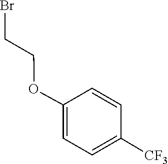 [SMILES: FC(F)(F)c1ccc(OCCBr)cc1]

To a solution of 4-(trifluoromethyl)phenol (1.0 g, 6.17 mmol) in N,N-dimethylformamide (10 mL) were added 1,2-dibromoethane (4.8 g, 25.55 mmol), K₂CO₃ (2.2 g, 15.92 mmol) and potassium iodide (100 mg, 0.60 mmol) at 20-30° C. Then the mixture was heated to 80° C. and stirred for 15 h. The reaction was quenched with water (50 mL), and extracted with ethyl acetate (40 mL×2). The combined organic phase was washed with brine (25 mL×3), dried over anhydrous Na₂SO₄, filtered and concentrated in vacuo to give the crude, which was purified by silica column chromatography (solvent gradient: 100% petroleum ether) to provide the title compound (690 mg, 2.56 mmol, 42% yield). ¹H NMR (400 MHz, CDCl₃) δ 7.57 (d, J=8.8 Hz, 2H), 6.99 (d, J=8.8 Hz, 2H), 4.37-4.32 (m, 2H), 3.69-3.63 (m, 2H).

### Step 2: Synthesis of the Title Compound

To a mixture of 1-(2-bromoethoxy)-4-(trifluoromethyl)benzene (100 mg, 0.37 mmol) and N,N-diisopropylethylamine (0.29 mL, 1.86 mmol) in acetonitrile (2 mL) was added 7-thia-2-azaspiro[3.5]nonane 7,7-dioxide hemioxalate (98 mg, 0.22 mmol) and KI (62 mg, 0.37 mmol). The reaction mixture was stirred at 80° C. for 16 h. The reaction mixture was quenched with water (10 mL), and extracted with ethyl acetate (30 mL×2). The combined organic phase was washed with brine (25 mL×3), dried over Na₂SO₄, filtered and concentrated under reduced pressure to give the crude, which was purified by pre-TLC (10% methanol in dichloromethane) to provide the title compound (61 mg, 0.161 mmol, 43% yield). LCMS (ESI) [M+H]⁺=364.1.

Compound 201: ¹H NMR (400 MHz, CDCl₃) δ ppm 7.55 (d, J=8.8 Hz, 2H), 6.95 (d, J=8.8 Hz, 2H), 4.03 (t, J=5.6 Hz, 2H), 3.20 (s, 4H), 2.99-2.96 (m, 4H), 2.90 (t, J=5.6 Hz, 2H), 2.34-2.31 (m, 4H).

### Example 202: 7-((1-(4-(Trifluoromethyl)phenoxy)cyclopentyl)methyl)-2-thia-7-azaspiro[3.5]nonane 2,2-dioxide

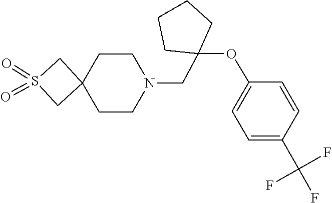 [SMILES: O=S1(=O)CC2(CCN(CC3(Oc4ccc(C(F)(F)F)cc4)CCCC3)CC2)C1]

### Step 1: Methyl 1-bromocyclopentanecarboxylate

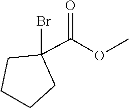

To a solution of cyclopentanecarboxylic acid (2.2 g, 19.27 mmol) in 1,2-dichloroethane (22 mL) was added bromine (0.99 mL, 19.27 mmol) and chlorosulfonic acid (2.23 mL, 22.4 mmol). The reaction mixture was stirred at 85° C. for 2 h. The reaction mixture was concentrated and the residue was dissolved in methanol (618 mg, 19.27 mmol). Then the mixture was stirred at 70° C. for another 12 h. The mixture was concentrated and the crude residue was diluted with methyl tertiary butyl ether (100 mL), washed with water (80 mL×3) and brine (80 mL). The organic layer was concentrated to give the product methyl 1-bromocyclopentanecarboxylate (3 g, 75% yield). The crude was used directly without further purification. ¹H NMR (400 MHz, CD₃OD) δ 3.80 (s, 3H), 2.34-2.26 (m, 4H), 2.04-1.90 (m, 2H), 1.87-1.73 (m, 2H).

### Step 2: Methyl 1-(4-(trifluoromethyl)phenoxy)cyclopentanecarboxylate

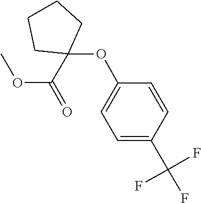 [SMILES: COC(=O)C1(Oc2ccc(C(F)(F)F)cc2)CCCC1]

To a solution of 4-(trifluoromethyl)phenol (1.3 g, 8.02 mmol) in acetonitrile (20 mL) was added methyl 1-bromocyclopentanecarboxylate (2.0 g, 9.6 6 mmol) and cesium carbonate (7.87 g, 24.15 mmol) at 20° C. The reaction mixture was stirred at 70° C. for 3 h. The mixture was then diluted with ethyl acetate (80 mL) and was washed with brine (30 mL×3), dried over anhydrous sodium sulfate, filtered and concentrated in vacuo. The residue was purified by silica column chromatography (0-10% ethyl acetate in petroleum ether) to provide the title compound (2 g, 72% yield). ¹H NMR (400 MHz, CD₃OD) δ 7.54 (d, J=8.4 Hz, 2H), 6.86 (d, J=8.4 Hz, 2H), 3.72 (s, 3H), 2.38-2.32 (m, 2H), 2.21-2.12 (m, 2H), 1.85-1.81 (m, 4H).

### Step 3: 1-[4-(Trifluoromethyl)phenoxy]cyclopentanecarboxylic acid

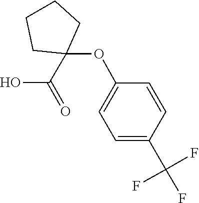 [SMILES: O=C(O)C1(Oc2ccc(C(F)(F)F)cc2)CCCC1]

To a solution of methyl 1-[4-(trifluoromethyl)phenoxy]cyclopentanecarboxylate (500.0 mg, 1.73 mmol) in tetrahydrofuran (2.5 mL) was added hydroxylithium hydrate (255 mg, 6.07 mmol) in water (1.5 mL). The reaction mixture was stirred at 20° C. for 2 h, was then adjusted to pH=2 with HCl (1 mol/L). The mixture was then diluted with water (20 mL) and the resulting mixture was extracted ethyl acetate (30×3 mL). The organic layers were combined and dried over anhydrous sodium sulfate, filtered and the organic layer and was concentrated under vacuum to afford provide the title compound (470 mg, 99% yield).

### Step 4: (2,2-Dioxido-2-thia-7-azaspiro[3.5]nonan-7-yl)(1-(4-(trifluoromethyl)phenoxy) cyclopentyl)methanone

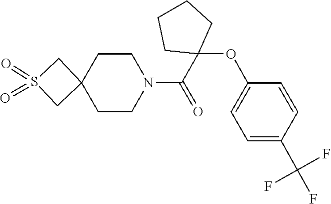 [SMILES: O=C(N1CCC2(CC1)CS(=O)(=O)C2)C1(Oc2ccc(C(F)(F)F)cc2)CCCC1]

To the mixture of 1-[4-(trifluoromethyl)phenoxy]cyclopentanecarboxylic acid (50.0 mg, 0.18 mmol), N,N-diisopropylethylamine (0.08 mL, 0.46 mmol), 2-thia-7-azaspiro[3.5]nonane 2-oxide hydrochloride (38.6 mg, 0.18 mmol) in dichloromethane (1 mL) was added HATU (104 mg, 0.27 mmol). The resulting mixture was stirred at 20° C. for 2 h. The reaction mixture was then diluted with brine (20 mL) and extracted with ethyl acetate (40 mL×2). The combined organic layers were concentrated under reduced pressure. The resulting residue was purified directly by silica column chromatography (solvent gradient: 0-50% ethyl acetate in petroleum ether) to provide the title compound (70 mg, 63% yield). LCMS (ESI) [M+H]⁺=432.1.

### Step 5: Synthesis of the Title Compound

To a cold (0° C.) solution of (2,2-dioxido-2-thia-7-azaspiro[3.5]nonan-7-yl)(1-(4-(trifluoromethyl)phenoxy) cyclopentyl)methanone (100 mg, 0.23 mmol) in tetrahydrofuran (2 mL) was added borane-THF (3 mL, 3 mmol) and the mixture was stirred at 70° C. for 4 h. The reaction was quenched with MeOH (1 mL), and the mixture was extracted with ethyl acetate (10 mL). The organics layers were washed with water (2×10 mL). The organics were dried over anhydrous Na₂SO₄, filtered and concentrated in vacuo. The resulting residue was purified by pre-HPLC (water (0.05% FA)-ACN, 40%˜70%) to provide the title compound as a formic acid salt (88.2 mg, 88.4% yield).

Compound 202: ¹H NMR (400 MHz, CD₃OD) δ 8.23 (s, 1H), 7.58 (d, J=8.4 Hz, 2H), 7.15 (d, J=8.4 Hz, 2H), 3.93 (s, 4H), 3.18 (s, 2H), 2.88 (s, 4H), 2.20-2.12 (m, 2H), 2.00 (t, J=5.2 Hz, 4H), 1.96-1.93 (m, 2H), 1.76-1.66 (m, 4H). LCMS (ESI) [M+H]⁺=418.1.

### Example 203* and 217*: (S)-7-(2-Methyl-3-(6-(trifluoromethyl)pyridin-3-yl)propyl)-2-thia-7-azaspiro[3.5]nonane 2,2-dioxide and (R)-7-(2-Methyl-3-(6-(trifluoromethyl)pyridin-3-yl)propyl)-2-thia-7-azaspiro[3.5]nonane 2,2-dioxide

### Step 1: (E)-Ethyl 2-methyl-3-(6-(trifluoromethyl)pyridin-3-yl)acrylate

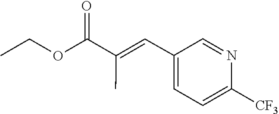 [SMILES: CCOC(=O)/C(C)=C/c1ccc(C(F)(F)F)nc1]

To a solution of triethyl 2-phosphonopropionate (10.2 g, 42.83 mmol) in tetrahydrofuran (50 mL) was added sodium hydride (1.71 g, 42.83 mmol) (60%) in portions at 0° C. The reaction mixture was stirred for 0.5 h. Then 6-(trifluoromethyl)nicotinaldehyde (5.0 g, 28.55 mmol) was added at 0° C. The reaction mixture was then stirred at 20° C. for 2 h. The reaction was quenched by saturated ammonium chloride solution (100 mL), diluted with water (20 mL), and extracted with ethyl acetate (500 mL×3). The organic layer was dried over anhydrous sodium sulfate, filtered and concentrated in vacuo. The resulting residue was purified by column silica chromatography (0-6% ethyl acetate in petroleum ether) to afford the title compound (7.0 g, 95% yield). LCMS (ESI) [M+H]⁺=260.0.

### Step 2: Ethyl 2-methyl-3-(6-(trifluoromethyl)pyridin-3-yl)propanoate

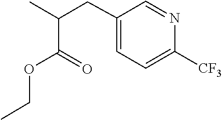 [SMILES: CCOC(=O)C(C)Cc1ccc(C(F)(F)F)nc1]

A solution of ethyl (E)-2-methyl-3-[6-(trifluoromethyl)-3-pyridyl]prop-2-enoate (7.0 g, 27 mmol) and 10% palladium on carbon (5747 mg, 5.4 mmol) in ethanol (100 mL) was stirred under H₂ (40 psi) at 25° C. for 2 h. The resulting mixture was filtered and the filtrate was concentrated to afford title compound (7000 mg, 99.2% yield). LCMS [M+H]⁺=262.1.

### Step 3: 2-Methyl-3-(6-(trifluoromethyl)pyridin-3-yl)propan-1-ol

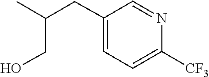

To a solution of ethyl 2-methyl-3-[6-(trifluoromethyl)-3-pyridyl]propanoate (7.0 g, 26.8 mmol) in tetrahydrofuran (100 mL) was added borane and lithium hydride (1.17 g, 53.59 mmol) at 0° C. The reaction was stirred at 20° C. for 48 h under N₂. The reaction was quenched by ammonium chloride solution (100 mL). The mixture extracted with ethyl acetate (500 mL×3). The combined organic layers were dried over anhydrous Na₂SO₄, filtered and concentrated. The residue was purified by silica flash chromatography (0-30% ethyl acetate in petroleum ether) to afford title compound (5000 mg, 83.4% yield). LCMS (ESI) [M+H]⁺=220.1.

### Step 4: (R)-2-Methyl-3-(6-(trifluoromethyl)pyridin-3-yl)propan-1-ol and (S)-2-methyl-3-(6-(trifluoromethyl)pyridin-3-yl)propan-1-ol

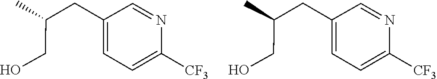

2-Methyl-3-[6-(trifluoromethyl)-3-pyridyl]propan-1-ol (13.0 g, 59.31 mmol) was separated using chiral SFC (SFC-9; 0.1% NH₃H₂O EtOH 15/15; 200 mL/min) to afford (R)-2-methyl-3-(6-(trifluoromethyl)pyridin-3-yl)propan-1-ol (4500 mg, 34.3% yield) (the first peak on SFC). LCMS (ESI) [M+H]⁺=220.0 and (S)-2-methyl-3-(6-(trifluoromethyl)pyridin-3-yl)propan-1-ol (4000 mg, 30.2% yield) (the second peak on SFC). LCMS (ESI) [M+H]⁺=220.0. The absolute stereochemistry was abitrairly assigned.

### Step 5: (S)-2-Methyl-3-(6-(trifluoromethyl)pyridin-3-yl)propyl methanesulfonate and (R)-2-Methyl-3-(6-(trifluoromethylpyridin-3-ylpropyl methanesulfonate

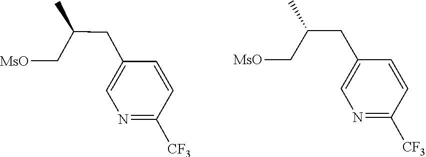 [SMILES: C[C@H](COS(C)(=O)=O)Cc1ccc(C(F)(F)F)nc1 ; C[C@@H](COS(C)(=O)=O)Cc1ccc(C(F)(F)F)nc1]

(S)-2-Methyl-3-[6-(trifluoromethyl)-3-pyridyl]propan-1-ol (stereochemistry arbitrarily assigned) (200 mg, 0.91 mmol) and N,N-diisopropylethylamine (0.37 mL, 2.01 mmol) were dissolved in dichloromethane (8 mL), and methanesulfonyl chloride (0.8 mL, 1 mmol) was added at 0° C. and the mixture was stirred at 25° C. for 0.5 h. The reaction mixture was quenched by water (25 mL) and the resulting mixture was extracted with dichloromethane (25 mL×3). The combined organic layers were washed with saturated sodium bicarbonate (20 mL×3) and brine (20 mL). The organic layer was dried over anhydrous sodium sulfate, filtered and concentrated to afford (S)-2-Methyl-3-(6-(trifluoromethyl)pyridin-3-yl)propyl methanesulfonate (stereochemistry arbitrarily assigned) (270 mg, 99.5% yield). [M+H]⁺=298.0. The other isomer, (R)-2-Methyl-3-(6-(trifluoromethyl)pyridin-3-yl)propyl methanesulfonate (stereochemistry arbitrarily assigned), was prepared similarly to the above synthetic procedure.

### Step 6A: Synthesis of the title compound (S)-7-(2-methyl-3-(6-(trifluoromethyl)pyridin-3

yl)propyl)-2-thia-7-azaspiro[3.5]nonane 2,2-dioxide (Compound 203*).

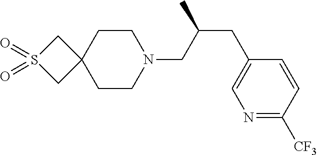 [SMILES: C[C@@H](Cc1ccc(C(F)(F)F)nc1)CN1CCC2(CC1)CS(=O)(=O)C2]

To (S)-2-Methyl-3-(6-(trifluoromethyl)pyridin-3-yl)propyl methanesulfonate (50.0 mg, 0.17 mmol) and N,N-diisopropylethylamine (32 mg, 0.25 mmol) in acetonitrile (5 mL) was added 2-thia-7-azaspiro[3.5]nonane 2-oxide hydrochloride (40 mg, 0.19 mmol) and potassium iodide (55 mg, 0.34 mmol). The mixture was stirred at 80° C. for 16 h. The resulting mixture was concentrated under vacuum. The residue was purified by pre-TLC (50% ethyl acetate in petroleum ether) to afford title compound (39.05 mg, 62% yield). Compound 203*: ¹H NMR (400 MHz, CD₃OD) δ 8.55 (s, 1H), 7.88 (d, J=8.0 Hz, 1H), 7.74 (d, J=8.0 Hz, 1H), 3.87 (s, 4H), 2.93-2.83 (m, 1H), 2.58 (dd, J=13.6, 7.6 Hz, 1H), 2.41 (brs, 4H), 2.20 (brs, 2H), 2.14-2.03 (m, 1H), 1.86 (d, J=5.2 Hz, 4H), 0.88 (d, J=6.4 Hz, 3H). LCMS (ESI): [M+H]⁺=377.2. The absolute stereochemistry was abitrairly assigned.

### Step 6B: Synthesis of the title compound (R)-7-(2-Methyl-3-(6-(trifluoromethyl)pyridin-3-yl)propyl)-2-thia-7-azaspiro[3.5]nonane 2,2-dioxide (Compound 217*)

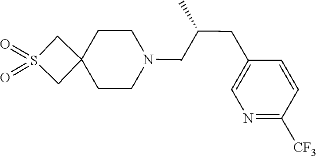 [SMILES: C[C@H](Cc1ccc(C(F)(F)F)nc1)CN1CCC2(CC1)CS(=O)(=O)C2]

The title compound was synthesized similarly to Step 6A, using (R)-2-methyl-3-(6-(trifluoromethyl)pyridin-3-yl)propyl methanesulfonate to provide the title compound (54.06 mg, 80.3% yield). LCMS (ESI): [M+H]⁺=377.1. Compound 217*: ¹H NMR (400 MHz, CD₃OD) δ 8.55 (d, J=1.6 Hz, 1H), 7.88 (dd, J=8.0, 1.6 Hz, 1H), 7.74 (d, J=8.0 Hz, 1H), 3.87 (s, 4H), 2.91-2.84 (m, 1H), 2.58-2.55 (m, 1H), 2.46-2.25 (m, 4H), 2.19-2.17 (m, 2H), 2.13-2.06 (m, 1H), 1.89-1.80 (m, 4H), 0.88 (d, J=6.4 Hz, 3H). The absolute stereochemistry was abitrairly assigned.

### Example 204: 7-(2-(2,4-Difluorophenoxy)ethyl)-2-thia-7-azaspiro[3.5]nonane 2,2-dioxide

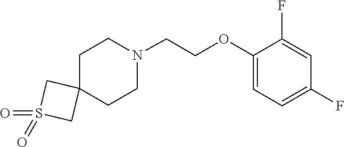 [SMILES: O=S1(=O)CC2(CCN(CCOc3ccc(F)cc3F)CC2)C1]

The title compound (66.23 mg, 84.6% yield) was synthesized similarly to example 201 using 2-thia-7-azaspiro[3.5]nonane 2,2-dioxide and 2,4-difluorophenol. LCMS (ESI) [M+H]⁺=332.1.

Compound 204: ¹H NMR (400 MHz, CD₃OD) δ 8.38 (s, 1H), 7.20-7.18 (m, 1H), 7.07-7.02 (m, 1H), 6.97-6.88 (m, 1H), 4.37 (t, J=4.8 Hz, 2H), 4.05 (s, 4H), 3.38 (t, J=4.8 Hz, 2H), 3.33-3.07 (m, 4H), 2.17 (t, J=5.6 Hz, 4H).

### Example 205: 7-(2-Methyl-2-((6-(trifluoromethyl)pyridin-3-yl)oxy)propyl)-2-thia-7-azaspiro[3.5]nonane 2,2-dioxide

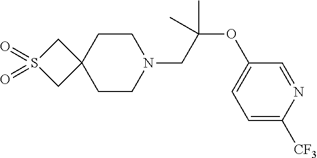 [SMILES: CC(C)(CN1CCC2(CC1)CS(=O)(=O)C2)Oc1ccc(C(F)(F)F)nc1]

### Step 1: Ethyl 2-methyl-2-((6-(trifluoromethyl)pyridin-3-yl)oxy)propanoate

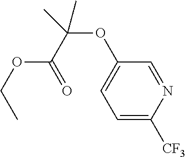 [SMILES: CCOC(=O)C(C)(C)Oc1ccc(C(F)(F)F)nc1]

To a solution of ethyl 2-bromo-2-methylpropanoate (2622 mg, 14.49 mmol) in acetonitrile (40 mL) was added 6-(trifluoromethyl)pyridin-3-ol (2.0 g, 9.66 mmol) and Cs₂CO₃ (7867 mg, 24.15 mmol) at 20° C. Then the reaction mixture was stirred at 70° C. for 6 h, diluted with ethyl acetate (70 mL). The mixture was washed with water, dried over anhydrous sodium sulfate, filtered and concentrated in vacuo. The residue was purified by purified by silica column chromatography (0-35% ethyl acetate in petroleum ether) to provide the title compound (2.38 g, 8.77 mmol, 91% yield). LCMS (ESI) [M+H]⁺=264.1.

### Step 2: 2-Methyl-2-((6-(trifluoromethyl)pyridin-3-yl)oxy)propanoic acid

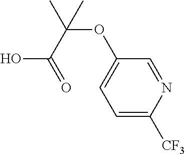 [SMILES: CC(C)(Oc1ccc(C(F)(F)F)nc1)C(=O)O]

To a solution of 2-methyl-2-[[6-(trifluoromethyl)-3-pyridyl]oxy]propanoate (500 mg, 1.9 mmol) in tetrahydrofuran (5 mL) and water (5 mL) was added hydroxyl lithium hydrate (318 mg, 7.6 mmol). The reaction mixture was stirred at 25° C. for 2 h and was then concentrated under vacuum. The residue was adjusted to pH 5 with HCl (1 mol/L). The resulting mixture was extracted with ethyl acetate (3×50 mL). The combined organic layers were dried over anhydrous Na₂SO₄, filtered and concentrated under vacuum to provide the title compound (455 mg, 1.79 mmol, 94% yield). LCMS (ESI) [M+H]⁺=250.1.

### Step 3: 1-(2,2-Dioxido-2-thia-7-azaspiro[3.5]nonan-7-yl)-2-methyl-2-((6-(trifluoromethyl)pyridin-3-yl)oxy)propan-1-one

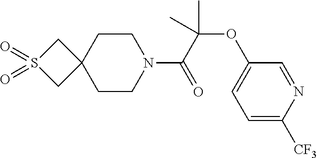 [SMILES: CC(C)(Oc1ccc(C(F)(F)F)nc1)C(=O)N1CCC2(CC1)CS(=O)(=O)C2]

To a solution of 2-thia-7-azaspiro[3.5]nonane 2,2-dioxide hydrochloride (50 mg, 0.24 mmol) in dichloromethane (2 mL) was added 2-methyl-2-[[6-(trifluoromethyl)-3-pyridyl]oxy]propanoic acid (50 mg, 0.20 mmol) and N,N-diisopropylethylamine (0.1 mL, 0.6 mmol) at 20° C. After stirring for 5 minutes, HATU (229 mg, 0.6 mmol) was added. The reaction mixture was stirred at 20° C. for 4 h. The reaction mixture was extracted with ethyl acetate (10 mL×2). The combined organic layers were washed with brine (10 mL×2), dried over anhydrous Na₂SO₄, filtered and concentrated under reduced pressure. The residue was purified by silica flash column chromatography (0-2% methanol in dichloromethane) to afford the title compound (70 mg, 0.124 mmol, 62% yield). LCMS (ESI) [M+H]⁺=407.1.

### Step 4: Synthesis of the Title Compound

A solution of 1-(2,2-dioxido-2-thia-7-azaspiro[3.5]nonan-7-yl)-2-methyl-2-((6-(trifluoromethyl)pyridin-3-yl)oxy)propan-1-one (70 mg, 0.20 mmol) in tetrahydrofuran (2 mL) was added borane-THF (5 mL, 5.0 mmol, 1M) at 0° C. within 5 mins, and then the mixture was stirred at 70° C. for 2 hours. The reaction was quenched with methanol (5 mL) and then ethyl acetate (20 mL) was added. The resulting mixture was washed with brine (30 mL×3), dried over Na₂SO₄, filtered and concentrated under reduced pressure. The residue was purified by pre-TLC (75% ethyl acetate in petroleum ether) to provide the title compound (54.93 mg, 0.1372 mmol, 70% yield). LCMS (ESI) [M+H]⁺=393.1.

Compound 205: ¹H NMR (400 MHz, CD₃OD) δ 8.38 (d, J=2.4 Hz, 1H), 7.89-7.51 (m, 2H), 3.89 (s, 4H), 2.61 (s, 6H), 1.90 (t, J=5.2 Hz, 4H), 1.39 (s, 6H).

### Example 206: 7-(2-(4-Chloro-3-fluorophenoxy)ethyl)-2-thia-7-azaspiro[3.5]nonane 2,2-dioxide

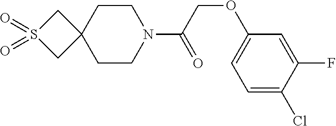 [SMILES: O=C(COc1ccc(Cl)c(F)c1)N1CCC2(CC1)CS(=O)(=O)C2]

The title compound (40 mg, 47% yield) was synthesized similarly to example 201 using 2-thia-7-azaspiro[3.5]nonane 2,2-dioxide and 4-chloro-3-fluorophenol. LCMS (ESI) [M+H]⁺=348.1.

Compound 206: ¹H NMR (400 MHz, CD₃OD) δ 7.41 (t, J=8.8 Hz, 1H), 6.97 (dd, J=3.2, 11.2 Hz, 1H), 6.88-6.86 (m, 1H), 4.31 (t, J=5.2 Hz, 2H), 4.04 (s, 4H), 3.31 (s, 2H), 3.12 (s, 4H), 2.14 (t, J=5.6 Hz, 4H).

### Examples 207A* and 207B*: (S)-7-(2-((6-(Trifluoromethyl)pyridin-3-yl)oxy)propyl)-2-thia-7-azaspiro[3.5]nonane 2,2-dioxide and (R)-7-(2-((6-(Trifluoromethyl)pyridin-3-yl)oxy)propyl)-2-thia-7-azaspiro[3.5]nonane 2,2-dioxide

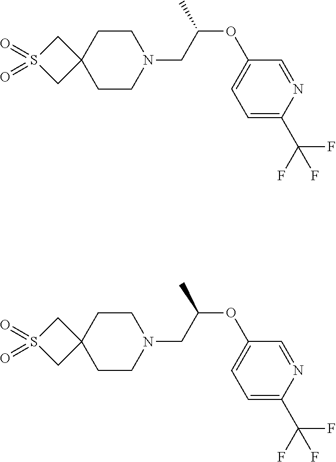 [SMILES: C[C@@H](CN1CCC2(CC1)CS(=O)(=O)C2)Oc1ccc(C(F)(F)F)nc1 ; C[C@H](CN1CCC2(CC1)CS(=O)(=O)C2)Oc1ccc(C(F)(F)F)nc1]

### Step 1: 2-((6-(Trifluoromethyl)pyridin-3-yl)oxy)propan-1-ol

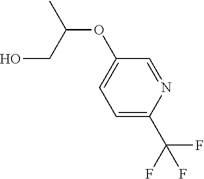

To a solution of ethyl 2-[[6-(trifluoromethyl)-3-pyridyl]oxy]propanoate (500 mg, 1.9 mmol) in dichloromethane (5 mL) was slowly added diisobutylaluminum hydride (3.0 mL, 3 mmol) at −78° C. and then the mixture was warmed to 25° C. and stirred for 16 hs. The reaction mixture was quenched by 2 N HCl aq (2 mL) and the mixture was extracted with ethyl acetate (50 mL×2). The combined organic phase was dried over Na₂SO₄, filtered, and concentrated in vacuo. The residue was purified by silica column chromatography (0-50% ethyl acetate in petroleum ether) to provide the title compound (350 mg, 73% yield) a mixture of enantiomers. LCMS (ESI): [M+H]⁺=222.1

### Step 2: 2-((6-(Trifluoromethyl)pyridin-3-yl)oxy)propyl methanesulfonate

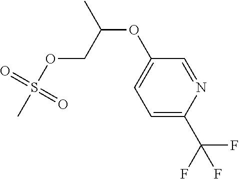 [SMILES: CC(COS(C)(=O)=O)Oc1ccc(C(F)(F)F)nc1]

To a mixture of 2-[[6-(trifluoromethyl)-3-pyridyl]oxy]propan-1-ol (350 mg, 1.58 mmol) and triethylamine (480 mg, 4.75 mmol) in dichloromethane (5 mL) was added methanesulfonyl chloride (199 mg, 1.74 mmol) at 0° C. and the mixture was stirred at 0° C. for 0.5 h. The reaction was quenched with water (2 mL) and the resulting mixture was extracted with dichloromethane (50 mL×3). The combined organics were washed with NaHCO₃ (10 mL×2), dried over anhydrous sodium sulfate, filtered and concentrated under vacuum to afford the title compound (330 mg, 70% yield) as a mixture of enantiomers. LCMS (ESI): [M+H]⁺=300.0.

### Step 3: 7-(2-((6-(Trifluoromethyl)pyridin-3-yl)oxy)propyl)-2-thia-7-azaspiro[3.5]nonane 2,2-dioxide

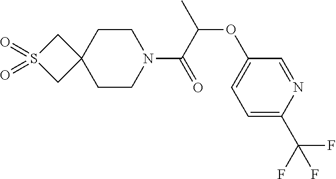 [SMILES: CC(Oc1ccc(C(F)(F)F)nc1)C(=O)N1CCC2(CC1)CS(=O)(=O)C2]

To a mixture of 2-[[6-(trifluoromethyl)-3-pyridyl]oxy]propyl methanesulfonate (150 mg, 0.50 mmol) and N,N-diisopropylethylamine (97 mg, 0.75 mmol) in acetonitrile (3 mL) was added 2-thia-7-azaspiro[3.5]nonane 2,2-dioxide hydrochloride (127 mg, 0.60 mmol) and potassium iodide (166 mg, 1 mmol) and then the mixture was stirred at 80° C. for 16 hours. The reaction mixture was concentrated under reduced pressure and the residue was purified by pre-TLC to afford the title compound (140 mg, 74% yield) as a mixture of enantiomers. LCMS (ESI): [M+H]⁺=379.1

### Step 4: Synthesis of the Title Compounds

7-(2-((6-(Trifluoromethyl)pyridin-3-yl)oxy)propyl)-2-thia-7-azaspiro[3.5]nonane 2,2-dioxide (140 mg, 0.37 mmol) was separated using chiral SFC (DAICEL CHIRALPAK AD-H (250 mm*30 mm, 10 μm), 0.1% NH₃H₂O EtOH; 15%˜15%, 50 mL/min) to afford the title Compound 207A* (the first peak on SFC, 36.16 mg, 26% yield) and the title Compound 207B* (the second peak on SFC, 46.78 mg, 33% yield), each of undefined/unassigned absolute stereochemistry. LCMS (ESI): [M+H]⁺=379.1

Example 207A*: ¹H NMR (400 MHz, CDCl₃) δ 8.38 (d, J=2.4 Hz, 1H), 7.61 (d, J=8.8 Hz, 1H), 7.31 (d, J=7.6 Hz, 1H), 4.63 (brs, 1H), 3.84 (s, 4H), 2.73-2.68 (m, 1H), 2.49 (brs, 5H), 1.87 (brs, 4H), 1.35 (d, J=6.4 Hz, 3H).

Example 207B*: ¹H NMR (400 MHz, CDCl₃) δ 8.38 (d, J=2.4 Hz, 1H), 7.61 (d, J=8.8 Hz, 1H), 7.31 (d, J=7.6 Hz, 1H), 4.63 (brs, 1H), 3.84 (s, 4H), 2.73-2.68 (m, 1H), 2.49 (brs, 5H), 1.87 (brs, 4H), 1.35 (d, J=6.4 Hz, 3H).

### Example 208: 7-(2-(3-Fluoro-4-(trifluoromethyl)phenoxy)-2-methylpropyl)-2-thia-7-azaspiro[3.5]nonane 2,2-dioxide

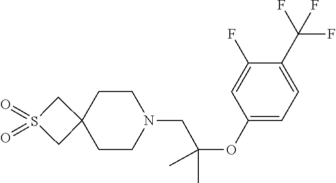 [SMILES: CC(C)(CN1CCC2(CC1)CS(=O)(=O)C2)Oc1ccc(C(F)(F)F)c(F)c1]

The title compound (68.68 mg, 0.1661 mmol, 59% yield) was synthesized similarly to Example 205 using 3-fluoro-4-(trifluoromethyl)phenol. LCMS (ESI) [M+H]⁺=410.2.

Compound 208: ¹H NMR (400 MHz, CDCl₃) δ ppm 7.49-7.44 (m, 1H), 6.85-6.80 (m, 2H), 3.86 (s, 4H), 2.59-2.53 (m, 6H), 1.92 (t, J=5.2 Hz, 4H), 1.36 (s, 6H).

### Example 209: 2-(2-Methyl-2-(4-(trifluoromethyl)phenoxy)propyl)-7-thia-2-azaspiro[3.5]nonane 7,7-dioxide

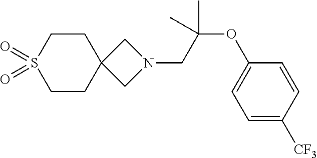 [SMILES: CC(C)(CN1CC2(CCS(=O)(=O)CC2)C1)Oc1ccc(C(F)(F)F)cc1]

The title compound (48.48 mg, 0.1152 mmol, 52% yield) was synthesized similarly to example 205 using 7-thia-2-azaspiro[3.5]nonane 7,7-dioxide. LCMS (ESI) [M+H]⁺=392.1.

Compound 209: ¹H NMR (400 MHz, CDCl₃) δ 7.53 (d, J=8.0 Hz, 2H), 7.05 (d, J=8.0 Hz, 2H), 3.25 (s, 4H), 2.99-2.98 (m, 4H), 2.71 (brs, 2H), 2.35 (brs, 4H), 1.31 (s, 6H).

### Example 210: 7-(2-((5-Fluoropyridin-3-yl)oxy)ethyl)-2-thia-7-azaspiro[3.5]nonane 2,2-dioxide

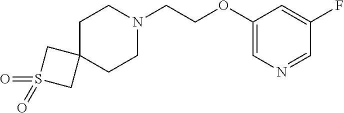 [SMILES: O=S1(=O)CC2(CCN(CCOc3cncc(F)c3)CC2)C1]

The title compound (47.29 mg, 0.15 mmol, 64% yield) was synthesized similarly to example 201 using 5-fluoropyridin-3-ol and 2-thia-7-azaspiro[3.5]nonane 2,2-dioxide. LCMS (ESI) [M+H]⁺=315.2.

Compound 210: ¹H NMR (400 MHz, CDCl₃) δ 8.18-8.12 (m, 2H), 6.96 (d, J=10.0 Hz, 1H), 4.16-4.12 (m, 2H), 3.87 (s, 4H), 2.83-2.80 (m, 2H), 2.53 (s, 4H), 1.95 (s, 4H).

### Examples 212A* and 212B*: (S)-7-(3-(3,4-Difluorophenyl)-2-methylpropyl)-2-thia-7-azaspiro[3.5]nonane 2,2-dioxide and (R)-7-(3-(3,4-difluorophenyl)-2-methylpropyl)-2-thia-7-azaspiro[3.5]nonane 2,2-dioxide

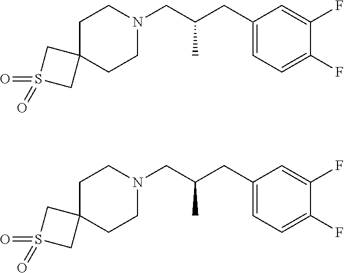 [SMILES: C[C@@H](Cc1ccc(F)c(F)c1)CN1CCC2(CC1)CS(=O)(=O)C2 ; C[C@H](Cc1ccc(F)c(F)c1)CN1CCC2(CC1)CS(=O)(=O)C2]

### Step 1: (E)-Ethyl 3-(3,4-difluorophenyl)-2-methylacrylate

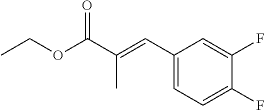 [SMILES: CCOC(=O)/C(C)=C/c1ccc(F)c(F)c1]

To a solution of ethyl 2-(diethoxyphosphoryl)propanoate (3688 mg, 15.48 mmol) in tetrahydrofuran (20 mL) was added sodium hydride (676 mg, 16.89 mmol) at 0° C. The reaction mixture stirred for 1 h, then 3,4-difluorobenzaldehyde (2 g, 14.07 mmol) was added and the reaction mixture was stirred at 25° C. for another 1 hour. The reaction mixture was quenched by NH₄C₁ (10 mL) and extracted with ethyl acetate (60 mL×3). The combined organic layers were dried over anhydrous sodium sulfate, filtered and concentrated under vacuum. The residue was purified by silica column chromatography (0-3% ethyl acetate in petroleum ether) to provide the title compound (2.17 g, 66% yield). LCMS (ESI): [M+H]⁺=227.1

### Step 2: Ethyl 3-(3,4-difluorophenyl)-2-methylpropanoate

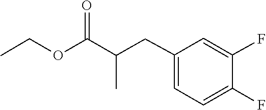 [SMILES: CCOC(=O)C(C)Cc1ccc(F)c(F)c1]

To a solution of (E)-ethyl 3-(3,4-difluorophenyl)-2-methylacrylate (1 g, 4.42 mmol) in ethanol (10 mL) was added 10% palladium on carbon (470 mg, 0.44 mmol) and the mixture was stirred under H₂ (15 Psi) at 25° C. for 16 hours. After filtration, the filtrate was concentrated under reduced pressure to provide the title compound (1 g, 99% yield) as a mixture of enantiomers. LCMS (ESI): [M+H]+=229.2.

### Step 3: 3-(3,4-Difluorophenyl)-2-methylpropanoic acid

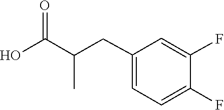

To a solution of ethyl 3-(3,4-difluorophenyl)-2-methylpropanoate (1 g, 4.38 mmol) in tetrahydrofuran (8 mL) and methanol (2 mL) was added lithium hydroxide monohydrate (552 mg, 13.14 mmol) in water (2 mL) and the mixture was stirred at 25° C. for 16 h. The reaction mixture was adjusted to pH 2 with HCl (1 mol/L). The resulting mixture was then extracted with ethyl acetate (50 mL×3). The combined organic layers were washed with brine (50 mL), dried over anhydrous sodium sulfate, filtered and concentrated under vacuum to provide the title compound (800 mg, 91% yield) as a mixture of enantiomers. ¹H NMR (400 MHz, CDCl₃) δ 7.12-6.97 (m, 2H), 6.92-6.89 (m, 1H), 3.04-3.00 (m, 1H), 2.75-2.64 (m, 2H), 1.20 (d, J=7.2 Hz, 3H).

### Step 4: 3-(3,4-Difluorophenyl)-1-(2,2-dioxido-2-thia-7-azaspiro[3.5]nonan-7-yl)-2-methylpropan-1-one

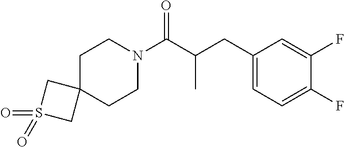 [SMILES: CC(Cc1ccc(F)c(F)c1)C(=O)N1CCC2(CC1)CS(=O)(=O)C2]

To the mixture of 3-(3,4-difluorophenyl)-2-methylpropanoic acid (130 mg, 0.65 mmol) and 2-thia-7-azaspiro[3.5]nonane 2,2-dioxide hydrochloride (154 mg, 0.73 mmol) in dichloromethane (7 mL) was added N,N-diisopropylethylamine (252 mg, 1.95 mmol) and HATU (741 mg, 1.95 mmol) at 0° C. The reaction mixture was stirred at 25° C. for 16 h. The resulting solution was diluted with water (5 mL) and extracted with dichloromethane (50 mL×2). The combined organic layer was dried over anhydrous sodium sulfate, filtered and concentrated under vacuum. The residue was purified by silica column chromatography (0→80% ethyl acetate in petroleum ether) to afford the title compound (200 mg, 86% yield) as a mixture of enantiomers. LCMS (ESI): [M+H]⁺=358.0.

### Step 5: 7-(3-(3,4-Difluorophenyl)-2-methylpropyl)-2-thia-7-azaspiro[3.5]nonane 2,2-dioxide

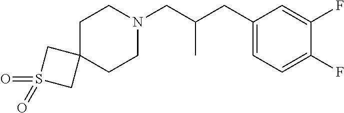 [SMILES: CC(Cc1ccc(F)c(F)c1)CN1CCC2(CC1)CS(=O)(=O)C2]

A solution of 3-(3,4-difluorophenyl)-1-(2,2-dioxido-2-thia-7-azaspiro[3.5]nonan-7-yl)-2-methylpropan-1-one (150 mg, 0.42 mmol) in tetrahydrofuran (3 mL) was added borane-THF (5 mL, 5 mmol) at 0° C. and stirred at 70° C. for 2 h. The reaction was quenched with methanol (5 mL) and stirred at 70° C. for 1 h. The resulting solution was concentrated under vacuum and the residue was purified by silica column chromatography (0→100% ethyl acetate in petroleum ether) to provide the title compound (110 mg, 76% yield) as a mixture of enantiomers. LCMS (ESI): [M+H]⁺=344.1

### Step 6: Synthesis of the Title Compounds

7-(3-(3,4-Difluorophenyl)-2-methylpropyl)-2-thia-7-azaspiro[3.5]nonane 2,2-dioxide (110 mg, 0.32 mmol) was separated using chiral SFC (Daicel Chiralpak AY-H (250 mm*30 mm, 10 μm) 0.1% NH₃H₂O EtOH; 15%, 60 mL/min) to afford the title Compound 212A* (first peak on SFC, 31.75 mg, 28% yield), LCMS (ESI): [M+H]⁺=344.2 and title Compound 212B*, second peak on SFC, 34.48 mg, 30% yield), each of undefined/unassigned absolute stereochemistry. LCMS (ESI): [M+H]⁺=344.2.

Compound 212*: ¹H NMR (400 MHz, CD₃OD) δ 7.17-7.07 (m, 2H), 6.97-6.94 (m, 1H), 3.88 (s, 4H), 2.78-2.71 (m, 1H), 2.48-2.33 (m, 4H), 2.21-2.10 (m, 2H), 2.04-1.86 (m, 6H), 0.85 (d, J=6.4 Hz, 3H).

Compound 212B*: ¹H NMR (400 MHz, CD₃OD) δ 7.17-7.05 (m, 2H), 6.97-6.93 (m, 1H), 3.88 (s, 4H), 2.78-2.71 (m, 1H), 2.48-2.32 (m, 4H), 2.21-2.11 (m, 2H), 2.04-1.83 (m, 6H), 0.84 (d, J=6.8 Hz, 3H).

### Example 213: 7-(3-(4-(Trifluoromethoxy)phenyl)propyl)-2-thia-7-azaspiro[3.5]nonane 2,2-dioxide

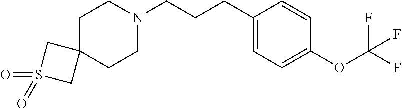 [SMILES: O=S1(=O)CC2(CCN(CCCc3ccc(OC(F)(F)F)cc3)CC2)C1]

### Step 1: Ethyl (E)-3-[4-(trifluoromethoxy)phenyl]prop-2-enoate

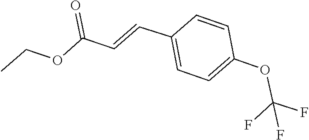 [SMILES: CCOC(=O)/C=C/c1ccc(OC(F)(F)F)cc1]

To a mixture of 4-(trifluoromethoxy)benzaldehyde (2.0 g, 10.52 mmol) in dichloromethane (20 mL) was added (carbethoxymethylene)triphenylphosphorane (4.03 g, 11.6 mmol) at 20° C. The reaction mixture was stirred for 16 h at that temperature. The reaction mixture was diluted with ethyl acetate (50 mL). The resulting mixture was washed with water (20 mL) and brine (20 mL). The organic layer was concentrated in vacuo and the residue was purified by silica column chromatography (0-10% ethyl acetate in petroleum ether) to provide the title compound (2500 mg, 91% yield). ¹H NMR (400 MHz, CD₃OD) δ 7.76-7.68 (m, 3H), 7.31 (d, J=8.0 Hz, 2H), 6.56 (d, J=16.0 Hz, 1H), 4.26 (q, J=7.2 Hz, 2H), 1.33 (t, J=7.2 Hz, 3H).

### Step 2: Ethyl 3-[4-(trifluoromethoxy)phenyl]propanoate

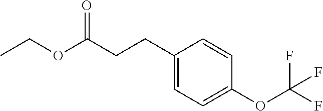 [SMILES: CCOC(=O)CCc1ccc(OC(F)(F)F)cc1]

To a solution of ethyl (E)-3-[4-(trifluoromethoxy)phenyl]prop-2-enoate (2.5 g, 9.61 mmol) in ethanol (30 mL) was added 10% palladium on carbon (2.044 g, 1.92 mmol). The reaction mixture was stirred under H₂ (15 psi) at 25° C. for 2 h. The mixture was filtered and concentrated in vacuo to provide the title compound (2500 mg, 93% yield).

### Step 3: 3-[4-(Trifluoromethoxy)phenyl]propanoic acid

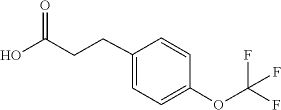 [SMILES: O=C(O)CCc1ccc(OC(F)(F)F)cc1]

To a solution of ethyl 3-[4-(trifluoromethoxy)phenyl]propanoate (2500 mg, 9.53 mmol) in tetrahydrofuran (20 mL) and ethanol (2 mL) was added hydroxyl lithium hydrate (1400 mg, 33.37 mmol) in water (4 mL). The reaction mixture was stirred at 20° C. for 2 h. The reaction mixture was adjusted to pH=2 with HCl (1 mol/L). Then the mixture was extracted with ethyl acetate (30×3 mL). The combined organic layers were dried over anhydrous sodium sulfate, filtered was concentrated under vacuum to provide the title compound (2200 mg, 99% yield).

### Step 4: 1-(2,2-Dioxido-2-thia-7-azaspiro[3.5]nonan-7-yl)-3-(4-(trifluoromethoxy)phenyl) propan-1-one

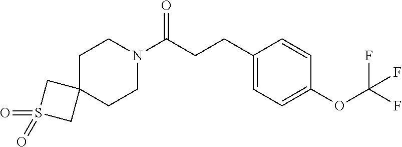 [SMILES: O=C(CCc1ccc(OC(F)(F)F)cc1)N1CCC2(CC1)CS(=O)(=O)C2]

To a solution of 4-(trifluoromethoxy)hydrocinnamic acid (66.37 mg, 0.28 mmol), 2-thia-7-azaspiro[3.5]nonane 2,2-dioxide hydrochloride (50 mg, 0.24 mmol) and N,N-diisopropylethylamine (0.1 mL, 0.59 mmol) in dichloromethane (1.0 mL) was added HATU (134.7 mg, 0.35 mmol). The resulting mixture was stirred at 20° C. for 2 h, was then diluted with brine (10 mL) and extracted with ethyl acetate (20 mL×2). The combined organic layers were concentrated under reduced pressure and the resulting residue was purified by silica column chromatography (0→50% ethyl acetate in petroleum ether) to provide the title compound (80 mg, 87% yield).

### Step 5: Synthesis of the Title Compound

To a cold (0° C.) solution of 1-(2,2-dioxido-2-thia-7-azaspiro[3.5]nonan-7-yl)-3-(4-(trifluoromethoxy)phenyl) propan-1-one (80 mg, 0.20 mmol) in tetrahydrofuran (1 mL) was added borane-THF (2.0 mL, 2 mmol). The reaction mixture was stirred at 70° C. for 4 h. The reaction was quenched with MeOH (1 mL) and diluted with ethyl acetate (20 mL). The organic layers were washed with brine (10 mL×2) and dried over anhydrous Na₂SO₄, filtered and concentrated in vacuo. The resulting residue was purified by pre-HPLC (water (0.05% FA)-ACN, 40%˜70%) to provide the title compound (55 mg, 69% yield). LCMS (ESI) [M+H]⁺=378.1.

Compound 213: ¹H NMR (400 MHz, CD₃OD) δ 7.33 (d, J=8.0 Hz, 2H), 7.20 (d, J=8.0 Hz, 2H), 4.00 (s, 4H), 3.05 (s, 4H), 2.94-2.81 (m, 2H), 2.73 (t, J=7.6 Hz, 2H), 2.10 (s, 4H), 1.93 (m, 2H).

### Example 214: 2-(2-(3,4-Difluorophenoxy)ethyl)-7-thia-2-azaspiro[3.5]nonane 7,7-dioxide

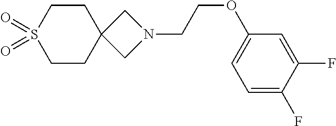 [SMILES: O=S1(=O)CCC2(CC1)CN(CCOc1ccc(F)c(F)c1)C2]

The title compound (53.4 mg, 73% yield) was synthesized similarly to example 201 using 2,4-difluorophenol. LCMS (ESI) [M+H]⁺=332.1.

Compound 214: ¹H NMR (400 MHz, CD₃OD) δ 7.17-7.14 (m, 1H), 6.90-6.84 (m, 1H), 6.74-6.68 (m, 1H), 3.98 (t, J=5.2 Hz, 2H), 3.27 (s, 4H), 3.05 (t, J=6.0 Hz, 4H), 2.92 (t, J=5.6 Hz, 2H), 2.30-2.26 (m, 4H).

### Examples 215A* and 215B*: (S)-7-(2-Methyl-3-(2-(trifluoromethyl)pyrimidin-5-yl)propyl)-2-thia-7-azaspiro[3.5]nonane 2,2-dioxide and (R)-7-(2-methyl-3-(2-(trifluoromethyl)pyrimidin-5-yl)propyl)-2-thia-7-azaspiro[3.5]nonane 2,2-dioxide

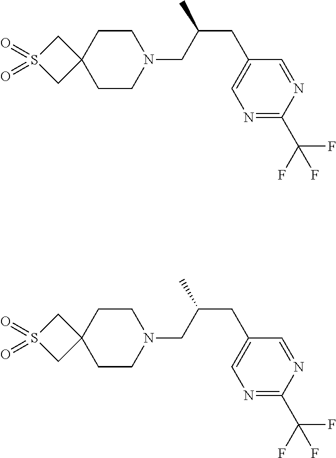 [SMILES: C[C@@H](Cc1cnc(C(F)(F)F)nc1)CN1CCC2(CC1)CS(=O)(=O)C2 ; C[C@H](Cc1cnc(C(F)(F)F)nc1)CN1CCC2(CC1)CS(=O)(=O)C2]

### Step 1: (S)-2-Methyl-3-(2-(trifluoromethyl)pyrimidin-5-yl)propan-1-ol and (R)-2-methyl-3-(2-(trifluoromethyl)pyrimidin-5-yl)propan-1-ol

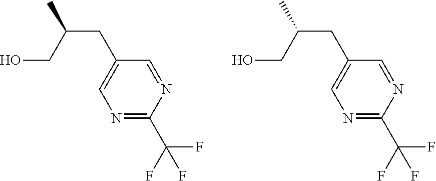

To a solution of ethyl 2-methyl-3-[2-(trifluoromethyl)pyrimidin-5-yl]propanoate (200 mg, 0.76 mmol) in dichloromethane (10 mL) was added diisobutylalumanylium hydride (1.53 mL, 1.53 mmol 1 M in toluene) in dropwise at −78° C. under nitrogen atmosphere. Then the mixture was stirred at 0° C. for 2 h. TLC showed the starting material was consumed completely and a new spot formed. The reaction was quenched by potassium sodium tartrate aqueous solution (5 mL) at 0° C. and extracted with ethyl acetate (50 mL×3). The combined organic layers were washed 3 times with brine, dried over Na₂SO₄, filtered and concentrated in vacuo. The residue was purified by silica flash chromatography (0-20% ethyl acetate in petroleum ether) to provide the title compound (150 mg, 85.7% yield). LCMS (ESI) [M+H]⁺=221.1.

### Step 2: 2-Methyl-3-(2-(trifluoromethyl)pyrimidin-5-yl)propyl methanesulfonate

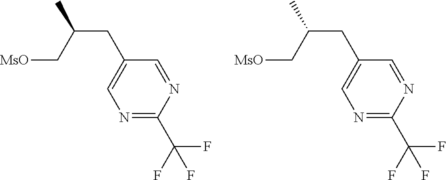 [SMILES: C[C@H](COS(C)(=O)=O)Cc1cnc(C(F)(F)F)nc1 ; C[C@@H](COS(C)(=O)=O)Cc1cnc(C(F)(F)F)nc1]

To a solution of (S)-2-methyl-3-(2-(trifluoromethyl)pyrimidin-5-yl)propan-1-ol and (R)-2-methyl-3-(2-(trifluoromethyl)pyrimidin-5-yl)propan-1-ol 1) and N,N-diisopropylethylamine (0.3 mL, 1.6 mmol) dissolved in dichloromethane (5 mL) was added methanesulfonyl chloride (91 mg, 0.79 mmol) at 0° C. The mixture was stirred at 25° C. for 0.5 h. The reaction was quenched with water (10 mL) and extracted with dichloromethane (20 mL×3). The combined organic layers were washed with saturated sodium bicarbonate (20 mL×3) and brine (20 mL), dried over anhydrous sodium sulfate, filtered and concentrated to provide the title compound (200 mg, 92.3% yield). LCMS (ESI) [M+H]⁺=299.0.

### Step 3: 7-(2-Methyl-3-(2-(trifluoromethyl)pyrimidin-5-yl)propyl)-2-thia-7-azaspiro[3.5]nonane 2,2-dioxide

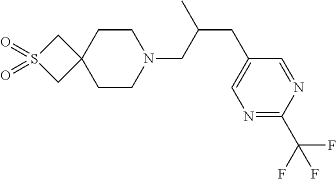 [SMILES: CC(Cc1cnc(C(F)(F)F)nc1)CN1CCC2(CC1)CS(=O)(=O)C2]

To a solution of [2-methyl-3-[2-(trifluoromethyl)pyrimidin-5-yl]propyl] methanesulfonate (120.0 mg, 0.40 mmol) and N,N-diisopropylethylamine (260 mg, 2.01 mmol) in acetonitrile (5 mL) was added 2-thia-7-azaspiro[3.5]nonane 2,2-dioxide hydrochloride (94 mg, 0.44 mmol) and potassium iodide (133 mg, 0.80 mmol). Then the mixture was stirred at 80° C. for 48 h. The resulting mixture was concentrated under reduced pressure and the residue was purified by silica flash chromatography on silica (0-80% ethyl acetate in petroleum ether) to provide the title compound (110 mg, 71% yield) as a mixture of enantiomers. LCMS (ESI) [M+H]⁺=378.2.

### Step 4: Synthesis of the Title Compounds

The mixture of enantiomers of 7-(2-methyl-3-(2-(trifluoromethyl)pyrimidin-5-yl)propyl)-2-thia-7-azaspiro[3.5]nonane 2,2-dioxide (110 mg, 0.29 mmol) was purified by chiral SFC (SFC-12; daicel chiralpak AD-H (250 mm*30 mm, 5 μm); CO₂/0.1% NH₃H₂O+MeOH 30/30; 60 mL/min) to provide Compound 215A (the first peak on SFC, 34.5 g, 30.1% yield) and Compound 215B (the second peak on SFC, 30.46 mg, 26% yield), each of undefined/unassigned absolute stereochemistry. LCMS (ESI) [M+H]⁺=378.2.

Compound 215A*: ¹H NMR (400 MHz, CD₃OD) δ 8.82 (s, 2H), 3.85 (s, 4H), 2.83-2.81 (m, 1H), 2.68-2.66 (m, 1H), 2.45-2.25 (m, 4H), 2.20-2.14 (m, 3H), 1.81-1.77 (m, 4H), 0.91 (d, J=6.0 Hz, 3H).

Compound 215B*: ¹H NMR (400 MHz, CD₃OD) δ 8.82 (s, 2H), 3.86 (s, 4H), 2.86-2.81 (m, 1H), 2.68-2.66 (m, 1H), 2.46-2.21 (m, 4H), 2.19-2.11 (m, 3H), 1.81-1.77 (m, 4H), 0.91 (d, J=6.0 Hz, 3H).

### Examples 216A* and 216B*: (S)-7-(2-Methyl-3-(4-(trifluoromethyl)phenyl)propyl)-2-thia-7-azaspiro[3.5]nonane 2,2-dioxide & (R)-7-(2-Methyl-3-(4-(trifluoromethyl)phenyl)propyl)-2-thia-7-azaspiro[3.5]nonane 2,2-dioxide

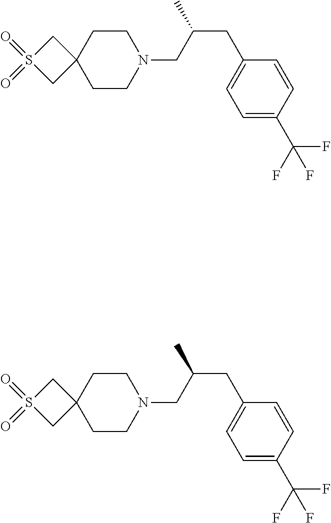 [SMILES: C[C@H](Cc1ccc(C(F)(F)F)cc1)CN1CCC2(CC1)CS(=O)(=O)C2 ; C[C@@H](Cc1ccc(C(F)(F)F)cc1)CN1CCC2(CC1)CS(=O)(=O)C2]

The title compounds were synthesized similarly to examples 215A and 215B using ethyl 2-methyl-3-(4-(trifluoromethyl)phenyl)propanoate.

The mixture of enantiomers (130 mg, 0.35 mmol) was purified by chiral SFC (SFC-17, Daicel Chiralpak IG(250 mm*30 mm, 10 μm), supercritical CO₂/0.1% NH₃H₂O, MeOH, 40/40, 70 mL/min) to provide Compound 216A* (the first peak on SFC, 41.0 mg, 31% yield) and Compound 216B* (the second peak on SFC, 35.8 mg, 27% yield), each of undefined/unassigned absolute stereochemistry. LCMS (ESI): [M+H]⁺=376.3.

Compound 216A*: ¹H NMR (400 MHz, CD₃OD) δ 7.56 (d, J=8.0 Hz, 2H), 7.36 (d, J=8.0 Hz, 2H), 3.88 (s, 4H), 2.88-2.84 (m, 1H), 2.45-2.25 (m, 4H), 2.20-2.17 (m, 3H), 2.09-2.00 (m, 1H), 1.91-1.88 (m, 4H), 0.86 (d, J=6.8 Hz, 3H).

Compound 216B*: ¹H NMR (400 MHz, CD₃OD) δ 7.56 (d, J=8.0 Hz, 2H), 7.36 (d, J=8.0 Hz, 2H), 3.88 (s, 4H), 2.90-2.82 (m, 1H), 2.45-2.25 (m, 4H), 2.20-2.17 (m, 3H), 2.09-2.00 (m, 1H), 1.91-1.89 (m, 4H), 0.86 (d, J=6.8 Hz, 3H).

### Example 218: 7-(2-(4-(Trifluoromethoxy)phenoxy)ethyl)-2-thia-7-azaspiro[3.5]nonane 2,2-dioxide

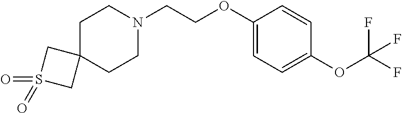 [SMILES: O=S1(=O)CC2(CCN(CCOc3ccc(OC(F)(F)F)cc3)CC2)C1]

### Step 1: 1-(2-Bromoethoxy)-4-(trifluoromethoxy)benzene

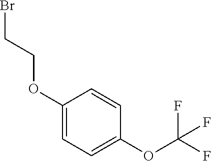 [SMILES: FC(F)(F)Oc1ccc(OCCBr)cc1]

The mixture of 4-(trifluoromethoxy)phenol (2.0 g, 11.23 mmol), 1,2-dibromoethane (6.35 g, 33.8 mmol), potassium carbonate (3.81 g, 27.56 mmol) and iodo potassium (184 mg, 1.11 mmol) were dissolved in acetonitrile (20 mL) at 20° C. and then the mixture was stirred at 60° C. for 2 h. The reaction mixture was diluted with water (50 mL), and extracted with ethyl acetate (40 mL×2). The combined organic layers were concentrated under reduced pressure and the resulting residue was purified by silica flash chromatography (100% petroleum ether) to provide the title compound (3 g, 10.52 mmol, 93.7% yield). ¹H NMR (400 MHz, CD₃OD) δ 7.20 (d, J=8.4 Hz, 2H), 7.01 (d, J=8.4 Hz, 2H), 4.32 (t, J=5.6 Hz, 2H), 3.70 (t, J=5.6 Hz, 2H).

### Step 2: Synthesis of the Title Compound

1-(2-Bromoethoxy)-4-(trifluoromethoxy)benzene (220.0 mg, 0.77 mmol), 2-thia-7-azaspiro[3.5]nonane 2,2-dioxide hydrochloride (80.0 mg, 0.38 mmol), N,N-diisopropylethylamine (0.32 mL, 1.94 mmol) and iodo potassium (80.0 mg, 0.5 mmol) were dissolved in acetonitrile (5 mL) and then the reaction mixture was stirred at 80° C. for 5 h. The reaction mixture was diluted with water (30 mL), and extracted with ethyl acetate (2×30 mL). The combined organic layers were concentrated under reduced pressure and the resulting residue was purified by reverse phase chromatography (Column, Welch Xtimate C18 150*25 mm*5 μm; water (0.2% formic acid)-CAN) to provide the title compound as the formic acid salt (68.76 mg, 0.17 mmol, 47% yield). LCMS (ESI): [M+H]⁺=380.1.

Compound 218: ¹H NMR (400 MHz, CD₃OD) δ 8.32 (s, 1H), 7.23 (d, J=9.2 Hz, 2H), 7.06 (d, J=9.2 Hz, 2H), 4.35 (s, 2H), 4.04 (s, 4H), 3.42 (s, 2H), 3.31-3.04 (m, 4H), 2.17 (t, J=5.2 Hz, 4H).

### Example 219: 2-(2-(3-Chloro-4-fluorophenoxy)ethyl)-7-thia-2-azaspiro[3.5]nonane 7,7-dioxide

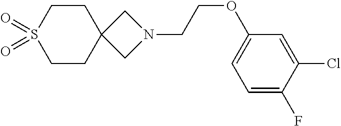 [SMILES: O=S1(=O)CCC2(CC1)CN(CCOc1ccc(F)c(Cl)c1)C2]

### Step 1: 4-(2-Bromoethoxy)-2-chloro-1-fluorobenzene

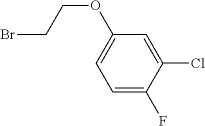 [SMILES: Fc1ccc(OCCBr)cc1Cl]

To a solution of 3-chloro-4-fluorophenol (1 g, 6.82 mmol) in acetonitrile (10 mL) were added 1,2-dibromoethane (3846 mg, 20.47 mmol), potassium carbonate (2358 mg, 17.06 mmol) and iodo potassium (113 mg, 0.68 mmol) at 25° C. and then the reaction mixture was stirred at 60° C. for 16 hours. The reaction mixture was diluted with water (30 mL), and extracted with ethyl acetate (40 mL×2). The combined organic layers were concentrated under reduced pressure and the resulting residue was purified by silica flash chromatography (100% petroleum ether) to provide the title compound (400 mg, 23.1% yield). ¹H NMR (400 MHz, CDCl₃) δ 7.07 (t, J=8.4 Hz, 1H), 6.96 (dd, J=6.0, 3.2 Hz, 1H), 6.81-6.77 (m, 1H), 4.25 (t, J=6.0 Hz, 2H), 3.63 (t, J=6.0 Hz, 2H).

### Step 2: Synthesis of the Title Compound

To a mixture of 4-(2-bromoethoxy)-2-chloro-1-fluoro-benzene (70 mg, 0.28 mmol) in acetonitrile (3 mL) were added 7-thia-2-azaspiro[3.5]nonane 7,7-dioxide hemioxalate (61 mg, 0.14 mmol), N,N-diisopropylethylamine (54 mg, 0.41 mmol) and potassium iodide (92 mg, 0.55 mmol) and then the mixture was stirred at 80° C. for 16 h. The mixture was filtered and filtrate was concentrated under reduced pressure to give the crude residue. The residue was purified by reverse phase chromatography (water (0.05% NH₃H₂O+10 mM NH₄HCO₃)—ACN, 40%˜70%) to provide the title compound (39.79 mg, 41% yield). LCMS (ESI): [M+H]⁺=348.1.

Compound 219: ¹H NMR (400 MHz, CD₃OD) δ 7.14 (t, J=9.2 Hz, 1H), 7.05 (dd, J=6.0, 2.8 Hz, 1H), 6.90-6.86 (m, 1H), 3.99 (t, J=5.2 Hz, 2H), 3.27 (s, 4H), 3.07-3.03 (m, 4H), 2.92 (t, J=5.2 Hz, 2H), 2.30-2.26 (m, 4H).

### Example 220: 7-(2-(4-Fluoro-3-(trifluoromethyl)phenoxy)-2-methylpropyl)-2-thia-7-azaspiro[3.5]nonane 2,2-dioxide

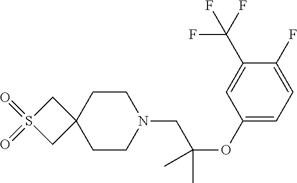 [SMILES: CC(C)(CN1CCC2(CC1)CS(=O)(=O)C2)Oc1ccc(F)c(C(F)(F)F)c1]

### Step 1: Methyl 2-[4-fluoro-3-(trifluoromethyl)phenoxy]-2-methyl-propanoate

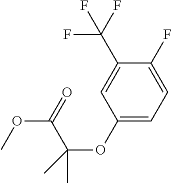 [SMILES: COC(=O)C(C)(C)Oc1ccc(F)c(C(F)(F)F)c1]

To a stirred solution of 4-fluoro-3-(trifluoromethyl)phenol (3.0 g, 16.66 mmol) and methyl 2-bromo-2-methylpropanoate (3.92 g, 21.65 mmol) in N,N-dimethylformamide (30 mL) was added potassium carbonate (6.91 g, 49.97 mmol) and then the mixture was stirred at 80° C. for 12 h. The reaction was quenched by water (50 mL) and extracted with ethyl acetate (60 mL×3). The combined organic layers were washed with brine (60 mL×3), dried over anhydrous Na₂SO₄, filtered and concentrated. The residue was purified by silica flash chromatography (0-10% ethyl acetate in petroleum ether) to provide the title compound (3.5 g, 12.5 mmol, 75% yield). ¹H NMR (400 MHz, CD₃OD) δ 7.26-7.21 (m, 1H), 7.15-7.12 (m, 2H), 3.76 (m, 3H), 1.57 (s, 6H).

### Step 2: 2-[4-Fluoro-3-(trifluoromethyl)phenoxy]-2-methyl-propanoic acid

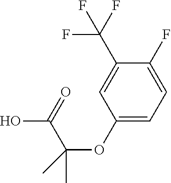 [SMILES: CC(C)(Oc1ccc(F)c(C(F)(F)F)c1)C(=O)O]

To a solution of methyl 2-[4-fluoro-3-(trifluoromethyl)phenoxy]-2-methyl-propanoate (1.5 g, 5.35 mmol) in water (3 mL), tetrahydrofuran (5 mL) and methyl alcohol (1 mL) was added hydroxylithium hydrate (2246 mg, 53.53 mmol) and then the mixture was stirred at 20° C. for 2 h. The reaction mixture was adjusted to pH=5 with HCl and extracted with ethyl acetate (20 mL×3). The combined organic layers were washed with brine (10 mL), dried over anhydrous Na₂SO₄, filtered and concentrated in vacuo to provide the title compound (1.4 g, 5.25 mmol, 98.3% yield).

### Step 3: 1-(2,2-Dioxido-2-thia-7-azaspiro[3.5]nonan-7-yl)-2-(4-fluoro-3-(trifluoromethyl)phenoxy)-2-methylpropan-1-one

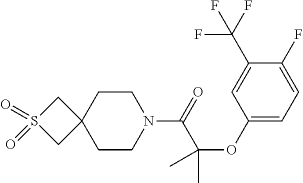 [SMILES: CC(C)(Oc1ccc(F)c(C(F)(F)F)c1)C(=O)N1CCC2(CC1)CS(=O)(=O)C2]

To the mixture of 2-[4-fluoro-3-(trifluoromethyl)phenoxy]-2-methyl-propanoic acid (50.0 mg, 0.19 mmol), N,N-diisopropylethylamine (13 mg, 0.38 mmol) and 2-thia-7-azaspiro[3.5]nonane 2,2-dioxide hydrochloride (40 mg, 0.19 mmol) in dichloromethane (3 mL) was added HATU (107 mg, 0.28 mmol). The resulting mixture was stirred at 20° C. for 2 h. The reaction mixture was diluted with brine (20 mL) and extracted with ethyl acetate (40 mL×2). The combined organic layers were concentrated in vacuo and the resulting residue was purified by silica flash chromatography (0→50% ethyl acetate in petroleum ether) to provide the title compound (70 mg, 0.165 mmol, 88% yield). LCMS (ESI): [M+H]⁺=424.1.

### Step 4: Synthesis of the Title Compound

To a cold (0° C.) solution of 1-(2,2-dioxido-2-thia-7-azaspiro[3.5]nonan-7-yl)-2-(4-fluoro-3-(trifluoromethyl)phenoxy)-2-methylpropan-1-one (70 mg, 0.165 mmol) in tetrahydrofuran (1 mL) was added borane-THF complex (1.0 mL, 1 mmol) and the mixture was stirred at 70° C. for 4 h. The reaction was quenched with methanol (1 mL) at ice bath and the resulting solution was concentrated in vacuo. The crude was purified by silica flash chromatography on silica (0→50% ethyl acetate in petroleum ether) to provide the title compound (48.01 mg, 0.117 mmol, 71% yield). LCMS (ESI): [M+H]⁺=410.0.

Compound 220: ¹H NMR (400 MHz, CD₃OD) δ 7.35-7.29 (m, 2H), 7.27-7.22 (m, 1H), 3.95 (s, 4H), 2.86-2.82 (s, 4H), 2.80 (s, 2H), 2.01 (t, J=5.2 Hz, 4H), 1.31 (s, 6H).

### Examples 221A* and 221B*: (S)-7-(2-(3,4-Difluorophenoxy)propyl)-2-thia-7-azaspiro[3.5]nonane 2,2-dioxide and (R)-7-(2-(3,4-difluorophenoxy)propyl)-2-thia-7-azaspiro[3.5 nonane 2,2-dioxide

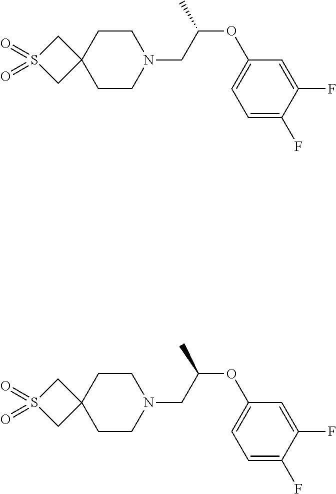 [SMILES: C[C@@H](CN1CCC2(CC1)CS(=O)(=O)C2)Oc1ccc(F)c(F)c1 ; C[C@H](CN1CCC2(CC1)CS(=O)(=O)C2)Oc1ccc(F)c(F)c1]

### Step 1: Ethyl (R)-2-(3,4-difluorophenoxy)propanoate and ethyl (S)-2-(3,4-difluorophenoxy)propanoate

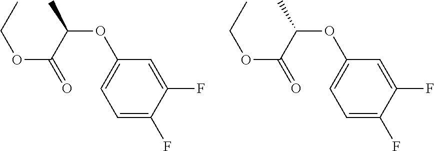 [SMILES: CCOC(=O)[C@@H](C)Oc1ccc(F)c(F)c1 ; CCOC(=O)[C@H](C)Oc1ccc(F)c(F)c1]

To a solution of 3,4-difluorophenol (2.0 g, 15.37 mmol) in N,N-Dimethylformamide (15 mL) was added cesium carbonate (11 g, 34.97 mmol) and ethyl 2-bromopropanoate (3 g, 17.7 mmol) and then the mixture was stirred at 80° C. for 15 h. Ethyl acetate (90 mL) was added and the resulting mixture was washed with brine (30 mL×3). The organic layer was dried over anhydrous Na₂SO₄, filtered and concentrated under reduced pressure to give the crude residue. The residue was purified by silica flash chromatography (0-10% ethyl acetate in petroleum ether) to provide the title compound (1.9 g, 53.7% yield). ¹H NMR (400 MHz, CD₃OD) δ 7.17-7.12 (m, 1H), 6.85-6.82 (m, 1H), 6.68-6.66 (m, 1H), 4.84-4.81 (m, 1H), 4.20 (q, J=7.2 Hz, 2H), 1.56 (d, J=6.8 Hz, 3H), 1.24 (t, J=7.2 Hz, 3H).

### Step 2: (R)-2-(3,4-Difluorophenoxy)propanoic acid and (S)-2-(3,4-difluorophenoxy)propanoic acid

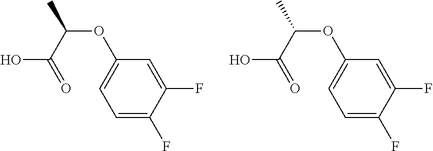

To a solution of ethyl 2-(3,4-difluorophenoxy)propanoate (500 mg, 2.17 mmol) in tetrahydrofuran (10 mL) and water (6 mL) was added hydroxylithium hydrate (455 mg, 10.86 mmol) and then the mixture was stirred at 20° C. for 2 h. The reaction mixture was adjusted to pH 2 with HCl (1N). The resulting mixture was extracted with ethyl acetate (30×3 mL) and the combined organic layers were dried over anhydrous sodium sulfate. The organic layer was concentrated under reduced pressure to provide the title compound (200 mg, 45.6% yield). The product was used directly without further purification.

### Step 3: (S)-2-(3,4-Difluorophenoxy)-1-(2,2-dioxido-2-thia-7-azaspiro[3.5]nonan-7-yl)propan-1-one and (R)-2-(3,4-difluorophenoxy)-1-(2,2-dioxido-2-thia-7-azaspiro[3.5]nonan-7-yl)propan-1-one

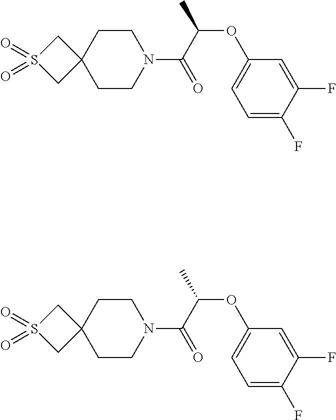 [SMILES: C[C@@H](Oc1ccc(F)c(F)c1)C(=O)N1CCC2(CC1)CS(=O)(=O)C2 ; C[C@H](Oc1ccc(F)c(F)c1)C(=O)N1CCC2(CC1)CS(=O)(=O)C2]

To a mixture of 2-(3,4-difluorophenoxy)propanoic acid (100 mg, 0.49 mmol), N,N-diisopropylethylamine (0.22 mL, 1.24 mmol) and 2-thia-7-azaspiro[3.5]nonane 2,2-dioxide hydrochloride (52 mg, 0.25 mmol) in dichloromethane (4 mL) was added HATU (282 mg, 0.74 mmol) and then the resulting mixture was stirred at 20° C. for 2 h. The reaction mixture was diluted with brine (20 mL) and extracted with ethyl acetate (40 mL×2). The combined organic layers were concentrated under reduced pressure and the resulting residue was purified by column chromatography on silica (0-60% ethyl acetate in petroleum ether) to provide the title compound (150 mg, 84.4% yield). LCMS (ESI) [M+H]⁺=360.1.

### Step 4: Synthesis of the Title Compounds

To a cold (0° C.) solution of 2-(3,4-difluorophenoxy)-1-(2,2-dioxido-2-thia-7-azaspiro[3.5]nonan-7-yl)propan-1-one (130 mg, 0.36 mmol) in tetrahydrofuran (2 mL) was added borane-THF complex (3 mL, 3 mmol, 1M) and then the mixture was stirred at 70° C. for 4 h. The reaction mixture was quenched with methanol (3 mL) at 0° C. and concentrated under reduced pressure to give the crude residue. The resulting residue was purified by silica column chromatography (0→100% ethyl acetate in petroleum ether) to provide the title compound (100 mg, 80% yield) as a mixture of enantiomers. LCMS (ESI) [M+H]⁺=346.1. The mixture of enantiomers of 7-(2-(3,4-difluorophenoxy)propyl)-2-thia-7-azaspiro[3.5]nonane 2,2-dioxide (100 mg, 0.29 mmol) was purified by SFC (daicel chiralcel oj-h (250 mm*30 mm, 5 μm), supercritical CO₂/0.1% NH₃H₂O+MeOH, 40/40, 60 mL/min) to provide Compound 221A* (the first peak on SFC, 27 mg, 26.5% yield) and to provide Compound 221B* (the second peak on SFC, 31 mg, 29.4% yield), each of undefined/unassigned absolute stereochemistry. LCMS (ESI) [M+H]⁺=346.1.

Compound 221A*: ¹H NMR (400 MHz, CD₃OD) δ 7.18-7.14 (m, 1H), 6.92-6.88 (m, 1H), 6.74-6.69 (m, 1H), 4.61-4.54 (m, 1H), 3.89 (s, 4H), 2.72-2.66 (m, 2H), 2.55-2.45 (m, 4H), 1.91-1.88 (m, 4H), 1.25 (d, J=6.0 Hz, 3H).

Compound 221B*: ¹H NMR (400 MHz, CD₃OD) δ 7.17-7.13 (m, 1H), 6.92-6.89 (m, 1H), 6.74-6.70 (m, 1H), 4.62-4.55 (m, 1H), 3.89 (s, 4H), 2.72-2.67 (m, 2H), 2.55-2.42 (m, 4H), 1.89-1.88 (m, 4H), 1.25 (d, J=6.0 Hz, 3H).

### Example 222: 7-((1-(4-(Trifluoromethyl)phenoxy)cyclobutyl)methyl)-2-thia-7-azaspiro[3.5]nonane 2,2-dioxide

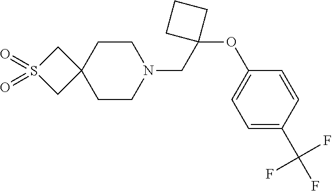 [SMILES: O=S1(=O)CC2(CCN(CC3(Oc4ccc(C(F)(F)F)cc4)CCC3)CC2)C1]

The title compound (53.26 mg, 92% yield) was synthesized similarly to example 202 using cyclobutanecarboxylic acid. LCMS (ESI) [M+H]⁺=404.1.

Compound 222: ¹H NMR (400 MHz, CD₃OD) δ 7.55 (d, J=9.2 Hz, 2H), 6.98 (d, J=8.8 Hz, 2H), 3.88 (s, 4H), 3.06 (s, 2H), 2.65 (s, 4H), 2.45-2.34 (m, 4H), 1.91 (t, J=5.6 Hz, 4H), 1.89-1.69 (m, 2H).

### Example 223: 2-(3-(6-(Trifluoromethyl)pyridin-3-yl)propyl)-7-thia-2-azaspiro[3.5]nonane 7,7-dioxide

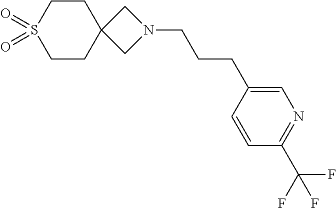 [SMILES: O=S1(=O)CCC2(CC1)CN(CCCc1ccc(C(F)(F)F)nc1)C2]

### Step 1: Ethyl 3-(6-(trifluoromethyl)pyridin-3-yl)propanoate

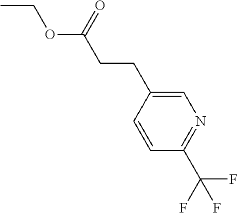 [SMILES: CCOC(=O)CCc1ccc(C(F)(F)F)nc1]

To a solution of ethyl (E)-3-[6-(trifluoromethyl)-3-pyridyl]prop-2-enoate (500 mg, 2.04 mmol) in ethanol (15 mL) was added 10% palladium on carbon (217 mg) and the mixture was stirred under H₂ (15 Psi) at 25° C. for 1 hour. After filtration and the filtrate was concentrated under reduced pressure to provide the title compound (400 mg, 79% yield). LCMS (ESI): [M+H]⁺=248.1

### Step 2: 3-(6-(Trifluoromethyl)pyridin-3-yl)propan-1-ol

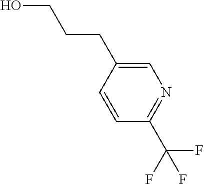 [SMILES: OCCCc1ccc(C(F)(F)F)nc1]

To a stirred solution of ethyl 3-(6-(trifluoromethyl)pyridin-3-yl)propanoate (400 mg, 1.62 mmol) in tetrahydrofuran (4 mL) was added lithiumaluminum hydride (184 mg, 4.85 mmol) at 0° C. and the mixture was stirred at 25° C. for 1 h. The mixture was quenched with 0.3 mL H₂O, 0.3 mL 15% NaOH aqueous solution, and 0.9 mLH₂O at 0° C. Then resulting mixture was filtered and the filtrate was concentrated under reduced pressure. The residue was purified by silica column chromatography (0-50% ethyl acetate in petroleum ether) to provide the title compound (250 mg, 75% yield).

### Step 3: 3-(6-(Trifluoromethyl)pyridin-3-yl)propyl methanesulfonate

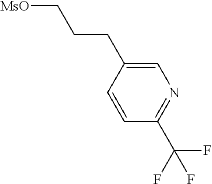 [SMILES: CS(=O)(=O)OCCCc1ccc(C(F)(F)F)nc1]

To a cool (0° C.) solution of 3-(6-(trifluoromethyl)pyridin-3-yl)propan-1-ol (250 mg, 1.22 mmol) and triethylamine (370 mg, 3.66 mmol) in dichloromethane (3 mL) was added methanesulfonyl chloride (154 mg, 1.34 mmol) and stirred for 0.5 hour. The reaction was quenched with water (5 mL) and extracted with ethyl acetate (10 mL×3). The combined organic layers were washed with brine (10 mL×2), dried over anhydrous sodium sulfate, filtered and concentrated under vacuum to provide the title compound (200 mg, 58% yield). LCMS (ESI): [M+H]⁺=284.0

### Step 4: Synthesis of the Title Compound

To a mixture of 3-(6-(trifluoromethyl)pyridin-3-yl)propyl methanesulfonate (100 mg, 0.35 mmol) and N,N-diisopropylethylamine (68 mg, 0.53 mmol) in acetonitrile (3 mL) was added 7-thia-2-azaspiro[3.5]nonane 7,7-dioxide oxalate (93 mg, 0.21 mmol) and potassium iodide (117 mg, 0.71 mmol). The reaction mixture was stirred at 80° C. for 16 h. The mixture was was concentrated in vacuo and the residue was purified by reverse phase chromatography (water (0.05% NH₃H₂O+10 mM NH₄HCO₃)—ACN, 35%˜65%) and lyophilized to provide the title compound (35.1 mg, 26% yield). LCMS (ESI): [M+H]⁺=363.2

Compound 223: ¹H NMR (400 MHz, CD₃OD) δ 8.57 (s, 1H), 7.90 (J=8.0 Hz, 1H), 7.74 (d, J=8.0 Hz, 1H), 3.19 (s, 4H), 3.06-3.02 (m, 4H), 2.78 (t, J=8.0 Hz, 2H), 2.60 (t, J=8.0 Hz, 2H), 2.25 (t, J=6.0 Hz, 4H), 1.78-1.70 (m, 2H).

### Example 225: 7-(3-(3,4-Difluorophenyl)propyl)-2-thia-7-azaspiro[3.5]nonane 2,2-dioxide

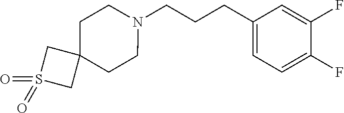 [SMILES: O=S1(=O)CC2(CCN(CCCc3ccc(F)c(F)c3)CC2)C1]

The title compound (36.45 mg, 52% yield) was synthesized similarly to example 213 using 3,4-difluorobenzaldehyde. LCMS (ESI) [M+H]⁺=330.1.

Compound 225: ¹H NMR (400 MHz, CD₃OD) δ 7.19-7.12 (m, 2H), 7.05-7.02 (m, 1H), 3.96 (d, J=14.0 Hz, 4H), 2.98-2.89 (m, 2H), 2.85-2.66 (m, 4H), 2.61 (t, J=8.0 Hz, 2H), 2.28 (s, 2H), 2.15-2.08 (m, 2H), 1.98-1.92 (s, 2H).

### Example 226: 7-(2-(4-(Trifluoromethyl)phenoxy)ethyl)-2-thia-7-azaspiro[3.5]nonane 2,2-dioxide

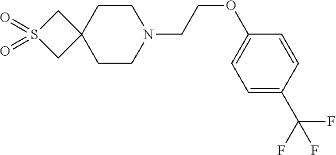 [SMILES: O=S1(=O)CC2(CCN(CCOc3ccc(C(F)(F)F)cc3)CC2)C1]

The title compound (40 mg, 47% yield) was synthesized similarly to example 201 using 4-(trifluoromethyl)phenol and 2-thia-7-azaspiro[3.5]nonane 2,2-dioxide. LCMS (ESI) [M+H]⁺=364.2.

Compound 226: ¹H NMR (400 MHz, CD₃OD) δ 7.61 (d, J=8.8 Hz, 2H), 7.12 (d, J=8.8 Hz, 2H), 4.29 (t, J=5.2 Hz, 2H), 3.98 (s, 4H), 3.13 (t, J=5.2 Hz, 2H), 3.00-2.90 (m, 4H), 2.05 (t, J=5.2 Hz, 4H).

### Examples 227A* and 227B*: (S)-7-(2-(4-(Trifluoromethyl)phenoxy)propyl)-2-thia-7-azaspiro[3.5]nonane 2,2-dioxide and (R)-7-(2-(4-(trifluoromethyl)phenoxy)propyl)-2-thia-7-azaspiro[3.5]nonane 2,2-dioxide

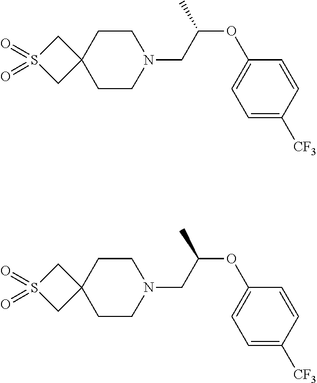 [SMILES: C[C@@H](CN1CCC2(CC1)CS(=O)(=O)C2)Oc1ccc(C(F)(F)F)cc1 ; C[C@H](CN1CCC2(CC1)CS(=O)(=O)C2)Oc1ccc(C(F)(F)F)cc1]

### Step 1: Ethyl 2-(4-(trifluoromethyl)phenoxy)propanoate

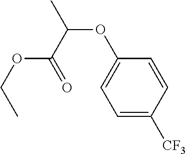 [SMILES: CCOC(=O)C(C)Oc1ccc(C(F)(F)F)cc1]

To a solution of 4-(trifluoromethyl)phenol (3.0 g, 18.51 mmol) in N,N-dimethylformamide (18 mL) was added cesium carbonate (13.71 g, 42.09 mmol) and ethyl 2-bromopropionate (3.86 g, 21.31 mmol) and the mixture was stirred at 80° C. for 15 h. The reaction mixture was diluted with ethyl acetate (90 mL) and the resulting mixture was washed with brine (30 mL×3), dried over anhydrous Na₂SO₄. The mixture was filtered and the filtrate was concentrated under reduced pressure. The residue was purified by silica column chromatography (0-20% ethyl acetate in petroleum ether) to provide the title compound (2.55 g, 9.72 mmol, 53% yield).

### Step 2: 2-(4-(Trifluoromethyl)phenoxy)propanoic acid

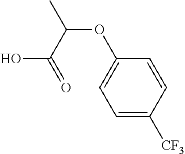 [SMILES: CC(Oc1ccc(C(F)(F)F)cc1)C(=O)O]

To a solution of ethyl 2-[4-(trifluoromethyl)phenoxy]propanoate (1.0 g, 3.81 mmol) in THF (12 mL) was added hydroxyl lithium hydrate (800 mg, 19.1 mmol) in water (4 mL) and the mixture was stirred at 20° C. for 2 h. The reaction mixture was adjusted to pH 2 with HCl (1 mol/L) and then the resulting mixture was extracted with ethyl acetate (30 mL×3). The combined organic layers were dried over anhydrous sodium sulfate, filtered and the filtrate was concentrated under vacuum to provide the title compound (820 mg, 3.50 mmol, 92% yield).

### Step 3: 1-(2,2-Dioxido-2-thia-7-azaspiro[3.5]nonan-7-yl)-2-(4-(trifluoromethyl)phenoxy) propan-1-one

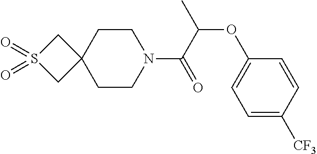 [SMILES: CC(Oc1ccc(C(F)(F)F)cc1)C(=O)N1CCC2(CC1)CS(=O)(=O)C2]

2-[4-(Trifluoromethyl)phenoxy]propanoic acid (100.0 mg, 0.43 mmol), 2λ\hat{ }{6}-thia-7-azaspiro[3.5]nonane 2,2-dioxide hydrochloride (99.0 mg, 0.4700 mmol) and N,N-diisopropylethylamine (0.3 mL, 1.71 mmol) dissolved in dichloromethane (2 mL) was added HATU (325 mg, 0.85 mmol). The resulting mixture was stirred at 20° C. for 2 h.

The mixture was concentrated under vacuum and the residue was purified by silica column chromatography (0-50% ethyl acetate in petroleum ether) to provide the title compound (165 mg, 0.422 mmol, 99% yield). LCMS (ESI) [M+H]⁺=392.2.

### Step 4: 7-(2-(4-(Trifluoromethyl)phenoxy)propyl)-2-thia-7-azaspiro[3.5]nonane 2,2-dioxide

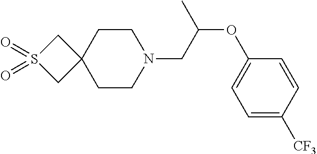 [SMILES: CC(CN1CCC2(CC1)CS(=O)(=O)C2)Oc1ccc(C(F)(F)F)cc1]

To a solution of 1-(2,2-dioxido-2-thia-7-azaspiro[3.5]nonan-7-yl)-2-(4-(trifluoromethyl)phenoxy) propan-1-one (165 mg, 0.42 mmol) in THF (2.0 mL) was added borane-THF (11.6 mL, 11.6 mmol) at 0° C. and the mixture was stirred at 80° C. for 4 h. The reaction mixture was quenched by methanol (10 mL) and the mixture was concentrated under reduced pressure. The residue was purified by silica column chromatography (0-100% ethyl acetate in petroleum ether) to provide the title compound (150 mg, 0.397 mmol, 94% yield) as a mixture of enantiomers. LCMS (ESI): [M+H]⁺=378.1.

### Step 5: Synthesis of the Title Compounds

7-(2-(4-(Trifluoromethyl)phenoxy)propyl)-2-thia-7-azaspiro[3.5]nonane 2,2-dioxide (180.0 mg, 0.48 mmol) was separated using chiral SFC (DAICEL CHIRALCEL OJ (250 mm*30 mm, 10 μm); 0.1% NH₃H₂O EtOH) to provide Compound 227A* (62 mg, 0.163 mmol, 34% yield, the first peak on SFC) and Compound 227B* (58.81 mg, 0.154 mmol, 32% yield, the second peak on SFC), each of undefined/unassigned absolute stereochemistry. LCMS (ESI): [M+H]⁺=378.1.

Compound 227A*: ¹H NMR (400 MHz, CDCl₃) δ 7.54 (d, J=8.4 Hz, 2H), 6.96 (d, J=8.4 Hz, 2H), 4.58 (s, 1H), 3.84 (s, 4H), 2.73-2.64 (m, 2H), 2.53-2.50 (m, 4H), 1.89 (brs, 4H), 1.32 (d, J=6.0 Hz, 4H).

Compound 227B*: ¹H NMR (400 MHz, CDCl₃) δ 7.54 (d, J=8.4 Hz, 2H), 6.96 (d, J=8.4 Hz, 2H), 4.58 (s, 1H), 3.84 (s, 4H), 2.73-2.64 (m, 2H), 2.53-2.50 (m, 4H), 1.89 (brs, 4H), 1.32 (d, J=6.0 Hz, 4H).

### Example 228: 7-(2-(3-Fluoro-4-(trifluoromethyl)phenoxy)ethyl)-2-thia-7-azaspiro[3.5]nonane 2,2-dioxide

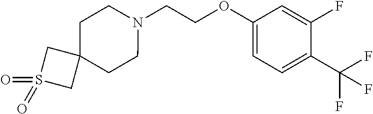 [SMILES: O=S1(=O)CC2(CCN(CCOc3ccc(C(F)(F)F)c(F)c3)CC2)C1]

The title compound (69.1 mg, 76% yield) was synthesized similarly to example 201 using 3-fluoro-4-(trifluoromethyl)phenol and 2-thia-7-azaspiro[3.5]nonane 2,2-dioxide. LCMS (ESI) [M+H]⁺=382.2.

Compound 228: ¹H NMR (400 MHz, CDCl₃) δ 7.53-7.48 (m, 1H), 6.78-6.68 (m, 2H), 4.11 (t, J=5.2 Hz, 2H), 3.87 (s, 4H), 2.81 (t, J=5.2 Hz, 2H), 2.53 (s, 4H), 1.94 (t, J=5.2 Hz, 4H).

### Example 229: 7-(2-((5-Fluoropyridin-2-yl)oxy)ethyl)-2-thia-7-azaspiro[3.5]nonane 2,2-dioxide

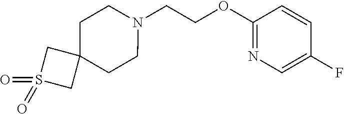 [SMILES: O=S1(=O)CC2(CCN(CCOc3ccc(F)cn3)CC2)C1]

The title compound (71.52 mg, 90% yield) was synthesized similarly to example 205 using 2-thia-7-azaspiro[3.5]nonane 2,2-dioxide and 5-fluoropyridin-2-ol. LCMS (ESI) [M+H]⁺=315.2.

Compound 229: ¹H NMR (400 MHz, CDCl₃) δ 7.97 (d, J=3.2 Hz, 1H), 7.37-7.32 (m, 1H), 6.74-6.71 (m, 1H), 4.40 (t, J=5.6 Hz, 2H), 3.86 (s, 4H), 2.78 (t, J=5.6 Hz, 2H), 2.54 (s, 4H), 1.94 (t, J=5.2 Hz, 4H).

### Example 230: 7-(2-((6-(Trifluoromethyl)pyridin-3-yl)oxy)ethyl)-2-thia-7-azaspiro[3.5]nonane 2,2-dioxide

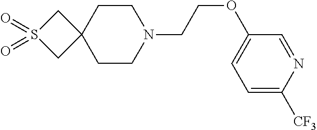 [SMILES: O=S1(=O)CC2(CCN(CCOc3ccc(C(F)(F)F)nc3)CC2)C1]

### Step 1: 5-(2-Bromoethoxy)-2-(trifluoromethyl)pyridine

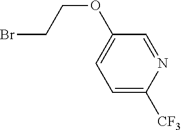 [SMILES: FC(F)(F)c1ccc(OCCBr)cn1]

To a solution of 6-(trifluoromethyl)pyridin-3-ol (4.0 g, 24.52 mmol) in acetone (20 mL) were added 1,2-dibromoethane (13.82 g, 73.57 mmol), potassium carbonate (8.47 g, 61.3 mmol) and iodopotassium (407 mg, 2.45 mmol) at 20° C. and then the reaction mixture was stirred at 60° C. for 1 h. The reaction mixture was diluted with ethyl acetate (200 mL) and washed with brine (100 mL). The organics were dried over anhydrous Na₂SO₄, filtered and concentrated in vacuo. The residue was purified by silica column chromatography (0-20% ethyl acetate in petroleum ether) to provide the title compound (5.0 g, 76% yield). ¹H NMR (400 MHz, CDCl₃) δ 8.35 (d, J=2.4 Hz, 1H), 7.59 (d, J=8.4 Hz, 1H), 7.32-7.22 (m, 1H), 4.36 (t, J=5.6 Hz, 2H), 3.64 (t, J=5.6 Hz, 2H).

### Step 2: Synthesis of the Title Compound

To a solution of 2-thia-7-azaspiro[3.5]nonane 2,2-dioxide hydrochloride (400 mg, 1.89 mmol) and 5-(2-bromoethoxy)-2-(trifluoromethyl)pyridine (1530 mg, 5.67 mmol) in acetonitrile (10 mL) was added potassium iodide (31 mg, 0.19 mmol) and N,N-diisopropylethylamine (1.67 mL, 9.45 mmol). The reaction mixture was stirred at 80° C. for 16 h. The reaction mixture was concentrated under reduced pressure and the residue was purified by silica column chromatography (0-5% methanol in ethyl acetate) to provide the title compound (520 mg, 75% yield). LCMS (ESI) [M+H]⁺=365.1.

Compound 230: ¹H NMR (400 MHz, CDCl₃) δ 8.38 (d, J=2.8 Hz, 1H), 7.62 (d, J=8.4 Hz, 1H), 7.29 (dd, J=2.4, 8.8 Hz, 1H), 4.18 (t, J=5.6 Hz, 2H), 3.86 (s, 4H), 2.83 (t, J=5.2 Hz, 2H), 2.53 (brs, 4H), 1.94 (t, J=5.2 Hz, 4H).

### Example 231: 7-(2-(4-Chloro-3-fluorophenoxy)-2-methylpropyl)-2-thia-7-azaspiro[3.5]nonane 2,2-dioxide

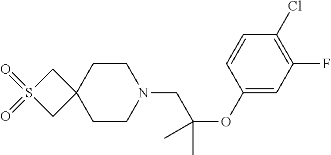 [SMILES: CC(C)(CN1CCC2(CC1)CS(=O)(=O)C2)Oc1ccc(Cl)c(F)c1]

The title compound (114.71 mg, 87% yield) was synthesized similarly to example 205 using 4-chloro-3-fluorophenol. LCMS (ESI) [M+H]⁺=376.0.

Compound 231: ¹H NMR (400 MHz, CD₃OD) δ 7.38-7.33 (m, 1H), 6.99-6.92 (m, 1H), 6.89-6.82 (m, 1H), 3.94 (s, 4H), 2.83 (brs, 4H), 2.77 (s, 2H), 2.08-1.94 (m, 4H), 1.34 (s, 6H).

### Examples 232A* and 232B*: (R)-2-(2-methyl-3-(4-(trifluoromethyl)phenyl)propyl)-7-thia-2-azaspiro[3.5]nonane 7,7-dioxide and (S)-2-(2-methyl-3-(4-(trifluoromethyl)phenyl)propyl)-7-thia-2-azaspiro[3.5]nonane 7,7-dioxide

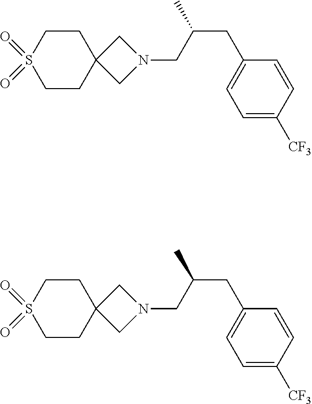 [SMILES: C[C@H](Cc1ccc(C(F)(F)F)cc1)CN1CC2(CCS(=O)(=O)CC2)C1 ; C[C@@H](Cc1ccc(C(F)(F)F)cc1)CN1CC2(CCS(=O)(=O)CC2)C1]

The title compounds were synthesized similarly to example 113 using ethyl 2-methyl-3-(4-(trifluoromethyl)phenyl)propanoate and 7-thia-2-azaspiro[3.5]nonane 7,7-dioxide.

2-(2-methyl-3-(4-(trifluoromethyl)phenyl)propyl)-7-thia-2-azaspiro[3.5]nonane 7,7-dioxide (150 mg, 0.40 mmol) was separated using chiral SFC (Daicel chiralpak AD-H (250 mm*30 mm, 10 μm), 0.1% NH₃H₂O EtOH, 15%, 50 m/min) to afford the Compound 232A* (the first peak on SFC, 52.9 mg, 34% yield), and to afford Compound 232B* (the second peak on SFC, 51.9 mg, 33% yield), each of undefined/unassigned absolute stereochemistry.

Compound 232A*: ¹H NMR (400 MHz, CDCl₃) δ 7.54 (d, J=8.0 Hz, 2H), 7.24 (d, J=8.0 Hz, 2H), 3.15-2.92 (m, 8H), 2.85-2.73 (m, 1H), 2.47-2.41 (m, 2H), 2.32 (s, 4H), 1.81-1.59 (m, 2H), 0.88 (d, J=5.6 Hz, 3H). LCMS (ESI) [M+H]⁺=376.2

Compound 232B*: ¹H NMR (400 MHz, CDCl₃) δ 7.53 (d, J=8.0 Hz, 2H), 7.25 (d, J=8.0 Hz, 2H), 3.11-2.92 (m, 8H), 2.84-2.73 (m, 1H), 2.47-2.39 (m, 2H), 2.31 (s, 4H), 1.79-1.63 (m, 2H), 0.86 (d, J=6.4 Hz, 3H). LCMS (ESI) [M+H]⁺=376.2

### Example 233: 7-(2-Methyl-2-(3-(trifluoromethyl)phenoxy)propyl)-2-thia-7-azaspiro[3.5]nonane 2,2-dioxide

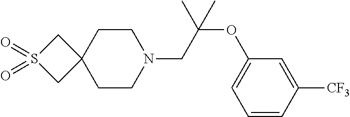 [SMILES: CC(C)(CN1CCC2(CC1)CS(=O)(=O)C2)Oc1cccc(C(F)(F)F)c1]

The title compound (40 mg, 48% yield) was synthesized similarly to example 205 using 3-(trifluoromethyl)phenol. LCMS (ESI) [M+H]⁺=392.1.

Compound 233: ¹HNMR (400 MHz, CD₃OD) δ 7.58-7.51 (m, 1H), 7.50-7.48 (m, 1H), 7.41-7.39 (m, 2H), 4.06 (s, 4H), 3.36 (brs, 2H), 2.68 (s, 4H), 2.23 (br s, 4H), 1.43 (s, 6H).

### Examples 234A* and 234B*: (R)-2-(2-methyl-3-(6-(trifluoromethyl)pyridin-3-yl)propyl)-7-thia-2-azaspiro[3.5]nonane 7,7-dioxide and (S)-2-(2-methyl-3-(6-(trifluoromethyl)pyridin-3-yl)propyl)-7-thia-2-azaspiro[3.5]nonane 7,7-dioxide

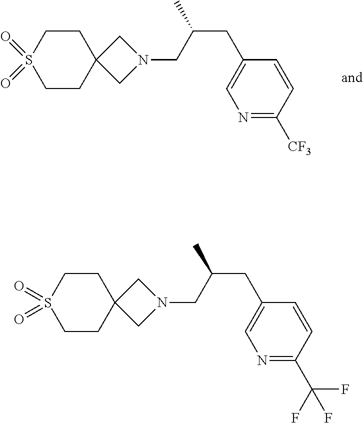 [SMILES: C[C@H](Cc1ccc(C(F)(F)F)nc1)CN1CC2(CCS(=O)(=O)CC2)C1 ; C[C@@H](Cc1ccc(C(F)(F)F)nc1)CN1CC2(CCS(=O)(=O)CC2)C1]

The title compounds were synthesized similarly to example 203 using ethyl 2-methyl-3-(6-(trifluoromethyl)pyridin-3-yl)propanoate and 7-thia-2-azaspiro[3.5]nonane 7,7-dioxide. Compound 234A*: 51.73 mg, 78% yield, LCMS (ESI) [M+H]⁺=377.2; Compound 234B*: 34.93 mg, 55% yield, LCMS (ESI) [M+H]⁺=377.2, each of undefined/unassigned absolute stereochemistry.

Compound 234A*: ¹H NMR (400 MHz, CD₃OD) δ 8.54 (s, 1H), 7.87 (d, J=8.0 Hz, 1H), 7.74 (d, J=8.0 Hz, 1H), 3.13 (s, 4H), 3.06-3.00 (m, 4H), 2.92-2.82 (m, 1H), 2.56-2.39 (m, 3H), 2.27-2.21 (m, 4H), 1.92-1.84 (m, 1H), 0.88 (d, J=6.4 Hz, 3H).

Compound 234B*: ¹H NMR (400 MHz, CD₃OD) δ 8.55 (s, 1H), 7.88 (d, J=7.6 Hz, 1H), 7.75 (d, J=7.6 Hz, 1H), 3.19 (m, 4H), 3.08-3.02 (m, 4H), 2.92-2.80 (m, 1H), 2.56-2.51 (m, 3H), 2.31-2.24 (m, 4H), 2.00-1.86 (m, 1H), 0.89 (d, J=6.8 Hz, 3H).

### Examples 235A* and 235B*: (R)-6-(3-(1-Isopropyl-3-(trifluoromethyl)-1H-pyrazol-5-yl)-2-methylpropyl)-2-thia-6-azaspiro[3.4]octane 2,2-dioxide and (S)-6-(3-(1-isopropyl-3-(trifluoromethyl)-1H-pyrazol-5-yl)-2-methylpropyl)-2-thia-6-azaspiro[3.4]octane 2,2-dioxide

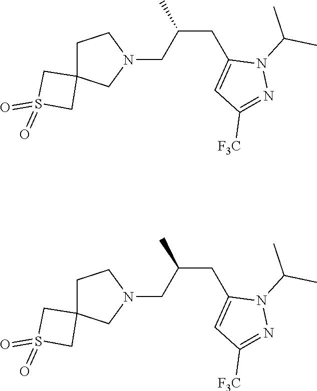 [SMILES: CC(C)n1nc(C(F)(F)F)cc1C[C@@H](C)CN1CCC2(C1)CS(=O)(=O)C2 ; CC(C)n1nc(C(F)(F)F)cc1C[C@H](C)CN1CCC2(C1)CS(=O)(=O)C2]

### Step 1: 1-Isopropyl-3-(trifluoromethyl)-1H-pyrazole

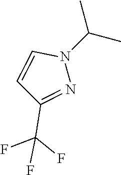

To a stirred suspension of 3-(trifluoromethyl)pyrazole (4 g, 29.39 mmol) in acetonitrile (50 mL) were added 2-iodopropane (15 g, 88.18 mmol) and Cs₂CO₃ (48 g, 146.97 mmol). The reaction mixture was stirred at 25° C. for 16 h. The reaction mixture was then filtered and the organic layer was diluted with water (50 mL). The mixture was then extracted with MTBE (50 mL×3). The combined organic layer were dried over anhydrous sodium sulfate, filtered and concentrated in vacuo to provide the title compound (4.2 g, 80% yield). ¹H NMR (400 MHz, CDCl₃) δ 7.46 (d, J=1.6 Hz, 1H), 6.50 (d, J=2.0 Hz, 1H), 4.61-4.51 (m, 1H), 1.53 (d, J=6.8 Hz, 6H).

### Step 2: 1-Isopropyl-3-(trifluoromethyl)-1H-pyrazole-5-carbaldehyde

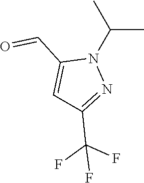

To a stirred solution of 1-isopropyl-3-(trifluoromethyl)pyrazole (1 g, 5.61 mmol) in THF (20 mL) was added n-butyllithium (3 mL, 7.5 mmol, 2.5 M in hexane) dropwise at −78° C. The mixture was stirred at same temperature for 1 hour. N,N-Dimethylformamide (1.5 mL, 19.47 mmol) was added dropwise and the reaction mixture was stirred at −78° C. for another 1 h. The reaction mixture was quenched by saturated NH₄C₁ solution (20 mL) and extracted with MTBE (50 mL×3). The combined organic layers were washed with brine (20 mL×2), dried over anhydrous sodium sulfate, filtered and concentrated under reduced pressure to provide the title compound (1.5 g). ¹H NMR (400 MHz, CDCl₃) δ 9.87 (s, 1H), 7.13 (s, 1H), 5.49-5.39 (m, 1H), 1.53 (d, J=6.8 Hz, 6H).

### Step 3: (E)-Ethyl 3-(1-isopropyl-3-(trifluoromethyl)-1H-pyrazol-5-yl)-2-methylacrylate

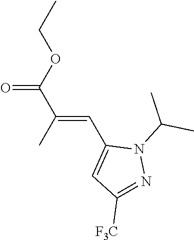 [SMILES: CCOC(=O)/C(C)=C/c1cc(C(F)(F)F)nn1C(C)C]

To an ice cooled solution of triethyl 2-phosphonopropionate (1907 mg, 8.01 mmol) in tetrahydrofuran (10 mL) was added NaH (349 mg, 8.73 mmol, 60% in mineral oil) slowly and stirred for 30 minutes. Then 2-isopropyl-5-(trifluoromethyl)pyrazole-3-carbaldehyde (1.5 g, 7.28 mmol) was added and the reaction mixture was stirred at 25° C. for another 1 h. The reaction mixture was quenched by NHCl₄ (30 mL) and extracted with ethyl acetate (50 mL×3). The combined organic layers were washed with brine (20 mL×2), dried over anhydrous sodium sulfate, filtered and concentrated in vacuo. The residue was purified by silica column chromatography (0-3% ethyl acetate in petroleum ether) to provide the title compound (900 mg, 43% yield), LCMS (ESI) [M+H]⁺=291.1.

### Step 4: Ethyl 3-(1-isopropyl-3-(trifluoromethyl)-1H-pyrazol-5-yl)-2-methylpropanoate

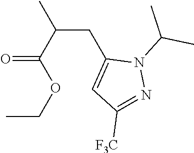 [SMILES: CCOC(=O)C(C)Cc1cc(C(F)(F)F)nn1C(C)C]

To a solution of ethyl (E)-3-[2-isopropyl-5-(trifluoromethyl)pyrazol-3-yl]-2-methyl-prop-2-enoate (900 mg, 3.1 mmol) in methanol (5 mL) was added 10% palladium on carbon (330 mg, 0.31 mmol). The reaction mixture was stirred under H₂ (15 psi) at 25° C. for 2 h, filtered and the the filtrate was concentrated in vacuo to afford the title compound (520 mg, 57% yield), LCMS (ESI): [M+H]⁺=293.1.

### Step 5: 3-(1-Isopropyl-3-(trifluoromethyl)-1H-pyrazol-5-yl)-2-methylpropan-1-ol

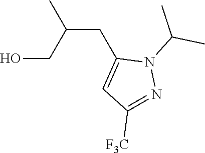 [SMILES: CC(CO)Cc1cc(C(F)(F)F)nn1C(C)C]

To a stirred solution of ethyl 3-[2-Isopropyl-5-(trifluoromethyl)pyrazol-3-yl]-2-methyl-propanoate (720 mg, 2.46 mmol) in tetrahydrofuran (5 mL) at 0° C. was added lithium aluminum hydride (280 mg, 7.39 mmol) and then the mixture was stirred at 25° C. for 1 hour. The reaction mixture was cooled to 0° C. 15% NaOH solution (1.2 mL) and water (1.2 mL) were added slowly to quench the reaction. The suspension mixture was filtered and the filtrate was concentrated in vacuo. The residue was purified by silica column chromatography (0-50% ethyl acetate in petroleum ether) to afford the title compound (500 mg, 81% yield), LCMS (ESI): [M+H]⁺=251.1.

### Step 6: 3-(1-Isopropyl-3-(trifluoromethyl)-1H-pyrazol-5-yl)-2-methylpropyl methanesulfonate

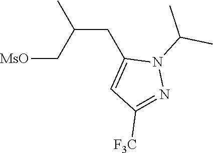 [SMILES: CC(COS(C)(=O)=O)Cc1cc(C(F)(F)F)nn1C(C)C]

To an ice cooled solution of 3-[2-isopropyl-5-(trifluoromethyl)pyrazol-3-yl]-2-methyl-propan-1-ol (500 mg, 2 mmol) and triethylamine (607 mg, 5.99 mmol) in dichloromethane (5 mL) was added methanesulfonyl chloride (252 mg, 2.2 mmol). The reaction mixture was stirred at 0° C. for 0.5 hour. The reaction mixture was quenched with water (5 mL) and extracted with dichloromethane (15 mL×3). The combined organic layers were dried over anhydrous sodium sulfate, filtered and concentrated in vacuo to afford the title compound (650 mg, 99% yield). LCMS (ESI) [M+H]⁺=329.1. The mixture of enantiomers was separated using chiral SFC (Phenomenex-Cellulose-2 (250 mm*30 mm, 5 μm), 0.1% NH₃H₂O; IPA; 25%, 60 mL/min) to provide Compound 235A* (the first peak on SFC, 36.1 mg, 30% yield) and Compound 235B* (the second peak on SFC, 43.8 mg, 35% yield), each of unassigned stereochemistry. LCMS (ESI): [M+H]⁺=408.3.

Compound 235A*: ¹H NMR (400 MHz, CD₃OD) δ 6.34 (s, 1H), 4.69-4.62 (m, 1H), 3.89 (s, 4H), 2.95-2.88 (m, 1H), 2.49-2.29 (m, 4H), 2.23-2.19 (m, 2H), 2.04-1.84 (m, 6H), 1.45 (dd, J=6.4, 2.0 Hz, 6H), 0.93 (d, J=6.4 Hz, 3H).

Compound 235B*: ¹H NMR (400 MHz, CD₃OD) δ 6.34 (s, 1H), 4.69-4.62 (m, 1H), 3.89 (s, 4H), 2.95-2.88 (m, 1H), 2.49-2.29 (m, 4H), 2.23-2.19 (m, 2H), 2.04-1.84 (m, 6H), 1.45 (dd, J=6.4, 2.0 Hz, 6H), 0.93 (d, J=6.4 Hz, 3H).

### Example 236: 7-(2-((2-(Trifluoromethyl)pyridin-4-yl)oxy)ethyl)-2-thia-7-azaspiro[3.5]nonane 2,2-dioxide

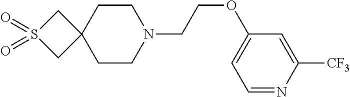 [SMILES: O=S1(=O)CC2(CCN(CCOc3ccnc(C(F)(F)F)c3)CC2)C1]

### Step 1: tert-Butyl 2-((2-(trifluoromethyl)pyridin-4-yl)oxy)acetate

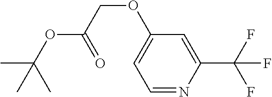 [SMILES: CC(C)(C)OC(=O)COc1ccnc(C(F)(F)F)c1]

To a stirring suspension of tert-butyl bromoacetate (1.0 mL, 6.14 mmol), 2-(trifluoromethyl)pyridin-4-ol (200.0 mg, 1.23 mmol) and silver(I)oxide (569 mg, 2.45 mmol) in dry N,N-dimethylformamide (4 mL) at 0° C. was added potassium iodide (41.6 mg, 0.25 mmol). Then the mixture was allowed to warm to 25° C. and stirred for another 15 h. The reaction mixture was concentrated under reduced pressure and the resulting residue was purified by silica column chromatography (0-20% ethyl acetate in petroleum ether) to afford the title compound (0.32 g, 1.15 mmol, 94% yield). LCMS (ESI) [M+H]⁺=278.1.

### Step 2: 2-((2-(Trifluoromethyl)pyridin-4-yl)oxy)acetic acid

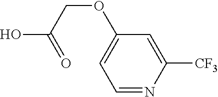 [SMILES: O=C(O)COc1ccnc(C(F)(F)F)c1]

A solution of tert-butyl 2-[[2-(trifluoromethyl)-4-pyridyl]oxy]acetate (100.0 mg, 0.36 mmol) in hydrochloric acid (0.9 mL, 3.6 mmol, 4 M in 1,4-dioxane). The mixture was stirred at 25° C. for 1 h. The mixture was concentrated in vacuo to provide the title compound (78 mg, 0.353 mmol, 98% yield). LCMS (ESI) [M+H]⁺=222.0.

### Step 3: 1-(2,2-Dioxido-2-thia-7-azaspiro[3.5]nonan-7-yl)-2-((2-(trifluoromethyl)pyridin-4-yl)oxy)ethanone

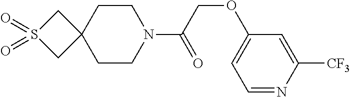 [SMILES: O=C(COc1ccnc(C(F)(F)F)c1)N1CCC2(CC1)CS(=O)(=O)C2]

To a mixture of 2-[[2-(trifluoromethyl)-4-pyridyl]oxy]acetic acid (78 mg, 0.353 mmol), 2-thia-7-azaspiro[3.5]nonane 2,2-dioxide hydrochloride (82.4 mg, 0.388 mmol), HATU (201 mg, 0.529 mmol) in dichloromethane (3 mL) was added N,N-diisopropylethylamine (182 mg, 1.41 mmol). The resulting mixture was stirred at 20° C. for 2 h. The mixture was concentrated and the residue was purified by silica flash chromatography (100% ethyl acetate) to provide the title compound (110 mg, 0.291 mmol, 82% yield). LCMS (ESI) [M+H]⁺=379.1.

### Step 4: Synthesis of the Title Compound

To a stirring solution of 1-(2,2-dioxido-2-thia-7-azaspiro[3.5]nonan-7-yl)-2-((2-(trifluoromethyl)pyridin-4-yl)oxy)ethanone (110 mg, 0.291 mmol) in tetrahydrofuran (2 mL) at 0° C. was added dropwise borane in tetrahydrofuran (1M, 8.0 mL, 8.0 mmol). The reaction mixture was stirred at 70° C. for 4 h. The reaction mixture was quenched by methyl alcohol (10 mL) at 0° C. and then the mixture was stirred at 70° C. for 2 h. The mixture was concentrated under vacuum and the residue was purified by reverse phase chromatography (water (0.05% NH₃H₂O+10 mM NH₄HCO₃)—ACN) to provide the title compound (29.9 mg, 0.0812 mmol, 27.9% yield). LCMS (ESI) [M+H]⁺=365.1.

Compound 236: ¹H NMR (400 MHz, CDCl₃) δ ppm 8.55 (d, J=5.6 Hz, 1H), 7.21 (d, J=2.4 Hz, 1H), 6.97 (dd, J=2.4, 5.6 Hz, 1H), 4.19 (t, J=5.6 Hz, 2H), 3.87 (s, 4H), 2.83 (t, J=5.6 Hz, 2H), 2.54 (brs, 4H), 1.95 (t, J=5.6 Hz, 4H).

### Examples 237* and 237B*: (S)-7-(3-Fluoro-3-(6-(trifluoromethyl)pyridin-3-yl)propyl)-2-thia-7-azaspiro[3.5]nonane 2,2-dioxide and (R)-7-(3-fluoro-3-(6-(trifluoromethyl)pyridin-3-yl)propyl)-2-thia-7-azaspiro[3.5]nonane 2,2-dioxide

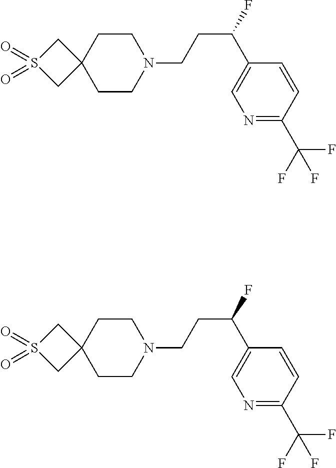 [SMILES: O=S1(=O)CC2(CCN(CC[C@H](F)c3ccc(C(F)(F)F)nc3)CC2)C1 ; O=S1(=O)CC2(CCN(CC[C@@H](F)c3ccc(C(F)(F)F)nc3)CC2)C1]

### Step 1: 1-(6-(Trifluoromethyl)pyridin-3-yl)but-3-en-1-ol

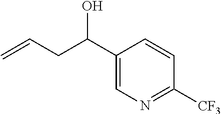

To a stirred solution of 6-(trifluoromethyl)nicotinaldehyde (3000 mg, 17.13 mmol) in tetrahydrofuran (30 mL) was added allylmagnesium bromide (20.56 mL, 20.56 mmol, 1M) at −78° C. The reaction mixture was stirred at −78° C. for 2 h. The reaction mixture was then quenched with saturated aqueous solution of NH₄C₁ (25 mL), and extracted with ethyl acetate (200 mL×2). The combined organic layers were dried over anhydrous Na₂SO₄, filtered and concentrated in vacuo. The resulting residue was purified by flash chromatography on silica (0-50% ethyl acetate in petroleum ether) to afford the title compound (2000 mg, 53.8% yield) as a mixture of enantiomers. ¹H NMR (400 MHz, CDCl₃) 8.69 (d, J=1.2 Hz, 1H), 7.94-7.88 (m, 1H), 7.68 (d, J=8.0 Hz, 1H), 5.87-5.72 (m, 1H), 5.26-5.15 (m, 2H), 4.89 (brs, 1H), 2.65-2.45 (m, 3H).

### Step 2: 5-(1-Fluorobut-3-en-1-yl)-2-(trifluoromethyl)pyridine

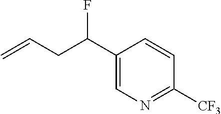

To a stirred solution of 1-[6-(trifluoromethyl)-3-pyridyl]but-3-en-1-ol (2100 mg, 9.67 mmol) in dichloromethane (30 mL) was added diethylaminosulfur trifluoride (1.66 mL, 12.57 mmol) at −78° C. and the mixture was stirred at −78° C. for 2 h. The reaction was quenched with saturated aqueous NaHCO₃ solution (15 mL) and extracted with ethyl acetate (30 mL×2). The combined organic layers were washed with brine (30 mL), dried over anhydrous Na₂SO₄, filtered and concentrated. The resulting residue was purified by silica flash chromatography (0-30% ethyl acetate in petroleum ether) to provide the title compound (1000 mg, 45.1% yield) as a mixture of enantiomers. LCMS (ESI) [M+H]⁺=220.1.

### Step 3: 3-Fluoro-3-(6-(trifluoromethyl)pyridin-3-yl)propanal

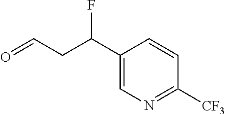 [SMILES: O=CCC(F)c1ccc(C(F)(F)F)nc1]

To a solution of 5-(1-fluorobut-3-enyl)-2-(trifluoromethyl)pyridine (300 mg, 1.37 mmol) in water (6 mL) and tetrahydrofuran (12 mL) was added osmium tetroxide (70 mg, 0.28 mmol) at 0° C. The reaction mixture was stirred for 20 minutes, then sodium periodate (1171 mg, 5.47 mmol) was added and the reaction mixture was stirred at 25° C. for 1 hour. The reaction mixture was quenched with sat. Na₂SO₃ solution and extracted with ethyl acetate (25 mL×3). The combined organic layers were washed with brine (30 mL), dried over anhydrous Na₂SO₄, filtered and concentrated in vacuo to afford the title compound (0.2 g, 66.1% yield) as a mixture of enantiomers. ¹H NMR (400 MHz, CDCl₃) 9.85 (s, 1H), 8.76 (s, 1H), 7.92 (d, J=8.4 Hz, 1H), 7.75 (d, J=8.4 Hz, 1H), 6.26-6.08 (m, 1H), 3.38-2.93 (m, 2H).

### Step 4: 7-(3-Fluoro-3-(6-(trifluoromethyl)pyridin-3-yl)propyl)-2-thia-7-azaspiro[3.5]nonane 2,2-dioxide

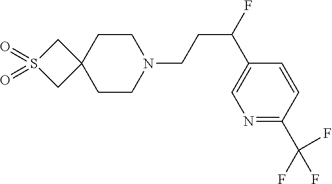 [SMILES: O=S1(=O)CC2(CCN(CCC(F)c3ccc(C(F)(F)F)nc3)CC2)C1]

To a solution of 3-fluoro-3-[6-(trifluoromethyl)-3-pyridyl]propanal (90.0 mg, 0.41 mmol) in methanol (5 mL) was added 2-thia-7-azaspiro[3.5]nonane 2,2-dioxide hydrochloride (80.0 mg, 0.38 mmol), acetic acid (0.11 mL, 1.89 mmol) and sodium cyanoborohydride (71 mg, 1.13 mmol) and the mixture was stirred at 70° C. for 1 hour. The reaction mixture was diluted with ethyl acetate, washed with saturated sodium bicarbonate (30 mL×2). The organic layers were dried over anhydrous Na₂SO₄, filtered and concentrated in vacuo. The residue was purified by silica flash chromatography (0-5% methanol in ethyl acetate) to afford the title compound (100 mg, 68.9% yield) as a mixture of enantiomers. LCMS (ESI) [M+H]⁺=381.1.

### Step 5: Synthesis of the Title Compounds

7-(3-Fluoro-3-(6-(trifluoromethyl)pyridin-3-yl)propyl)-2-thia-7-azaspiro[3.5]nonane 2,2-dioxide (112 mg, 0.29 mmol) was separated using chiral SFC (daicel chiralpak AD (250 mm*30 mm, 10 μm); Neu-EtOH 20/20; 70 mL/min) to afford the title Compound 237A* (35.72 mg, 30.9% yield, the first peak on SFC) and the title Compound 237B* (32.57 mg, 28.5% yield) (the second peak on SFC), each of unassigned stereochemistry. LCMS (ESI) [M+H]⁺=381.1.

Compound 237A*: ¹H NMR (400 MHz, CDCl₃) δ 8.69 (s, 1H), 7.87 (d, J=8.0 Hz, 1H), 7.73 (d, J=8.0 Hz, 1H), 5.78-5.63 (m, 1H), 3.85 (s, 4H), 2.58-2.41 (m, 6H), 2.24-1.97 (m, 2H), 1.90 (t, J=5.2 Hz, 4H).

Compound 237B*: ¹H NMR (400 MHz, CDCl₃) δ 8.69 (s, 1H), 7.87 (d, J=6.8 Hz, 1H), 7.73 (d, J=8.0 Hz, 1H), 5.78-5.53 (m, 1H), 3.85 (s, 4H), 2.58-2.41 (m, 6H), 2.24-2.13 (m, 1H), 2.09-1.95 (m, 1H), 1.90 (t, J=5.2 Hz, 4H).

### Example 238: 7-(2-(4-(6-(Trifluoromethyl)pyridin-3-yl)phenoxy)ethyl)-2-thia-7-azaspiro[3.5]nonane 2,2-dioxide

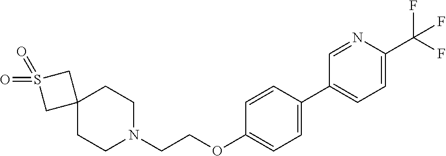 [SMILES: O=S1(=O)CC2(CCN(CCOc3ccc(-c4ccc(C(F)(F)F)nc4)cc3)CC2)C1]

The title compound was synthesized similarly to example 241 (42.65 mg, 31.2% yield) using 4-bromophenol instead of 6-bromopyridin-3-ol. LCMS (ESI) [M+H]⁺=441.1.

Compound 238: ¹H NMR (400 MHz, CD₃OD) δ 8.96 (d, J=1.6 Hz, 1H), 8.25 (d, J=3.2 Hz, 1H), 7.88 (d, J=8.4 Hz, 1H), 7.74 (d, J=8.8 Hz, 2H), 7.17 (d, J=8.8 Hz, 2H), 4.35 (t, J=5.2 Hz, 2H), 4.02 (s, 4H), 3.25 (t, J=5.2 Hz, 2H), 3.03 (s, 4H), 2.11 (t, J=5.6 Hz, 4H), 2.21-2.05 (m, 4H).

### Example 239: 7-[2-[4-[6-(Trifluoromethyl)-3-pyridyl]phenoxy]ethyl]-26-thia-7-azaspiro[3.5]nonane 2,2-dioxide

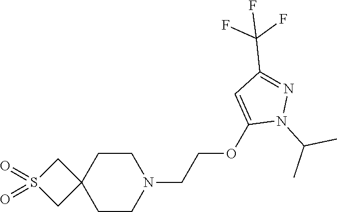 [SMILES: CC(C)n1nc(C(F)(F)F)cc1OCCN1CCC2(CC1)CS(=O)(=O)C2]

### Step 1: 2-Isopropyl-5-(trifluoromethyl)pyrazol-3-ol

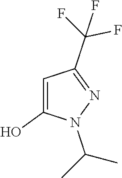

To a stirred solution of ethyl 4,4,4-trifluoroacetoacetate (1.0 g, 5.43 mmol) in ethanol (10 mL) was added isopropylhydrazine hydrochloride (0.6 g, 5.43 mmol) and HCl (1 mL, 1 mmol) and the reaction mixture was stirred at 60° C. for 2.5 h. The reaction mixture was concentrated in vacuo and the residue was diluted with water (25 mL). The resulting residue was extracted with ethyl acetate:MeOH (10:1, 200 mL x 3). The combined organic layers were washed by brine (30 mL), dried over anhydrous sodium sulfate, filtered, concentrated and the residue was purified by silica column chromatography (50% ethyl acetate in petroleum ether) to provide the title compound (0.50 g, 47.4% yield).

### Step 2: 5-(2-Bromoethoxy)-1-isopropyl-3-(trifluoromethyl)pyrazole

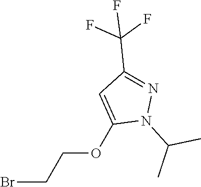 [SMILES: CC(C)n1nc(C(F)(F)F)cc1OCCBr]

To a solution of 2-isopropyl-5-(trifluoromethyl)pyrazol-3-ol (400 mg, 2.06 mmol) and potassium carbonate (711.87 mg, 5.15 mmol) in acetone (2 mL) were added 1,2-dibromoethane (0.54 mL, 6.18 mmol) at 20° C. Then the reaction mixture was stirred at 60° C. for 15 hours. The reaction mixture was diluted with water (10 mL), and extracted with ethyl acetate (10 mL×2). The combined organic layers were concentrated under reduced pressure, and the resulting residue was purified by silica column chromatography (0-20% ethyl acetate in petroleum ether) to afford the title compound (0.50 g, 81% yield).

### Step 3: Synthesis of the Title Compound

To a solution of 2-thia-7-azaspiro[3.5]nonane 2,2-dioxide hydrochloride (77.34 mg, 0.37 mmol) and 5-(2-bromoethoxy)-1-isopropyl-3-(trifluoromethyl)pyrazole (100.0 mg, 0.33 mmol) in acetonitrile (2.0 mL) was added N,N-diisopropylethylamine (0.14 mL, 0.83 mmol) and potassium iodide (5.51 mg, 0.03 mmol) at 80° C. and stirred for 15 h. The reaction mixture was concentrated and the resulting residue was purified by pre-HPLC (water (0.05% FA)—ACN, 40-70%) to afford the title compound (66.12 mg, 50% yield). LCMS (ESI) [M+H]⁺=396.1

Compound 239: ¹H NMR (400 MHz, CD₃OD) δ 6.03 (s, 1H), 4.66-4.60 (m, 1H), 4.31 (t, J=5.2 Hz, 2H), 3.93 (s, 4H), 2.94 (t, J=5.2 Hz, 2H), 2.68 (s, 4H), 1.97 (t, J=5.6 Hz, 4H), 1.42 (d, J=6.4 Hz, 6H).

### Example 240: 7-[2-[(5-Chloro-6-cyclohexyl-3-pyridyl)oxy]ethyl]-26-thia-7-azaspiro[3.5]nonane 2,2-dioxide

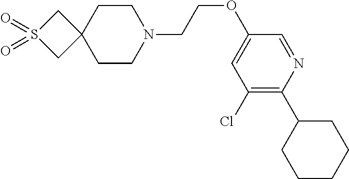 [SMILES: O=S1(=O)CC2(CCN(CCOc3cnc(C4CCCCC4)c(Cl)c3)CC2)C1]

### Step 1: 2-Bromo-5-(2-bromoethoxy)-3-chloro-pyridine

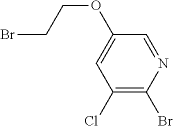 [SMILES: Clc1cc(OCCBr)cnc1Br]

To a solution of 1,2-dibromoethane (1351 mg, 7.2 mmol) in acetonitrile (10 mL) were added K₂CO₃ (828 mg, 6 mmol) and 6-bromo-5-chloropyridin-3-ol (500 mg, 2.4 mmol) at 20° C. Then the reaction mixture was heated to 60° C. and stirred for 15 h. The reaction solution was concentrated under reduced pressure and the resulting residue was purified by silica column chromatography (0-10% ethyl acetate in petroleum ether) to provide the title compound (629 mg, 1.95 mmol, 81.5% yield). LCMS (ESI) [M+H]⁺=315.9.

### Step 2: 7-[2-[(6-Bromo-5-chloro-3-pyridyl)oxy]ethyl]-26-thia-7-azaspiro[3.5]nonane 2,2-dioxide

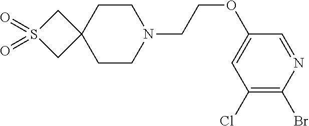 [SMILES: O=S1(=O)CC2(CCN(CCOc3cnc(Br)c(Cl)c3)CC2)C1]

To a solution of 2-bromo-5-(2-bromoethoxy)-3-chloro-pyridine (150 mg, 0.48 mmol) and 2-thia-7-azaspiro[3.5]nonane 2,2-dioxide hydrochloride (151 mg, 0.71 mmol) in acetonitrile (6 mL) was added N,N-diisopropylethylamine (0.39 mL, 2.38 mmol) and potassium iodide (7 mg, 0.05 mmol) at 20° C. The reaction mixture was stirred at 80° C. for 15 h. The reaction mixture was extracted with ethyl acetate (50 mL×2). The combined organic layers were concentrated in vacuo and the resulting residue was purified by prep-TLC (10% methanol in dichloromethane) to provide the title compound (161 mg, 0.39 mmol, 82.6% yield). LCMS (ESI) [M+H]⁺=411.0.

### Step 3: 7-[2-[[5-Chloro-6-(cyclohexen-1-yl)-3-pyridyl]oxy]ethyl]-26-thia-7-azaspiro[3.5]nonane 2,2-dioxide

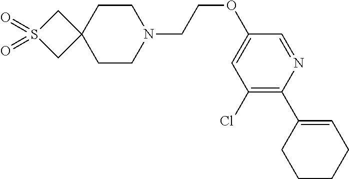 [SMILES: O=S1(=O)CC2(CCN(CCOc3cnc(C4=CCCCC4)c(Cl)c3)CC2)C1]

To a solution of 7-[2-[(6-bromo-5-chloro-3-pyridyl)oxy]ethyl]-26-thia-7-azaspiro[3.5]nonane 2,2-dioxide (60 mg, 0.15 mmol) in degased 1,4-dioxane (8 mL)) and water (1.6 mL) was added 2-(cyclohex-1-en-1-yl)-4,4,5,5-tetramethyl-1,3,2-dioxaborolane (22 mg, 0.11 mmol), K₂CO₃ (60 mg, 0.44 mmol) and Pd(dppf)Cl₂ (10 mg, 0.01 mmol). The reaction mixture was then stirred at 100° C. under N₂ for 1 h. The reaction mixture was extracted with ethyl acetate (50 mL×2). The combined organic layers were concentrated in vacuo and the residue was purified by prep-TLC (10% methanol in dichloromethane) to provide the title compound (40 mg, 0.09 mmol, 61.1% yield). LCMS (ESI) [M+H]⁺=411.1.

### Step 4: Synthesis of the Title Compound

To the solution of 7-[2-[[5-chloro-6-(cyclohexen-1-yl)-3-pyridyl]oxy]ethyl]-26-thia-7-azaspiro[3.5]nonane 2,2-dioxide (50 mg, 0.12 mmol) in tetrahydrofuran (4 mL) was added rhodium on carbon (33 mg, 0.02 mmol) under H₂. The mixture was stirred under H₂ (15 psi) at 20° C. for 5 h. The reaction mixture was filtered and the filtrate was concentrated under reduced pressure and the resulting residue was purified by prep-TLC (10% methanol in dichloromethane) to provide the title compound, 43.37 mg, 0.11 mmol, 92% yield). LCMS (ESI) [M+H]⁺=413.1.

Compound 240: ¹H NMR (400 MHz, CDCl₃) δ 8.18 (d, J=2.4 Hz, 1H), 7.21 (d, J=2.4 Hz, 1H), 4.11 (s, 2H), 3.87 (s, 4H), 3.15-3.07 (m, 1H), 2.79 (s, 2H), 2.59-2.42 (m, 3H), 1.95 (s, 3H), 1.89-1.72 (m, 6H), 1.54 (s, 2H), 1.48-1.26 (m, 4H).

### Example 241: 7-[2-[[6-[6-(Trifluoromethyl)-3-pyridyl]-3-pyridyl]oxy]ethyl]-26-thia-7-azaspiro[3.5]nonane 2,2-dioxide

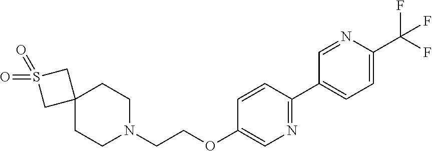 [SMILES: O=S1(=O)CC2(CCN(CCOc3ccc(-c4ccc(C(F)(F)F)nc4)nc3)CC2)C1]

### Step 1: 6-[6-(Trifluoromethyl)-3-pyridylpyridin-3-ol

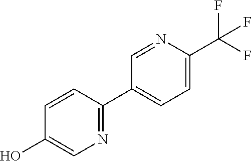 [SMILES: Oc1ccc(-c2ccc(C(F)(F)F)nc2)nc1]

To a mixture of 6-bromopyridin-3-ol (200 mg, 1.15 mmol), 2-trifluoromethyl-5-pyridine boric acid (329 mg, 1.72 mmol) and potassium carbonate (477 mg, 3.45 mmol) in 1,4-dioxane (4 mL) and water (1 mL) were added 1,1′-bis(diphenylphosphino)ferrocene palladium dichloride (84 mg, 0.11 mmol), then the reaction mixture was stirred under nitrogen atmosphere at 90° C. for 16 h. The reaction mixture was concentrated in vacuo, the residue was purified by silica flash column chromatography (0-50% ethyl acetate in petroleum ether) to afford the title compound (130 mg, 0.5064 mmol, 44.1% yield). LCMS (ESI) [M+H]⁺=241.0.

### Step 2: 5-(2-Bromoethoxy)-6′-(trifluoromethyl)-2,3′-bipyridine

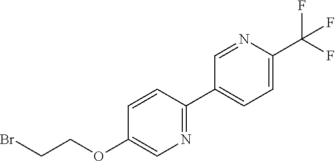 [SMILES: FC(F)(F)c1ccc(-c2ccc(OCCBr)cn2)cn1]

A mixture of 6-[6-(trifluoromethyl)-3-pyridyl]pyridin-3-ol (130 mg, 0.54 mmol), 1,2-dibromoethane (1.53 g, 8.12 mmol) and potassium carbonate (224 mg, 1.62 mmol) in acetonitrile (5 mL) was stirred at 80° C. for 15 h. The reaction mixture was concentrated in vacuo and the residue was purified by silica flash column chromatography (0-20% of ethyl acetate in petroleum ether) to afford the title compound (89 mg, 0.1822 mmol, 33.7% yield). LCMS (ESI) [M+H]⁺=346.9.

### Step 3: Synthesis of the Title Compound

A mixture of 2-thia-7-azaspiro[3.5]nonane 2,2-dioxide hydrochloride (40 mg, 0.19 mmol), 5-(2-bromoethoxy)-2-[6-(trifluoromethyl)-3-pyridyl]pyridine (79 mg, 0.23 mmol), N,N-diisopropylethylamine (0.16 mL, 0.94 mmol) and potassium iodide (31 mg, 0.19 mmol) in acetonitrile (3 mL) was stirred at 80° C. for 16 h. The reaction mixture was concentrated in vacuo, and the residue was purified with pre-TLC (10% methanol in dichloromethane) to afford the title compound, 43.1 mg, 0.0954 mmol, 42.4% yield). LCMS (ESI) [M+H]⁺=442.0.

Compound 241: ¹H NMR (400 MHz, CD₃OD) δ 9.28 (s, 1H), 8.57 (d, J=8.4 Hz, 1H), 8.44 (d, J=3.2 Hz, 1H), 8.00 (d, J=8.8 Hz, 1H), 7.90 (d, J=8.4 Hz, 1H), 4.29 (t, J=5.2 Hz, 2H), 3.93 (s, 4H), 2.87 (t, J=5.6 Hz, 2H), 2.61 (s, 4H), 1.95 (t, J=5.2 Hz, 4H).

### Example 242: 7-[2-[[5-Chloro-6-(trifluoromethyl)-3-pyridyl]oxy]ethyl]-26-thia-7-azaspiro[3.5]nonane 2,2-dioxide

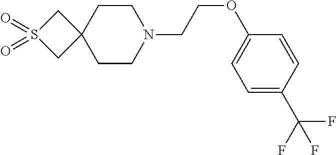 [SMILES: O=S1(=O)CC2(CCN(CCOc3ccc(C(F)(F)F)cc3)CC2)C1]

The title compound (35.99 mg, 0.08 mmol, 91% yield) was synthesized similarly to example 201 using 2-thia-7-azaspiro[3.5]nonane 2,2-dioxide and 4-(trifluoromethyl)phenol. LCMS (ESI) [M+H]⁺=399.0.

Compound 242: ¹H NMR (400 MHz, CD₃OD) δ 8.29 (d, J=2.4 Hz, 1H), 7.70 (d, J=2.0 Hz, 1H), 4.29 (t, J=5.2 Hz, 2H), 3.92 (s, 4H), 2.85 (t, J=5.2 Hz, 2H), 2.58 (s, 4H), 1.93 (t, J=5.2 Hz, 4H).

### Example 243: 7-[2-[[2-Methyl-6-(trifluoromethyl)-3-pyridyl]oxy]ethyl]-26-thia-7-azaspiro[3.5]nonane 2,2-dioxide

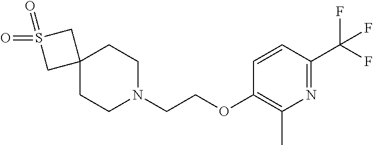 [SMILES: Cc1nc(C(F)(F)F)ccc1OCCN1CCC2(CC1)CS(=O)(=O)C2]

### Step 1: 2-Methyl-6-(trifluoromethyl)pyridin-3-ol

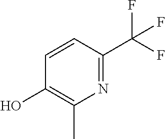

To a solution of 3-bromo-2-methyl-6-(trifluoromethyl)pyridine (200 mg, 0.83 mmol) in N,N-dimethylformamide (4 mL) was added cesium carbonate (814 mg, 2.5 mmol), benzaldoxime (151 mg, 1.25 mmol), [2-(2-aminophenyl)phenyl]-methylsulfonyloxy-palladium and ditert-butyl-[6-methoxy-3-methyl-2-(2,4,6-triisopropylphenyl)phenyl]phosphane (70 mg, 0.083 mmol). The reaction mixture was stirred at 80° C. for 16 h. The mixture was then diluted with ethyl acetate (40 mL), washed with brine (10 mL×3). The organic layer was concentrated in vacuo and the resulting residue was purified by silica flash column chromatography (0-50% ethyl acetate in petroleum ether) to give the title compound (100 mg, 68% yield). ¹H NMR (400 MHz, CD₃OD) δ 7.47 (d, J=8.4 Hz, 1H), 7.21 (d, J=8.4 Hz, 1H), 2.45 (s, 3H).

### Step 2: 3-(2-Bromoethoxy)-2-methyl-6-(trifluoromethyl)pyridine

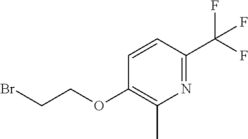

To a solution of 2-methyl-6-(trifluoromethyl)pyridin-3-ol (60.0 mg, 0.34 mmol) in acetonitrile (1 mL) was added potassium carbonate (117.05 mg, 0.85 mmol) and 1,2-dibromoethane (0.09 mL, 1.02 mmol) at 20° C. The reaction mixture was then stirred at 60° C. for 15 hours. The reaction mixture was diluted with water (10 mL), and extracted with ethyl acetate (10 mL×2). The combined organic layers were concentrated under reduced pressure, and the resulting residue was purified by silica column chromatography (0-20% ethyl acetate in petroleum ether) to afford the title compound (60 mg, 58% yield).

### Step 3: Synthesis of the Title Compound

To a solution of 3-(2-bromoethoxy)-2-methyl-6-(trifluoromethyl)pyridine (60.0 mg, 0.21 mmol) and 2-thia-7-azaspiro[3.5]nonane 2,2-dioxide hydrochloride (49 mg, 0.23 mmol) in acetonitrile (2 mL) were added N,N-diisopropylethylamine (0.09 mL, 0.53 mmol) and iodopotassium (3.51 mg, 0.02 mmol). The reaction mixture was stirred at 80° C. for 15 h and was concentrated in vacuo. The resulting residue was purified by pre-HPLC (water (0.05% FA)—ACN, 40%˜70%) to afford the title compound (60.6 mg, 73.5% yield). LCMS (ESI) [M+H]⁺=379.1.

Compound 243: ¹H NMR (400 MHz, CD₃OD) δ 8.27 (s, 1H), 7.63 (d, J=8.0 Hz, 1H), 7.48 (d, J=8.4 Hz, 1H), 4.46 (t, J=4.4 Hz, 2H), 4.04 (s, 4H), 3.47 (brs, 2H), 3.26-3.14 (m, 4H), 2.52 (s, 3H), 2.17 (t, J=5.2 Hz, 4H).

### Example 244: 7-(2-((6-Cyclohexyl-5-(trifluoromethyl)pyridin-3-yl)oxy)ethyl)-2-thia-7-azaspiro[3.5]nonane 2,2-dioxide

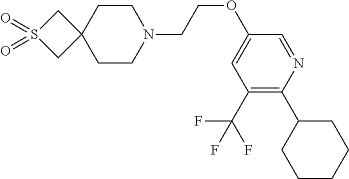 [SMILES: O=S1(=O)CC2(CCN(CCOc3cnc(C4CCCCC4)c(C(F)(F)F)c3)CC2)C1]

### Step 1: 2-Chloro-5-((4-methoxybenzyl)oxy)-3-(trifluoromethyl)pyridine

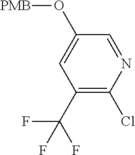

To a mixture of 5-bromo-2-chloro-3-(trifluoromethyl)pyridine (2 g, 7.68 mmol) and 1,10-phenanthroline (550 mg, 3.07 mmol) in toluene (40 mL) were added cesium carbonate (7.5 g, 23.04 mmol) and 4-methoxybenzyl alcohol (5.3 g, 38.4 mmol). The reaction mixture was then stirred under nitrogen atmosphere at 90° C. for 18 h. The reaction mixture was concentrated in vacuo. The residue was purified by silica flash column chromatography (0-50% ethyl acetate in petroleum ether) to give the title compound (920 mg, 2.896 mmol, 38% yield). LCMS (ESI) [M+H]⁺=318.0.

### Step 2: 2-(Cyclohex-1-en-1-yl)-5-((4-methoxybenzyl)oxy)-3-(trifluoromethyl)pyridine

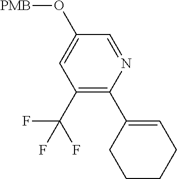

To a mixture of cyclohexene-1-boronic acid pinacolester (630 mg, 3.02 mmol) and potassium carbonate (1.05 g, 7.55 mmol) in 1,4-dioxane (20 mL) and water (4 mL) were added 1,1′-bis(diphenylphosphino)ferrocene palladium dichloride (185 mg, 0.25 mmol) and 2-chloro-5-[(4-methoxyphenyl)methoxy]-3-(trifluoromethyl)pyridine (800 mg, 2.52 mmol). The reaction mixture was then stirred under nitrogen atmosphere at 90° C. for 4 h. The reaction mixture was concentrated in vacuo and the resulting residue was purified by silica flash column chromatography (0-40% ethyl acetate in petroleum ether) to give the title compound (800 mg, 2.20 mmol, 87% yield). LCMS (ESI) [M+H]⁺=364.2.

### Step 3: 6-Cyclohexyl-5-(trifluoromethyl)pyridin-3-ol

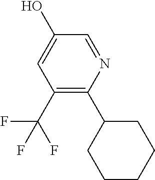 [SMILES: Oc1cnc(C2CCCCC2)c(C(F)(F)F)c1]

To a solution of 2-(cyclohexen-1-yl)-5-[(4-methoxyphenyl)methoxy]-3-(trifluoromethyl)pyridine (780 mg, 2.15 mmol) in ethanol (20 mL) was added 10% palladium in carbon (230 mg, 0.21 mmol). The reaction mixture was then stirred under H₂ (15 psi) at 25° C. for 4 h. The mixture was filtered and the filtrate was concentrated. The residue was purified by silica flash chromatography (0-50% ethyl acetate in petroleum ether) to afford the title compound (245 mg, 1.0 mmol, 47% yield). LCMS (ESI) [M+H]⁺=246.2.

### Step 4: 5-(2-Bromoethoxy)-2-cyclohexyl-3-(trifluoromethyl)pyridine

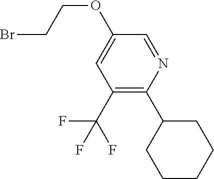 [SMILES: FC(F)(F)c1cc(OCCBr)cnc1C1CCCCC1]

2-[4-(Trifluoromethyl)cyclohexyl]pyrimidin-5-ol (245 mg, 1 mmol), cesium carbonate (970 mg, 3.0 mmol) and 1,2-dibromoethane (1.30 g, 6.97 mmol) were dissolved in acetonitrile (5 mL) the reaction mixture was stirred at 80° C. for 3 h, then cooled to 25° C. and quenched with brine (20 mL). The resulting mixture was extracted with ethyl acetate (20 mL×3). The combined organic layer was dried over anhydrous Na₂SO₄, filtered and concentrated in vacuo. The residue was purified by silica gel column chromatography (0-30% of ethyl acetate in petroleum ether) to afford the title compound (240 mg, 0.671 mmol, 67% yield). LCMS (ESI) [M+H]⁺=352.1.

### Step 5: Synthesis of the Title Compound

To a solution of 2-thia-7-azaspiro[3.5]nonane 2,2-dioxide hydrochloride (50 mg, 0.24 mmol) and N,N-diisopropylethylamine (0.2 mL, 1.07 mmol) in acetonitrile (2 mL) were added 5-(2-bromoethoxy)-2-cyclohexyl-3-(trifluoromethyl)pyridine (92 mg, 0.26 mmol) and iodopotassium (35 mg, 0.21 mmol) at 20° C. The reaction mixture was then stirred at 80° C. for 16 h. The reaction mixture was purified by pre-HPLC to afford the title compound (48.4 mg, 0.106 mmol, 45% yield). LCMS (ESI) [M+H]⁺=447.1.

Compound 244: ¹H NMR (400 MHz, CD₃OD) δ 8.42 (d, J=2.4 Hz, 1H), 7.57 (d, J=2.8 Hz, 1H), 4.24 (t, J=5.2 Hz, 2H), 3.91 (s, 4H), 3.01-2.91 (m, 1H), 2.84 (t, J=5.2 Hz, 2H), 2.58 (br s, 4H), 1.93 (t, J=5.6 Hz, 4H), 1.90-1.81 (m, 2H), 1.78-1.63 (m, 5H), 1.47-1.31 (m, 3H).

### Examples 245A* and 245B*:7-(2-((2-((cis)-4-(Trifluoromethyl)cyclohexyl)pyridin-4-yl)oxy)ethyl)-2-thia-7-azaspiro[3.5]nonane 2,2-dioxide and 7-(2-((2-((trans)-4-(trifluoromethyl)cyclohexyl)pyridin-4-yl)oxy)ethyl)-2-thia-7-azaspiro[3.5]nonane 2,2-dioxide

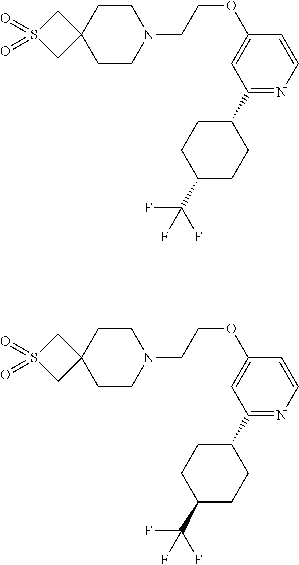 [SMILES: O=S1(=O)CC2(CCN(CCOc3ccnc([C@H]4CC[C@@H](C(F)(F)F)CC4)c3)CC2)C1 ; O=S1(=O)CC2(CCN(CCOc3ccnc([C@H]4CC[C@H](C(F)(F)F)CC4)c3)CC2)C1]

These compounds were synthesized similarly to example 241.

7-(2-((2-(4-(trifluoromethyl)cyclohexyl)pyridin-4-yl)oxy)ethyl)-2-thia-7-azaspiro[3.5]nonane 2,2-dioxide (150 mg, 0.34 mmol) was purified by chiral SFC (Daicel Chiralpak AD (250 mm*30 mm, 10 μm); 0.1% NH₃H₂O, EtOH, 30%) to provide Compound 245A* (the first peak on SFC, 65.7 mg, 0.14 mmol, 41.6% yield) and Compound 245B* (the second peak on SFC, 22.3 mg, 0.05 mmol, 14.6% yield), each of unassigned stereochemistry. LCMS (ESI) [M+H]⁺=447.1.

Compound 245*: ¹H NMR (400 MHz, CD₃OD) δ 8.28 (d, J=6.0 Hz, 1H), 6.89 (d, J=2.4 Hz, 1H), 6.84 (dd, J=2.4, 5.6 Hz, 1H), 4.22 (t, J=5.6 Hz, 2H), 3.92 (s, 4H), 2.91-2.82 (m, 3H), 2.58 (s, 4H), 2.43-2.33 (m, 1H), 2.11-2.01 (m, 2H), 1.94 (t, J=5.6 Hz, 4H), 1.86-1.76 (m, 6H).

Compound 245B*: ¹H NMR (400 MHz, CD₃OD) δ 8.25 (d, J=5.6 Hz, 1H), 6.88 (d, J=2.4 Hz, 1H), 6.84 (dd, J=2.4, 6.0 Hz, 1H), 4.22 (t, J=5.2 Hz, 2H), 3.92 (s, 4H), 2.83 (t, J=5.6 Hz, 2H), 2.70-2.58 (m, 5H), 2.50-2.19 (m, 1H), 2.09-2.00 (m, 4H), 1.93 (t, J=5.6 Hz, 4H), 1.68-1.58 (m, 2H), 1.55-1.44 (m, 2H).

### Example 246: 7-(2-((5-Chloro-6-((trans)-4-(trifluoromethyl)cyclohexyl)pyridin-3-yl)oxy)ethyl)-2-thia-7-azaspiro[3.5]nonane 2,2-dioxide

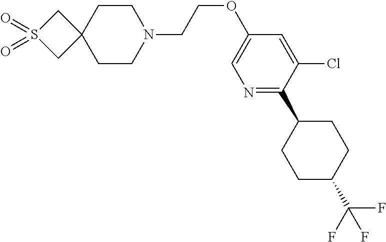 [SMILES: O=S1(=O)CC2(CCN(CCOc3cnc([C@H]4CC[C@H](C(F)(F)F)CC4)c(Cl)c3)CC2)C1]

### The Trans Stereochemistry was Assigned Based on 2D NMR Analysis

### Step 1: 5-Chloro-6-(4-(trifluoromethyl)cyclohex-1-en-1-yl)pyridin-3-ol

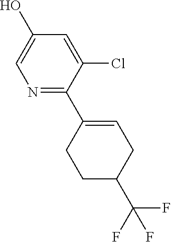 [SMILES: Oc1cnc(C2=CCC(C(F)(F)F)CC2)c(Cl)c1]

A solution of K₂CO₃ (60 mg, 4.32 mmol), 1,1′-bis(diphenylphosphino)ferrocene palladium dichloride (53 mg, 0.07 mmol), 2-bromo-3-chloro-5-hydroxypyridine (300 mg, 1.44 mmol), 4,4,5,5-tetramethyl-2-[4-(trifluoromethyl)-1-cyclohexen-1-yl]-1,3,2-dioxaborolane (1192 mg, 4.32 mmol) in 1,4-dioxane (10 mL) and water (2.0 mL) was stirred at 90° C. under N₂ for 16 h. The reaction mixture was concentrated in vacuo and the residue was purified by silica flash chromatography (0-10% methyl alcohol in dichloromethane) to afford the title compound (200 mg, 0.7203 mmol, 50% yield). LCMS (ESI) [M+H]⁺=278.0

### Step 2: 5-Chloro-6-(4-(trifluoromethyl)cyclohexyl)pyridin-3-ol

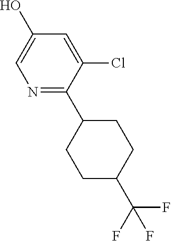 [SMILES: Oc1cnc(C2CCC(C(F)(F)F)CC2)c(Cl)c1]

To a solution of 5-chloro-6-[4-(trifluoromethyl)cyclohexen-1-yl]pyridin-3-ol (200.0 mg, 0.72 mmol) in THF (2 mL) was added rhodium/carbon (247 mg, 0.14 mmol). The reaction mixture was stirred under H₂ (15 psi) at 20° C. for 16 h. The reaction mixture was filtered and concentrated in vacuo. The residue was purified by silica flash chromatography (0-5% ethyl acetate in petroleum ether) to afford the title compound (150 mg, 0.5363 mmol, 75% yield). LCMS (ESI) [M+H]⁺=280.1.

### Step 3: 5-(2-Bromoethoxy)-3-chloro-2-(4-(trifluoromethyl)cyclohexyl)pyridin

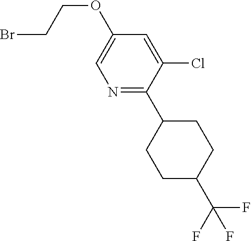 [SMILES: FC(F)(F)C1CCC(c2ncc(OCCBr)cc2Cl)CC1]

To a solution of 5-chloro-6-[4-(trifluoromethyl)cyclohexyl]pyridin-3-ol (80 mg, 0.29 mmol) in acetonitrile (10 mL) were added potassium carbonate (119 mg, 0.86 mmol) and 1,2-dibromoethane (806 mg, 4.29 mmol) at 20° C. The reaction mixture was then heated to 80° C. and stirred for 1 h. The reaction mixture was diluted with water (15 mL) and extracted with ethyl acetate (30 mL×3). The combined organics were washed with brine (20 mL×2), dried over anhydrous sodium sulfate, filtered and concentrated in vacuo. The residue was purified by silica flash chromatography (0-10% ethyl acetate in petroleum ether) to afford the title compound (100 mg, 0.246 mmol, 86% yield). LCMS (ESI) [M+H]⁺=387.4.

### Step 4: 7-(2-((5-Chloro-6-((trans)-4-(Trifluoromethyl)cyclohexyl)pyridin-3-yl)oxy)ethyl)-2-thia-7-azaspiro[3.5]nonane 2,2-dioxide

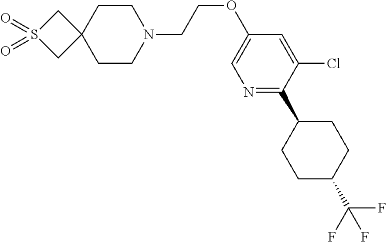 [SMILES: O=S1(=O)CC2(CCN(CCOc3cnc([C@H]4CC[C@H](C(F)(F)F)CC4)c(Cl)c3)CC2)C1]

The title compound was synthesized similarly to the last synthetic example 241 using 2-thia-7-azaspiro[3.5]nonane 2,2-dioxide. The mixture of cis/trans isomers was purified by pre-TLC (50% (ethanol/ethyl acetate=3:1) in petroleum ether) to afford the title Compound 246 (the trans isomer, stereochemistry assigned by NMR) (39.17 mg, 0.0782 mmol, 33.1% yield). LCMS (ESI) [M+H]⁺=481.2. Compound 246: ¹H NMR (400 MHz, CD₃OD) δ 8.17 (s, 1H), 7.46 (s, 1H), 4.19 (t, J=5.2 Hz, 2H), 3.92 (s, 4H), 3.22-3.11 (m, 1H), 2.84 (t, J=5.2 Hz, 2H), 2.60 (brs, 4H), 2.27-2.13 (m, 1H), 2.07-2.04 (m, 2H), 1.97-1.88 (m, 6H), 1.73-1.63 (m, 2H), 1.51-1.46 (m, 2H).

### Examples 247A* and 247B*: 7-(2-((6-((trans)-4-(Trifluoromethyl)cyclohexyl)pyridin-3-yl)oxy)ethyl)-2-thia-7-azaspiro[3.5]nonane 2,2-dioxide and 7-(2-((6-((cis)-4-(trifluoromethyl)cyclohexyl)pyridin-3-yl)oxy)ethyl)-2-thia-7-azaspiro[3.5]nonane 2,2-dioxide

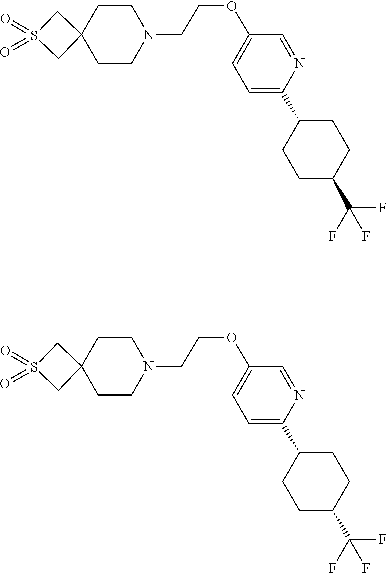 [SMILES: O=S1(=O)CC2(CCN(CCOc3ccc([C@H]4CC[C@H](C(F)(F)F)CC4)nc3)CC2)C1 ; O=S1(=O)CC2(CCN(CCOc3ccc([C@H]4CC[C@@H](C(F)(F)F)CC4)nc3)CC2)C1]

### Step 1: 6-(4-(Trifluoromethyl)cyclohex-1-en-1-yl)pyridin-3-ol

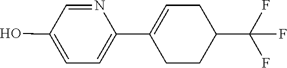 [SMILES: Oc1ccc(C2=CCC(C(F)(F)F)CC2)nc1]

To a solution of 6-bromopyridin-3-ol (700.0 mg, 4.02 mmol) and 4,4,5,5-tetramethyl-2-(4-(trifluoromethyl)cyclohex-1-en-1-yl)-1,3,2-dioxaborolane (1.11 g, 4.02 mmol) in 1,4-dioxane (20 mL) and water (4 mL) were added 1,1′-bis(diphenylphosphino)ferrocene palladium dichloride (294 mg, 0.40 mmol) and K₂CO₃ (1.67 g, 12.07 mmol). The reaction mixture was stirred at 90° C. under N₂ for 16. The reaction mixture was concentrated in vacuo, and the residue was purified by silica flash column chromatography (0-3% methanol in dichloromethane) to give the title compound (900 mg, 3.70 mmol, 80.5% yield). LCMS (ESI) [M+H]⁺=243.9

### Step 2: 6-(4-(Trifluoromethyl)cyclohexyl)pyridin-3-ol

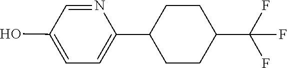 [SMILES: Oc1ccc(C2CCC(C(F)(F)F)CC2)nc1]

To a solution of 6-(4-(trifluoromethyl)cyclohex-1-en-1-yl)pyridin-3-ol (900.0 mg, 3.7 mmol) in ethanol (20 mL) was added Pd/C (393.79 mg, 0.37 mmol). The reaction mixture was stirred at 25° C. for 2 h under H₂ (15 psi). The reaction was filtered and the filtrate was concentrated in vacuo to afford the title compound (800 mg, 3.26 mmol, 88.2% yield). LCMS (ESI) [M+H]⁺=245.7.

### Step 3: 5-(2-Bromoethoxy)-2-(4-(trifluoromethyl)cyclohexyl)pyridine

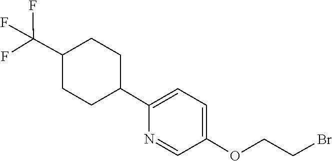 [SMILES: FC(F)(F)C1CCC(c2ccc(OCCBr)cn2)CC1]

To a solution of 6-(4-(trifluoromethyl)cyclohexyl)pyridin-3-ol (800.0 mg, 3.26 mmol) in N,N-dimethylformamide (10 mL) was added 1,2-dibromoethane (9.19 g, 48.93 mmol) and K₂CO₃ (1.35 g, 9.79 mmol) at 20° C. The reaction mixture was then heated to 80° C. and stirred for 16 h. The reaction was diluted with water (10 mL) and extracted with ethyl acetate (20 mL×3). The combined organics were washed with brine (20 mL×4), dried over anhydrous sodium sulfate, filtered and concentrated in vacuo. The residue was purified by silica flash column chromatography (0-30% ethyl acetate in petroleum ether) to give the title compound (330 mg, 0.94 mmol, 28.7% yield). LCMS (ESI) [M+H]⁺=353.6.

### Step 4: 7-(2-((6-(4-(Trifluoromethyl)cyclohexyl)pyridin-3-yl)oxy)ethyl)-2-thia-7-azaspiro[3.5]nonane 2,2-dioxide

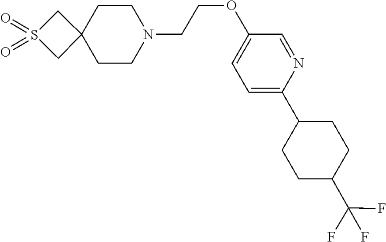 [SMILES: O=S1(=O)CC2(CCN(CCOc3ccc(C4CCC(C(F)(F)F)CC4)nc3)CC2)C1]

To a solution of 5-(2-bromoethoxy)-2-(4-(trifluoromethyl)cyclohexyl)pyridine (330.0 mg, 0.94 mmol) and 2-thia-7-azaspiro[3.5]nonane 2,2-dioxide hydrochloride (218.21 mg, 1.03 mmol) in acetonitrile (5 mL) were added N,N-diisopropylethylamine (605 mg, 4.68 mmol) and KI (16 mg, 0.09 mmol) at 20° C. The mixture was stirred at 80° C. for 16 h. The reaction was diluted with water (5 mL) and extracted with ethyl acetate (20 mL×3). The organics were dried over anhydrous sodium sulfate, filtered and concentrated in vacuo). The residue was purified by silica flash chromatography (solvent gradient: 0-100% (33% ethyl alcohol in ethyl acetate) in petroleum ether) to afford the title compound (220 mg, 0.49 mmol, 52.6% yield). LCMS (ESI) [M+H]⁺=447.1. The mixture of isomers (220.0 mg, 0.49 mmol) were purified by chiral SFC (Daicel Chiralpak AD (250 mm*30 mm, 10 um); 0.1% NH₃H₂O; MeOH, 50%; 70 mL/min) to provide the compound 247A (the first peak on SFC, Compound 247A, 33.4 mg, 0.072 mmol, 14.6% yield) and compound 247B (the second peak on SFC, Compound 247B, 50 mg, 0.11 mmol, 22.3% yield), each of undefined/unassigned stereochemistry, LCMS (ESI) [M+H]⁺=447.2.

Compound 247A*: ¹H NMR (400 MHz, CD₃OD) δ 8.14 (d, J=3.2 Hz, 1H), 7.38 (dd, J=3.2, 8.8 Hz, 1H), 7.25 (d, J=8.4 Hz, 1H), 4.18 (t, J=5.6 Hz, 2H), 3.91 (s, 4H), 2.82 (t, J=5.6 Hz, 2H), 2.70-2.58 (m, 5H), 2.23-2.17 (m, 1H), 2.07 (d, J=11.6 Hz, 2H), 2.00 (d, J=8.8 Hz, 2H), 1.93 (t, J=5.6 Hz, 4H), 1.64-1.55 (m, 2H), 1.53-1.47 (m, 2H).

Compound 247B*: ¹H NMR (400 MHz, CD₃OD) δ 8.18 (d, J=2.4 Hz, 1H), 7.40-7.36 (m, 1H), 7.30-7.28 (m, 1H), 4.19 (t, J=5.2 Hz, 2H), 3.92 (s, 4H), 2.93-2.89 (m, 1H), 2.83 (t, J=5.6 Hz, 2H), 2.59 (s, 4H), 2.88-2.33 (m, 1H), 2.09-2.04 (m, 2H), 1.94 (t, J=5.6 Hz, 4H), 1.85-1.76 (m, 6H).

### Examples 248A and 248B: 7-(2-((2-((trans)-3-(Trifluoromethyl)cyclohexyl)pyrimidin-5-yl)oxy)ethyl)-2-thia-7-azaspiro[3.5]nonane 2,2-dioxide (Compound 248A) and 7-(2-((2-((cis)-3-(trifluoromethyl)cyclohexyl)pyrimidin-5-yl)oxy)ethyl)-2-thia-7-azaspiro[3.5]nonane 2,2-dioxide (Compound 248B)

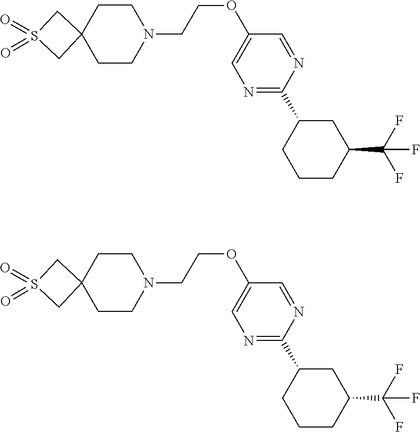 [SMILES: O=S1(=O)CC2(CCN(CCOc3cnc([C@H]4CCC[C@H](C(F)(F)F)C4)nc3)CC2)C1 ; O=S1(=O)CC2(CCN(CCOc3cnc([C@H]4CCC[C@@H](C(F)(F)F)C4)nc3)CC2)C1]

### Relative Stereochemistry Assigned Based on 2D NMR Analysis

### Step 1: 1-Iodo-3-(trifluoromethyl)cyclohexane

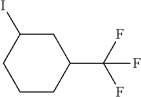 [SMILES: FC(F)(F)C1CCCC(I)C1]

To a stirring solution of 3-(trifluoromethyl)cyclohexanol (2 g, 11.89 mmol) in tetrahydrofuran (50 mL) at 0° C. were added imidazole (1.62 g, 23.79 mmol) and triphenylphosphine (6.24 g, 23.79 mmol) under N₂, and then iodine (6.0 g, 23.79 mmol) in tetrahydrofuran (10 mL) was added in dropwise. The reaction mixture was stirred at 25° C. for 16 hours. The reaction was quenched by Na₂S₂O₃ solution 30 mL and water 50 mL. The resulting solution was extracted with dichloromethane (50 mL*2). The combined organic layers were dried over anhydrous sodium sulfate, filtered and concentrated under vacuum. The residue was purified by silica flash chromatography (0-5% ethyl acetate in petroleum ether) to afford the title compound (1.85 g, 6.65 mmol, 56% yield). ¹H NMR (400 MHz, CDCl₃) δ 5.04-4.66 (m, 1H), 2.74-2.54 (m, 1H), 2.32-2.14 (m, 1H), 2.14-2.02 (m, 1H), 1.99-1.82 (m, 2H), 1.79-1.57 (m, 2H), 1.55-1.35 (m, 2H).

### Step 2: 2-(3-(Trifluoromethyl)cyclohexyl)pyrimidin-5-ol

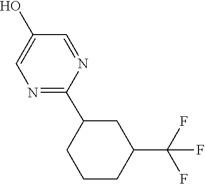 [SMILES: Oc1cnc(C2CCCC(C(F)(F)F)C2)nc1]

To a mixture of 2-bromopyrimidin-5-ol (800 mg, 4.57 mmol) and iodopotassium (760 mg, 4.57 mmol) in N,N-dimethylacetamide (30 mL) was added dibromonickel, 1,2-dimethoxyethane (140 mg, 0.46 mmol), 4-ethylpyridine (0.25 mL, 2.29 mmol), 1-iodo-3-(trifluoromethyl)cyclohexane (1270 mg, 4.57 mmol), 4,4′-di-tert-butyl-2,2′-dipyridyl (135 mg, 0.50 mmol) and manganese (500 mg, 9.14 mmol) in glovebox. The mixture was stirred at 80° C. for 16 hours. The mixture was diluted with ethyl acetate (50 mL) and the resulting mixture was washed with 1M HCl (20 mL*3), brine (20 mL*2). The organics were dried over Na₂SO₄, filtered and concentrated under reduced pressure to give a residue. The residue was purified by silica gel column chromatography (0-50% ethyl acetate in petroleum ether) to afford the title compound (160 mg, 0.6498 mmol, 14.2% yield). LCMS (ESI) [M+H]⁺=247.1.

### Step 3: 5-(2-Bromoethoxy)-2-(3-(trifluoromethyl)cyclohexyl)pyrimidine

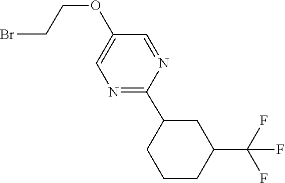 [SMILES: FC(F)(F)C1CCCC(c2ncc(OCCBr)cn2)C1]

2-[3-(Trifluoromethyl)cyclohexyl]pyrimidin-5-ol (160 mg, 0.65 mmol), cesium carbonate (635 mg, 1.95 mmol), 1,2-dibromoethane (122 mg, 0.65 mmol) were dissolved in acetonitrile (4 mL) and the mixture was stirred at 80° C. for 3 hours. Then the reaction was cooled to 25° C. and quenched with brine (20 mL). The mixture was extracted with ethyl acetate (20 mL×3). The organic layer was dried over Na₂SO₄, filtered and concentrated in vacuum. The residue was purified by silica gel column chromatography (0-30% of ethyl acetate in petroleum ether) to afford the title compound (155 mg, 0.4389 mmol, 68% yield). LCMS (ESI) [M+H]⁺=355.0

### Step 4: 7-(2-((2-(3-(Trifluoromethyl)cyclohexyl)pyrimidin-5-yl)oxy)ethyl)-2-thia-7-azaspiro[3.5]nonane 2,2-dioxide

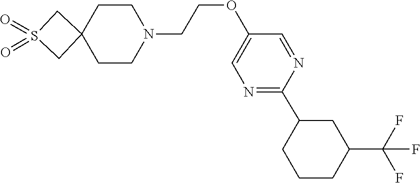 [SMILES: O=S1(=O)CC2(CCN(CCOc3cnc(C4CCCC(C(F)(F)F)C4)nc3)CC2)C1]

2-Thia-7-azaspiro[3.5]nonane 2,2-dioxide hydrochloride (80 mg, 0.38 mmol), 5-(2-bromoethoxy)-2-[3-(trifluoromethyl)cyclohexyl]pyrimidine (147 mg, 0.42 mmol) and N,N-diisopropylethylamine (0.3 mL, 1.72 mmol) and iodo potassium (57 mg, 0.34 mmol) were dissolved in acetonitrile (5 mL) at 20° C. and the mixture was stirred at 80° C. for 16 h. The crude mixture was purified by pre-HPLC (Welch Xtimate C18 100*25 mm*3 μm) to afford the title compound (49 mg, 0.106 mmol). LCMS (ESI) [M+H]⁺=448.0

### Step 6—Synthesis of the Title Compounds

7-[2-[2-[3-(Trifluoromethyl)cyclohexyl]pyrimidin-5-yl]oxyethyl]-26-thia-7-azaspiro[3.5]nonane 2,2-dioxide (80 mg, 0.18 mmol) was separated using chiral SFC (Daicel Chiralpak AD (250 mm*30 mm, 10 μm)); 0.1% NH₃H₂O; EtOH=30:70; 60 mL/min) to afford the Compound 248A as the trans isomer (the first peak on SFC, 33.44 mg, 0.0725 mmol, 40.5% yield) and Compound 248B as the cis isomer (the second peak on SFC, 32.73 mg, 0.0709 mmol, 39.7% yield). LCMS (ESI) [M+H]⁺=448.0. The relative cis-trans stereochemistry was assigned based on 2D NMR analysis.

Compound 248A: ¹H NMR (400 MHz, CD₃OD) δ 8.45 (s, 2H), 4.26 (t, J=5.2 Hz, 2H), 3.91 (s, 4H), 3.35 (brs, 1H), 2.91 (brs, 1H), 2.83 (t, J=5.6 Hz, 2H), 2.58 (brs, 3H), 2.33 (d, J=9.2 Hz, 1H), 2.15 (d, J=13.2 Hz, 1H), 2.00 (d, J=8.0 Hz, 3H), 1.93 (t, J=5.6 Hz, 4H), 1.69-1.63 (m, 1H), 1.58-1.46 (m, 2H), 1.36-1.27 (m, 2H).

Compound 248B: ¹H NMR (400 MHz, CD₃OD) δ 8.45 (s, 2H), 4.26 (t, J=5.2 Hz, 2H), 3.91 (s, 4H), 3.35 (s, 1H), 2.89 (d, J=12.2 Hz, 1H), 2.83 (t, J=5.2 Hz, 2H), 2.58 (brs, 3H), 2.33 (d, J=8.4 Hz, 1H), 2.15 (d, J=13.2 Hz, 1H), 2.00 (d, J=7.6 Hz, 3H), 1.93 (t, J=5.2 Hz, 4H), 1.69-1.63 (m, 1H), 1.57-1.45 (m, 2H), 1.37-1.32 (m, 1H).

### Example 249: 7-(2-((5-Fluoro-6-((trans)-4-(trifluoromethyl)cyclohexyl)pyridin-3-yl)oxy)ethyl)-2-thia-7-azaspiro[3.5]nonane 2,2-dioxide

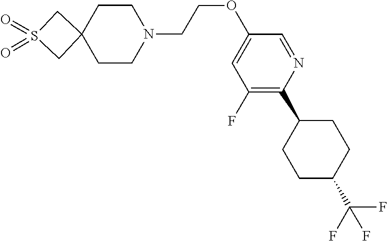 [SMILES: O=S1(=O)CC2(CCN(CCOc3cnc([C@H]4CC[C@H](C(F)(F)F)CC4)c(F)c3)CC2)C1]

### The Trans Stereochemistry was Assigned Based on 2D NMR Analysis

### Step 1: 5-(2-Bromoethoxy)-2-chloro-3-fluoropyridine

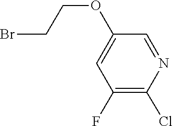 [SMILES: Fc1cc(OCCBr)cnc1Cl]

To a solution of 6-chloro-5-fluoropyridin-3-ol (500.0 mg, 3.39 mmol) in acetonitrile (5 mL) was added 1,2-dibromoethane (15 mL, 169.46 mmol)) and K₂CO₃ (1.41 g, 10.17 mmol) at 20° C. Then the reaction mixture was stirred at 60° C. for 8 h. The reaction was diluted with water (10 mL) and extracted with ethyl acetate (20 mL×3). The combined organics were washed with brine (20 mL×4), dried over sodium sulfate, filtered and concentrated. The residue was purified by flash column chromatography on silica (0-10% ethyl acetate in petroleum ether) to give the title compound (700 mg, 2.75 mmol, 81.2% yield). LCMS (ESI) [M+H]⁺=255.9.

### Step 2: 7-(2-((6-Chloro-5-fluoropyridin-3-yl)oxy)ethyl)-2-thia-7-azaspiro[3.5]nonane 2,2-dioxide

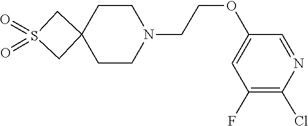 [SMILES: O=S1(=O)CC2(CCN(CCOc3cnc(Cl)c(F)c3)CC2)C1]

To a solution of 5-(2-bromoethoxy)-2-chloro-3-fluoropyridine (500.0 mg, 1.96 mmol) and 2-thia-7-azaspiro[3.5]nonane 2,2-dioxide (516.49 mg, 2.95 mmol) in acetonitrile (10 mL) was added N,N-diisopropylethylamine (1.27 g, 9.82 mmol) and potassium iodide (326.16 mg, 1.96 mmol) at 20° C. The mixture was stirred at 80° C. for 16 hours. The reaction was diluted with water (10 mL) and extracted with ethyl acetate (20 mL×3). The organics were dried over anhydrous sodium sulfate, filtered and concentrated. The residue was purified by flash chromatography on silica (0-100% ethyl acetate in petroleum ether) to afford the title compound (550 mg, 1.58 mmol, 80.2% yield). LCMS (ESI) [M+H]⁺=349.5.

### Step 3: 7-(2-((5-Fluoro-6-(4-(trifluoromethyl)cyclohex-1-en-1-yl)pyridin-3-yl)oxy)ethyl)-2-thia-7-azaspiro[3.5]nonane 2,2-dioxide

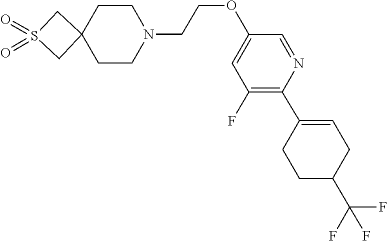 [SMILES: O=S1(=O)CC2(CCN(CCOc3cnc(C4=CCC(C(F)(F)F)CC4)c(F)c3)CC2)C1]

To a solution of 7-(2-((6-chloro-5-fluoropyridin-3-yl)oxy)ethyl)-2-thia-7-azaspiro[3.5]nonane 2,2-dioxide (300.0 mg, 0.86 mmol) and 4,4,5,5-tetramethyl-2-(4-(trifluoromethyl)cyclohex-1-en-1-yl)-1,3,2-dioxaborolane (285 mg, 1.03 mmol) in 1,4-dioxane (10 mL) and water (2 mL) were added 1,1′-bis(diphenylphosphino)ferrocene palladium dichloride (63 mg, 0.09 mmol) and K₂CO₃ (357 mg, 2.58 mmol). Then the reaction mixture was stirred under N₂ atmosphere at 90° C. for 16 hours. The reaction mixture was concentrated in vacuo, and the residue was purified by flash column chromatography on silica (0-10% methanol in dichloromethane) to give the title compound (300 mg, 0.65 mmol, 75.4% yield). LCMS (ESI) [M+H]⁺=463.1.

### Step 4—Synthesis of the Title Compound

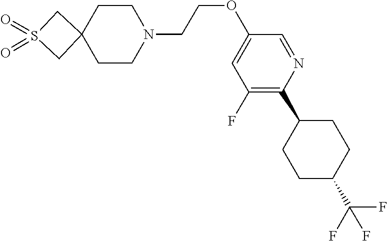 [SMILES: O=S1(=O)CC2(CCN(CCOc3cnc([C@H]4CC[C@H](C(F)(F)F)CC4)c(F)c3)CC2)C1]

To a solution of 7-(2-((5-fluoro-6-(4-(trifluoromethyl)cyclohex-1-en-1-yl)pyridin-3-yl)oxy)ethyl)-2-thia-7-azaspiro[3.5]nonane 2,2-dioxide (300.0 mg, 0.65 mmol) in ethanol (10 mL) was added Pd/C (138 mg, 0.13 mmol). The mixture was stirred under H₂ (15 psi) at 25° C. for 16 h. The reaction mixture was filtered and the filtrate was concentrated under reduced pressure to afford a mixture of cis/trans isomers, which was purified by chiral SFC (Daicel Chiralpak AD (250 mm*30 mm, 10 μm); 0.1% NH₃H₂O; EtOH; 40%) to provide the title compound as the trans isomer (the first peak on SFC, stereochemistry assigned by NMR) (15.5 mg, 0.033 mmol, 6.1% yield). LCMS (ESI) [M+H]⁺=465.2; ¹H NMR (400 MHz, CDCl₃) δ 8.08 (d, J=2.4 Hz, 1H), 6.93 (d, J=10.8 Hz, 1H), 4.12-3.95 (m, 2H), 3.88 (s, 4H), 2.97 (t, J=12.0 Hz, 1H), 2.89-2.26 (m, 6H), 2.16-2.12 (m, 1H), 2.08 (d, J=12.8 Hz, 2H), 1.94 (d, J=10.0 Hz, 5H), 1.75-1.65 (m, 3H), 1.51-1.44 (m, 2H).

### Biological Assay Examples

### 1. Mouse OPC Preparation

To assess effects of treatments on OPCs, all treatments were assayed in two or more independent platings of epiblast stem cell-derived OPCs (EpiSC). EpiSC-derived OPCs were obtained using in vitro differentiation protocols and culture conditions described previously (Najm et al, 2011, Nature Methods). OPCs were expanded and frozen down in aliquots. OPCs were thawed into growth conditions for at least one passage before use in further assays.

### 2. Determination of EC₅₀ Values of Compounds

### A: In Vitro Phenotypic Screening of OPCs

EpiSC-derived OPCs were grown and expanded in poly-L-ornithine (PO) and laminin-coated flasks in N2B27 media (DMEM/F12 (Gibco), N2-MAX (R&D Systems), B-27 (ThermoFisher), and GlutaMax (Gibco)) supplemented with FGF2 (10 pg/mL, R&D systems, 233-FB-025) and PDGF-AA (10 pg/mL, R&D systems, 233-AA-050) before harvesting for experiments. The cells were seeded onto poly-L-ornithine or poly-D-lysine coated CellCarrier Ultra plates (PerkinElmer) coated with laminin (Sigma, L2020) at a density of 150,000/cm² in N2B27 media without growth factors. For dose-response testing, a 1000× compound stock in dimethyl sulphoxide (DMSO) was added to assay plates, resulting in 8-point dose curves with final concentrations between 1000 nM and 0.5 nM. Positive controls and DMSO vehicle controls were included in each assay plate. Cells were incubated under standard conditions (37° C., 5% CO₂) for 3 days and fixed with 4% paraformaldehyde (PFA) in phosphate buffered saline (PBS) for 20 min. Fixed plates were washed with PBS, permeabilized with 0.1% Triton X-100, and blocked with 10% donkey serum (v/v) in PBS for 40 min. Then, cells were labelled with MBP antibodies (Abcam, ab7349; 1:200) for 2 h at room temperature, washed with PBS, and stained with Alexa Fluor conjugated secondary antibodies (1:500) for 45 min. Nuclei were visualized by DAPI staining (Sigma; 1 μg/ml), followed by further PBS washes.

### B: High-Content Imaging and Analysis

Cells and cell culture plates were imaged on the Operetta High Content Imaging and Analysis system (PerkinElmer). Analysis (PerkinElmer Harmony and Columbus software) began by identifying intact nuclei stained by DAPI. The peri-nuclear region of each cell was then cross-referenced with the mature myelin protein (MBP) stain to identify oligodendrocyte nuclei, and from this the percentage of oligodendrocytes was calculated. EC₅₀ values were calculated using The Levenberg-Marquardt algorithm to fit a Hill equation to dose-response data (0.5 nM to 1000 nM). The results are provided in Table 4 (mOPC EC₅₀).

### 3. Determination of Potency and Enzyme Target

### A. GC/MS-Based Sterol Profiling

Sterols were monitored using a modified Folch wash protocol (Hubler et al, 2018, Nature). EpiSC-derived OPCs were plated at 100,000 cells per well in PO— and laminin-coated 96-well plates in N2B27 media without growth factors. After 24 hours, cells were rinsed with saline and plates were frozen. Cholesterol-d7 standard was then added to each well before drying under nitrogen stream and derivatization with 55 μl of bis(trimethylsilyl) trifluoroacetamide. After derivatization, 2 μl were analyzed by gas chromatography/mass spectrometry using an Agilent 5973 Network Mass Selective Detector equipped with a 6890 gas chromatograph system and a HP-5MS capillary column (30 m x 0.25 mm×0.25 mm). Samples were analyzed in full scan mode using electron impact ionization; ion fragment peaks were integrated to calculate sterol abundance, and quantitation was relative to cholesterol-d7. The following ion fragments were used to quantitate each metabolite: cholesterol-d7 (465), FF-Mas (482), cholesterol (368), zymostenol (458), zymosterol (456), Desmosterol (456, 343), 7-dehydrocholesterol (456, 325), lanosterol (393), lathosterol (458), 14-dehydrozymostenol (456, 351). For reference, Table 2 shows sterol GC-MS analytes and their relationship with inhibitors of cholesterol biosynthesis. All standards were obtained from Avanti Polar Lipids unless otherwise indicated. Calibration curves were generated by injecting varying concentrations of sterol standards and maintaining a fixed amount of cholesterol-D7. For normalized zymostenol accumulation results, the total amount of zymostenol measured after drug treatment was divided by the total amount of zymostenol accumulated after 24 hr treatment with 100 nM positive control reference. EC₅₀ values were calculated using The Levenberg-Marquardt algorithm to fit a Hill equation to dose-response data (8 doses from 0.15 nM to 333 nM). EC₅₀ values for zymostenol (mZymo GCMS EC₅₀) are provided in Table 4.

TABLE 2

| Analyte increased | Cholesterol enzyme inhibited |
|---|---|
| Lanosterol | CYP51 |
| FF-MAS | TM7SF2 (Sterol 14 Reductase) |
| 14-dehydrozymostenol | TM7SF2 (Sterol 14 Reductase) |
| Zymosterol | EBP |
| Zymostenol | EBP |
| 7-dehydrocholesterol | DHCR7 |

### 4. Determination of Binding Affinity to EBP

Membrane preparation: To examine compound binding affinity to EBP, human EBP was overexpressed in human embryonic kidney 293 cells. Cell pellet was lysed in 10 times weight binding buffer (50 mM Tris, 5 mM MgCl₂, 0.1 mM EDTA, 1× protease inhibitor cocktail, pH 7.5) on ice by using a dounce homogenizer. The solution was centrifuged at 25,000 g for 50 min at 4° C. The membrane pellet was re-suspended in binding buffer and run through a 25 5/8 gauge needle. After checking the concentration by Bradford assay, the whole cell membrane solution was adjusted to 20 mg/mL and stored at −80° C.

Determination of equilibrium dissociation constant Kd of radioligand: Membrane prepared as described above was pre-incubated with PVT-WGA SPA beads (Perkinelmer Cat #RPNQ0003) at a ratio of 0.3 mg beads with 5 μg membrane per 25 μL binding buffer at 20° C. for 2 hours with gentle shaking. This binding solution was centrifuged at 400 g for 5 minutes to collect the bead/membrane mixture. After re-suspending the pellet in binding buffer at the same calculated volume with 0.01% BSA (Sigma A1933), the bead/membrane mixture was added in 384-well low-binding surface plate (PerkinElmer Cat #6057480) at 25 i/well. Radioligand at different concentrations with and without the non-radio-labeled same ligand 5 uM (for nonspecific and total signal, respectively) was added to bring final volume to 50 i/well with DMSO concentration at 0.1%. At equilibrium (3 hours after ligand addition), radiometric signal CPM was counted by using a Microbeta2 microplate counter (Perkinelmer). The Kd was determined by nonlinear regression fitting of specific signal plot against the concentration of radioligand [3H]-Ifenprodil (Table 3).

TABLE 3

| Target | Radioligand | Kd | [L] |
|---|---|---|---|
| EBP | [3H]-Ifenprodil, | 15.86 nM | 10 nM |
| | (Perkinelmer Cat# NET1089250UC) | | |

[L], concentration of radioligand used in assay

Competition binding assay to determine compound affinity: The same conditions of the radioligand Kd study were used for compound single dose percentage inhibition and equilibrium dissociation constant Ki examinations, except 50 nL compound DMSO stock was pre-added in 384-well low-binding surface plate (PerkinElmer Cat #6057480) by Echo 550 (Labcyte) to reach the final concentration for single dose test at 1 uM, and dose response test from 0.06 nM to 5 uM (8 dose, 5 times dilution). A pre-incubated bead/membrane mixture was added in compound plate at 0.3 mg beads and 5 g membrane per well. Radioligand [3H]-Ifenprodil was added to reach optimized concentration [L] and bring assay volume to 50 μl. At equilibrium (3 hours after ligand addition), radiometric signal was counted as described above. The percentage inhibition of compound at each testing concentration was calculated by normalizing each condition's CPM readout to full block (5 uM non-radiolabeled ligand) and non-block (DMSO) control conditions. Compound binding inhibition IC₅₀ was determined by nonlinear regression fitting of percentage inhibition plot against compound concentration. Compound Ki was calculated from the equation Ki=IC₅₀/(1+[L]/Kd), which [L] was radioligand concentration used in assay. All tests had N bigger or equal to 2. The data from this experiment is shown in Table 4 (hEBP SPA Ki).

### 5. Determination of Binding Affinity to EBP-7-Dehydrocholesterol Reductase

### Materials and Instruments

| Materials | Vendor | Cat# |
|---|---|---|
| Compound 118A*, [3H] | Moravek | MT-1003106 |
| Ifenprodil (+)-tartrate salt | Sigma | I2892 |
| Bovine Serum Albumin (BSA) | Sigma | A1933 |
| Ethylenediaminetetraacetic acid tetrasodium | Sigma | E5391 |
| salt hydrate (EDTA) | | |
| Magnesium Chloride Hexahydrate (MgCl2•6H2O) | Sigma | M2670 |
| Tris(hydroxymethyl)aminomethane (Tris) | Alfa Aesar | A18494 |
| Polyethylenimine, branched (PEI) | Sigma | 408727 |

### Key Instruments and Consumables

| Item | Supplier | Cat# |
|---|---|---|
| MicroBeta2 Microplate Counter | PerkinElmer | 2450-0060 |
| UniFilter-96 GF/B | PerkinElmer | 6005177 |
| TopSeal | Biotss | SF-800 |
| MicroBeta Filtermate-96 | PerkinElmer | D961962 |
| Seven Compact pH meter | Mettler Toledo | S220 |
| Ultrapure Water Meter | Sichuan Ulupure | UPH-III-20T |
| Benchtop Centrifuge | Hunan Xiangyi | L550 |
| Microplate Shaker | Allsheng | MX100-4A |
| 384-Well Polypropylene Microplate | Labcyte | PP-0200 |
| 96-Well U bottom Plate | Corning | 7007 |
| Echo | LABCYTE | 550 |

### Assay Buffer Preparation

| Reagent | Concentration |
|---|---|
| Tris | 50 mM |
| MgCl₂ | 5 mM |
| EDTA | 0.1 mM |
| BSA | 0.01% (w/v) |

Adjust pH to 7.4 followed by 0.2 μM sterile filtration

Membrane Preparation: human emopamil binding protein and human 7-dehydrocholesterol reductase co-expressing cells were generated by transient transfecting host human embryonic kidney (HEK) 293 cells with 2 DNA constructs containing each protein's coding sequence. Cells were suspension cultured at 37° C. with 5% CO₂ in FREESTYLE 293 Expression Medium (Thermofisher). Whole cell membrane was prepared by harvesting the cell pellet, adding cold membrane buffer (50 mM Tris, pH7.5, 1× Roche COMPLETE EDTA-free protease inhibitor cocktail) 10 times volume of the cell pellets weight, lysing cell pellet on ice by using Dounce homogenizer, spinning at 200 g 4° C. for 15 min, collecting supernatant and spinning again at 25000 g 4° C. for 50 min, transferring pellet to Dounce homogenizer, re-suspending pellet by homogenizing in membrane buffer on ice to reach ˜25 mg/mL, then keeping whole cell membrane aliquots at −80° C.

Compounds were prepared in a 96-well U bottom plate using an Echo550 machine and 10 mM compound DMSO stock solution, followed by an 8-dose 5-fold serial dilutions protocol with final testing compound concentration ranging from 0.06 to 5000 nM, with DMSO back fill to 100 nL/well and n=2. DMSO and Ifenprodil 5 uM wells were added in each plate as 0 and 100% inhibition reference controls with n=8 for each condition. The UniFilter-96 GF/B plates were pre-treated by adding 50 μl/well of 0.3% (v/v) PEI to UniFilter-96 GF/B plates. The plates were sealed and incubated at 4° C. for 3 hrs. Then, the plates were washed with ice-cold assay buffer 3 times. The radioligand binding assay was prepared by adding assay buffer diluted hEBP-DHCR7 membrane at 66.7 μg/ml x 150 μl/well into the 96-well compound plate to reach 10 μg membrane per well. Then, the assay buffer diluted [3H]-Compound 118A* was added at 25 nM×50 μl/well. Following this, the plate was centrifuged at 1000 rpm for 30 secs. The plate was then sealed and agitated at 600 rpm at 22° C. for 5 min, and then incubated at 22° C. for 3 hrs. The incubation was stopped by transferring the binding solution to the pre-treated UniFilter-96 GF/B plate, vacuum filtrated, and then washed four times with ice-cold assay buffer. Following this, the plates were dried at 37° C. for 45 min. The plates were then sealed at the bottom. 40 μl/well of scintillation cocktail was added to the plates. A MicroBeta2 microplate counter was then used to read the plate and analyze the data. For reference and test compounds, the results are expressed as % Inhibition, using the normalization equation: N=100-100×(U−C2)/(C1−C2), where U is the unknown value, C1 is the average of high controls, and C2 is the average value of low controls. The IC50 was determined by fitting percentage of inhibition as a function of compound concentrations with Hill equation using XLfit. Results are expressed as hEBP-DHCR7 Ki (uM) in Table 4. Ki was calculated as described above.

TABLE 4

| | hEBP | hEBP- | mZymo | |
|---|---|---|---|---|
| | SPA | DHCR7 | GCMS | mOPC EC₅₀ |
| No. | Ki (uM) | Ki (uM) | EC₅₀ (uM) | (uM) |
| 102 | ND | 0.00831 | 0.0025 | 0.020 |
| 104 | ND | ND | 0.00048 | 0.0017 |
| 105 | ND | 0.0129 | 0.066 | 0.26 |
| 106 | ND | 0.00838 | 0.0085 | 0.047 |
| 107 | 0.023 | 0.0103 | 0.0095 | 0.022 |
| 108 | ND | 0.00972 | 0.0055 | 0.0075 |
| 109A | 0.032 | 0.0125 | 0.0016 | 0.0018 |
| 109B* | ND | 0.00654 | 0.00040 | 0.00055 |
| 109C* | ND | 0.00514 | 0.0021 | 0.0030 |
| 110 | ND | 0.00844 | 0.022 | 0.030 |
| 111A | ND | 0.00857 | 0.0013 | 0.020 |
| 111B* | ND | ND | 0.0014 | 0.0015 |
| 111C* | ND | ND | 0.0071 | 0.0061 |
| 112 | 0.028 | 0.0135 | 0.0013 | 0.033 |
| 113 | ND | 0.00945 | 0.00041 | 0.018 |
| 114A* | 0.035 | 0.0183 | 0.00037 | 0.0024 |
| 114B* | 0.034 | 0.00585 | 0.00082 | 0.00072 |
| 115A* | ND | 0.0189 | 0.0086 | 0.042 |
| 115B* | ND | 0.0135 | 0.015 | 0.029 |
| 116A* | ND | 0.0105 | 0.00049 | 0.0016 |
| 116B* | ND | 0.00931 | 0.00081 | 0.0020 |
| 117A* | ND | 0.00943 | ND | 0.0015 |
| 117B* | ND | 0.00986 | 0.00035 | 0.00046 |
| 118A* | 0.026 | 0.0221 | 0.0024 | 0.0085 |
| 118B* | ND | 0.0505 | 0.054 | 0.14 |
| 119A* | ND | 0.00727 | 0.00052 | 0.00072 |
| 119B* | ND | 0.0105 | 0.0040 | 0.0022 |
| 120 | ND | 0.0177 | 0.004 | 0.0064 |
| 121A* | ND | 0.115 | 0.0061 | 0.023 |
| 121B* | ND | 0.14 | 0.15 | 0.26 |
| 122A* | ND | 0.0156 | 0.0015 | 0.010 |
| 122B* | 0.018 | 0.0137 | 0.0012 | 0.0021 |
| 123A* | ND | 0.0119 | 0.014 | 0.054 |
| 123B* | ND | 0.011 | 0.0024 | 0.035 |
| 124A* | ND | 0.0185 | 0.0062 | 0.032 |
| 124B* | ND | 0.0253 | 0.014 | 0.054 |
| 125 | 0.056 | 0.00402 | 0.00062 | 0.00072 |
| 126A* | 0.030 | 0.0474 | 0.0038 | 0.010 |
| 126B* | 0.12 | 0.0676 | 0.014 | 0.23 |
| 127 | ND | 0.0213 | 0.013 | 0.015 |
| 128A* | 0.056 | 0.0296 | 0.023 | 0.37 |
| 128B* | 0.045 | 0.0257 | 0.0048 | ND |
| 129 | 0.021 | 0.0118 | 0.019 | 0.19 |
| 130 | 0.016 | 0.0132 | 0.0029 | 0.010 |
| 131 | 0.031 | 0.118 | 0.0016 | 0.0078 |
| 132 | 0.026 | 0.0147 | 0.0072 | ND |
| 133 | 0.089 | 0.0866 | 0.072 | ND |
| 135 | 0.029 | 0.0396 | 0.032 | 0.14 |
| 136 | 0.029 | 0.0142 | 0.0036 | 0.012 |
| 138 | 0.016 | 0.00504 | 0.0010 | 0.0023 |
| 139 | 0.030 | 0.00923 | 0.0016 | 0.00046 |
| 140 | 0.018 | 0.0199 | 0.0021 | 0.0050 |
| 141A* | 0.044 | 0.0256 | 0.0055 | 0.021 |
| 141B* | 0.061 | 0.0584 | 0.049 | 0.48 |
| 142 | 0.035 | 0.0161 | 0.014 | ND |
| 143 | 0.027 | 0.0126 | 0.0028 | 0.026 |
| 144 | 0.053 | 0.0966 | 0.080 | 0.39 |
| 145A* | 0.056 | 0.0691 | 0.0081 | 0.069 |
| 145B* | 0.10 | 0.106 | 0.055 | ND |
| 148A* | ND | 0.0236 | 0.0049 | 0.031 |
| 148B* | 0.032 | 0.0286 | 0.0072 | 0.073 |
| 149 | ND | 0.0246 | 0.0016 | 0.0063 |
| 150 | ND | 0.0299 | 0.0049 | 0.025 |
| 152 | ND | 0.016 | 0.0033 | 0.011 |
| 153 | ND | 0.00723 | 0.00087 | 0.0016 |
| 154A* | 0.059 | 0.176 | 0.200 | ND |
| 154B* | 0.053 | 0.143 | 0.250 | ND |
| 155 | 0.014 | 0.187 | 0.003 | ND |
| 156 | ND | 0.0139 | 0.0015 | ND |
| 157 | 0.025 | 0.0172 | 0.012 | 0.0094 |
| 158 | ND | 0.010 | 0.002 | 0.0029 |
| 201 | 0.013 | 0.0257 | 0.0032 | 0.0055 |
| 202 | ND | 0.0147 | 0.042 | 0.22 |
| 203* | ND | 0.0519 | 0.0064 | 0.022 |
| 204 | ND | 0.0289 | 0.0023 | 0.0068 |
| 205 | ND | 0.0194 | 0.0056 | 0.024 |
| 206 | ND | 0.0132 | 0.0015 | 0.0032 |
| 207A* | ND | 0.0336 | 0.031 | ND |
| 207B* | ND | 0.0653 | 0.0098 | 0.063 |
| 208 | ND | 0.0128 | 0.00069 | 0.00152 |
| 209 | ND | 0.0139 | 0.0099 | 0.045 |
| 210 | ND | 0.504 | 0.052 | 0.282 |
| 212A* | ND | 0.0156 | 0.00236 | 0.0047 |
| 212B* | ND | 0.0101 | 0.0021 | 0.004 |
| 213 | ND | 0.012 | 0.0050 | 0.0047 |
| 214 | ND | 0.0408 | 0.015 | 0.0504 |
| 215A* | 0.021 | 0.105 | 0.013 | 0.023 |
| 215B* | 0.025 | 0.136 | 0.017 | 0.051 |
| 216A* | ND | 0.00998 | 0.00095 | 0.0014 |
| 216B* | ND | 0.0156 | 0.00099 | 0.0033 |
| 217* | ND | 0.0396 | 0.0083 | 0.026 |
| 218 | ND | 0.00654 | 0.00093 | 0.0025 |
| 219 | ND | 0.0221 | 0.0069 | 0.0222 |
| 220 | ND | 0.0119 | 0.00097 | ND |
| 221A* | 0.01 | 0.0255 | 0.0016 | 0.0049 |
| 221B* | ND | 0.0106 | 0.0046 | 0.013 |
| 222 | ND | 0.0037 | 0.0037 | 0.016 |
| 223 | ND | 0.555 | 0.12 | 0.355 |
| 225 | ND | 0.0243 | 0.0122 | 0.0235 |
| 226 | ND | 0.00929 | 0.00088 | 0.0022 |
| 227A* | ND | 0.0153 | 0.0032 | 0.0076 |
| 227B* | ND | 0.0098 | 0.0021 | 0.0024 |
| 228 | ND | 0.00795 | 0.00094 | 0.0028 |
| 229 | ND | 0.11 | 0.034 | 0.135 |
| 230 | 0.022 | 0.749 | 0.011 | 0.022 |
| 231 | ND | 0.00731 | 0.00032 | 0.00090 |
| 232A* | ND | 0.0139 | 0.0020 | 0.0056 |
| 232B* | ND | 0.0127 | 0.0057 | 0.015 |
| 233 | ND | 0.0132 | 0.0015 | 0.0057 |
| 234A* | ND | 0.25 | 0.049 | ND |
| 234B* | ND | 0.193 | 0.035 | 0.21 |
| 235A* | ND | 0.0197 | 0.0061 | 0.0199 |
| 235B* | ND | 0.0171 | 0.0026 | 0.00389 |
| 236 | ND | 0.017 | 0.00264 | 0.00389 |
| 237A* | ND | 0.0896 | 0.0301 | 0.0269 |
| 237B* | ND | 0.117 | 0.017 | 0.0691 |
| 238 | ND | 0.0141 | 0.00289 | 0.0137 |
| 239 | ND | 0.0419 | 0.0398 | 0.374 |
| 240 | ND | 0.00196 | 0.0755 | 0.346 |
| 241 | ND | 0.069 | 0.0149 | ND |
| 242 | ND | 0.0131 | 0.00451 | 0.0163 |
| 243 | ND | 0.053 | 0.00661 | ND |
| 244 | ND | 0.0107 | 0.0235 | ND |
| 245A* | ND | 0.016 | 0.0105 | ND |
| 245B* | ND | 0.0197 | 0.00837 | ND |
| 246 | ND | 0.0122 | 0.00126 | 0.00262 |
| 247A* | ND | 0.00572 | 0.00316 | 0.0228 |
| 247B* | ND | 0.00897 | 0.0686 | ND |
| 248A | ND | 0.0149 | 0.0244 | 0.0948 |
| 248B | ND | 0.0316 | 0.0256 | 0.0741 |
| 249 | ND | 0.0316 | 0.0256 | 0.0741 |

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A compound of Formula I: 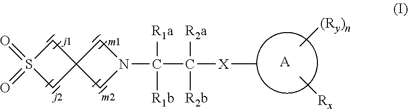
or a pharmaceutically acceptable salt thereof, wherein,
j₁, j₂, m₁, and m₂ are each independently 1 or 2, wherein the sum of j₁ and j₂ and the sum of m₁ and m₂ are each no more than 4, and the total sum of j₁, j₂, m₁, and m₂ is no more than 6;
R₁ₐ, R_{1b}, R₂ₐ, and R_{2b} are each independently selected from the group consisting of hydrogen, halogen, C₁-C₆ alkyl, halo-C₁-C₆ alkyl, halo-C₁-C₆ alkoxy, and C₁-C₆ alkoxy;
X is O or C(R₁₀)(R₂₀), wherein R₁₀ and R₂₀ are each independently selected from the group consisting of hydrogen, halogen, C₁-C₆ alkyl, halo-C₁-C₆ alkyl, halo-C₁-C₆ alkoxy, and C₁-C₆ alkoxy;
or,
R₁ₐ and R_{1b}, R₂ₐ and R_{2b}, or R₁₀ and R₂₀, taken together with the atom to which they are attached form a C₃-C₅ cycloalkyl, optionally substituted with halogen, hydroxy, halo-C₁-C₆ alkoxy, C₁-C₆ alkoxy, —SO₂(C₁-C₆ alkyl), —CN, or —NRR′;
Ring A is phenyl, or 5- to 6-membered heteroaryl comprising one, two, or three heteroatoms independently selected from the group consisting of O, N, and S;
each R_{y} is independently halogen, C₁-C₆ alkyl, halo-C₁-C₆ alkyl, hydroxy, C₁-C₆ alkoxy, halo-C₁-C₆ alkoxy, C₃-C₇ cycloalkyl, C₃-C₇halocycloalkyl, —SO₂(C₁-C₆ alkyl), —CN, or —NRR′;
n is 0, 1, 2, 3, or 4;
Rₓ is halogen, hydroxy, C₁-C₁₀ alkyl, halo-C₁-C₆ alkyl, C₁-C₆ alkoxy, halo-C₁-C₆ alkoxy, 5- to 7-membered heterocyclyl, C₃-C₇ cycloalkyl, C₆-C₁₀ aryl, 5- to 6-membered heteroaryl, —SO₂(C₁-C₆ alkyl), —CN, or —NRR′, wherein said heterocyclyl, cycloalkyl, aryl, or heteroaryl is optionally substituted with one or more substituents independently selected from the 2 of 18 group consisting of halogen, C₁-C₆ alkyl, hydroxy, C₁-C₆ alkoxy, halo-C₁-C₆ alkoxy, halo-C₁-C₆ alkyl, —SO₂(C₁-C₆ alkyl), —CN, and —NR″R′″; and
R, R′, R″, and R′″ are each independently hydrogen, C₁-C₆ alkyl, or halo-C₁-C₆ alkyl.

2. The compound of claim 1, or a pharmaceutically acceptable salt thereof, wherein one of m₁ and m₂ is 1 and the other is 2.

3. (canceled)

4. The compound of claim 1, or a pharmaceutically acceptable salt thereof, wherein m₁ and m₂ are each 2.

5. (canceled)

6. The compound of claim 1, or a pharmaceutically acceptable salt thereof, wherein j₁ and j₂ are each 1.

7. (canceled)

8. (canceled)

9. The compound of claim 1, wherein the compound is of Formula Ia: 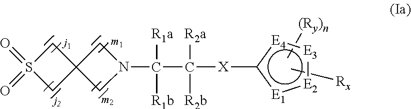
or a pharmaceutically acceptable salt thereof, wherein E₁, E₂, E₃, and E₄ are each independently selected from the group consisting of CH, C—Rₓ, C—R_{y}, N—Rₓ, N—R_{y}, N, NH, O, and S, wherein one, two, or three of E₁, E₂, E₃, and E₄ are N, NH, N—Rₓ, N—R_{y}, O, or S.

10. The compound of claim 9, or a pharmaceutically acceptable salt thereof, wherein E₁ is CH; E₂ is N; E₃ is N—Rₓ; and E₄ is CH; or
E₁ is CH; E₂ is C—Rₓ; E₃ is N; and E₄ is N—R_{y}.

11. (canceled)

12. The compound of claim 1, wherein the compound is of Formula Ib: 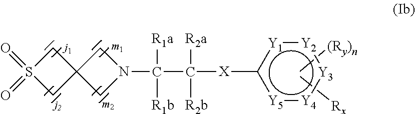
or a pharmaceutically acceptable salt thereof, wherein Y₁, Y₂, Y₃, Y₄, and Y₅ are each independently selected from the group consisting of CH, C—R_{y}, C—Rₓ, N, or S, wherein no more than 3 of Y₁, Y₂, Y₃, Y₄, and Y₅ are N, or S.

13. The compound of claim 12, or a pharmaceutically acceptable salt thereof, wherein:
(a) Y₁, Y₂, Y₄, and Y₅ are each CH; and Y₃ is C—Rₓ,
(b) Y₁, Y₂, and Y₅ are each CH; Y₄ is N; and Y₃ is C—Rₓ;
(c) Y₁, Y₂, and Y₅ are each CH; Y₄ is C—R_{y}; and Y₃ is C—Rₓ;
(d) Y₁, Y₂, and Y₅ are each CH; Y₄ is C—Rₓ; and Y₃ is N;
(e) Y₂ and Y₄ are each N; Y₁ and Y₅ are each CH; and Y₃ is C—Rₓ;
(f) Y₅ is N; Y₃ is C—Rₓ; and Y₁, Y₂, and Y₄ are each CH;
(g) Y₄ is C—Rₓ; Y₂ is N; and Y₁, Y₃, and Y₅ are each CH;
(h) Y₁, Y₂, Y₃, and Y₄, are each CH; and Y₅ is C—Rₓ;
(i) Y₁ and Y₅ are CH; Y₄ is C—R_{y}; Y₃ is C—Rₓ; and Y₂ is N; or
(j) Y₁ is CH; Y₅ is C—R_{y}; Y₄ is N; Y₃ is C—Rₓ; and Y₂ is CH.

14.-23. (canceled)

24. The compound of claim 1, or a pharmaceutically acceptable salt thereof, wherein R_{y} is chloro, trifluoromethyl, fluoro, methyl, or isopropyl.

25. (canceled)

26. The compound of claim 1, or a pharmaceutically acceptable salt thereof, wherein Rₓ is fluoro, trifluoromethyl, trifluoroethyl, difluoromethyl, fluoromethyl, or trifluoromethoxy.

27.-28. (canceled)

29. The compound of claim 1, or a pharmaceutically acceptable salt thereof, wherein Rₓ is C₃-C₇ cycloalkyl optionally substituted with one or more substituents independently selected from the group consisting of halogen, C₁-C₆ alkyl, hydroxy, C₁-C₆ alkoxy, halo-C₁-C₆ alkoxy, and halo-C₁-C₆ alkyl.

30. The compound of claim 29, or a pharmaceutically acceptable salt thereof, wherein Rₓ is selected from the group consisting of cyclopropyl, cyclobutyl, cyclopentyl, and cyclohexyl, optionally substituted with one or two substituents, each independently selected from the group consisting of fluoro, methyl, hydroxy, and trifluoromethyl.

31. (canceled)

32. The compound of claim 30, or a pharmaceutically acceptable salt thereof, wherein Rₓ is unsubstituted cyclohexyl, unsubstituted cyclopropyl, unsubstituted cyclobutyl,

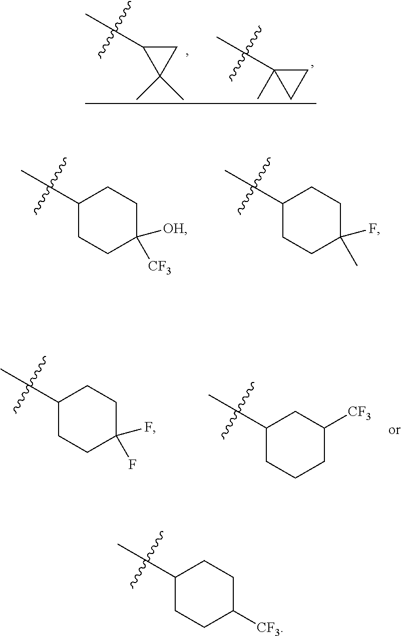

33.-44. (canceled)

45. The compound of claim 1, or a pharmaceutically acceptable salt thereof, wherein X is O.

46. The compound of claim 1, or a pharmaceutically acceptable salt thereof, wherein X is C(R₁₀)(R₂₀), wherein R₁₀ and R₂₀ are each independently hydrogen.

47. (canceled)

48. (canceled)

49. The compound of claim 1, or a pharmaceutically acceptable salt thereof, wherein R₂ₐ and R_{2b} are each independently methoxy, fluoro, hydrogen or methyl.

50. The compound of claim 49, or a pharmaceutically acceptable salt thereof, wherein R₂ₐ is hydrogen and R_{2b} is methyl.

51. The compound of claim 49, or a pharmaceutically acceptable salt thereof, wherein R₂ₐ and R_{2b} are each methyl.

52. (canceled)

53. (canceled)

54. The compound of claim 1, or a pharmaceutically acceptable salt thereof, wherein R₁ₐ and R_{1b} are each hydrogen.

55.-58. (canceled)

59. The compound of claim 12, or a pharmaceutically acceptable salt thereof, wherein the compound is of Formula Ib, wherein:
four of Y₁, Y₂, Y₃, Y₄, and Y₅ are CH, and one is C—Rₓ;
Rₓ is selected from the group consisting of halo-C₁-C₆ alkyl, halogen, halo-C₁-C₆ alkoxy, C₁-C₁₀ alkyl, pyridinyl, and C₃-C₇ cycloalkyl,
wherein said C₃-C₇ cycloalkyl or pyridinyl is optionally substituted one or more times with C₁-C₆ alkyl or halo-C₁-C₆ alkyl;
n is 0;
X is C(R₁₀)(R₂₀) or O, wherein R₁₀ and R₂₀ are each independently hydrogen, methyl, or fluoro;
R₁ₐ and R_{1b} are each independently hydrogen or methyl; and
R₂ₐ and R_{2b} are each independently hydrogen, fluoro, methoxy, or methyl; or, R₂ₐ and R_{2b}, together with the carbon atom to which they are attached, form a C₃-C₅ cycloalkyl.

60.-74. (canceled)

75. The compound of claim 1, wherein the compound is selected from the group consisting of: 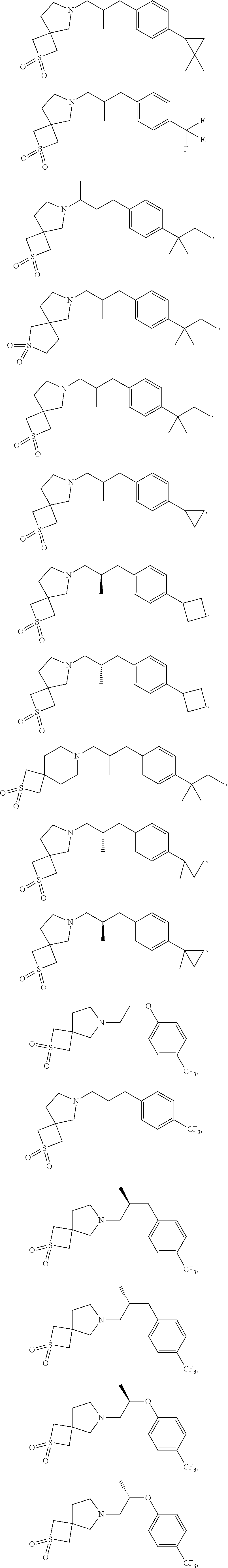 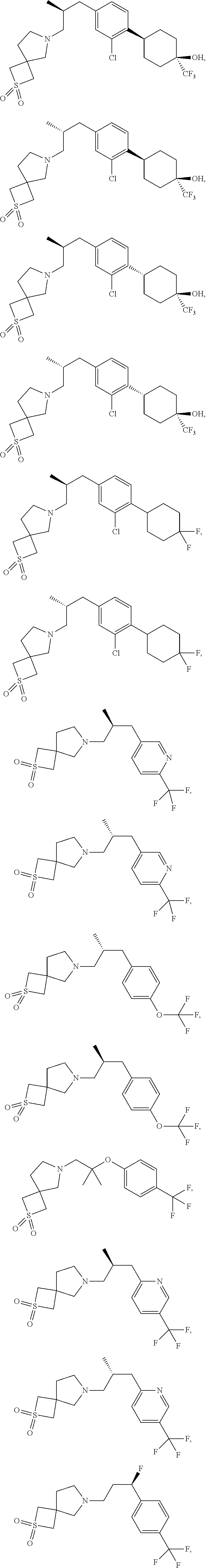 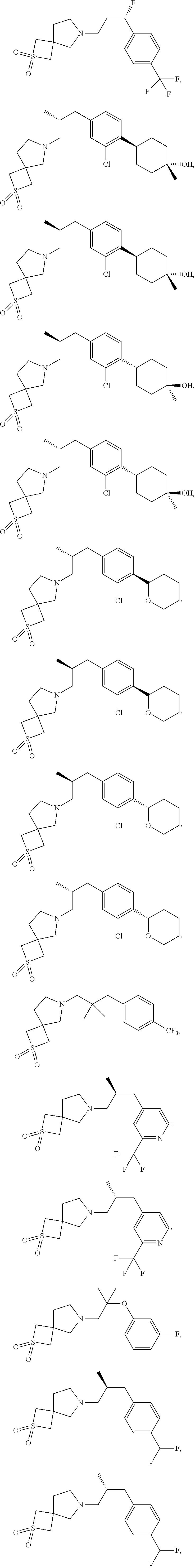 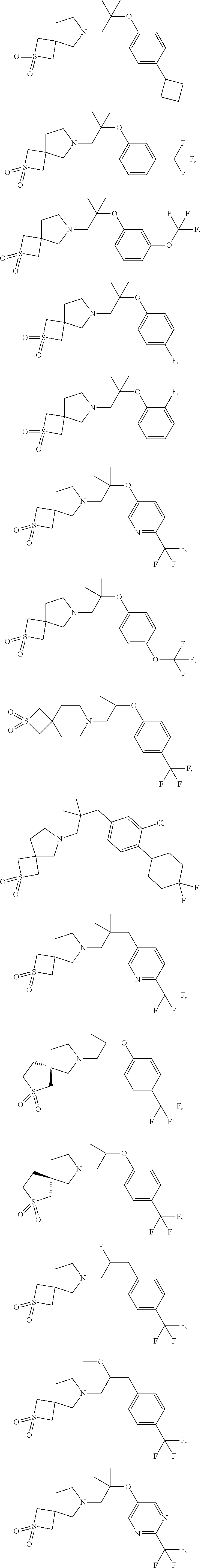 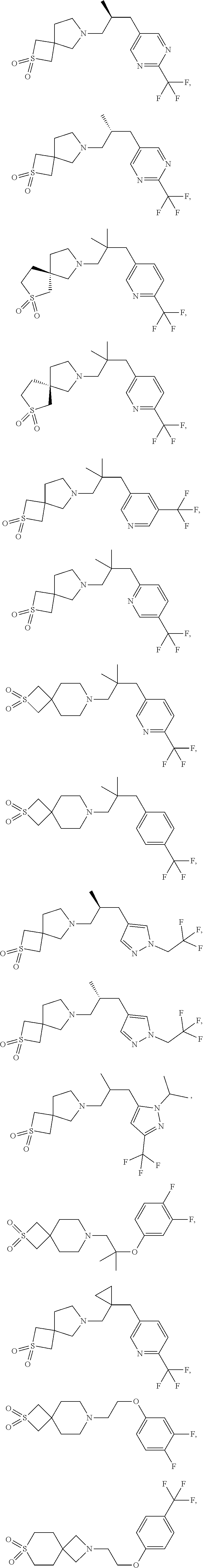 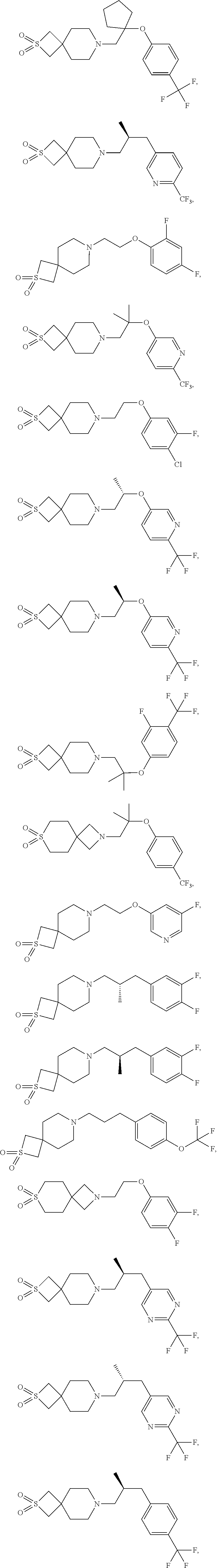 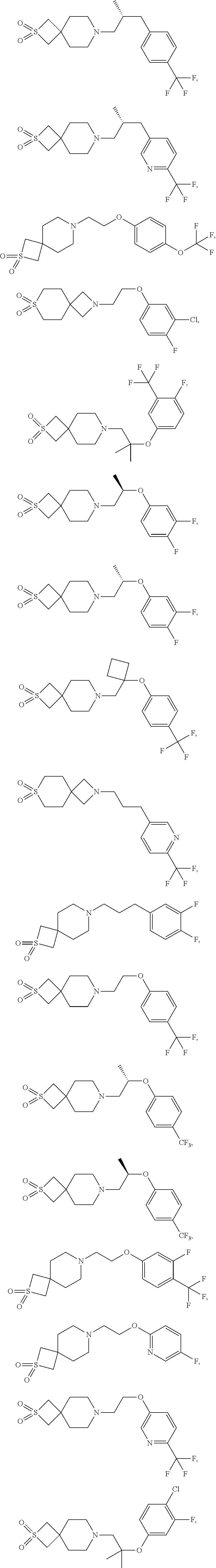 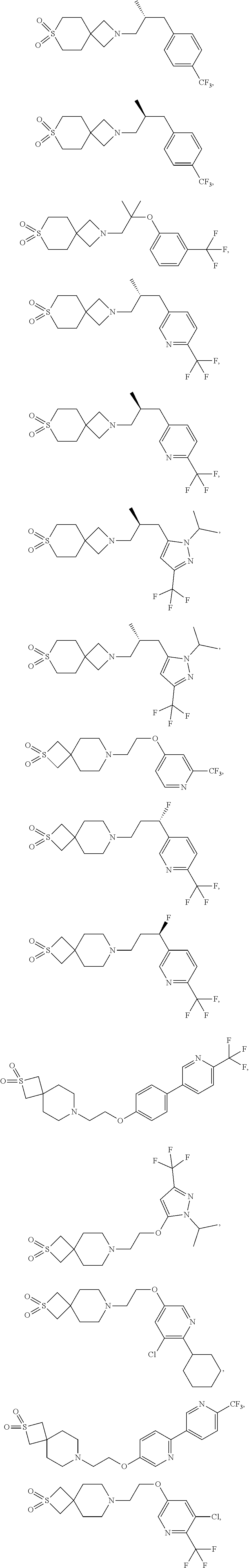 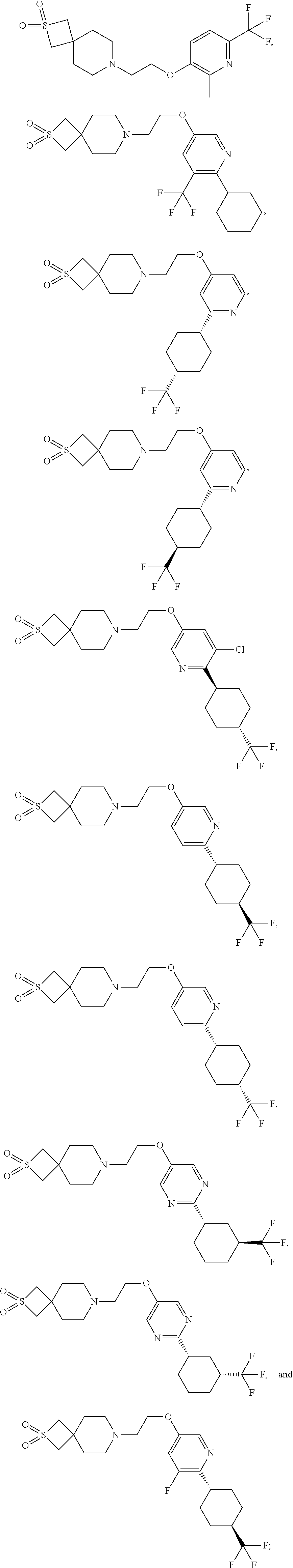
or a pharmaceutically acceptable salt thereof.

76. A pharmaceutical composition comprising a compound claim 1 or a pharmaceutically acceptable salt thereof, and a pharmaceutically acceptable excipient.

77. A method of treating a disorder in a subject in need thereof, the method comprising administering to the subject in need thereof a therapeutically effective amount of a compound of claim 1, or a pharmaceutically acceptable salt thereof.

78.-79. (canceled)

80. A method of promoting myelination in a subject in need thereof, comprising administering to the subject a therapeutically effective amount of a compound of claim 1, or a pharmaceutically acceptable salt thereof.

81.-86. (canceled)
